(19) United States
(12) Patent Application Publication
(10) Pub. No.: US 2024/0374571 A1
(43) Pub. Date: Nov. 14, 2024

(54) PHARMACEUTICAL COMBINATIONS COMPRISING A TEAD INHIBITOR AND USES THEREOF FOR THE TREATMENT OF CANCERS

(71) Applicant: Novartis AG, Basel (CH)

(72) Inventors: Emilie CHAPEAU, Huningue (FR); Laurent L'EPICIER-SANSREGRET, Basel (CH); Tobias SCHMELZLE, Riehen (CH)

(21) Appl. No.: 18/687,616

(22) PCT Filed: Aug. 30, 2022
(86) PCT No.: PCT/IB2022/058104

§ 371 Date: Feb. 28, 2024

Related U.S. Application Data
(60) Provisional application No. 63/366,829, filed on Jun. 22, 2022.
(60) Provisional application No. 63/268,481, filed on Feb. 24, 2022.
(60) Provisional application No. 63/239,512, filed on Sep. 1, 2021.

(51) Int. Cl. A61K 31/416; A61K 31/4439; A61K 31/497; A61K 31/506; A61K 31/519; A61K 31/5377; A61K 31/55; A61P 35/00

(52) U.S. Cl. CPC A61K 31/416; A61K 31/4439; A61K 31/497; A61K 31/506; A61K 31/519; A61K 31/5377; A61K 31/55; A61P 35/00

(57) ABSTRACT

The invention relates to a pharmaceutical combination comprising a TEAD inhibitor in combination with a first and optionally a second therapeutically active agent. The present invention also relates to methods of treating cancer involving administering to a subject in need thereof the TEAD inhibitor in combination with the first and optionally the second therapeutically active agent.

### FIELD OF THE DISCLOSURE

The present invention relates to a pharmaceutical combination comprising a TEAD inhibitor in combination with a first and optionally a second therapeutically active agent. The present invention also relates to methods of treating cancer involving administering to a subject in need thereof the TEAD inhibitor in combination with the first and optionally the second therapeutically active agent.

### BACKGROUND

The advent of targeted therapies for cancer has increased patient lifespan for various malignancies and helped to appreciate the complexity of tumors through the study of drug resistance mechanisms. The fact that clinical responses to targeted agents are generally incomplete and/or transient results from a multitude of factors that can be broadly put into two classes: toxicities that prevent optimal dosing of drugs and consequently limit target engagement (Brana and Siu 2012, Chapman, Solit et al. 2014), and the ability of cancers to adapt and maintain their proliferative potential against perturbations (Druker 2008, Chandarlapaty 2012, Doebele, Pilling et al. 2012, Duncan, Whittle et al. 2012, Katayama, Shaw et al. 2012, Lito, Rosen et al. 2013, Sullivan and Flaherty 2013, Solit and Rosen 2014). Combinations of drugs can address both these factors by improving overall efficacies and at the same time targeting tumor robustness and complexity to counter resistance (Robert, Karaszewska et al. 2015, Turner, Ro et al. 2015). It is not yet clear how many drugs are required and which processes need to be targeted in combination to overcome specific types of cancer. But it is almost certain that different pathways or drivers need to be inhibited, most likely requiring two or more drugs (Bozic, Reiter et al. 2013). In spite of numerous treatment options for patients with specific types of cancer, there remains a need for effective and safe combination therapies that can be administered for the treatment of cancer.

### SUMMARY

It is an object of the present invention to provide for a medicament to improve treatment of a cancer, in particular to improve treatment of cancer through inhibition of cell growth (proliferation) and/or induction of apoptosis (cell death). It is an object of the present invention to find novel combination therapies, which selectively synergize the inhibition of proliferation and/or the induction of apoptosis.

Surprisingly, it has been found that a pharmaceutical combination comprising i) a TEAD inhibitor, ii) a first additional therapeutically active agent, and optionally iii) a second additional therapeutically active agent wherein the first additional therapeutically active agent and the second additional therapeutically active agent (where present) are independently selected from the group consisting of a KRAS G12/G13 inhibitor, a SHP2 inhibitor, an EGFR inhibitor, a PI3K inhibitor, a MEK inhibitor, an ERK inhibitor, an MDM2 inhibitor, a Raf-inhibitor, a CDK4/6 inhibitor and a cMET inhibitor, can both synergistically inhibit proliferation and/or induce apoptosis in cancers, as demonstrated in the Examples.

Therefore, according to a first aspect of the invention, there is hereby provided a method of treating cancer in a subject in need thereof, wherein the method comprises administering to the subject a therapeutically effective amount of a TEAD inhibitor in combination with a first additional therapeutically active agent, and optionally a second additional therapeutically active agent, wherein the first additional therapeutically active agent and the second additional therapeutically active agent (where present) are independently selected from the group consisting of a KRAS G12/G13 inhibitor, a SHP2 inhibitor, an EGFR inhibitor, a PI3K inhibitor, a MEK inhibitor, an ERK inhibitor, an MDM2 inhibitor, a Raf-inhibitor, a CDK4/6 inhibitor and a cMET inhibitor.

According to a second aspect of the invention, there is hereby provided a TEAD inhibitor for use in the treatment of cancer, wherein the treatment further comprises administration of a first additional therapeutically active agent, and optionally a second therapeutically active agent, wherein the first additional therapeutically active agent and the second additional therapeutically active agent (where present) are independently selected from the group consisting of a KRAS G12/G13 inhibitor, a SHP2 inhibitor, an EGFR inhibitor, a PI3K inhibitor, a MEK inhibitor, an ERK inhibitor, an MDM2 inhibitor, a Raf-inhibitor, a CDK4/6 inhibitor and a cMET inhibitor.

According to a third aspect of the invention, there is hereby provided a pharmaceutical combination comprising i) a TEAD inhibitor, ii) a first additional therapeutically active agent, and optionally iii) a second additional therapeutically active agent, wherein the first additional therapeutically active agent and the second additional therapeutically active agent (where present) are independently selected from the group consisting of a KRAS G12/G13 inhibitor, a SHP2 inhibitor, an EGFR inhibitor, a PI3K inhibitor, a MEK inhibitor, an ERK inhibitor, an MDM2 inhibitor, a Raf-inhibitor, a CDK4/6 inhibitor and a cMET inhibitor.

According to a fourth aspect of the invention, there is hereby provided a cMET inhibitor for use in the treatment of cancer, wherein the treatment further comprises administration of a TEAD inhibitor.

According to a fifth aspect of the invention, there is hereby provided a SHP2 inhibitor for use in the treatment of cancer, wherein the treatment further comprises administration of a TEAD inhibitor.

According to a sixth aspect of the invention, there is hereby provided a MEK inhibitor for use in the treatment of cancer, wherein the treatment further comprises administration of a TEAD inhibitor.

According to a seventh aspect of the invention, there is hereby provided an ERK inhibitor for use in the treatment of cancer, wherein the treatment further comprises administration of a TEAD inhibitor.

According to an eighth aspect of the invention, there is hereby provided a Raf inhibitor for use in the treatment of cancer, wherein the treatment further comprises administration of a TEAD inhibitor.

According to a ninth aspect of the invention, there is hereby provided an EGFR inhibitor for use in the treatment of cancer, wherein the treatment further comprises administration of a TEAD inhibitor.

According to a tenth aspect of the invention, there is hereby provided a PI3K inhibitor for use in the treatment of cancer, wherein the treatment further comprises administration of a TEAD inhibitor.

According to an eleventh aspect of the invention, there is hereby provided an MDM2 inhibitor for use in the treatment of cancer, wherein the treatment further comprises administration of a TEAD inhibitor.

According to a twelfth aspect of the invention, there is hereby provided a CDK4/6 inhibitor for use in the treatment of cancer, wherein the treatment further comprises administration of a TEAD inhibitor.

### BRIEF DESCRIPTION OF THE DRAWINGS

FIG. 1: Female nude mice bearing H2122 or 2094-HX subcutaneous xenograft lung tumors were treated p.o. daily (QD) with Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide), Compound C (1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one), AMG510 (also known as sotorasib), or in combinations as indicated in the legends, or vehicle control. Values are mean±SEM (standard error of the mean); sample size n=4-7 mice per group. Tumor volumes are represented as a percent of change from the measure at day 0 of treatment. Left panel, efficacy results with the H2122 xenograft model. Diamond indicates results with a combination of Compound A (220 mg/kg qd) and Compound C (100 mg/kg qd). Right panel, efficacy results with the 2094-HX Patient-Derived-Xenograft (PDX) human lung model. The treatments were stopped at day 44 of the study, and tumor regrowth was observed off treatment for animals treated with the single agents compared with animals treated with the combinations.

FIG. 2: Female nude mice bearing 9 different colorectal cancer (CRC) subcutaneous PDX tumors were treated p.o. with agents as indicated in legends. A mouse clinical trial (MCT) format was used with n=2 for untreated, n=1 for each treatment. Left panel: the reduction of tumor growth is shown using the not doubled in volume parameter, expressed as a percentage of values for all mice. Right panel: Values are mean±SEM; the percent of the average of best tumor volume (TV) change versus baseline is shown.

FIG. 3: Female nude mice bearing Lu99 lung or ACC-MESO1 mesothelioma subcutaneous xenograft tumors were treated p.o. daily (QD) with Compound A or Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide), twice daily with TNO155, or in combinations as indicated in the legends, or vehicle control. Values are mean f SEM; sample size n=5-9 mice per group. Tumor volumes are shown in the top graphs. The percent of body weight change versus baseline is shown in bottom graphs.

FIG. 4: Female nude mice bearing Lu99 lung subcutaneous xenograft tumors were treated p.o. with single agents or combinations of agents as indicated in legends. Treatments were administrated daily (QD) for Compound A or Compound B, daily for Compound C, and twice daily for TNO155. Values are mean±SEM; sample size n=6 mice per group. Tumor volumes are shown in the left graphs. The percent of body weight change versus baseline is shown in right graphs.

FIG. 5: Female nude mice bearing Lu99 or 2094-HX lung subcutaneous xenograft tumors were treated p.o. with single agents or combinations of agents as indicated in legends. Values are mean±SEM; sample size n=4-6 mice per group. Tumor volumes are shown in the left graphs. The percent of body weight change versus baseline is shown in right graphs.

FIG. 6: Female nude mice bearing subcutaneous xenograft tumors were treated p.o. with single agents or combinations of agents as indicated in legends. A) Anti-tumor efficacy and tolerability evaluation on the NCI-H2052 mesothelioma subcutaneous xenograft model. Values are mean f SEM; sample size n=6 mice per group. B) Anti-tumor efficacy and tolerability evaluation on the 2094-HX lung subcutaneous xenograft model. Values are mean±SEM; sample size n=5-6 mice per group. C) A MCT was performed at with 23 PDAC PDX models was used with n=1-2 per model and treatment. The reduction of tumor growth is shown using the not doubled in volume parameter, expressed as a percentage of the mouse population.

FIG. 7: Female nude mice bearing HT-29 BRAF mutant on V600E CRC subcutaneous xenograft tumors were treated p.o. with single agents or combinations of agents as indicated in legends. Compound A was used at 100 mg/kg daily in the two first weeks of treatment and at 200 mg/kg in the two last weeks of treatments in both single agent and triple combination arms. Values are mean±SEM; sample size n=5 mice per group. Tumor volumes are shown in the left panel. The percent of body weight change versus baseline is shown in right panel.

FIG. 8: Female nude mice bearing 5238-HX (left panel: efficacy) or female nude rats bearing HT-29 (middle panel: efficacy, and right panel: tolerability) BRAF V600E CRC subcutaneous xenograft tumors were treated p.o. with single agents or combinations of agents as indicated in legends (Dab: Dabrafenib; Tram: Trametinib). Values are mean f SEM; sample size n=6-14 mice per group (5238-HX), n=6 rats per group (HT-29).

FIG. 9: Female FVB mice bearing 24284-MA syngeneic subcutaneous tumors were treated p.o. with single agents or combinations of agents as indicated in legends. Values are mean f SEM; sample size n=5-6 mice per group.

FIG. 10: Compound G YAP/TEAD inhibitor was combined with the EGFR inhibitor EGF816 in the lung cancer cell line PC9 which carries an activating EGFR mutation.

FIG. 11: In vitro viability of the MCF7 breast cancer cell line (PIK3CA mutant) was assessed using the CellTiterGlo assay following a 6-day treatment with YAP/TEAD inhibitors Compound A or Compound B and the PI3K inhibitor NVP-QAU421 (or QAU421) ((S)-N¹-(5-(2-(tert-butyl)pyrimidin-4-yl)-4-methylthiazol-2-yl)pyrrolidine-1,2-dicarboxamide). Growth inhibition %: 0-99=delayed proliferation, 100=growth arrest/stasis, 101-200=reduction in cell number/cell death.

FIG. 12: In vitro viability of the lung KRAS G12C-mutant cell line LU-99 was assessed using the CellTiterGlo following 6-day treatment with the YAP/TEAD inhibitor Compound D (2-((2S,4S)-5-chloro-2-((((1 r,4S)-4-hydroxycyclohexyl)amino)methyl)-2-phenyl-2,3-dihydrobenzofuran-4-yl)-3-fluoro-4-methoxybenzamide) combined with various combinations of MAPK pathway inhibitors (MEK inhibitor MEKINIST/Trametinib/NVP-CFF272 (CFF272), BRAF/CRAF inhibitor NVP-LXH254 (LXH254) and ERK inhibitor NVP-LTT462 (LTT462)). Growth inhibition %: 0-99=delayed proliferation, 100=growth arrest/stasis, 101-200=reduction in cell number/cell death.

FIG. 13: In vitro viability of the lung BRAF mutant colorectal cell line SW-1417 was assessed using the CellTiterGlo following 6-day treatment with the YAP/TEAD inhibitor Compound E (2-((2S,4S)-5-chloro-6-fluoro-2-((((1s,4R)-4-hydroxycyclohexyl)amino)methyl)-2-phenyl-2,3-dihydrobenzofuran-4-yl)-4-(difluoromethoxy)-3-fluorobenzamide) combined with various combinations of MAPK pathway inhibitors (BRAF inhibitor TAFINLAR/Dabrafenib/NVP-LIQ288/LIQ288, MEK inhibitor MEKINIST/Trametinib/NVP-CFF272/CFF272, BRAF/CRAF inhibitor NVP-LXH254 (LXH254) and ERK inhibitor LTT462 (NVP-LTT462)). Growth inhibition %: 0-99=delayed proliferation, 100=growth arrest/stasis, 101-200=reduction in cell number/cell death.

FIG. 14: In vitro viability of the CDKN2A-deleted mesothelioma cell line MSTO-211H was assessed using the CellTiterGlo following 3-day treatment with the YAP/TEAD inhibitor Compound D combined with the p53-HDM2 inhibitor NVP-HDM201 (HDM201). Growth inhibition %: 0-99=delayed proliferation, 100=growth arrest/stasis, 101-200=reduction in cell number/cell death.

FIG. 15: In vitro viability of the mesothelioma cell line MSTO-211H was assessed using the CellTiterGlo following 3-day treatment with the YAP/TEAD inhibitor Compound D combined with the CDK4/6 inhibitor NVP-LEE011 (LEE011 or ribociclib). Growth inhibition %: 0-99=delayed proliferation, 100=growth arrest/stasis, 101-200=reduction in cell number/cell death.

FIG. 16: In vitro viability of the lung cancer cell line HCC44 was assessed using the CellTiterGlo following 7-day treatment with (top) the YAP/TEAD inhibitor Compound A combined with the KRAS G12C inhibitor JDQ443 and (bottom) the YAP/TEAD inhibitor Compound A combined with the KRAS G12C inhibitor JDQ443 and the SHP2 inhibitor TNO155. Growth inhibition %: 0-99=delayed proliferation, 100=growth arrest/stasis, 101-200=reduction in cell number/cell death.

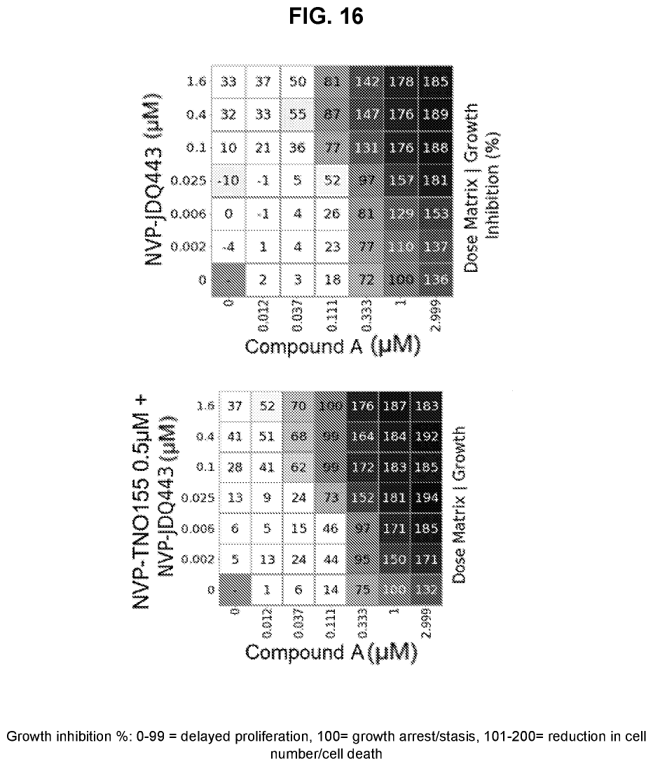

FIG. 17: In vitro viability of the lung cancer cell line HCC44 was assessed using the CellTiterGlo following 7-day treatment with (top) the YAP/TEAD inhibitor Compound A combined with the KRAS G12C inhibitor Sotorasib and (bottom) the YAP/TEAD inhibitor Compound A combined with the KRAS G12C inhibitor Sotorasib and the SHP2 inhibitor TNO155. Growth inhibition %: 0-99=delayed proliferation, 100=growth arrest/stasis, 101-200=reduction in cell number/cell death.

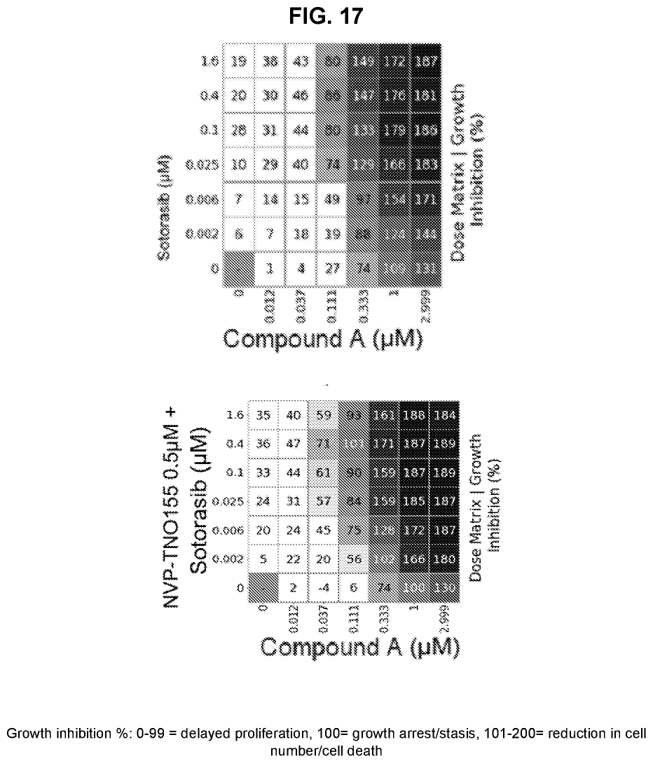

FIG. 18: In vitro viability of the lung cancer cell line HCC44 was assessed using the CellTiterGlo following 7-day treatment with (top left) the YAP/TEAD inhibitor Compound A combined with the KRAS G12C inhibitor Adagrasib, (top right) the YAP/TEAD inhibitor Compound A combined with the KRAS G12C inhibitor Adagrasib and the SHP2 inhibitorTNO155, and (bottom) the YAP/TEAD inhibitor Compound A combined with the KRAS G12C inhibitor Adagrasib and the SHP2 inhibitor RMC-4550. Growth inhibition %: 0-99=delayed proliferation, 100=growth arrest/stasis, 101-200=reduction in cell number/cell death.

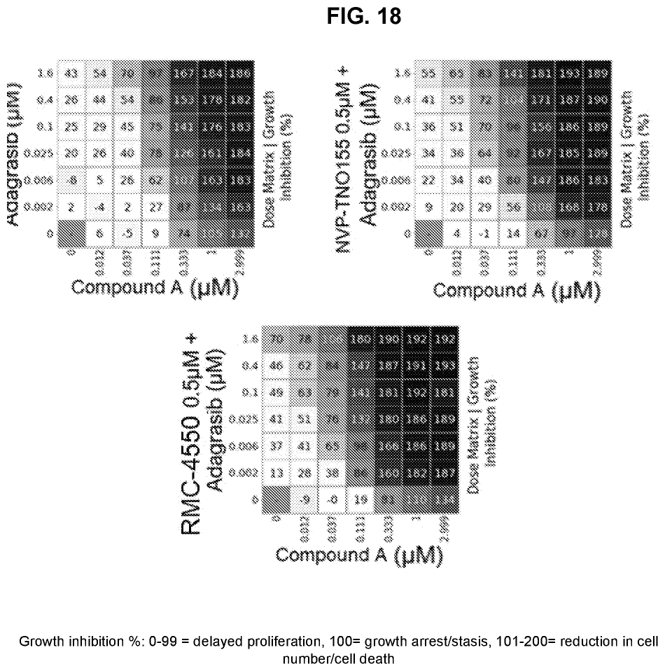

FIG. 19: In vitro viability of the lung cancer cell line HCC44 was assessed using the CellTiterGlo following 7-day treatment with the YAP/TEAD inhibitor Compound A combined with the SHP2 inhibitor TNO-155. Growth inhibition %: 0-99=delayed proliferation, 100=growth arrest/stasis, 101-200=reduction in cell number/cell death.

FIG. 20: In vitro viability of the lung cancer cell line HCC44 was assessed using the CellTiterGlo following 7-day treatment with (top) the YAP/TEAD inhibitor Compound B combined with the KRAS G12C inhibitorJDQ443 and (bottom) the YAP/TEAD inhibitor Compound B combined with the KRAS G12C inhibitor JDQ443 and the SHP2 inhibitor TNO155. Growth inhibition %: 0-99=delayed proliferation, 100=growth arrest/stasis, 101-200=reduction in cell number/cell death.

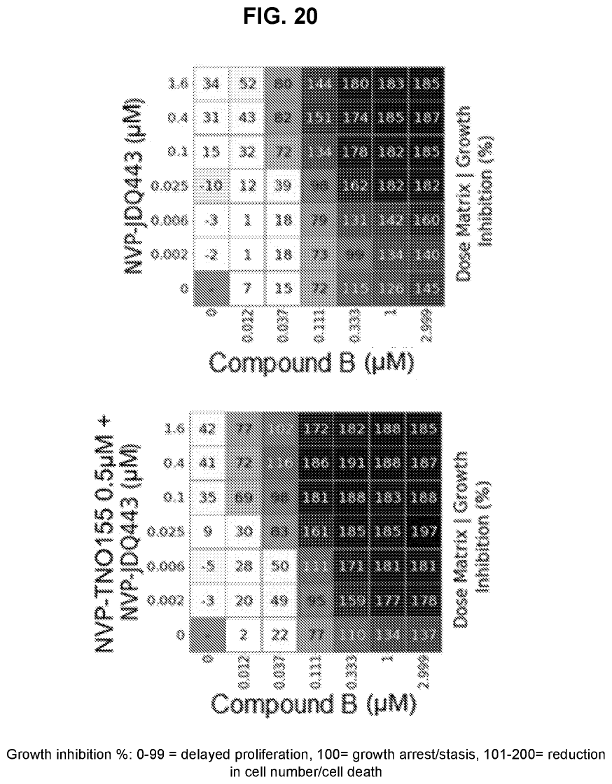

FIG. 21: In vitro viability of the lung cancer cell line HCC44 was assessed using the CellTiterGlo following 7-day treatment with (top) the YAP/TEAD inhibitor Compound B combined with the KRAS G12C inhibitor Sotorasib and (bottom) the YAP/TEAD inhibitor Compound B combined with the KRAS G12C inhibitor Sotorasib and the SHP2 inhibitor TNO155. Growth inhibition %: 0-99=delayed proliferation, 100=growth arrest/stasis, 101-200=reduction in cell number/cell death.

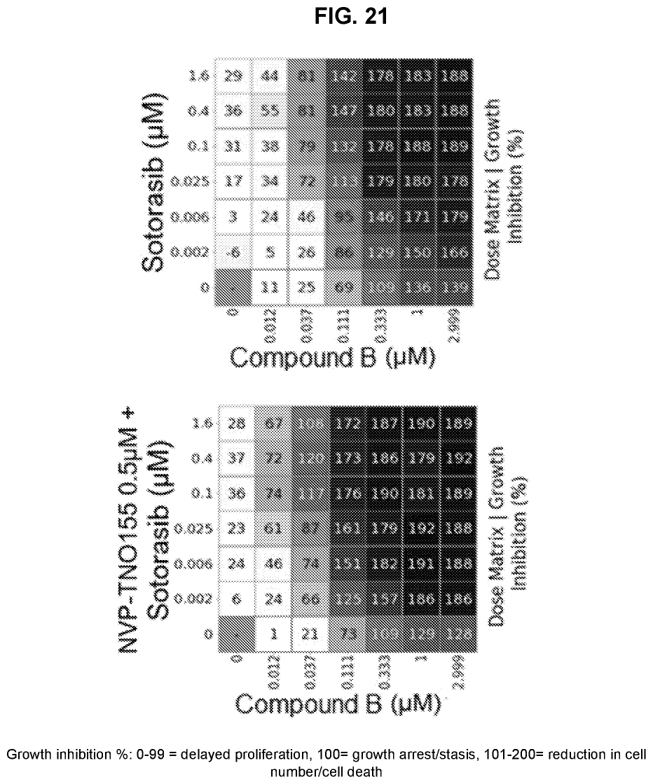

FIG. 22: In vitro viability of the lung cancer cell line HCC44 was assessed using the CellTiterGlo following 7-day treatment with (top left) the YAP/TEAD inhibitor Compound B combined with the KRAS G12C inhibitor Adagrasib, (top right) the YAP/TEAD inhibitor Compound B combined with the KRAS G12C inhibitor Adagrasib and the SHP2 inhibitor TNO155, and (bottom) the YAP/TEAD inhibitor Compound B combined with the KRAS G12C inhibitor Adagrasib and the SHP2 inhibitor RMC-4550. Growth inhibition %: 0-99=delayed proliferation, 100=growth arrest/stasis, 101-200=reduction in cell number/cell death.

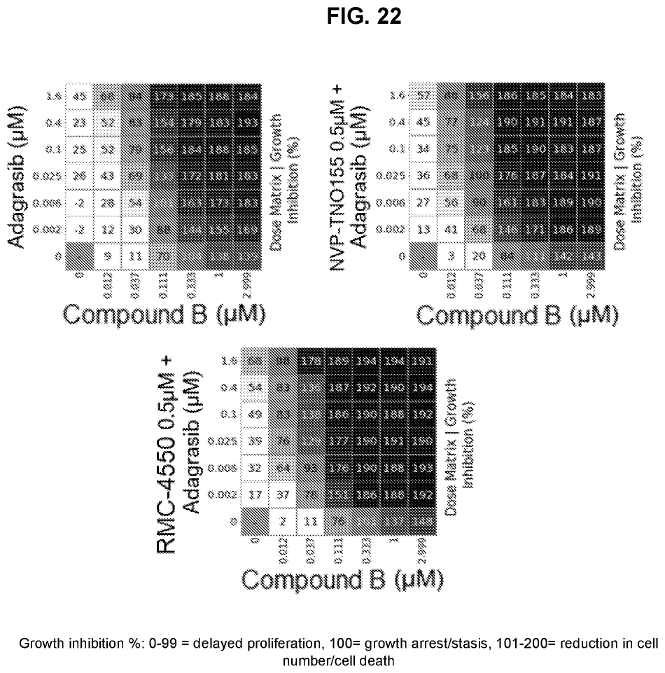

FIG. 23: In vitro viability of the lung cancer cell line HCC44 was assessed using the CellTiterGlo following 7-day treatment with the YAP/TEAD inhibitor Compound B combined with the SHP2 inhibitor TNO-155. Growth inhibition %: 0-99=delayed proliferation, 100=growth arrest/stasis, 101-200=reduction in cell number/cell death.

FIG. 24: In vitro viability of the lung cancer cell line HCC44 was assessed using the CellTiterGlo following 7-day treatment with (top) the YAP/TEAD inhibitor Compound H combined with the KRAS G12C inhibitor JDQ443 and (bottom) the YAP/TEAD inhibitor Compound H combined with the KRAS G12C inhibitor JDQ443 and the SHP2 inhibitor TNO155. Growth inhibition %: 0-99=delayed proliferation, 100=growth arrest/stasis, 101-200=reduction in cell number/cell death.

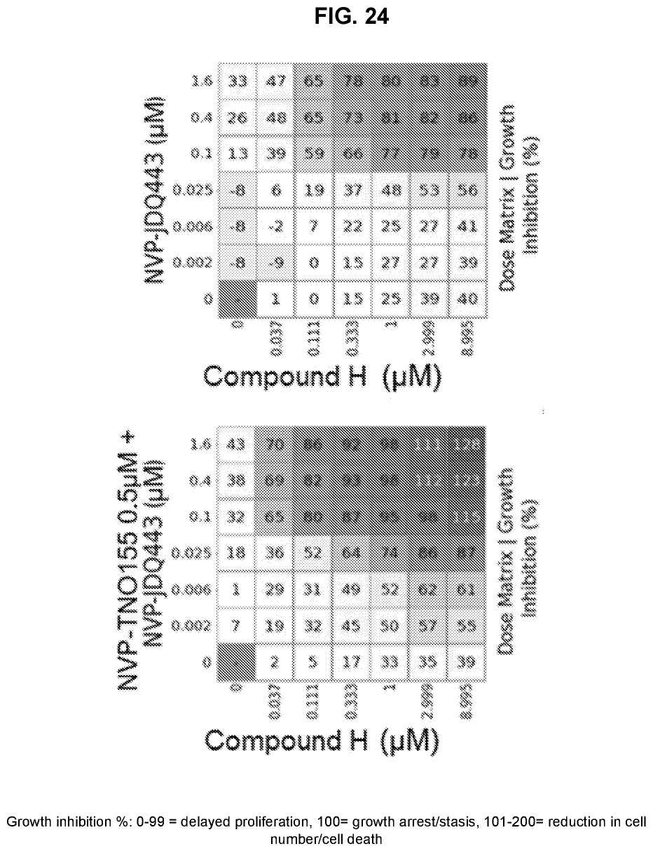

FIG. 25: In vitro viability of the lung cancer cell line HCC44 was assessed using the CellTiterGlo following 7-day treatment with (top) the YAP/TEAD inhibitor Compound H combined with the KRAS G12C inhibitor Sotorasib and (bottom) the YAP/TEAD inhibitor Compound H combined with the KRAS G12C inhibitor Sotorasib and the SHP2 inhibitor TNO155. Growth inhibition %: 0-99=delayed proliferation, 100=growth arrest/stasis, 101-200=reduction in cell number/cell death.

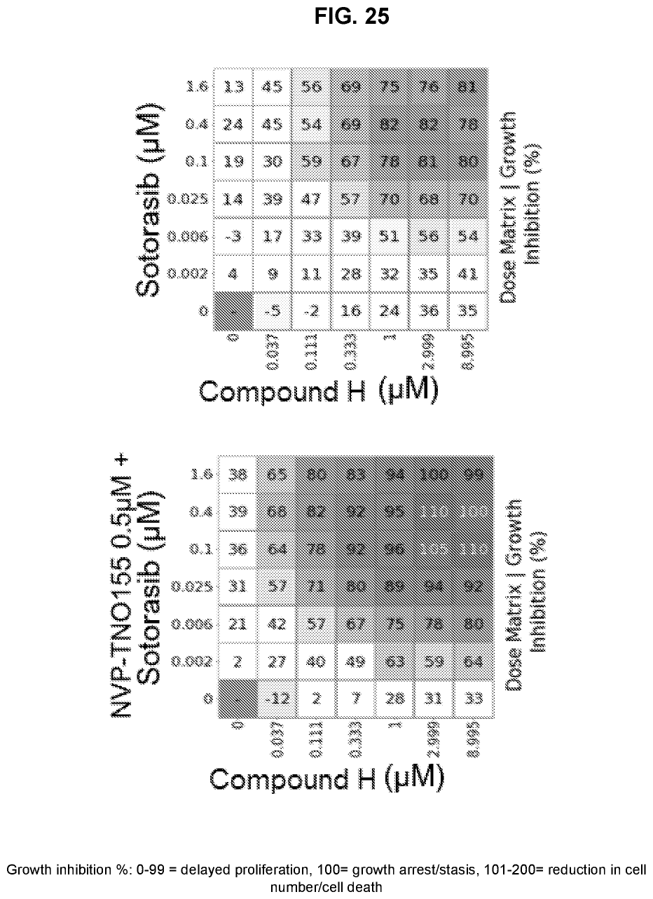

FIG. 26: In vitro viability of the lung cancer cell line HCC44 was assessed using the CellTiterGlo following 7-day treatment with (top) the YAP/TEAD inhibitor Compound H combined with the KRAS G12C inhibitor Adagrasib, and (bottom) the YAP/TEAD inhibitor Compound H combined with the KRAS G12C inhibitor Adagrasib and the SHP2 inhibitor RMC-4550. Growth inhibition %: 0-99=delayed proliferation, 100=growth arrest/stasis, 101-200=reduction in cell number/cell death.

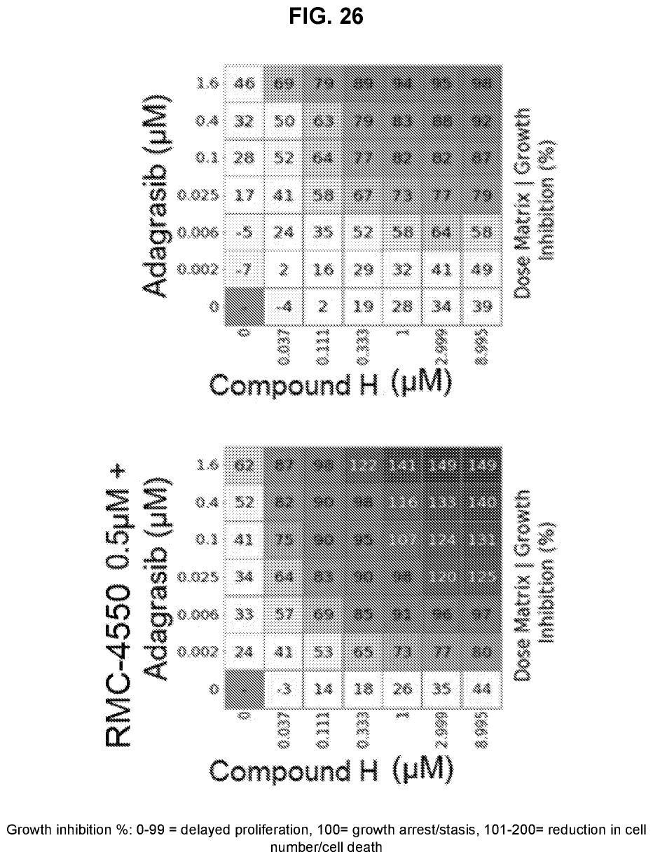

FIG. 27: In vitro viability of the colorectal cancer cell line SW1463 was assessed using the CellTiterGlo following 7-day treatment with (top) the YAP/TEAD inhibitor Compound A combined with the KRAS G12C inhibitor JDQ443 and (bottom) the YAP/TEAD inhibitor Compound A combined with the KRAS G12C inhibitor JDQ443 and the SHP2 inhibitor TNO155. Growth inhibition %: 0-99=delayed proliferation, 100=growth arrest/stasis, 101-200=reduction in cell number/cell death.

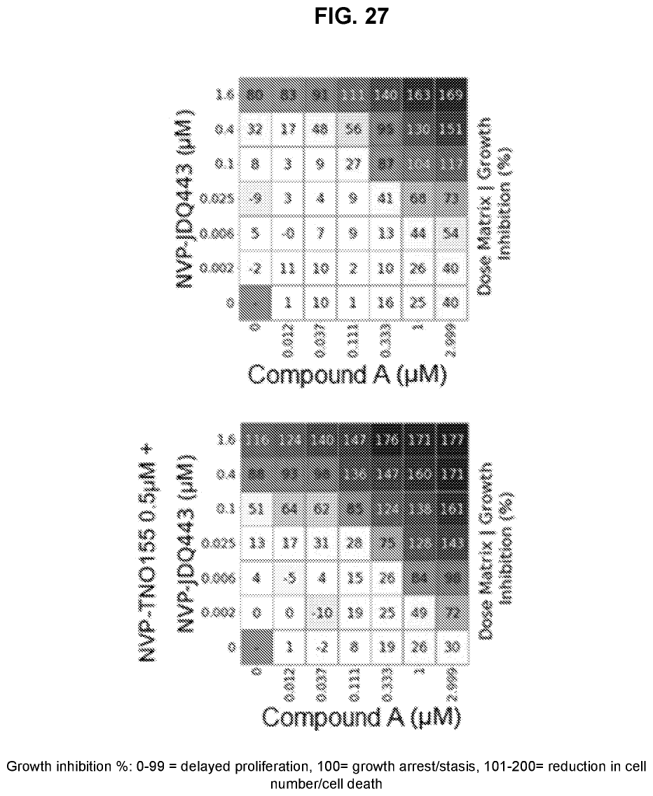

FIG. 28: In vitro viability of the colorectal cancer cell line SW1463 was assessed using the CellTiterGlo following 7-day treatment with (top) the YAP/TEAD inhibitor Compound A combined with the KRAS G12C inhibitor Sotorasib and (bottom) the YAP/TEAD inhibitor Compound A combined with the KRAS G12C inhibitor Sotorasib and the SHP2 inhibitor TNO155. Growth inhibition %: 0-99=delayed proliferation, 100=growth arrest/stasis, 101-200=reduction in cell number/cell death.

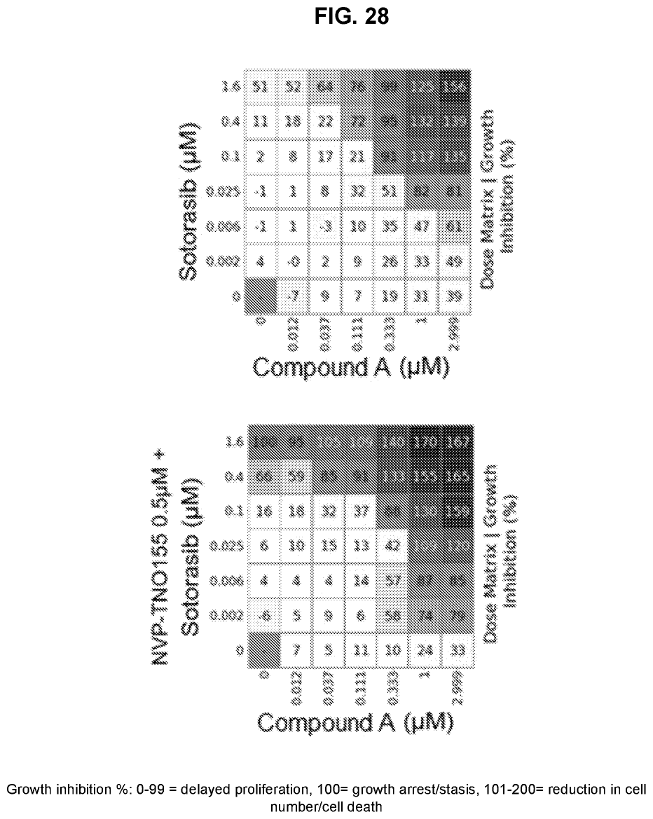

FIG. 29: In vitro viability of the colorectal cancer cell line SW1463 was assessed using the CellTiterGlo following 7-day treatment with (top left) the YAP/TEAD inhibitor Compound A combined with the KRAS G12C inhibitor Adagrasib, (top right) the YAP/TEAD inhibitor Compound A combined with the KRAS G12C inhibitor Adagrasib and the SHP2 inhibitor TNO155, and (bottom) the YAP/TEAD inhibitor Compound A combined with the KRAS G12C inhibitor Adagrasib and the SHP2 inhibitor RMC-4550. Growth inhibition %: 0-99=delayed proliferation, 100=growth arrest/stasis, 101-200=reduction in cell number/cell death.

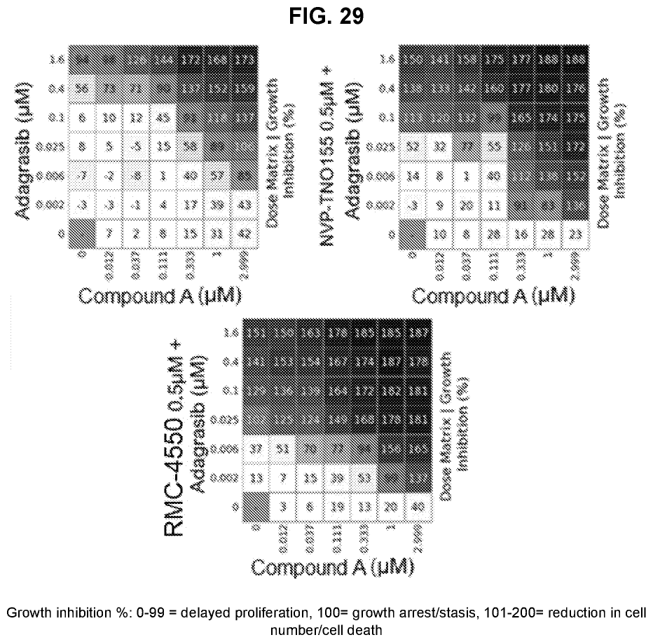

FIG. 30: In vitro viability of the colorectal cancer cell line SW1463 was assessed using the CellTiterGlo following 7-day treatment with the YAP/TEAD inhibitor Compound A combined with the SHP2 inhibitor TNO-155. Growth inhibition %: 0-99=delayed proliferation, 100=growth arrest/stasis, 101-200=reduction in cell number/cell death.

FIG. 31: In vitro viability of the colorectal cancer cell line SW1463 was assessed using the CellTiterGlo following 7-day treatment with (top) the YAP/TEAD inhibitor Compound B combined with the KRAS G12C inhibitor JDQ443 and (bottom) the YAP/TEAD inhibitor Compound B combined with the KRAS G12C inhibitor JDQ443 and the SHP2 inhibitor TNO155. Growth inhibition %: 0-99=delayed proliferation, 100=growth arrest/stasis, 101-200=reduction in cell number/cell death.

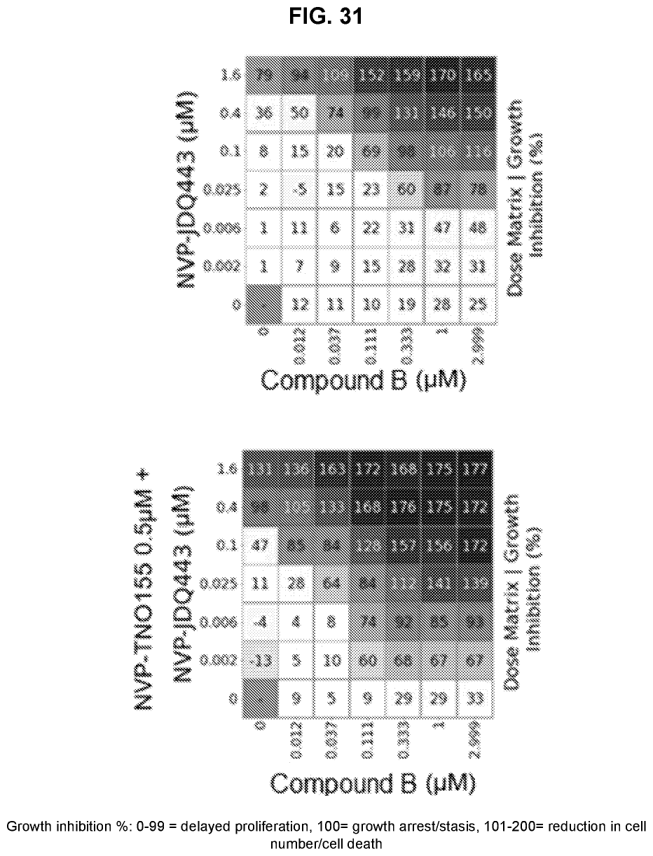

FIG. 32: In vitro viability of the colorectal cancer cell line SW1463 was assessed using the CellTiterGlo following 7-day treatment with (top) the YAP/TEAD inhibitor Compound B combined with the KRAS G12C inhibitor Sotorasib and (bottom) the YAP/TEAD inhibitor Compound B combined with the KRAS G12C inhibitor Sotorasib and the SHP2 inhibitor TNO155. Growth inhibition %: 0-99=delayed proliferation, 100=growth arrest/stasis, 101-200=reduction in cell number/cell death.

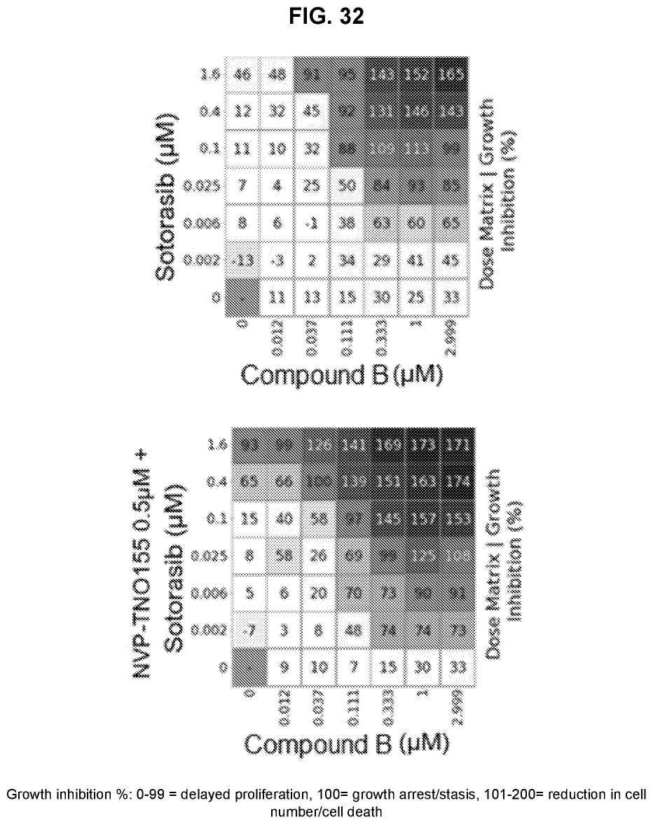

FIG. 33: In vitro viability of the colorectal cancer cell line SW1463 was assessed using the CellTiterGlo following 7-day treatment with (top left) the YAP/TEAD inhibitor Compound B combined with the KRAS G12C inhibitor Adagrasib, (top right) the YAP/TEAD inhibitor Compound B combined with the KRAS G12C inhibitor Adagrasib and the SHP2 inhibitor TNO155, and (bottom) the YAP/TEAD inhibitor Compound B combined with the KRAS G12C inhibitor Adagrasib and the SHP2 inhibitor RMC-4550. Growth inhibition %: 0-99=delayed proliferation, 100=growth arrest/stasis, 101-200=reduction in cell number/cell death.

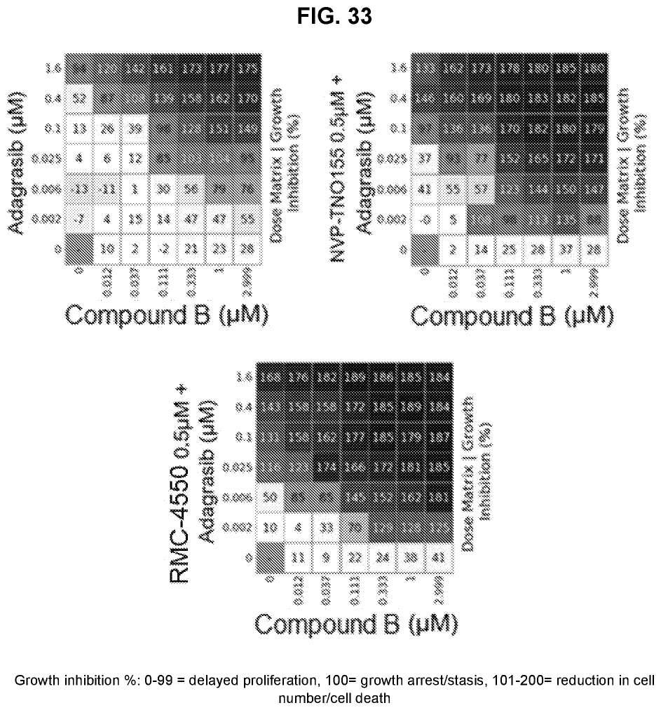

FIG. 34: In vitro viability of the colorectal cancer cell line SW1463 was assessed using the CellTiterGlo following 7-day treatment with (top) the YAP/TEAD inhibitor Compound B combined with the SHP2 inhibitor TNO-155. Growth inhibition %: 0-99=delayed proliferation, 100=growth arrest/stasis, 101-200=reduction in cell number/cell death.

FIG. 35: In vitro viability of the colorectal cancer cell line SW1463 was assessed using the CellTiterGlo following 7-day treatment with (top) the YAP/TEAD inhibitor Compound H combined with the KRAS G12C inhibitor JDQ443 and (bottom) the YAP/TEAD inhibitor Compound H combined with the KRAS G12C inhibitor JDQ443 and the SHP2 inhibitor TNO155. Growth inhibition %: 0-99=delayed proliferation, 100=growth arrest/stasis, 101-200=reduction in cell number/cell death.

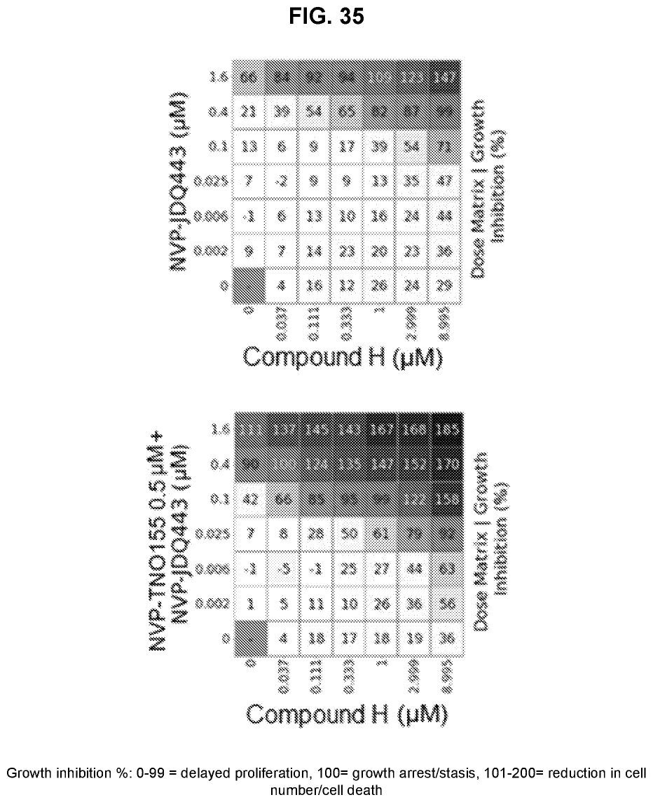

FIG. 36: In vitro viability of the colorectal cancer cell line SW1463 was assessed using the CellTiterGlo following 7-day treatment with (top) the YAP/TEAD inhibitor Compound H combined with the KRAS G12C inhibitor Sotorasib and (bottom) the YAP/TEAD inhibitor Compound H combined with the KRAS G12C inhibitor Sotorasib and the SHP2 inhibitor TNO155. Growth inhibition %: 0-99=delayed proliferation, 100=growth arrest/stasis, 101-200=reduction in cell number/cell death.

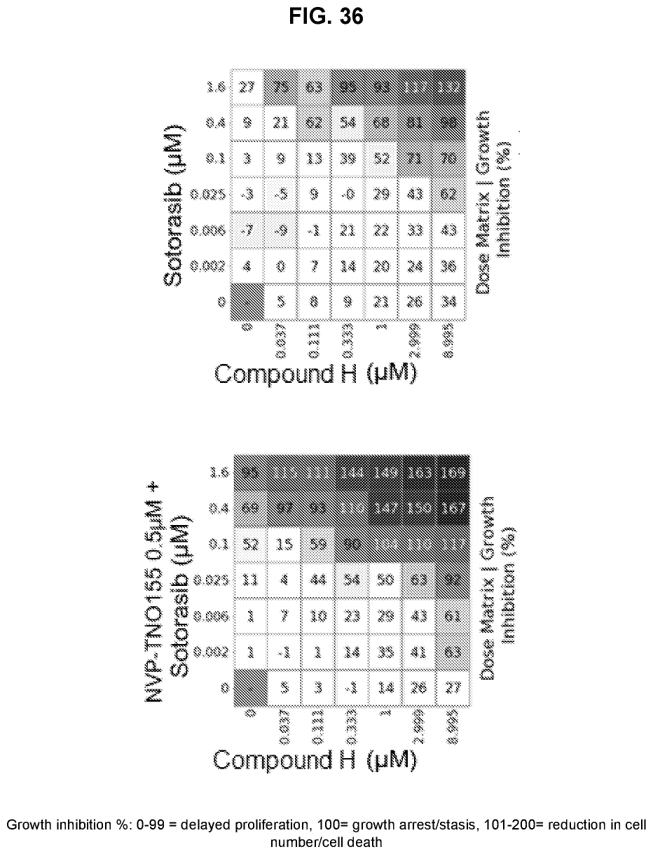

FIG. 37: In vitro viability of the colorectal cancer cell line SW1463 was assessed using the CellTiterGlo following 7-day treatment with (top) the YAP/TEAD inhibitor Compound H combined with the KRAS G12C inhibitor Adagrasib, and (bottom) the YAP/TEAD inhibitor Compound H combined with the KRAS G12C inhibitor Adagrasib and the SHP2 inhibitor RMC-4550. Growth inhibition %: 0-99=delayed proliferation, 100=growth arrest/stasis, 101-200=reduction in cell number/cell death.

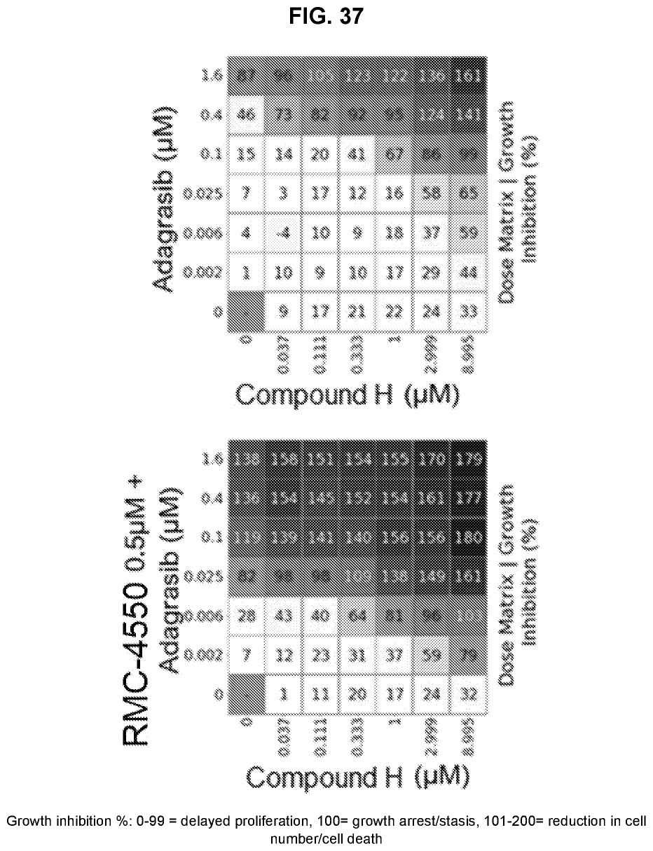

FIG. 38: In vitro viability of the lung cancer cell line NCI-H2122 was assessed using the CellTiterGlo following 7-day treatment with (top) the YAP/TEAD inhibitor Compound A combined with the KRAS G12C inhibitor JDQ-443 and (bottom) the YAP/TEAD inhibitor Compound A combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

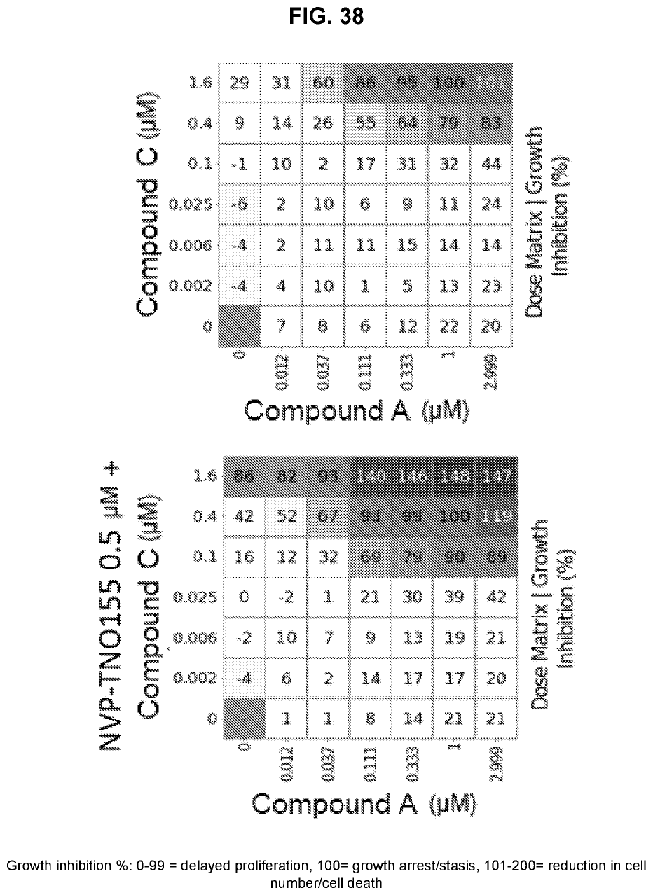

FIG. 39: In vitro viability of the lung cancer cell line NCI-H2122 was assessed using the CellTiterGlo following 7-day treatment with (top) the YAP/TEAD inhibitor Compound B combined with the KRAS G12C inhibitor JDQ-443 and (bottom) the YAP/TEAD inhibitor Compound B combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

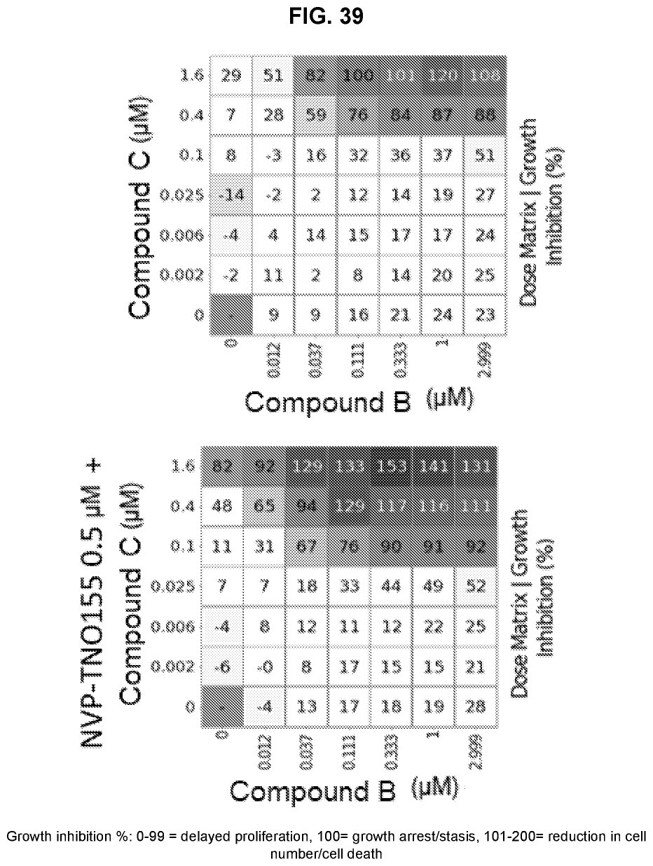

FIG. 40: In vitro viability of the lung cancer cell line NCI-H2122 was assessed using the CellTiterGlo following 7-day treatment with (top) the YAP/TEAD inhibitor Compound H combined with the KRAS G12C inhibitor JDQ-443 and (bottom) the YAP/TEAD inhibitor Compound H combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

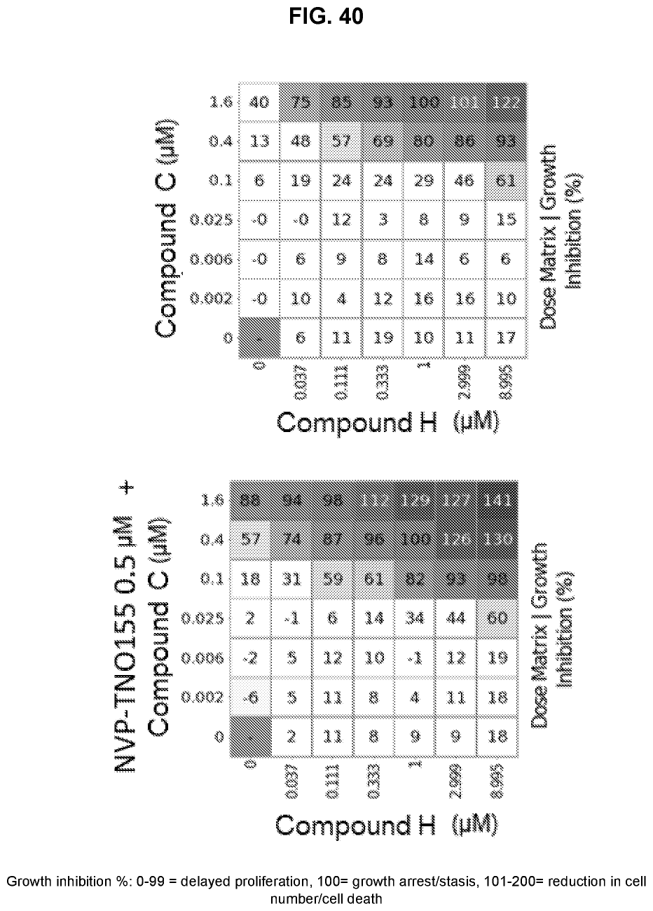

FIG. 41: In vitro viability of the lung cancer cell line NCI-H1373 was assessed using the CellTiterGlo following 7-day treatment with (top) the YAP/TEAD inhibitor Compound A combined with the KRAS G12C inhibitor JDQ-443 and (bottom) the YAP/TEAD inhibitor Compound A combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

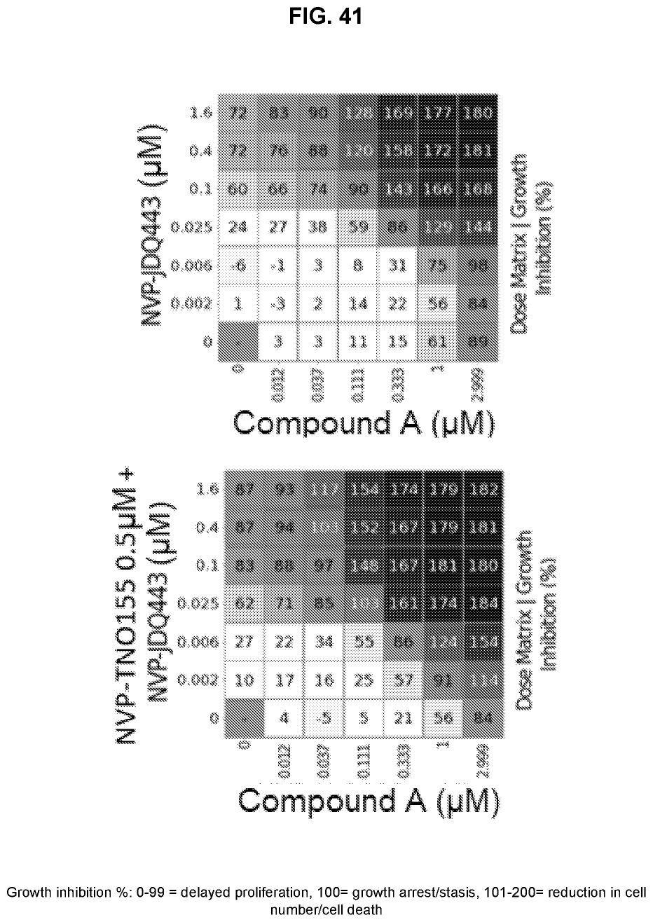

FIG. 42: In vitro viability of the lung cancer cell line NCI-H1373 was assessed using the CellTiterGlo following 7-day treatment with the YAP/TEAD inhibitor Compound A combined with the SHP2 inhibitor TNO155. Growth inhibition %: 0-99=delayed proliferation, 100=growth arrest/stasis, 101-200=reduction in cell number/cell death.

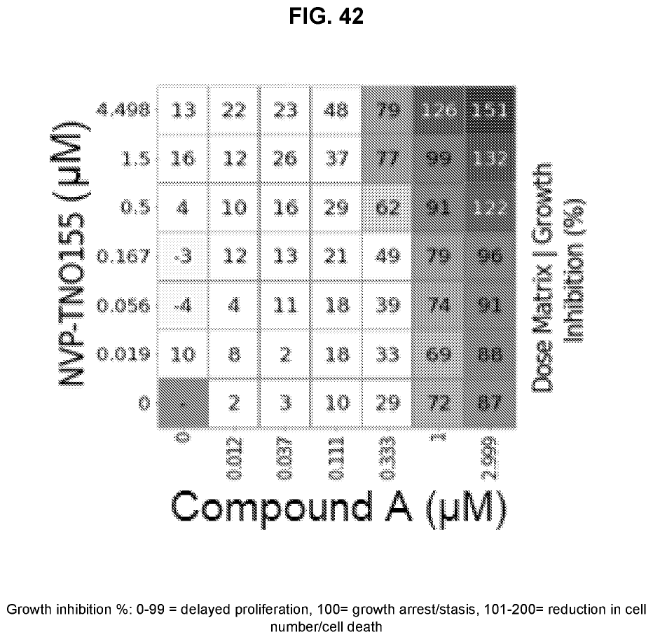

FIG. 43: In vitro viability of the lung cancer cell line NCI-H1373 was assessed using the CellTiterGlo following 7-day treatment with (top) the YAP/TEAD inhibitor Compound B combined with the KRAS G12C inhibitor JDQ-443 and (bottom) the YAP/TEAD inhibitor Compound B combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

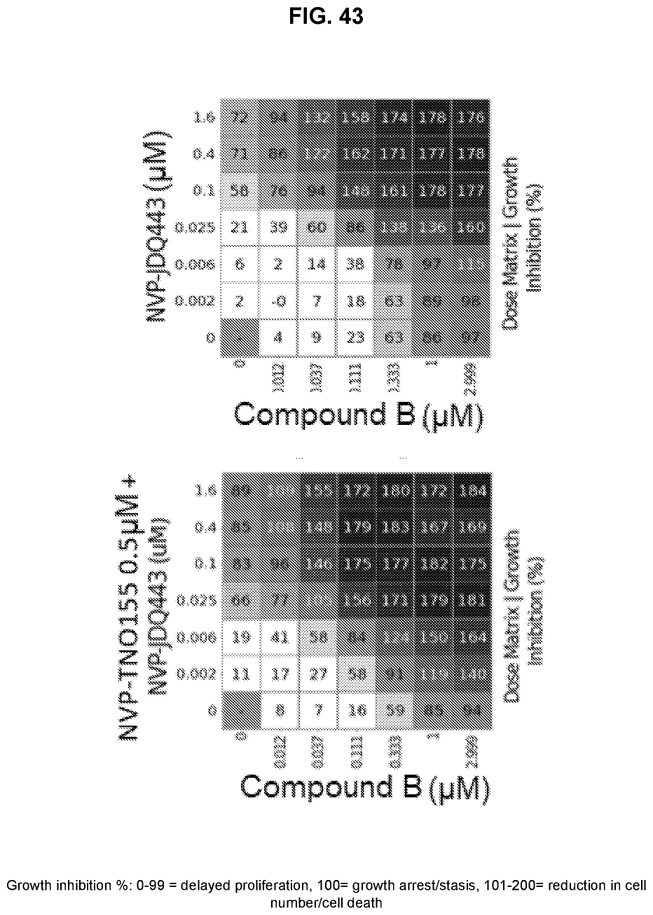

FIG. 44: In vitro viability of the lung cancer cell line NCI-H1373 was assessed using the CellTiterGlo following 7-day treatment with the YAP/TEAD inhibitor Compound B combined with the SHP2 inhibitor TNO155. Growth inhibition %: 0-99=delayed proliferation, 100=growth arrest/stasis, 101-200=reduction in cell number/cell death.

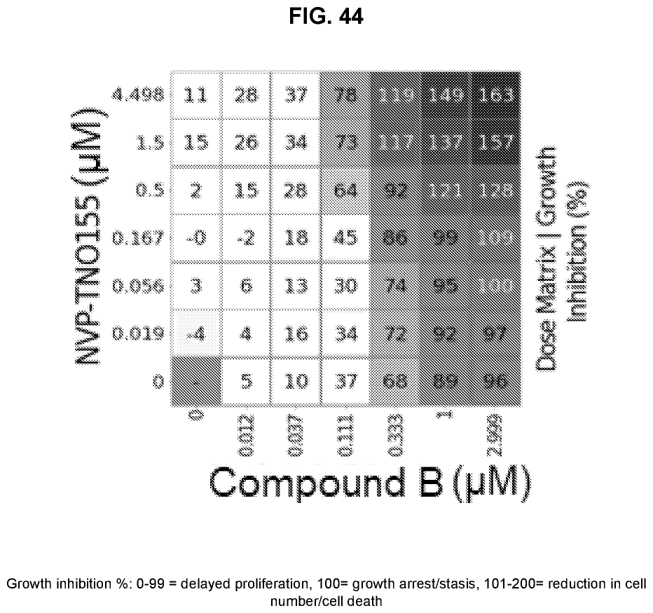

FIG. 45: In vitro viability of the lung cancer cell line NCI-H1373 was assessed using the CellTiterGlo following 7-day treatment with (top) the YAP/TEAD inhibitor Compound H combined with the KRAS G12C inhibitor JDQ-443 and (bottom) the YAP/TEAD inhibitor Compound H combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

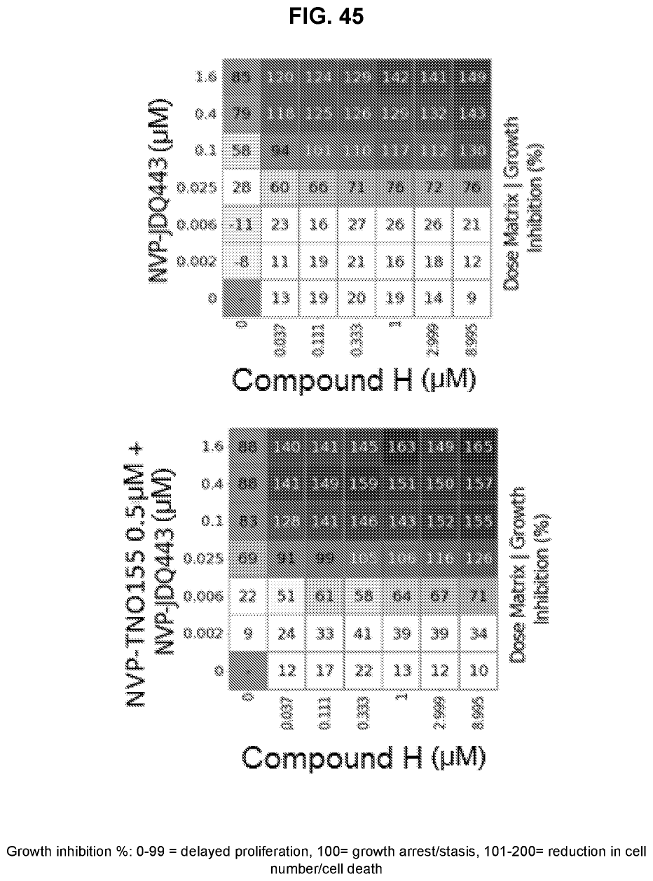

FIG. 46: In vitro viability of the lung cancer cell line NCI-H1373 was assessed using the CellTiterGlo following 7-day treatment with the YAP/TEAD inhibitor Compound H combined with the SHP2 inhibitor TNO155. Growth inhibition %: 0-99=delayed proliferation, 100=growth arrest/stasis, 101-200=reduction in cell number/cell death.

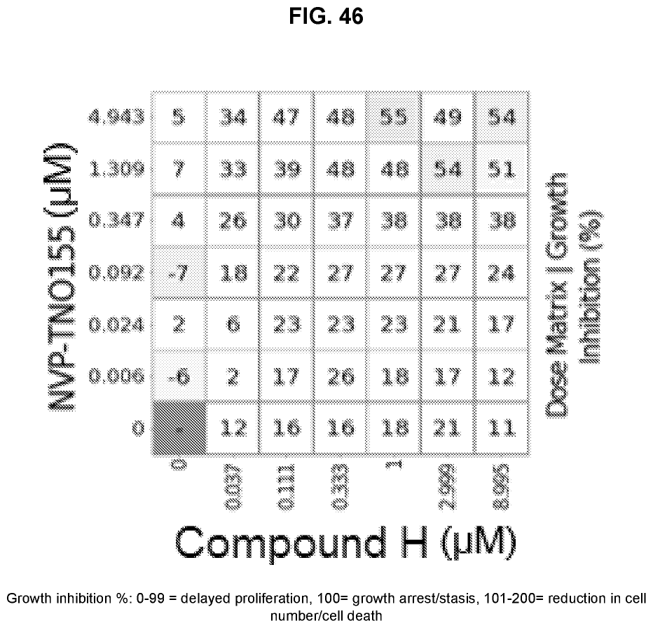

FIG. 47: In vitro viability of the lung cancer cell line HCC-1171 was assessed using the CellTiterGlo following 7-day treatment with (top) the YAP/TEAD inhibitor Compound A combined with the KRAS G12C inhibitor JDQ-443 and (bottom) the YAP/TEAD inhibitor Compound A combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

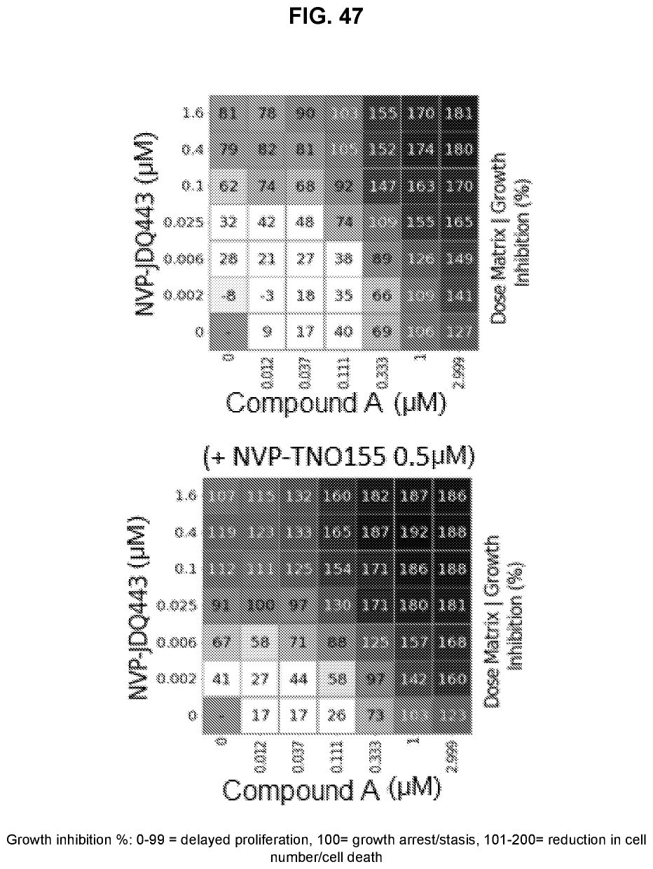

FIG. 48: In vitro viability of the lung cancer cell line HCC-1171 was assessed using the CellTiterGlo following 7-day treatment with the YAP/TEAD inhibitor Compound A combined with the SHP2 inhibitor TNO155. Growth inhibition %: 0-99=delayed proliferation, 100=growth arrest/stasis, 101-200=reduction in cell number/cell death.

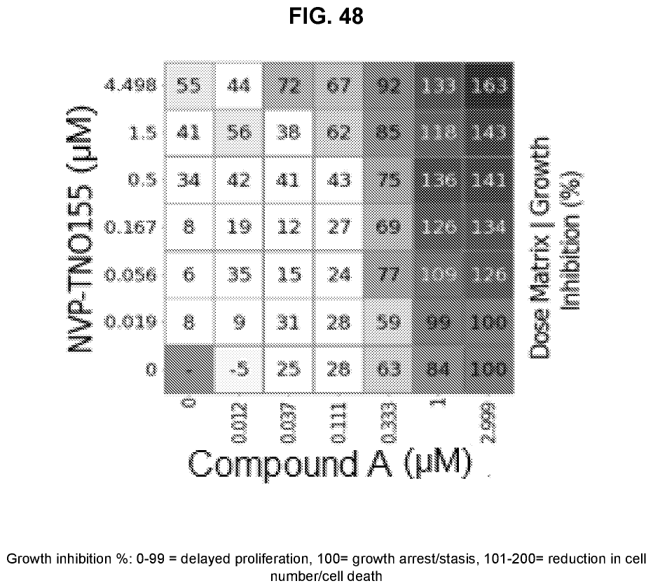

FIG. 49: In vitro viability of the lung cancer cell line HCC-1171 was assessed using the CellTiterGlo following 7-day treatment with (top) the YAP/TEAD inhibitor Compound B combined with the KRAS G12C inhibitor JDQ-443 and (bottom) the YAP/TEAD inhibitor Compound B combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

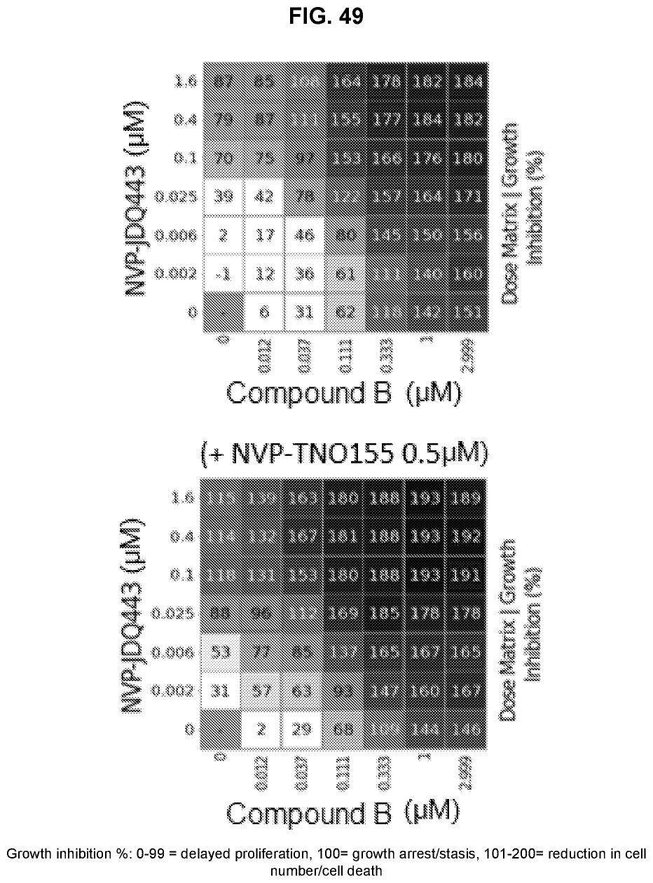

FIG. 50: In vitro viability of the lung cancer cell line HCC-1171 was assessed using the CellTiterGlo following 7-day treatment with the YAP/TEAD inhibitor Compound B combined with the SHP2 inhibitor TNO155. Growth inhibition %: 0-99=delayed proliferation, 100=growth arrest/stasis, 101-200=reduction in cell number/cell death.

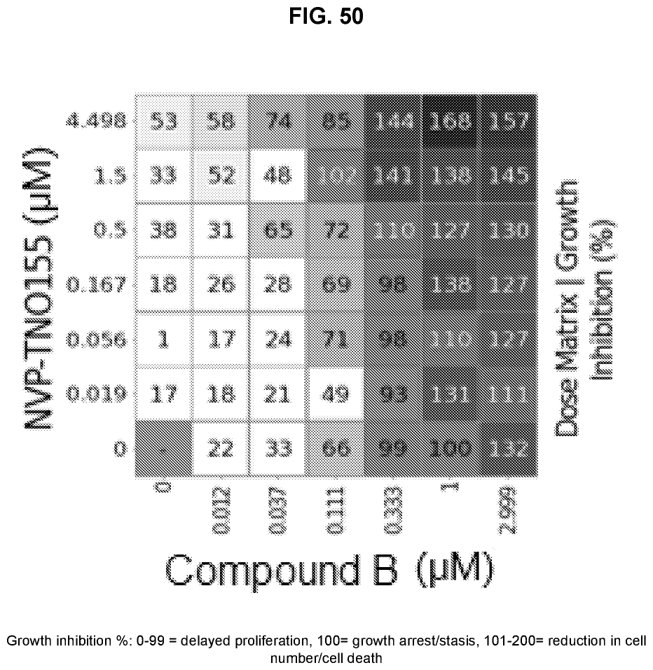

FIG. 51: In vitro viability of the lung cancer cell line HCC-1171 was assessed using the CellTiterGlo following 7-day treatment with (top) the YAP/TEAD inhibitor Compound H combined with the KRAS G12C inhibitor JDQ-443 and (bottom) the YAP/TEAD inhibitor Compound H combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

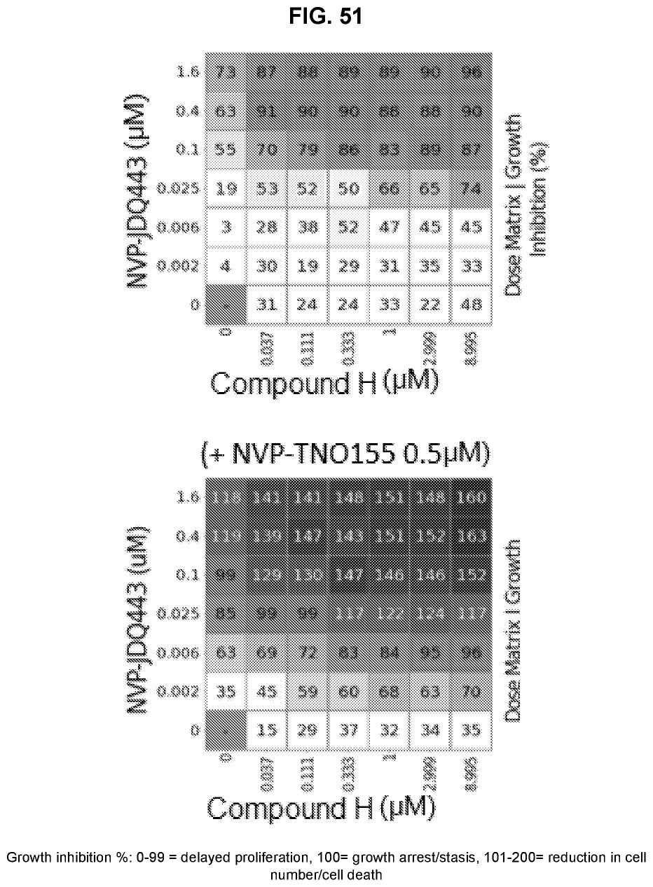

FIG. 52: In vitro viability of the lung cancer cell line HCC-1171 was assessed using the CellTiterGlo following 7-day treatment with the YAP/TEAD inhibitor Compound H combined with the SHP2 inhibitor TNO155. Growth inhibition %: 0-99=delayed proliferation, 100=growth arrest/stasis, 101-200=reduction in cell number/cell death.

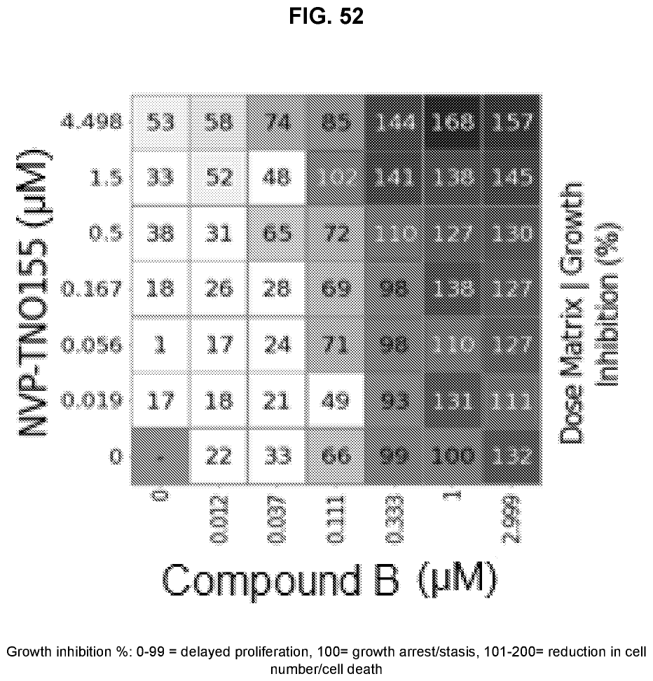

FIG. 53: In vitro viability of the lung cancer cell line NCI-H358 was assessed using the CellTiterGlo following 7-day treatment with (top) the YAP/TEAD inhibitor Compound A combined with the KRAS G12C inhibitor JDQ-443 and (bottom) the YAP/TEAD inhibitor Compound A combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

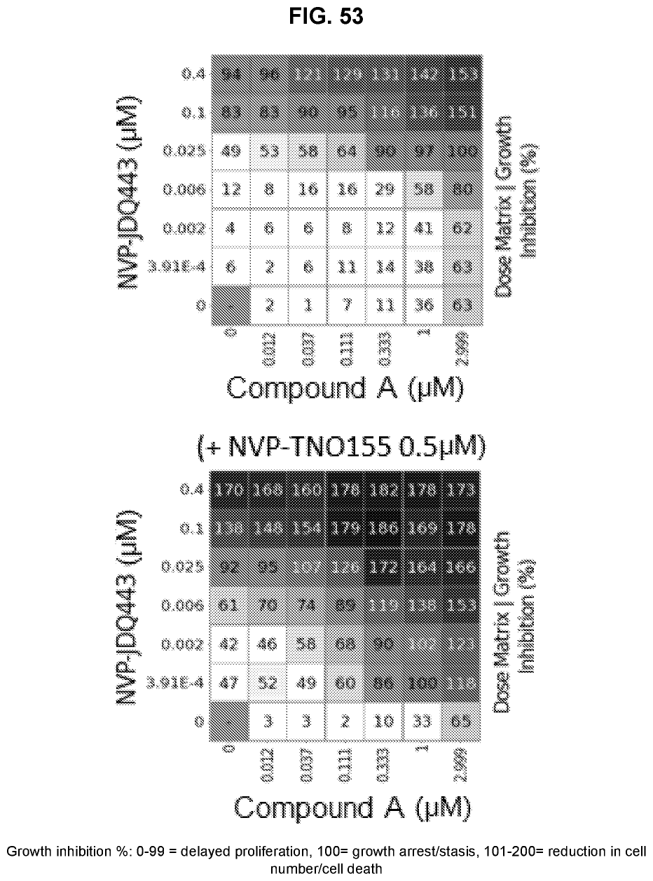

FIG. 54: In vitro viability of the lung cancer cell line NCI-H358 was assessed using the CellTiterGlo following 7-day treatment with the YAP/TEAD inhibitor Compound A combined with the SHP2 inhibitor TNO155. Growth inhibition %: 0-99=delayed proliferation, 100=growth arrest/stasis, 101-200=reduction in cell number/cell death.

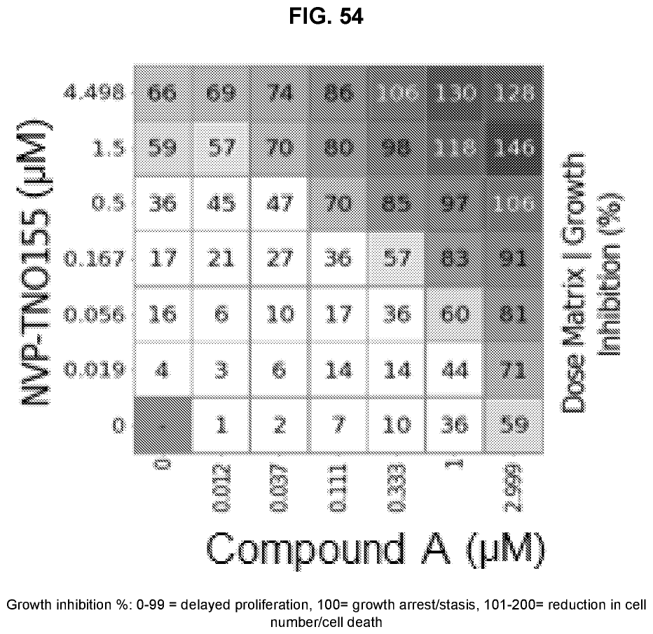

FIG. 55: In vitro viability of the lung cancer cell line NCI-H358 was assessed using the CellTiterGlo following 7-day treatment with (top) the YAP/TEAD inhibitor Compound B combined with the KRAS G12C inhibitor JDQ-443 and (bottom) the YAP/TEAD inhibitor Compound B combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

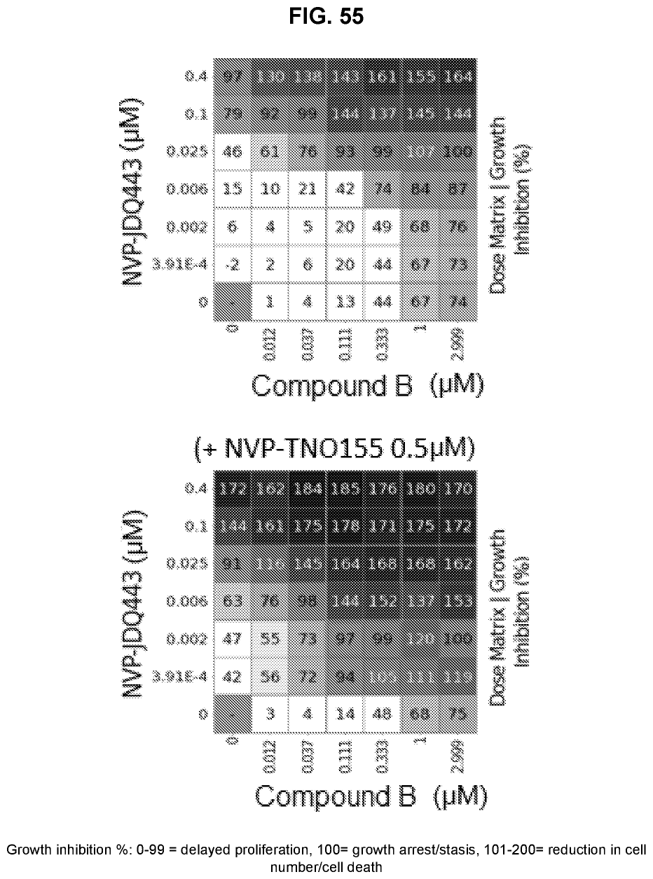

FIG. 56: In vitro viability of the lung cancer cell line NCI-H358 was assessed using the CellTiterGlo following 7-day treatment with the YAP/TEAD inhibitor Compound B combined with the SHP2 inhibitor TNO155. Growth inhibition %: 0-99=delayed proliferation, 100=growth arrest/stasis, 101-200=reduction in cell number/cell death.

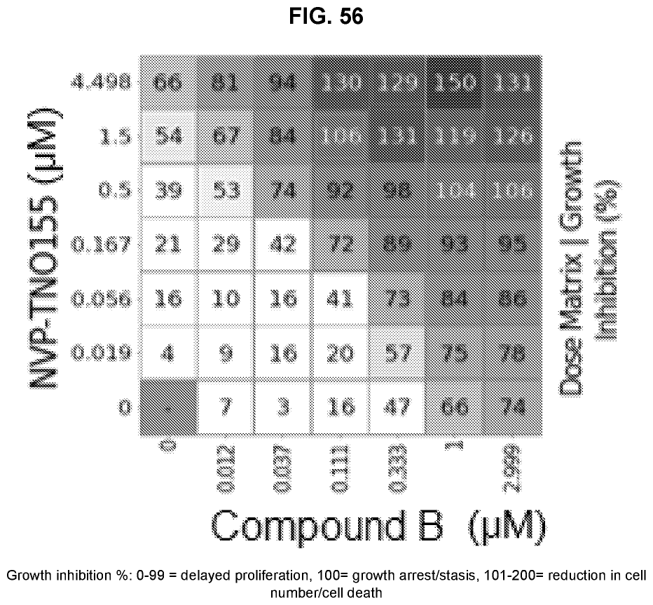

FIG. 57: In vitro viability of the lung cancer cell line NCI-H358 was assessed using the CellTiterGlo following 7-day treatment with (top) the YAP/TEAD inhibitor Compound H combined with the KRAS G12C inhibitor JDQ-443 and (bottom) the YAP/TEAD inhibitor Compound H combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

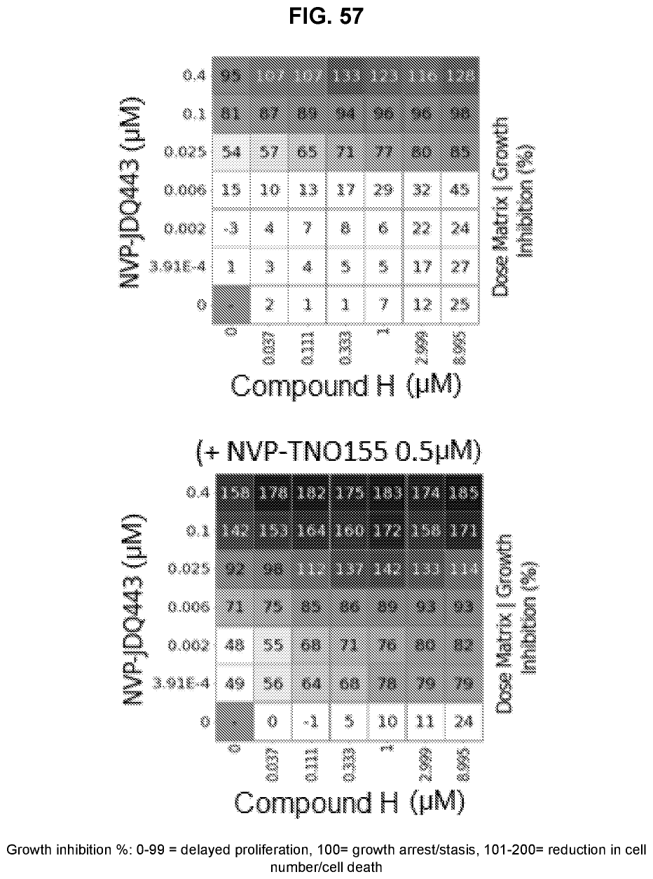

FIG. 58: In vitro viability of the lung cancer cell line NCI-H358 was assessed using the CellTiterGlo following 7-day treatment with the YAP/TEAD inhibitor Compound H combined with the SHP2 inhibitor TNO155. Growth inhibition %: 0-99=delayed proliferation, 100=growth arrest/stasis, 101-200=reduction in cell number/cell death.

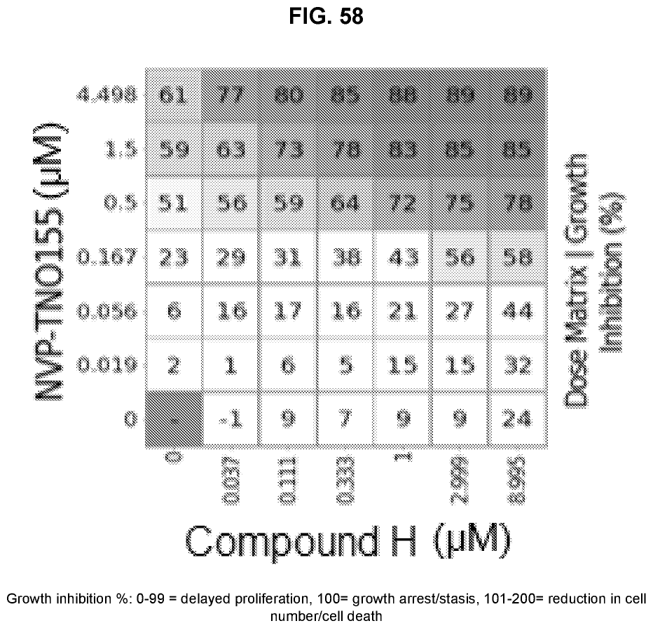

FIG. 59: In vitro viability of the lung cancer cell line LU65 was assessed using the CellTiterGlo following 7-day treatment with (top) the YAP/TEAD inhibitor Compound A combined with the KRAS G12C inhibitor sotorasib and (bottom) the YAP/TEAD inhibitor Compound A combined with the KRAS G12C inhibitor sotorasib and the SHP2 inhibitor TNO155. Growth inhibition %: 0-99=delayed proliferation, 100=growth arrest/stasis, 101-200=reduction in cell number/cell death.

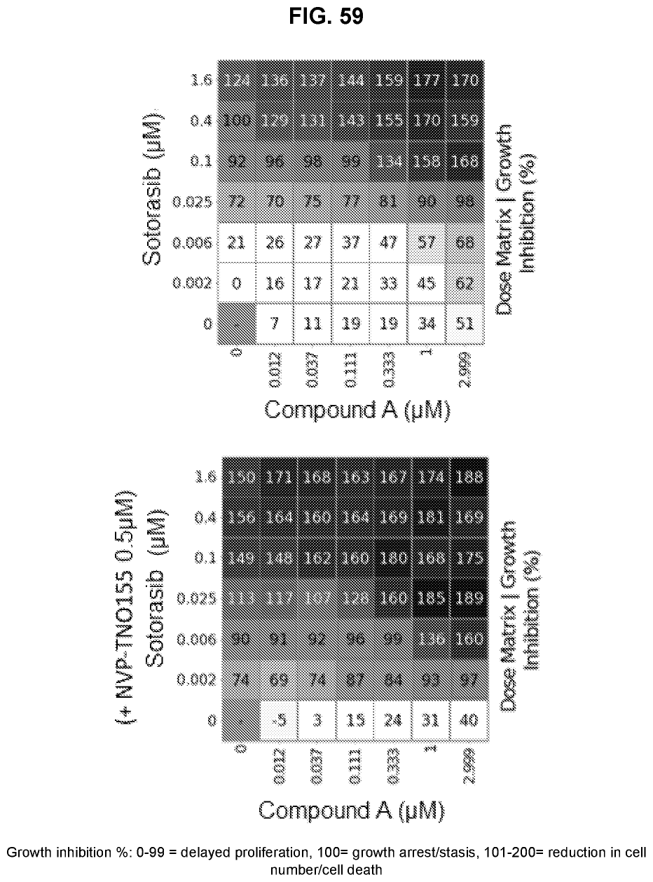

FIG. 60: In vitro viability of the lung cancer cell line LU65 was assessed using the CellTiterGlo following 7-day treatment with (top) the YAP/TEAD inhibitor Compound A combined with the KRAS G12C inhibitor adagrasib and (bottom) the YAP/TEAD inhibitor Compound A combined with the KRAS G12C inhibitor adagrasib and the SHP2 inhibitor TNO155. Growth inhibition %: 0-99=delayed proliferation, 100=growth arrest/stasis, 101-200=reduction in cell number/cell death.

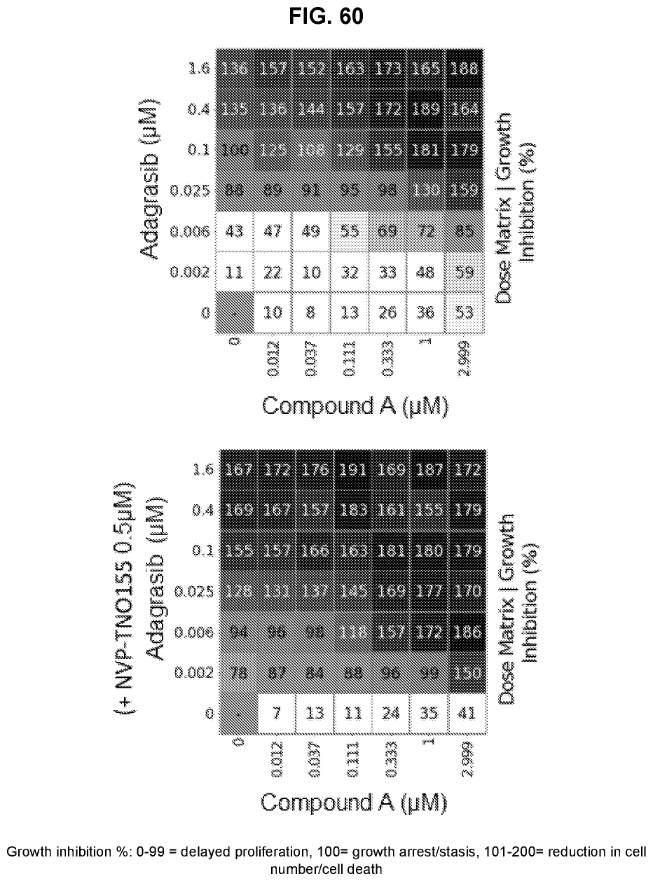

FIG. 61: In vitro viability of the lung cancer cell line LU65 was assessed using the CellTiterGlo following 7-day treatment with the YAP/TEAD inhibitor Compound A combined with the KRAS G12C inhibitor JDQ443. Growth inhibition %: 0-99=delayed proliferation, 100=growth arrest/stasis, 101-200=reduction in cell number/cell death.

FIG. 62: In vitro viability of the lung cancer cell line LU65 was assessed using the CellTiterGlo following 7-day treatment with the YAP/TEAD inhibitor Compound B combined with the KRAS G12C inhibitor JDQ443. Growth inhibition %: 0-99=delayed proliferation, 100=growth arrest/stasis, 101-200=reduction in cell number/cell death.

FIG. 63: In vitro viability of the lung cancer cell line LU65 was assessed using the CellTiterGlo following 7-day treatment with (top) the YAP/TEAD inhibitor Compound H combined with the KRAS G12C inhibitorJDQ443 and (bottom) the YAP/TEAD inhibitor Compound H combined with the KRAS G12C inhibitor JDQ443 and the SHP2 inhibitor TNO155. Growth inhibition %: 0-99=delayed proliferation, 100=growth arrest/stasis, 101-200=reduction in cell number/cell death.

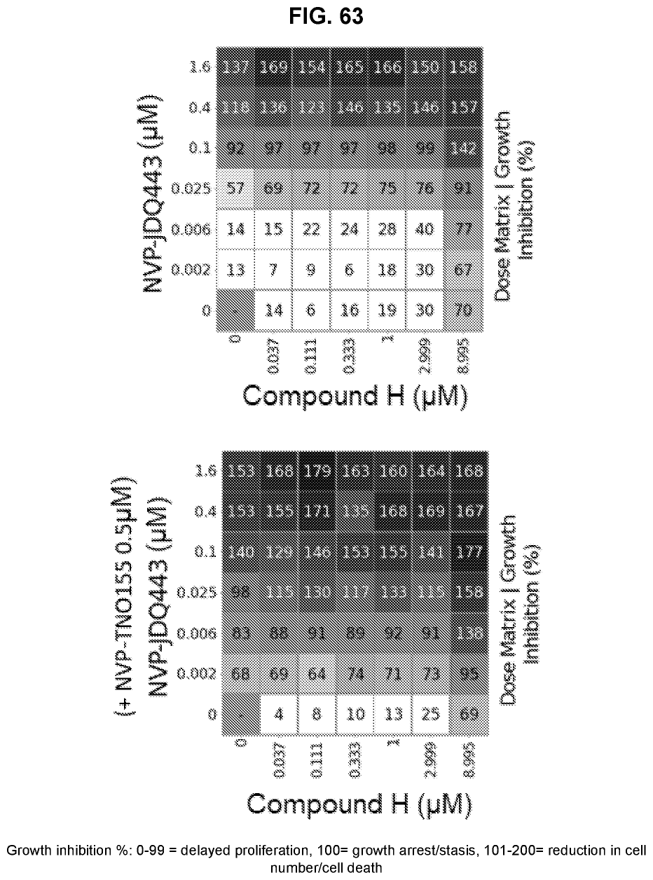

FIG. 64: In vitro viability of the colorectal cancer cell line SW837 was assessed using the CellTiterGlo following 7-day treatment with (top) the YAP/TEAD inhibitor Compound A combined with the KRAS G12C inhibitor JDQ-443 and (bottom) the YAP/TEAD inhibitor Compound A combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

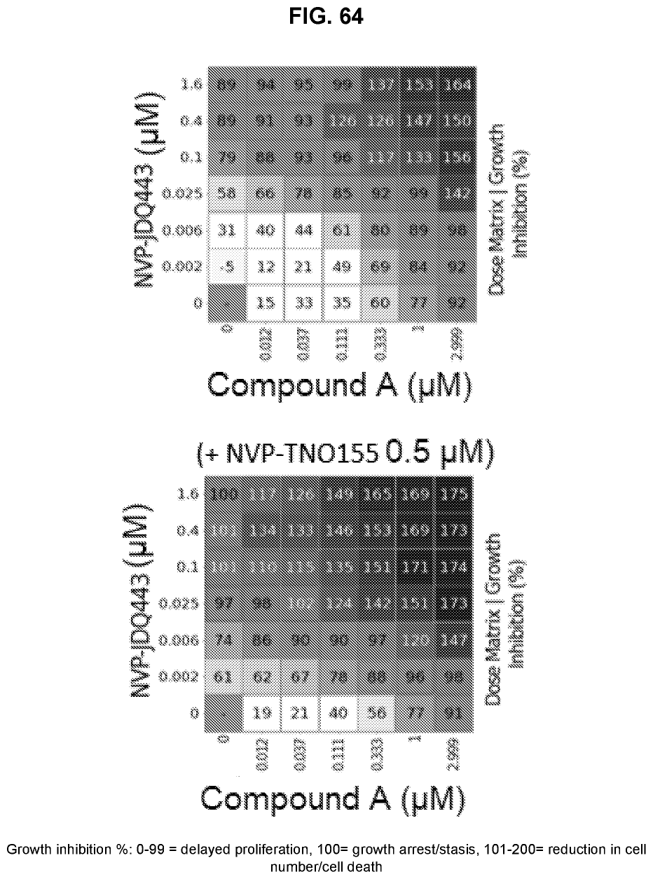

FIG. 65: In vitro viability of the colorectal cancer cell line SW837 was assessed using the CellTiterGlo following 7-day treatment with the YAP/TEAD inhibitor Compound A combined with the SHP2 inhibitor TNO155. Growth inhibition %: 0-99=delayed proliferation, 100=growth arrest/stasis, 101-200=reduction in cell number/cell death.

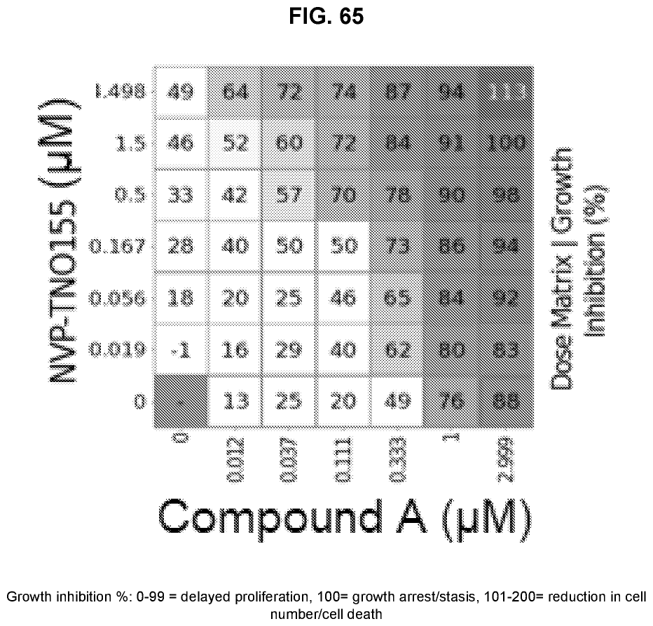

FIG. 66: In vitro viability of the colorectal cancer cell line SW837 was assessed using the CellTiterGlo following 7-day treatment with (top) the YAP/TEAD inhibitor Compound B combined with the KRAS G12C inhibitor JDQ-443 and (bottom) the YAP/TEAD inhibitor Compound B combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

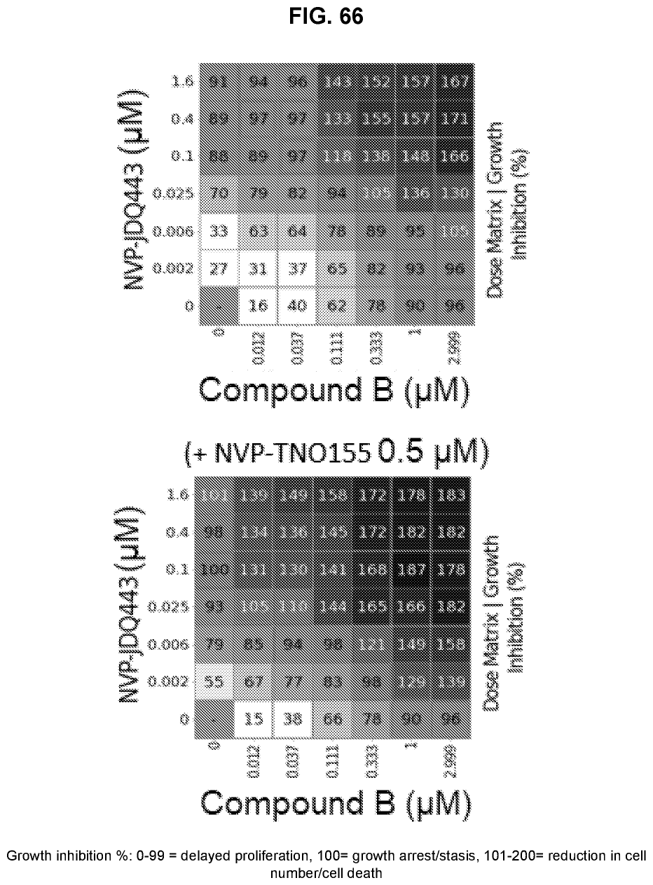

FIG. 67: In vitro viability of the colorectal cancer cell line SW837 was assessed using the CellTiterGlo following 7-day treatment with the YAP/TEAD inhibitor Compound B combined with the SHP2 inhibitor TNO155. Growth inhibition %: 0-99=delayed proliferation, 100=growth arrest/stasis, 101-200=reduction in cell number/cell death.

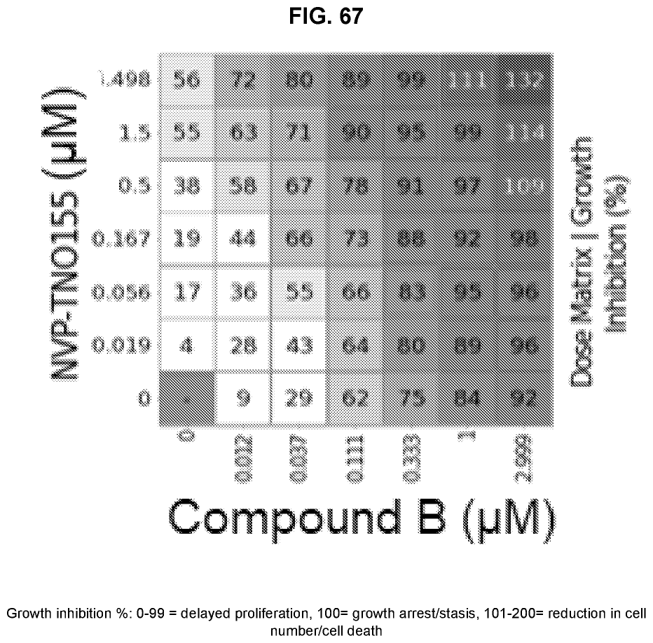

FIG. 68: In vitro viability of the colorectal cancer cell line SW837 was assessed using the CellTiterGlo following 7-day treatment with (top) the YAP/TEAD inhibitor Compound H combined with the KRAS G12C inhibitor JDQ-443 and (bottom) the YAP/TEAD inhibitor Compound H combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

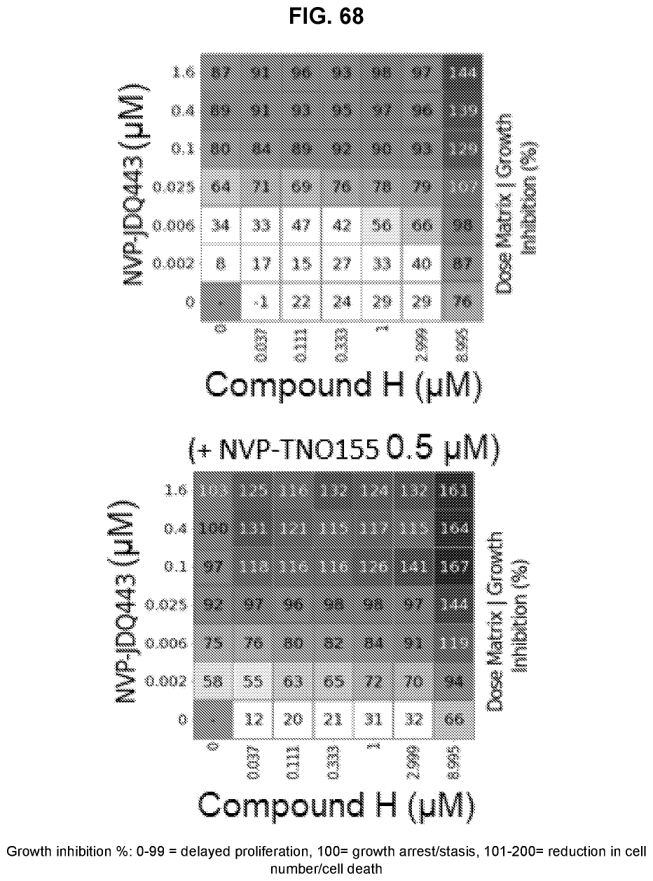

FIG. 69: In vitro viability of the colorectal cancer cell line SW837 was assessed using the CellTiterGlo following 7-day treatment with the YAP/TEAD inhibitor Compound H combined with the SHP2 inhibitor TNO155. Growth inhibition %: 0-99=delayed proliferation, 100=growth arrest/stasis, 101-200=reduction in cell number/cell death.

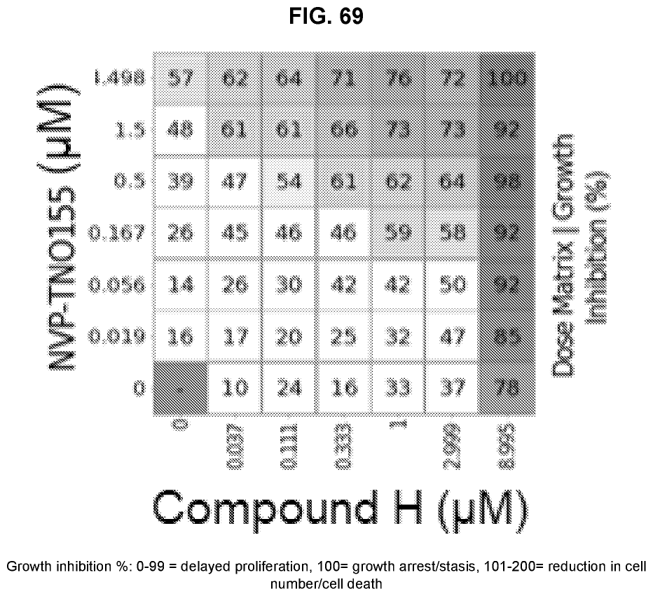

FIG. 70: In vitro viability of the colorectal cancer cell line LIM2099 was assessed using the CellTiterGlo following 7-day treatment with (top) the YAP/TEAD inhibitor Compound A combined with the KRAS G12C inhibitor JDQ-443 and (bottom) the YAP/TEAD inhibitor Compound A combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

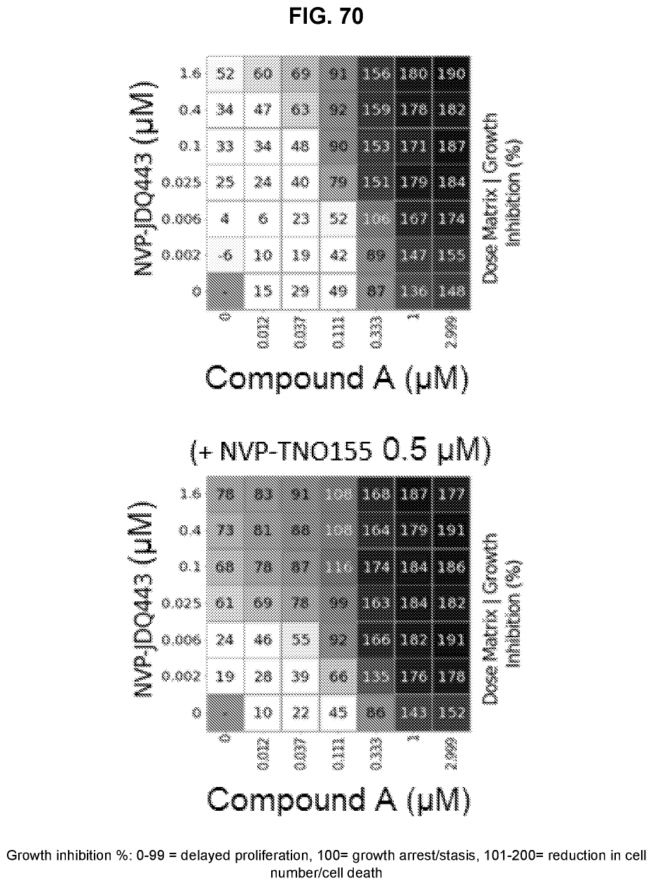

FIG. 71: In vitro viability of the colorectal cancer cell line LIM2099 was assessed using the CellTiterGlo following 7-day treatment with the YAP/TEAD inhibitor Compound A combined with the SHP2 inhibitor TNO155. Growth inhibition %: 0-99=delayed proliferation, 100=growth arrest/stasis, 101-200=reduction in cell number/cell death.

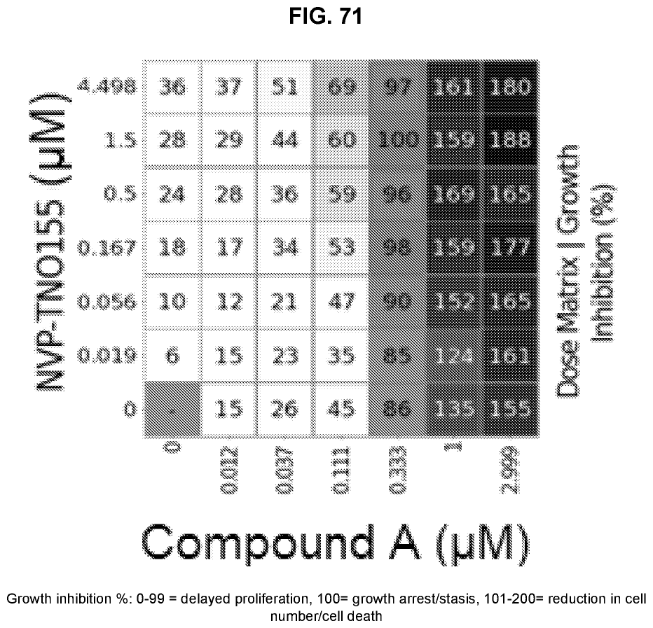

FIG. 72: In vitro viability of the colorectal cancer cell line LIM2099 was assessed using the CellTiterGlo following 7-day treatment with (top) the YAP/TEAD inhibitor Compound B combined with the KRAS G12C inhibitor JDQ-443 and (bottom) the YAP/TEAD inhibitor Compound B combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

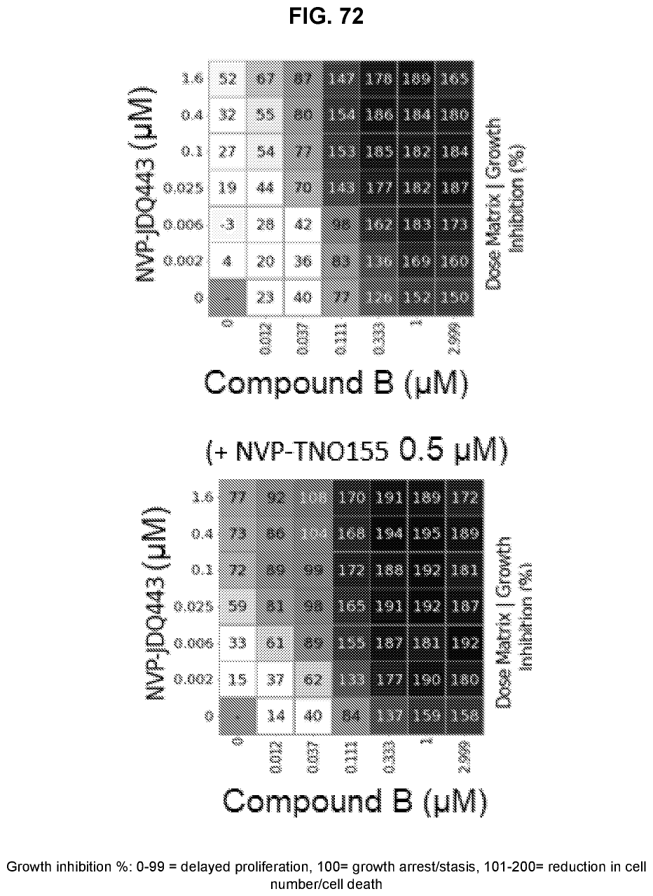

FIG. 73: In vitro viability of the colorectal cancer cell line LIM2099 was assessed using the CellTiterGlo following 7-day treatment with the YAP/TEAD inhibitor Compound B combined with the SHP2 inhibitor TNO155. Growth inhibition %: 0-99=delayed proliferation, 100=growth arrest/stasis, 101-200=reduction in cell number/cell death.

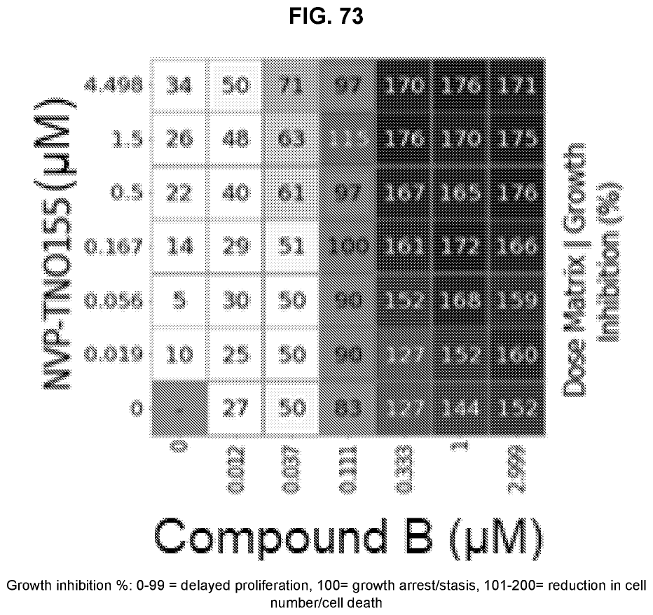

FIG. 74: In vitro viability of the colorectal cancer cell line LIM2099 was assessed using the CellTiterGlo following 7-day treatment with (top) the YAP/TEAD inhibitor Compound H combined with the KRAS G12C inhibitor JDQ-443 and (bottom) the YAP/TEAD inhibitor Compound H combined with the KRAS G12C inhibitor JDQ-443 and the SHP2 inhibitor TNO155. Growth inhibition %: 0-99=delayed proliferation, 100=growth arrest/stasis, 101-200=reduction in cell number/cell death.

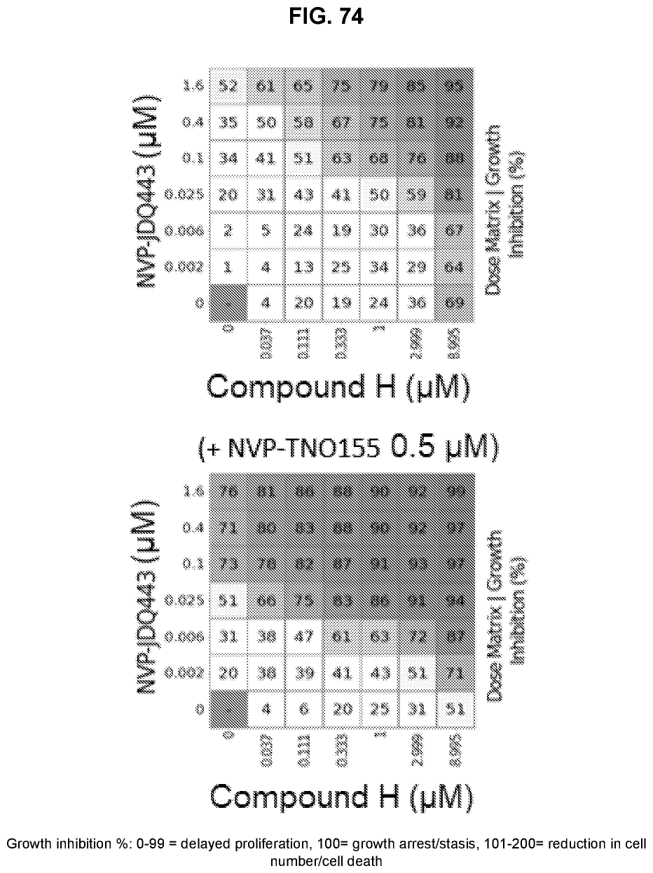

FIG. 75: In vitro viability of the colorectal cancer cell line LIM2099 was assessed using the CellTiterGlo following 7-day treatment with the YAP/TEAD inhibitor Compound H combined with the SHP2 inhibitor TNO155. Growth inhibition %: 0-99=delayed proliferation, 100=growth arrest/stasis, 101-200=reduction in cell number/cell death.

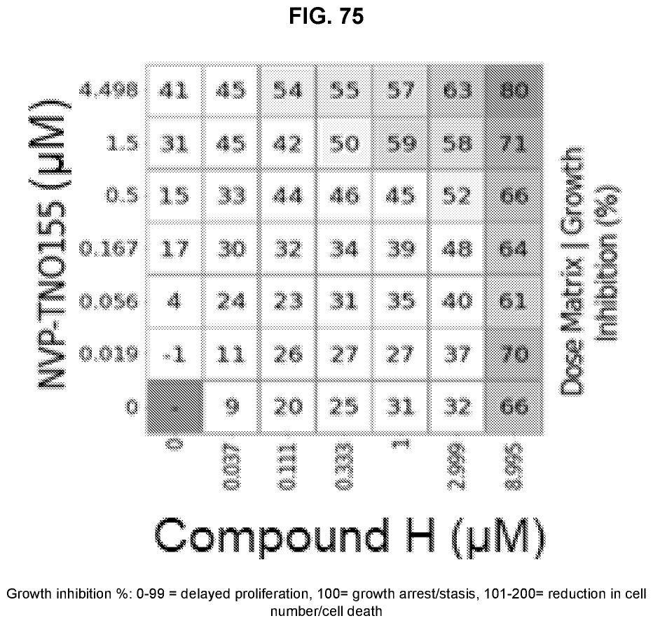

FIG. 76: In vitro viability of the lung cancer cell line NCI-H1792 was assessed using the CellTiterGlo following 7-day treatment with (top) the YAP/TEAD inhibitor Compound A combined with the CDK4/6 inhibitor NVP-LEE011 (LEE011 or ribociclib), and (bottom) the YAP/TEAD inhibitor Compound A combined with the CDK4/6 inhibitor NVP-LEE011 (LEE011 or ribociclib), and the KRAS G12C inhibitor JDQ443. Growth inhibition %: 0-99=delayed proliferation, 100=growth arrest/stasis, 101-200=reduction in cell number/cell death.

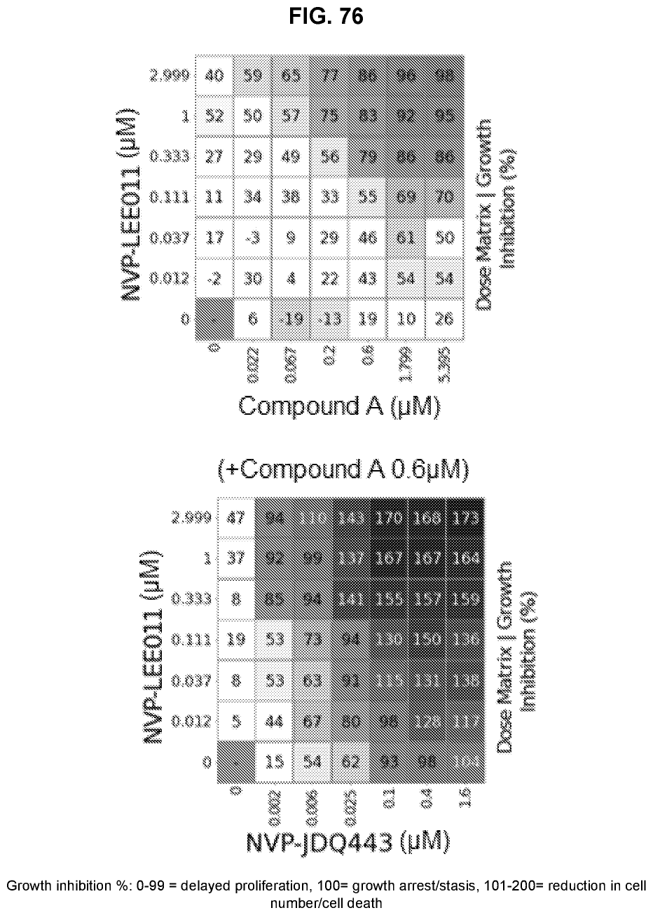

### DETAILED DESCRIPTION

As mentioned above, an object of the present invention is to find novel combination therapies, which selectively synergize in inhibiting proliferation and/or in inducing apoptosis.

The invention therefore provides the following numbered embodiments: Embodiment 1. A method of treating cancer in a subject in need thereof, wherein the method comprises administering to the subject a therapeutically effective amount of a TEAD inhibitor in combination with a first additional therapeutically active agent, and optionally a second additional therapeutically active agent, wherein the first additional therapeutically active agent and the second additional therapeutically active agent (where present) are independently selected from the group consisting of a KRAS G12/G13 inhibitor, a SHP2 inhibitor, an EGFR inhibitor, a PI3K inhibitor, a MEK inhibitor, an ERK inhibitor, an MDM2 inhibitor, a Raf-inhibitor, a CDK4/6 inhibitor and a cMET inhibitor.

Embodiment 2. A TEAD inhibitor for use in the treatment of cancer, wherein the treatment further comprises administration of a first additional therapeutically active agent, and optionally a second therapeutically active agent, wherein the first additional therapeutically active agent and the second additional therapeutically active agent (where present) are independently selected from the group consisting of a KRAS G12/G13 inhibitor, a SHP2 inhibitor, an EGFR inhibitor, a PI3K inhibitor, a MEK inhibitor, an ERK inhibitor, an MDM2 inhibitor, a Raf-inhibitor, a CDK4/6 inhibitor and a cMET inhibitor.

Embodiment 3. A combination comprising i) a TEAD inhibitor, ii) a first additional therapeutically active agent, and optionally iii) a second additional therapeutically active agent, wherein the first additional therapeutically active agent and the second additional therapeutically active agent (where present) are independently selected from the group consisting of a KRAS G12/G13 inhibitor, a SHP2 inhibitor, an EGFR inhibitor, a PI3K inhibitor, a MEK inhibitor, an ERK inhibitor, an MDM2 inhibitor, a Raf-inhibitor, a CDK4/6 inhibitor and a cMET inhibitor.

Embodiment 4. The method according to Embodiment 1 the TEAD inhibitor for use according to Embodiment 2, or the combination according to Embodiment 3, wherein the first additional therapeutically active agent is a KRAS G12/G13 inhibitor (e.g. a KRAS G12C inhibitor), e.g. where the second therapeutically active agent is absent.

Embodiment 5. The method according to Embodiment 4, the TEAD inhibitor for use according to Embodiment 4, or the combination according to Embodiment 4, wherein a second additional therapeutically active agent is present, and is a SHP2 inhibitor.

Embodiment 6. The method according to Embodiment 1, the TEAD inhibitor for use according to Embodiment 2, or the combination according to Embodiment 3, wherein the first additional therapeutically active agent is a SHP2 inhibitor, e.g. where the second therapeutically active agent is absent.

Embodiment 7. The method according to Embodiment 1, the TEAD inhibitor for use according to Embodiment 2, or the combination according to Embodiment 3, wherein the first additional therapeutically active agent is an EGFR inhibitor, e.g. where the second therapeutically active agent is absent.

Embodiment 8. The method according to Embodiment 1, the TEAD inhibitor for use according to Embodiment 2, or the combination according to Embodiment 3, wherein the first additional therapeutically active agent is a PI3K inhibitor, e.g. where the second therapeutically active agent is absent.

Embodiment 9. The method according to Embodiment 1, the TEAD inhibitor for use according to Embodiment 2, or the combination according to Embodiment 3, wherein the first additional therapeutically active agent is an MDM2 inhibitor, e.g. where the second therapeutically active agent is absent.

Embodiment 10. The method according to Embodiment 1, the TEAD inhibitor for use according to Embodiment 2, or the combination according to Embodiment 3, wherein the first additional therapeutically active agent is a CDK4/6 inhibitor, e.g. where the second therapeutically active agent is absent.

Embodiment 10a. The method according to Embodiment 10, the TEAD inhibitor for use according to Embodiment 10, or the combination according to Embodiment 10, wherein the first additional therapeutically active agent is a CDK416 inhibitor (e.g. ribociclib or a pharmaceutically acceptable salt thereof), and where the second therapeutically active agent is a KRAS G12C inhibitor (e.g. JDQ443 or a pharmaceutically acceptable salt thereof).

Embodiment 10b. The method according to Embodiment 10a, the TEAD inhibitor for use according to Embodiment 10a, or the combination according to Embodiment 10a, wherein the TEAD inhibitor is Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide) or a pharmaceutically acceptable salt.

Embodiment 10c. The method according to Embodiment 10a, the TEAD inhibitor for use according to Embodiment 10a, or the combination according to Embodiment 10a, wherein the TEAD inhibitor is Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) or a pharmaceutically acceptable salt thereof.

Embodiment 11. The method according to Embodiment 1, the TEAD inhibitor for use according to Embodiment 2, or the combination according to Embodiment 3, wherein the first additional therapeutically active agent is a MEK inhibitor e.g. where the second therapeutically active agent is absent.

Embodiment 12. The method according to Embodiment 1, the TEAD inhibitor for use according to Embodiment 2, or the combination according to Embodiment 3, wherein the first additional therapeutically active agent is an ERK inhibitor e.g. where the second therapeutically active agent is absent.

Embodiment 13. The method according to Embodiment 11 or Embodiment 12, the TEAD inhibitor for use according to Embodiment 11 or Embodiment 12, or the combination according to Embodiment 11 or Embodiment 12, wherein a second additional therapeutically active agent is present, and wherein the second additional therapeutically active agent is a Raf inhibitor.

Embodiment 14. The method according to Embodiment 1, the TEAD inhibitor for use according to Embodiment 2, or the combination according to Embodiment 3, wherein the first additional therapeutically active agent is a cMET inhibitor, e.g. where the second therapeutically active agent is absent.

Embodiment 15. A cMET inhibitor for use in the treatment of cancer, wherein the treatment further comprises administration of a TEAD inhibitor.

Embodiment 16. A KRAS G12/G13 inhibitor (e.g. a KRAS G12C inhibitor) for use in the treatment of cancer, wherein the treatment further comprises administration of a TEAD inhibitor.

Embodiment 17. The KRAS G12/G13 inhibitor for use according to Embodiment 16, wherein the treatment further comprises administration of a SHP2 inhibitor.

Embodiment 18. A SHP2 inhibitor for use in the treatment of cancer, wherein the treatment further comprises administration of a TEAD inhibitor.

Embodiment 19. The SHP2 inhibitor for use according to Embodiment 18, wherein the treatment further comprises administration of a KRAS G12/G13 inhibitor (e.g. a KRAS G12C inhibitor).

Embodiment 20. A MEK inhibitor for use in the treatment of cancer, wherein the treatment further comprises administration of a TEAD inhibitor.

Embodiment 21. An ERK inhibitor for use in the treatment of cancer, wherein the treatment further comprises administration of a TEAD inhibitor.

Embodiment 22. The MEK inhibitor for use according to Embodiment 20 or the ERK inhibitor for use according to Embodiment 21, wherein the treatment further comprises administration of a Raf inhibitor.

Embodiment 23. A Raf inhibitor for use in the treatment of cancer, wherein the treatment further comprises administration of a TEAD inhibitor.

Embodiment 24. The Raf inhibitor for use according to Embodiment 23, wherein the treatment further comprises administration of a MEK inhibitor.

Embodiment 25. The Raf inhibitor for use according to Embodiment 23, wherein the treatment further comprises administration of an ERK inhibitor.

Embodiment 26. An EGFR inhibitor for use in the treatment of cancer, wherein the treatment further comprises administration of a TEAD inhibitor.

Embodiment 27. A PI3K inhibitor for use in the treatment of cancer, wherein the treatment further comprises administration of a TEAD inhibitor.

Embodiment 28. An MDM2 inhibitor for use in the treatment of cancer, wherein the treatment further comprises administration of a TEAD inhibitor.

Embodiment 29. A CDK4/6 inhibitor for use in the treatment of cancer, wherein the treatment further comprises administration of a TEAD inhibitor.

Embodiment 30. The method according to any one of Embodiments 1 and 4 to 14, the TEAD inhibitor for use according to any one of Embodiments 2 and 4 to 14, the combination according to any one of claims 3 to 14, the cMET inhibitor for use according to Embodiment 15, the KRAS G12/G13 inhibitor for use according to Embodiment 16 or Embodiment 17, the SHP2 inhibitor for use according to Embodiment 18 or Embodiment 19, the MEK inhibitor for use according to Embodiment 20 or Embodiment 22, the ERK inhibitor for use according to Embodiment 21 or Embodiment 22, the Raf inhibitor for use according to any one of Embodiments 23 to 25, the EGFR inhibitor for use according to Embodiment 26, the PI3K inhibitor for use according to Embodiment 27, the MDM2 inhibitor for use according to Embodiment 28, or the CDK4/6 inhibitor for use according to Embodiment 29, wherein the TEAD inhibitor is a YAP/TAZ-TEAD protein-protein interaction inhibitor.

Embodiment 31. The method according to Embodiment 30, the TEAD inhibitor for use according to Embodiment 30, the combination according to Embodiment 30, the cMET inhibitor for use according to Embodiment 30, the KRAS G12/G13 inhibitor for use according to Embodiment 30, the SHP2 inhibitor for use according to Embodiment 30, the MEK inhibitor for use according to Embodiment 30, the ERK inhibitor for use according to Embodiment 30, the Raf inhibitor for use according to Embodiment 30, the EGFR inhibitor for use according to Embodiment 30, the PI3K inhibitor for use according to Embodiment 30, the MDM2 inhibitor for use according to Embodiment 30, or the CDK4/6 inhibitior for use according to Embodiment 30, wherein the TEAD inhibitor is a TEAD inhibitor of formula (I), or a pharmaceutically acceptable salt thereof, for example Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide) or a pharmaceutically acceptable salt thereof of or Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) or a pharmaceutically acceptable salt thereof.

Embodiment 31a. The method according to Embodiment 31, the TEAD inhibitor for use according to Embodiment 31, the combination according to Embodiment 31, the cMET inhibitor for use according to Embodiment 31, the KRAS G12/G13 inhibitor for use according to Embodiment 31, the SHP2 inhibitor for use according to Embodiment 31, the MEK inhibitor for use according to Embodiment 31, the ERK inhibitor for use according to Embodiment 31, the Raf inhibitor for use according to Embodiment 31, the EGFR inhibitor for use according to Embodiment 31, the PI3K inhibitor for use according to Embodiment 31, the MDM2 inhibitor for use according to Embodiment 31, or the CDK4/6 inhibitior for use according to Embodiment 31, wherein the TEAD inhibitor is Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide) or a pharmaceutically acceptable salt.

Embodiment 31 b. The method according to Embodiment 31, the TEAD inhibitor for use according to Embodiment 31, the combination according to Embodiment 31, the cMET inhibitor for use according to Embodiment 31, the KRAS G12/G13 inhibitor for use according to Embodiment 31, the SHP2 inhibitor for use according to Embodiment 31, the MEK inhibitor for use according to Embodiment 31, the ERK inhibitor for use according to Embodiment 31, the Raf inhibitor for use according to Embodiment 31, the EGFR inhibitor for use according to Embodiment 31, the PI3K inhibitor for use according to Embodiment 31, the MDM2 inhibitor for use according to Embodiment 31, or the CDK4/6 inhibitior for use according to Embodiment 31, wherein the TEAD inhibitor is Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) or a pharmaceutically acceptable salt.

Embodiment 31c. The method according to Embodiment 31, the TEAD inhibitor for use according to Embodiment 31, the combination according to Embodiment 31, the cMET inhibitor for use according to Embodiment 31, the KRAS G12/G13 inhibitor for use according to Embodiment 31, the SHP2 inhibitor for use according to Embodiment 31, the MEK inhibitor for use according to Embodiment 31, the ERK inhibitor for use according to Embodiment 31, the Raf inhibitor for use according to Embodiment 31, the EGFR inhibitor for use according to Embodiment 31, the PI3K inhibitor for use according to Embodiment 31, the MDM2 inhibitor for use according to Embodiment 31, or the CDK4/6 inhibitior for use according to Embodiment 31, wherein the TEAD inhibitor is

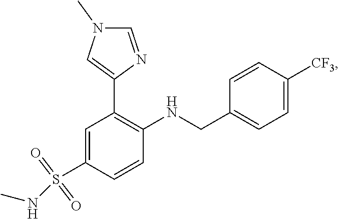 [SMILES: CNS(=O)(=O)c1ccc(NCc2ccc(C(F)(F)F)cc2)c(-c2cn(C)cn2)c1]

or a pharmaceutically acceptable salt thereof.

Embodiment 32. The method according to any one of Embodiments 1, 4, 5, 30 and 31, the TEAD inhibitor for use according to any one of Embodiments 2, 4, 5, 30 and 31, the combination according to any one of Embodiments 3 to 5, 30 and 31, the KRAS G12/G13 inhibitor for use according to any one of Embodiments 16, 17, 30 and 31, or the SHP2 inhibitor for use according to any one of Embodiments 19, 30 and 31, wherein the KRAS G12/G13 inhibitor is a KRAS G12C inhibitor selected from Compound C, sotorasib (Amgen), adagrasib (Mirati), D-1553 (InventisBio), B11701963 (Boehringer), GDC6036 (Roche), JNJ74699157 (J&J), X-Chem KRAS (X-Chem), LY3537982 (Lilly), B11823911 (Boehringer), AS KRAS G12C (Ascentage Pharma), SF KRAS G12C (Sanofi), RMC032 (Revolution Medicine), JAB-21822 (Jacobio Pharmaceuticals), AST-KRAS G12C (Allist Pharmaceuticals), AZ KRAS G12C (Astra Zeneca), NYU-12VC1 (New York University), and RMC6291 (Revolution Medicines), or a pharmaceutically acceptable salt thereof.

Embodiment 32a. The method according to Embodiment 32, the TEAD inhibitor for use according to Embodiment 32, the combination according to Embodiment 32, the KRAS G12/G13 inhibitor for use according to Embodiment 32, or the SHP2 inhibitor for use according to Embodiment 32, wherein the KRAS G12/G13 inhibitor is a KRAS G12C inhibitor selected from Compound C, sotorasib (Amgen) (also known as AMG510) and adagrasib (Mirati), or a pharmaceutically acceptable salt thereof.

Embodiment 33. The method according to Embodiment 32, the TEAD inhibitor for use according to Embodiment 32, the combination according to Embodiment 32, the KRAS G12/G13 inhibitor for use according to Embodiment 32, or the SHP2 inhibitor for use according to Embodiment 32, wherein the KRAS G12/G13 inhibitor is the KRAS G12C inhibitor Compound C (1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one) or sotorasib (AMG510), or a pharmaceutically acceptable salt thereof.

Embodiment 34. The method according to Embodiment 33, the TEAD inhibitor for use according to Embodiment 33, the combination according to Embodiment 33, the KRAS G12/G13 inhibitor for use according to Embodiment 33, or the SHP2 inhibitor for use according to Embodiment 33, wherein the KRAS G12/G13 inhibitor is the KRAS G12C inhibitor Compound C, or a pharmaceutically acceptable salt thereof.

Embodiment 34a. The method according to Embodiment 33, the TEAD inhibitor for use according to Embodiment 33, the combination according to Embodiment 33, the KRAS G12/G13 inhibitor for use according to Embodiment 33, or the SHP2 inhibitor for use according to Embodiment 33, wherein the KRAS G12/G13 inhibitor is sotorasib (AMG510), or a pharmaceutically acceptable salt thereof.

Embodiment 34b. The method according to Embodiment 32, the TEAD inhibitor for use according to Embodiment 32, the combination according to Embodiment 32, the KRAS G12/G13 inhibitor for use according to Embodiment 32, or the SHP2 inhibitor for use according to Embodiment 32, wherein the KRAS G12/G13 inhibitor is adagrasib, or a pharmaceutically acceptable salt thereof.

Embodiment 35. The method according to any one of Embodiments 1, 5, 6, and 30 to 34, the TEAD inhibitor for use according to any one of Embodiments 2, 5, 6, and 30 to 34, the combination according to any one of Embodiments 3, 5, 6 and 30 to 34, the KRAS G12/G13 inhibitor for use according to any one of Embodiments 17 and 30 to 34, or the SHP2 inhibitor for use according to any one of Embodiments 18, 19 and 30 to 34, wherein the SHP2 inhibitor is selected from the group consisting of TNO155 (Novartis), JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37), or a pharmaceutically acceptable salt thereof.

Embodiment 35a. The method according to Embodiment 35, the TEAD inhibitor for use according to Embodiment 35, the combination for use according to Embodiment 35, the KRAS G12/G13 inhibitor for use according to Embodiment 35 or the SHP2 inhibitor for use according to Embodiment 35 wherein the SHP2 inhibitor is selected from the group consisting of TNO155, RMC4550 and RMC4630, or a pharmaceutically acceptable salt thereof.

Embodiment 36. The method according to Embodiment 35, the TEAD inhibitor for use according to Embodiment 35, the combination for use according to Embodiment 35, the KRAS G12/G13 inhibitor for use according to Embodiment 35 or the SHP2 inhibitor for use according to Embodiment 35 wherein the SHP2 inhibitor is TNO155, or a pharmaceutically acceptable salt thereof.

Embodiment 36a. The method according to Embodiment 35, the TEAD inhibitor for use according to Embodiment 35, the combination for use according to Embodiment 35, the KRAS G12/G13 inhibitor for use according to Embodiment 35 or the SHP2 inhibitor for use according to Embodiment 35 wherein the SHP2 inhibitor is RMC4550 or RMC4630, or a pharmaceutically acceptable salt thereof.

Embodiment 37. The method according to any one of Embodiments 1, 14, 30 and 31, the TEAD inhibitor for use according to any one of Embodiments 2, 4, 30 and 31, the combination according to any one of Embodiments 3, 4, 30 and 31 or the cMET inhibitor for use according to Embodiment 15, wherein the cMET inhibitor is selected from the group consisting of crizotinib, capmatinib, tepotinib, AMG337, cabozantinib, savolitinib (AZD6094, HMPL-504), tivantinib, foretinib, volitinib, SU11274, PHA 665752, SGX523, BAY-853474, KRC-408, T-1840383, MK-2461, BMS-777607, JNJ-38877605, tivantinib (ARQ 197), PF-04217903, MGCD265, BMS-754807, BMS-794833, AMG-458, NVP-BVU972, AMG-208, golvatinib, norcantharidin, S49076, SAR125844, merestinib (LY2801653), onartuzumab, emibetuzumab, SAIT301, ABT-700, DN30, LY3164530, rilotumumab, ficlatuzumab, TAK701, and YYB-101, or a pharmaceutically acceptable salt thereof.

Embodiment 38. The method according to Embodiment 37, the TEAD inhibitor for use according to Embodiment 37, the combination according to Embodiment 37 or the cMET inhibitor for use according to Embodiment 37, wherein the cMET inhibitor is i) tepotinib, or ii) capmatinib, or a pharmaceutically acceptable salt thereof.

Embodiment 39. The method according to any one of Embodiments 1, 14, 30 and 31, the TEAD inhibitor for use according to any one of Embodiments 2, 4, 30 and 31, the combination according to any one of Embodiments 3, 4, 30 and 31 or the EGFR inhibitor for use according to Embodiment 26, wherein the EGFR inhibitor is selected from the group consisting of cetuximab, panitumuab, erlotinib, gefitinib, osimertinib and nazartinib, or a pharmaceutically acceptable salt thereof.

Embodiment 40. The method according to Embodiment 39, the TEAD inhibitor for use according to Embodiment 39, the combination according to Embodiment 39 or the EGFR inhibitor for use according to Embodiment 39, wherein the EGFR inhibitor is nazartinib (also known as EGF816), or a pharmaceutically acceptable salt thereof.

Embodiment 41. The method according to any one of Embodiments 1, 14, 30 and 31, the TEAD inhibitor for use according to any one of Embodiments 2, 4, 30 and 31, the combination according to any one of Embodiments 3, 4, 30 and 31 or the PI3K inhibitor for use according to Embodiment 27, wherein the PI3K inhibitor is selected from the group consisting of AMG511, buparlisib, Idelalisib, Copanlisib, Duvelisib, Alpelisib, QAU421 and Umbralisib, or a pharmaceutically acceptable salt thereof.

Embodiment 42. The method according to any one of Embodiments 1, 14, 30 and 31, the TEAD inhibitor for use according to any one of Embodiments 2, 4, 30 and 31, the combination according to any one of Embodiments 3, 4, 30 and 31 or the MDM2 inhibitor for use according to Embodiment 28, wherein the MDM2 inhibitor is selected from the group consisting of nutlin-3a, idasanutlin (also known as RG7388), RG7112, AMG-232 (also known as KRT-232), APG-115, BI-907828, milademetan and HDM201 (also known as siremadlin), or a pharmaceutically acceptable salt thereof.

Embodiment 43. The method according to Embodiment 42, the TEAD inhibitor for use according to Embodiment 42, the combination according to Embodiment 42, or the MDM2 inhibitor for use according to Embodiment 42, wherein the MDM2 inhibitor is HDM201, or a pharmaceutically acceptable salt thereof.

Embodiment 44. The method according to any one of Embodiments 1, 14, 30 and 31, the TEAD inhibitor for use according to any one of Embodiments 2, 4, 30 and 31, the combination according to any one of Embodiments 3, 4, 30 and 31 or the CDK4/6 inhibitor for use according to Embodiment 29 wherein the CDK4/6 inhibitor is selected from the group consisting of ribociclib, palbociclib and abemaciclib, or a pharmaceutically acceptable salt thereof.

Embodiment 45. The method according to Embodiment 44, the TEAD inhibitor for use according to Embodiment 44, the combination according to Embodiment 44, or the CDK4/6 inhibitor for use according to 44, wherein the CDK4/6 inhibitor is ribociclib, or a pharmaceutically acceptable salt thereof.

Embodiment 46. The method according to any one of Embodiments 1, 14, 30 and 31, the TEAD inhibitor for use according to any one of Embodiments 2, 4, 30 and 31, the combination according to any one of Embodiments 3, 4, 30 and 31 or the MEK inhibitor for use according to Embodiment 20 or Embodiment 22 or the Raf inhibitor for use according to Embodiment 24, wherein the MEK inhibitor is selected from the group consisting of pimasertib, PD-0325901, selumetinib, trametinib, binimetinib and cobimetinib, or a pharmaceutically acceptable salt thereof.

Embodiment 47. The method according to Embodiment 46, the TEAD inhibitor for use according to Embodiment 46, the combination according to Embodiment 46, the MEK inhibitor for use according to 46, or the Raf inhibitor for use according to Embodiment 46 wherein the MEK inhibitor is trametinib, or a pharmaceutically acceptable salt thereof.

Embodiment 48. The method according to any one of Embodiments 1, 14, 30 and 31, the TEAD inhibitor for use according to any one of Embodiments 2, 4, 30 and 31, the combination according to any one of Embodiments 3, 4, 30 and 31 or the ERK inhibitor for use according to Embodiment 21 or Embodiment 22, or the Raf inhibitor for use according to Embodiment 25 wherein the ERK inhibitor is selected from the group consisting of ulixertinib, GDC-0994, KO-947, Vtx-11e, SCH-772984, MK2853, LY3214996, MK08353, LTT462 and BVD-523, or a pharmaceutically acceptable salt thereof.

Embodiment 49. The method according to Embodiment 48, the TEAD inhibitor for use according to Embodiment 48, the combination according to Embodiment 48, the ERK inhibitor for use according to 48, or the Raf inhibitor for use according to Embodiment 48, wherein the ERK inhibitor is LTT462 (rineterkib) or ulixertinib, or a pharmaceutically acceptable salt thereof.

Embodiment 50. The method according to any one of Embodiments 1, 14, 30, 31 and 46 to 49, the TEAD inhibitor for use according to any one of Embodiments 2, 4, 30, 31 and 46 to 49, the combination according to any one of Embodiments 3, 4, 30, 31 and 46 to 49, the MEK inhibitor for use according to Embodiment 22, the ERK inhibitor for use according to Embodiment 22 or the Raf inhibitor for use according to any one of Embodiments 23 to 25, wherein the Raf inhibitor is selected from the group consisting of belvarafenib, naporafenib (also known as LXH254), Encorafenib, vemurafenib and dabrafenib, or a pharmaceutically acceptable salt thereof.

Embodiment 51. The method according to Embodiment 50, the TEAD inhibitor for use according to Embodiment 50, the combination according to Embodiment 50, the MEK inhibitor for use according to Embodiment 50, the ERK inhibitor for use according to Embodiment 50 or the Raf inhibitor for use according to Embodiment 50, wherein the Raf inhibitor is dabrafenib or LXH254 (naporafenib), or a pharmaceutically acceptable salt thereof.

Embodiment 52. The method according to any one of Embodiments 1, 4 to 14 and 30 to 51, the TEAD inhibitor for use according to any one of Embodiments 2, 4 to 14 and 30 to 51, the cMET inhibitor for use according to any one of Embodiments 15, 30, 31, 37 and 38, the KRAS G12/G13 inhibitor for use according to any one of Embodiments 16, 17 and 30 to 36, the SHP2 inhibitor for use according to any one of Embodiments 18, 19 and 30 to 36, the MEK inhibitor for use according to any one of Embodiments 20, 22, 30, 31, 46, 47, 50 and 51, the ERK inhibitor for use according to any one of Embodiments 21, 22, 30, 31 and 48 to 51, the Raf inhibitor for use according to any one of Embodiments 23 to 25, 30, 31, and 46 to 51, the EGFR inhibitor for use according to any one of Embodiments 26, 30, 31, 39 and 40, the PI3K inhibitor for use according to any one of Embodiments 27, 30, 31 and 41, the MDM2 inhibitor for use according to any one of Embodiments 28, 30, 31, 42 and 43, or the CDK4/6 inhibitor for use according to any one of Embodiments 29 to 31, 44 and 45, wherein the cancer is a TEAD dependent cancer.

Embodiment 53. The method according to any one of Embodiments 1, 4 to 14 and 30 to 52, the TEAD inhibitor for use according to any one of Embodiments 2, 4 to 14 and 30 to 52, the cMET inhibitor for use according to any one of Embodiments 15, 30, 31, 37, 38 and 52, the KRAS G12/G13 inhibitor for use according to any one of Embodiments 16, 17, 30 to 36 and 52, the SHP2 inhibitor for use according to any one of Embodiments 18, 19, 30 to 36, and 52 the MEK inhibitor for use according to any one of Embodiments 20, 22, 30, 31, 46, 47, and 50 to 52, the ERK inhibitor for use according to any one of Embodiments 21, 22, 30, 31 and 48 to 52, the Raf inhibitor for use according to any one of Embodiments 23 to 25, 30, 31, and 46 to 52, the EGFR inhibitor for use according to any one of Embodiments 26, 30, 31, 39, 40 and 52, the PI3K inhibitor for use according to any one of Embodiments 27, 30, 31, 41 and 52, the MDM2 inhibitor for use according to any one of Embodiments 28, 30, 31, 42, 43 and 52, or the CDK4/6 inhibitor for use according to any one of Embodiments 29 to 31, 44, 45 and 52, wherein the cancer is selected from the group consisting of breast cancer, lung cancer, ovarian cancer, kidney cancer, uterine cancer, colorectal cancer, malignant pleural mesothelioma, pancreatic cancer, prostate cancer, gastric cancer, gastrointestinal stromal tumor, esophageal cancer, liver cancer, medullobastoma, head and neck cancer, sarcoma, squamous cell carcinoma, epithelioid hemangioendothelioma, ependymal tumor and bone cancer, e.g. wherein the cancer is malignant pleural mesothelioma.

Embodiment 53a. The method according to Embodiment 53, the TEAD inhibitor for use according to Embodiment 53, the cMET inhibitor for use according to Embodiment 53, the KRAS G12/G13 inhibitor for use according to Embodiment 53, the SHP2 inhibitor for use according to Embodiment 53, the MEK inhibitor for use according to Embodiment 53, the ERK inhibitor for use according to Embodiment 53, the Raf inhibitor for use according to Embodiment 53, the EGFR inhibitor for use according to Embodiment 53, the PI3K inhibitor for use according to Embodiment 53, the MDM2 inhibitor for use according to Embodiment 53, or the CDK4/6 inhibitor for use according to Embodiment 53, wherein the cancer is colorectal cancer or lung cancer.

Embodiment 53b. The method according to any one of Embodiments 1, 4 to 14 and 30 to 53a, the TEAD inhibitor for use according to any one of Embodiments 2, 4 to 14 and 30 to 53a, the cMET inhibitor for use according to any one of Embodiments 15, 30, 31, 37, 38 and 53a, the KRAS G12/G13 inhibitor for use according to any one of Embodiments 16, 17, 30 to 36 and 53a, the SHP2 inhibitor for use according to any one of Embodiments 18, 19, 30 to 36, and 53a the MEK inhibitor for use according to any one of Embodiments 20, 22, 30, 31, 46, 47, and 50 to 53a, the ERK inhibitor for use according to any one of Embodiments 21, 22, 30, 31 and 48 to 53a, the Raf inhibitor for use according to any one of Embodiments 23 to 25, 30, 31, and 46 to 53a, the EGFR inhibitor for use according to any one of Embodiments 26, 30, 31, 39, 40 and 53a, the PI3K inhibitor for use according to any one of Embodiments 27, 30, 31, 41 and 53a, the MDM2 inhibitor for use according to any one of Embodiments 28, 30, 31, 42, 43 and 53a, or the CDK4/6 inhibitor for use according to any one of Embodiments 29 to 31, 44, 45 and 53a, wherein the cancer is a KRAS G12C mutant cancer.

Embodiment 54. The method according to any one of Embodiments 1, 4 to 14 and 30 to 53, the TEAD inhibitor for use according to any one of Embodiments 2, 4 to 14 and 30 to 53, the cMET inhibitor for use according to any one of Embodiments 15, 30, 31, 37, 38, 52 and 53 the KRAS G12/G13 inhibitor for use according to any one of Embodiments 16, 17, 30 to 36, 52 and 53, the SHP2 inhibitor for use according to any one of Embodiments 18, 19, 30 to 36, 52 and 53, the MEK inhibitor for use according to any one of Embodiments 20, 22, 30, 31, 46, 47 and 50 to 53, the ERK inhibitor for use according to any one of Embodiments 21, 22, 30, 31 and 48 to 53, the Raf inhibitor for use according to any one of Embodiments 23 to 25, 30, 31, and 46 to 53, the EGFR inhibitor for use according to any one of Embodiments 26, 30, 31, 39, 40, 52 and 53, the PI3K inhibitor for use according to any one of Embodiments 27, 30, 31, 41, 52 and 53, the MDM2 inhibitor for use according to any one of Embodiments 28, 30, 31, 42, 43, 52 and 53, or the CDK4/6 inhibitor for use according to any one of Embodiments 29 to 31, 44, 45, 52 and 53 wherein the TEAD inhibitor is administered on each of the first 3 days of a 7 day treatment cycle, and wherein the treatment is composed of at least two treatment cycles.

Embodiment 55. The method according to Embodiment 54, the TEAD inhibitor according to Embodiment 54, the cMET inhibitor according to Embodiment 54, the KRAS G12/G13 inhibitor for use according to Embodiment 54, the SHP2 inhibitor for use according to Embodiment 54, the MEK inhibitor for use according to Embodiment 54, the ERK inhibitor for use according to Embodiment 54, the Raf inhibitor for use according to Embodiment 54, the EGFR inhibitor for use according to Embodiment 54, the PI3K inhibitor for use according to Embodiment 54, the MDM2 inhibitor for use according to Embodiment 54, or the CDK416 inhibitor for use according to Embodiment 54, wherein the daily dose of the TEAD inhibitor on each administration day is from 15 mg to 100 mg.

Embodiment 56. The method according to Embodiment 55, the TEAD inhibitor according to Embodiment 55, the cMET inhibitor according to Embodiment 55, the KRAS G12/G13 inhibitor for use according to Embodiment 55, the SHP2 inhibitor for use according to Embodiment 54, the MEK inhibitor for use according to Embodiment 55, the ERK inhibitor for use according to Embodiment 55, the Raf inhibitor for use according to Embodiment 55, the EGFR inhibitor for use according to Embodiment 55, the PI3K inhibitor for use according to Embodiment 55, the MDM2 inhibitor for use according to Embodiment 55, or the CDK4/6 inhibitor for use according to Embodiment 55, wherein the daily dose of the TEAD inhibitor on each administration day is 15, 30, 45, 60, 75 mg, 90 mg or 100 mg.

It has been determined that the combinations of the invention could be used to efficiently treat cancer, e.g. due to a synergistic effect in inhibition of cell proliferation and/or induction of apoptosis.

In an embodiment of any one of the aspects of the invention, the TEAD inhibitor is a YAP/TAZ-TEAD protein-protein interaction inhibitor.

In an embodiment of any one of the aspects of the invention, the TEAD inhibitor is a TEAD inhibitor of formula (I), or a pharmaceutically acceptable salt thereof, for example Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide) or a pharmaceutically acceptable salt thereof of or Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) or a pharmaceutically acceptable salt thereof,

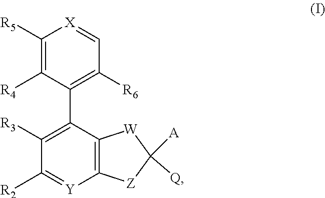

wherein
W is selected from O; and CH-R_{w};
X is selected from CH; and N;
Y is selected from CH; and N;
Z is selected from CH₂; O; and NH;
wherein when Y is N, W is CH-R_{w}, and Z is O;
A is selected from
(i) phenyl, which phenyl is optionally substituted with halo; or haloC₁-C₃alkoxy;
(ii) a 5- or 6-membered aromatic heterocyclic ring comprising at least one heteroatom selected from N, O, and S, which aromatic heterocyclic ring is optionally substituted with hydroxy; C₁-C₃alkoxy; or oxo; and
(iii) a halobenzodioxole moiety of formula

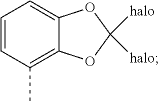

R_{w} is selected from (i) hydrogen; (ii) hydroxy; (iii) C₁-C₃alkoxy; (iv) hydroxy-C₁-C₃alkyl; (v) C₁-C₃alkyl; and (vi) C₁-C₃alkoxy-C₁-C₃alkyl; Q is selected from (i) —C(R⁷)₂-N(R⁸)-R₁; (ii) 9- or 10-membered partially saturated heteroaryl comprising at least one N heteroatom; and (iii) 4-, 5- or 6-membered saturated heterocyclic ring comprising at least one heteroatom or heteroatom group selected from N, O, S, —S(═O) and —S(═O)₂, with the proviso that at least one N heteroatom is present, wherein the heterocyclic ring is unsubstituted or substituted with one or more substituents independently selected from hydroxy, C₁-C₃alkyl, C₁-C₃alkoxy, halo and C₁-C₃alkylene forming a bridge between two ring atoms of the saturated heterocyclic ring, thus forming a bridged bicyclic structure;
R₁ is selected from (i) hydrogen; (ii) C₁-C₆alkyl which is optionally deuterated; and (iii) (CH₂)₀₋₂R₁ₐ;
R₁ₐ is selected from (i) hydroxyC₁-C₄alkyl; (ii) C₁-C₃alkoxy; (iii) a 5- or 6-membered saturated heterocyclic ring comprising at least one heteroatom selected from N and O, which saturated heterocyclic ring is optionally substituted once or more than once independently with C₁-C₃alkyl; (CH₂)₀₋₁C(O)di(C₁-C₃alkyl)amino; SO₂C₁-C₃alkyl; C(O)C₁-C₃alkyl; or oxo; (iv) C₃-C₆cycloalkyl optionally substituted once or more than once independently with hydroxy; hydroxyC₁-C₄alkyl; C₁-C₆alkoxy; C(O)OC₁-C₃alkyl; CO₂H; SO₂C₁-C₃alkyl; haloC₁-C₃alkyl; NHR^{1b}; (CH₂)₀₋₁C(O)NR^{1c}R^{1d}; C₁-C₆alkyl; haloC₁-C₃alkoxy-C₁-C₃alkyl; halo; a 5- or 6-membered aromatic heterocyclic ring comprising at least one heteroatom selected from N, O, and S; or with two R^{1e} groups, wherein the two R^{1e} attached at the same carbon atom form together with the carbon atom to which they are attached a 5-membered saturated heterocyclic ring comprising at least one heteroatom selected from N and O, or a C₃-C₆cycloalkyl, which saturated heterocyclic ring or cycloalkyl are optionally substituted with hydroxy or oxo; R^{1b} is selected from (i) C(O)C₁-C₃alkyl; and (ii) SO₂C₁-C₃alkyl; R^{1c} and R^{1d} are each independently selected from (i) hydrogen; (ii) C₁-C₃alkyl; and (iii) hydroxyC₁-C₄alkyl;
R₂ is selected from (i) hydrogen; and (ii) halo;
R₃ is selected from (i) halo; (ii) haloC₁-C₃alkyl; and (iii) cyano;
R₄ is selected from (i) hydrogen; (ii) halo; and (iii) C₁-C₃alkyl;
R₅ is selected from (i) hydrogen; (ii) C₁-C₆alkoxy optionally substituted with C₃-C₆cycloalkyl; CO₂H; SO₂C₁-C₃alkyl; a 5- or 6-membered aromatic heterocyclic ring comprising at least one heteroatom selected from N, O, and S; or a 5- or 6-membered saturated heterocyclic ring comprising at least one heteroatom selected from N and O, which ring is optionally substituted with C(O)C₁-C₃alkyl;
(iii) halo; (iv) hydroxyC₁-C₆alkoxy, wherein the alkoxy is optionally deuterated; (v) haloC₁-C₆alkoxy optionally substituted with hydroxy; (vi)S-haloC₁-C₃alkyl optionally substituted with hydroxy; (vii) C₁-C₃alkoxyC₁-C₃alkoxy; (viii) NR^{5a}R^{5b}; (ix) C₁-C₃alkyl; (x) a 5- or 6-membered aromatic heterocyclic ring comprising at least one heteroatom selected from N, O, and S; and (xi) hydroxy;
R^{5a} and R^{5b} are each independently selected from (i) hydrogen; and (ii) C₁-C₃alkyl; or
R^{5a} and R^{5b} together with the nitrogen atom to which they are attached form a 5- or 6-membered saturated heterocyclic ring, which saturated heterocyclic ring optionally in addition carries a hydroxy group;
R₆ is selected from (i) hydrogen; (ii) cyano; (iii) C(O)NHR^{6a}; (iv) NHR^{6b}; and (v) C₁-C₃alkoxy substituted with NH₂ or hydroxy;
R^{6a} is selected from (i) hydrogen; (ii) C₁-C₃alkyl; (iii) C₃-C₆cycloalkyl; (iv) a 5- or 6-membered aromatic heterocyclic ring comprising at least one heteroatom selected from N, O, and S which aromatic heterocyclic ring is optionally substituted with C₁-C₃alkyl;
R^{6b} is C₁-C₃alkyl substituted with NH₂ or hydroxy;
R⁷ is each independently selected from hydrogen and C₁-C₃alkyl; and
R⁶ is hydrogen or C₁-C₃-alkyl.

In an embodiment of any one of the aspects of the invention, where a KRAS G12/G13 inhibitor is present, the KRAS G12/G13 inhibitor is a KRAS G12C inhibitor.

In an embodiment of any one of the aspects of the invention, where a KRAS G12/G13 inhibitor is present, the the KRAS G12/G13 inhibitor is a KRAS G12C inhibitor selected from Compound C, sotorasib (Amgen), adagrasib (Mirati), D-1553 (InventisBio), B11701963 (Boehringer), GDC6036 (Roche), JNJ74699157 (J&J), X-Chem KRAS (X-Chem), LY3537982 (Lilly), B11823911 (Boehringer), AS KRAS G12C (Ascentage Pharma), SF KRAS G12C (Sanofi), RMC032 (Revolution Medicine), JAB-21822 (Jacobio Pharmaceuticals), AST-KRAS G12C (Allist Pharmaceuticals), AZ KRAS G12C (Astra Zeneca), NYU-12VC1 (New York University), and RMC6291 (Revolution Medicines), or a pharmaceutically acceptable salt thereof.

In an embodiment of any one of the aspects of the invention, where a KRAS G12/G13 inhibitor is present, the KRAS G12/G13 inhibitor is the KRAS G12C inhibitor Compound C (1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one) or AMG510, or a pharmaceutically acceptable salt thereof. 25 In an embodiment of any one of the aspects of the invention, where a KRAS G12/G13 inhibitor is present, the KRAS G121G13 inhibitor is the KRAS G12C inhibitor Compound C, or a pharmaceutically acceptable salt thereof.

In an embodiment of any one of the aspects of the invention, where a SHP2 inhibitor is present, the SHP2 inhibitor is selected from the group consisting of TNO155 (Novartis), JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37), or a pharmaceutically acceptable salt thereof.

In an embodiment of any one of the aspects of the invention, where a SHP2 inhibitor is present, the SHP2 inhibitor is TNO155, or a pharmaceutically acceptable salt thereof.

In an alternative embodiment, the SHP2 inhibitor, where present, is RMC-4550 or a pharmaceutically acceptable salt thereof,

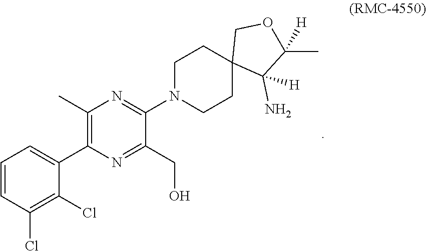 [SMILES: Cc1nc(N2CCC3(CC2)CO[C@@H](C)[C@H]3N)c(CO)nc1-c1cccc(Cl)c1Cl]

In an alternative embodiment, the SHP2 inhibitor, where present, is RMC-4630 or a pharmaceutically acceptable salt thereof,

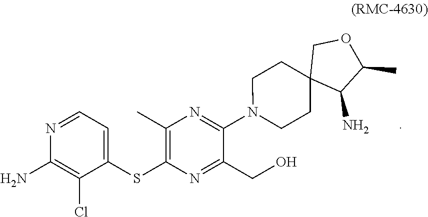 [SMILES: Cc1nc(N2CCC3(CC2)CO[C@@H](C)[C@H]3N)c(CO)nc1Sc1ccnc(N)c1Cl]

In an embodiment of any one of the aspects of the invention, where a cMET inhibitor is present, the cMET inhibitor is selected from the group consisting of crizotinib, capmatinib, tepotinib, AMG337, cabozantinib, savolitinib (AZD6094, HMPL-504), tivantinib, foretinib, volitinib, SU11274, PHA 665752, SGX523, BAY-853474, KRC-408, T-1840383, MK-2461, BMS-777607, JNJ-38877605, tivantinib (ARQ 197), PF-04217903, MGCD265, BMS-754807, BMS-794833, AMG-458, NVP-BVU972, AMG-208, golvatinib, norcantharidin, S49076, SAR125844, merestinib (LY2801653), onartuzumab, emibetuzumab, SAIT301, ABT-700, DN30, LY3164530, rilotumumab, ficlatuzumab, TAK701, and YYB-101, or a pharmaceutically acceptable salt thereof.

In an embodiment of any one of the aspects of the invention, where a cMET inhibitor is present, the cMET inhibitor is i) tepotinib, or ii) capmatinib, or a pharmaceutically acceptable salt thereof.

In an embodiment of any one of the aspects of the invention, where an EGFR inhibitor is present, the EGFR inhibitor is selected from the group consisting of cetuximab, panitumuab, erlotinib, gefitinib, osimertinib and nazartinib, or a pharmaceutically acceptable salt thereof.

In an embodiment of any one of the aspects of the invention, where an EGFR inhibitor is present, the EGFR inhibitor is nazartinib (also known as EGF816), or a pharmaceutically acceptable salt thereof.

In an embodiment of any one of the aspects of the invention, where a PI3K inhibitor is present, the PI3K inhibitor is selected from the group consisting of AMG511, buparlisib, Idelalisib, Copanlisib, Duvelisib, Alpelisib, QAU421 and Umbralisib, or a pharmaceutically acceptable salt thereof.

In an embodiment of any one of the aspects of the invention, where an MDM2 inhibitor is present, the MDM2 inhibitor is selected from the group consisting of nutlin-3a, idasanutlin (also known as RG7388), RG7112, AMG-232 (also known as KRT-232), APG-115, BI-907828, milademetan and HDM201 (also known as siremadlin), or a pharmaceutically acceptable salt thereof.

In an embodiment of any one of the aspects of the invention, where an MDM2 inhibitor is present, the MDM2 inhibitor is HDM201, or a pharmaceutically acceptable salt thereof.

In an embodiment of any one of the aspects of the invention, where a CDK4/6 inhibitor is present, the CDK4/6 inhibitor is selected from the group consisting of ribociclib, palbociclib and abemaciclib, or a pharmaceutically acceptable salt thereof.

In an embodiment of any one of the aspects of the invention, where a CDK4/6 inhibitor is present, the CDK4/6 inhibitor is ribociclib, or a pharmaceutically acceptable salt thereof.

In an embodiment of any one of the aspects of the invention, where a MEK inhibitor is present, the MEK inhibitor is selected from the group consisting of pimasertib, PD-0325901, selumetinib, trametinib, binimetinib and cobimetinib, or a pharmaceutically acceptable salt thereof.

In an embodiment of any one of the aspects of the invention, where a MEK inhibitor is present, the MEK inhibitor is trametinib, or a pharmaceutically acceptable salt thereof.

In an embodiment of any one of the aspects of the invention, where an ERK inhibitor is present, the ERK inhibitor is selected from the group consisting of ulixertinib, GDC-0994, KO-947, Vtx-11e, SCH-772984, MK2853, LY3214996, MK08353, LTT462 and BVD-523, or a pharmaceutically acceptable salt thereof.

In an embodiment of any one of the aspects of the invention, where an ERK inhibitor is present, the ERK inhibitor is LTT462 (rineterkib) or ulixertinib, or a pharmaceutically acceptable salt thereof.

In an embodiment of any one of the aspects of the invention, where a Raf inhibitor is present, the Raf inhibitor is selected from the group consisting of belvarafenib, naporafenib (also known as LXH254), Encorafenib, vemurafenib and dabrafenib, or a pharmaceutically acceptable salt thereof.

In an embodiment of any one of the aspects of the invention, where a Raf inhibitor is present, the Raf inhibitor is dabrafenib or LXH254 (naporafenib), or a pharmaceutically acceptable salt thereof.

In an embodiment, the cancer is a TEAD dependent cancer.

In an embodiment, the cancer is selected from the group consisting of breast cancer, lung cancer, ovarian cancer, kidney cancer, uterine cancer, colorectal cancer, malignant pleural mesothelioma, pancreatic cancer, prostate cancer, gastric cancer, gastrointestinal stromal tumor, esophageal cancer, liver cancer, medullobastoma, head and neck cancer, sarcoma, squamous cell carcinoma, epithelioid hemangioendothelioma, ependymal tumor and bone cancer.

In an embodiment, the TEAD inhibitor is administered on each of the first 3 days of a 7 day treatment cycle, and wherein the treatment is composed of at least two treatment cycles. In an embodiment, the daily dose of the TEAD inhibitor on each administration day is from 15 mg to 100 mg. In an embodiment, the daily dose of the TEAD inhibitor on each administration day is 15, 30, 45, 60, 75 mg, 90 mg or 100 mg.

### Definitions

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, patients, cancers and the like, this is taken to mean also a single compound, patient, or the like.

References in this specification to “the invention” are intended to reflect embodiments of the several inventions disclosed in this specification, and should not be taken as unnecessarily limiting of the claimed subject matter.

The term “synergistic effect” as used herein refers to action of two or three therapeutic agents producing an effect, for example, slowing the progression of a proliferative disease, particularly cancer, or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median effect equation (Chou, T. C. and Talalay, P., Adv. Enzyme Regul. 22: 27-55 (1984)). Each equation referred to above can be applied to experimental data to generate a corresponding graph to aid in assessing the effects of a drug combination. The corresponding graphs associated with the equations referred to above are the concentration-effect curve, isobologram curve and combination index curve, respectively.

The term “pharmaceutically acceptable salts” refers to salts that retain the biological effectiveness and properties of the compound and which typically are not biologically or otherwise undesirable. The compound may be capable of forming acid addition salts by virtue of the presence of an amino group.

Unless otherwise specified, or clearly indicated by the text, reference to therapeutic agents useful in the pharmaceutical combination of the present invention includes both the free base of the compounds, and all pharmaceutically acceptable salts of the compounds.

The term “combination” or “pharmaceutical combination” is defined herein to refer to either a fixed combination in one dosage unit form, a non-fixed combination or a kit of parts for the combined administration where the therapeutic agents may be administered together, independently at the same time or separately within time intervals, which preferably allows that the combination partners show a cooperative, e.g. synergistic effect. Thus, the single compounds of the pharmaceutical combination of the present invention could be administered simultaneously or sequentially.

Furthermore, the pharmaceutical combination of the present invention may be in the form of a fixed combination or in the form of a non-fixed combination.

The term “fixed combination” means that the therapeutic agents, e.g., the single compounds of the combination, are in the form of a single entity or dosage form.

The term “non-fixed combination” means that the therapeutic agents, e.g., the single compounds of the combination, are administered to a patient as separate entities or dosage forms either simultaneously or sequentially with no specific time limits, wherein preferably such administration provides therapeutically effective levels of the two therapeutic agents in the body of the subject, e.g., a mammal or human in need thereof.

The pharmaceutical combinations can further comprise at least one pharmaceutically acceptable carrier. Thus, the present invention relates to a pharmaceutical composition comprising the pharmaceutical combination of the present invention and at least one pharmaceutically acceptable carrier.

As used herein, the term “carrier” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Generally, the term “pharmaceutical composition” is defined herein to refer to a mixture or solution containing at least one therapeutic agent to be administered to a subject, e.g., a mammal or human. The present pharmaceutical combinations can be formulated in a suitable pharmaceutical composition for enteral or parenteral administration are, for example, those in unit dosage forms, such as sugar-coated tablets, tablets, capsules or suppositories, or ampoules. If not indicated otherwise, these are prepared in a manner known per se, for example by means of various conventional mixing, comminution, direct compression, granulating, sugar-coating, dissolving, lyophilizing processes, or fabrication techniques readily apparent to those skilled in the art. It will be appreciated that the unit content of a combination partner contained in an individual dose of each dosage form need not in itself constitute an effective amount since the necessary effective amount may be reached by administration of a plurality of dosage units. The pharmaceutical composition may contain, from about 0.1% to about 99.9%, preferably from about 1% to about 60%, of the therapeutic agent(s). One of ordinary skill in the art may select one or more of the aforementioned carriers with respect to the particular desired properties of the dosage form by routine experimentation and without any undue burden. The amount of each carriers used may vary within ranges conventional in the art. The following references disclose techniques and excipients used to formulate oral dosage forms. See The Handbook of Pharmaceutical Excipients, 4th edition, Rowe et al., Eds., American Pharmaceuticals Association (2003); and Remington: the Science and Practice of Pharmacy, 20th edition, Gennaro, Ed., Lippincott Williams & Wilkins (2003). These optional additional conventional carriers may be incorporated into the oral dosage form either by incorporating the one or more conventional carriers into the initial mixture before or during granulation or by combining the one or more conventional carriers with granules comprising the combination of agents or individual agents of the combination of agents in the oral dosage form. In the latter embodiment, the combined mixture may be further blended, e.g., through a V-blender, and subsequently compressed or molded into a tablet, for example a monolithic tablet, encapsulated by a capsule, or filled into a sachet. Clearly, the pharmaceutical combinations of the present invention can be used to manufacture a medicine.

The present invention relates to such pharmaceutical combinations or pharmaceutical compositions that are particularly useful as a medicine.

Specifically, the combinations or compositions of the present invention can be applied in the treatment of cancer.

The present invention also relates to use of pharmaceutical combinations or pharmaceutical compositions of the present invention for the preparation of a medicament for the treatment of a cancer, and to a method for treating cancer in a subject in need thereof comprising administering to the subject a therapeutically effective amount of a pharmaceutical combination according to the present invention, or the pharmaceutical composition according to the present invention.

The term “treatment” as used herein comprises a treatment relieving, reducing or alleviating at least one symptom in a subject, increasing progression-free survival, overall survival, extending duration of response or delaying progression of a disease. For example, treatment can be the diminishment of one or several symptoms of a disorder or complete eradication of a disorder, such as cancer. Within the meaning of the present invention, the term “treatment” also denotes to arrest, delay the onset (i.e., the period prior to clinical manifestation of a disease) and/or reduce the risk of developing or worsening a disease in a patient, e.g., a mammal, particularly the patient is a human. The term “treatment” as used herein comprises an inhibition of the growth of a tumor incorporating a direct inhibition of a primary tumor growth and/or the systemic inhibition of metastatic cancer cells.

A “subject,” “individual” or “patient” is used interchangeably herein, which refers to a vertebrate, preferably a mammal, more preferably a human. Mammals include, but are not limited to, mice, simians, humans, farm animals, sport animals, and pets.

The term “a therapeutically effective amount” of a compound (e.g. chemical entity or biologic agent) of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one embodiment a therapeutically effective amount in vivo may range depending on the route of administration, between about 0.1-500 mg/kg, or between about 1-100 mg/kg.

As used herein, the term “inhibit”, “inhibition” or “inhibiting” refers to the reduction or suppression of a given condition, symptom, or disorder, or disease, or a significant decrease in the baseline activity of a biological activity or process.

The optimal dosage of each combination partner for treatment of a cancer can be determined empirically for each individual using known methods and will depend upon a variety of factors, including, though not limited to, the degree of advancement of the disease; the age, body weight, general health, gender and diet of the individual; the time and route of administration; and other medications the individual is taking. Optimal dosages may be established using routine testing and procedures that are well known in the art. The amount of each combination partner that may be combined with the carrier materials to produce a single dosage form will vary depending upon the individual treated and the particular mode of administration. In some embodiments the unit dosage forms containing the combination of agents as described herein will contain the amounts of each agent of the combination that are typically administered when the agents are administered alone.

Frequency of dosage may vary depending on the compound used and the particular condition to be treated or prevented. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

As used herein the term “TEAD dependent cancer” refers to any cancer in which TEAD (i.e. TEAD1, TEAD2, TEAD3 and/or TEAD4,), or a mutant or variant thereof, is known to be relevant, for example, in cancers where the Hippo pathway is genetically altered.

As used herein, the term “TEAD inhibitor” refers to a compound which has activity as an inhibitor of TEAD (i.e. TEAD1, TEAD2, TEAD3 and/or TEAD4), or a mutant or variant thereof, that can be assayed in vitro, in vivo or in a cell line. In an example, IC₅₀ [μM] is <10, for example <5, for example <2, for example <1, for example <0.5, for example <0.2, for example <0.1, in the biochemical assay as described in the Examples, and/or the reporter gene cellular assay as described in the Examples, and/or the proliferation cellular assay as described in the Examples.

A YAP/TAZ-TEAD protein-protein interaction inhibitor as described herein refers to a TEAD inhibitor which inhibits TEAD activity by inhibiting the interaction of the YAP/TAZ complex with TEAD. Hyperactivation of YAP/TAZ, resulting in the activation of TEAD, has been reported in many cancers, e.g. malignant pleural mesothelioma. Thus inhibiting the interaction between YAP/TAZ and TEAD is a promising mechanism by which to inhibit TEAD activity.

YAP/TAZ-TEAD protein-protein interaction inhibitors of the invention include, but are not limited to, compounds of formula (I), the synthesis of which is described herein.

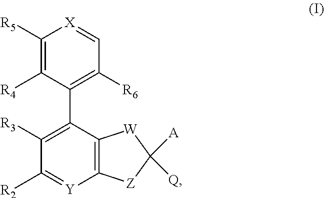

wherein
W is selected from O; and CH-R_{w};
X is selected from CH; and N;
Y is selected from CH; and N;
Z is selected from CH₂; O; and NH;
wherein when Y is N, W is CH-R_{w}, and Z is O;
A is selected from
(i) phenyl, which phenyl is optionally substituted with halo; or haloC₁-C₃alkoxy;
(ii) a 5- or 6-membered aromatic heterocyclic ring comprising at least one heteroatom selected from N, O, and S, which aromatic heterocyclic ring is optionally substituted with hydroxy; C₁-C₃alkoxy; or oxo; and
(iii) a halobenzodioxole moiety of formula

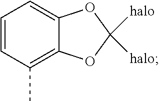

R_{w} is selected from (i) hydrogen; (ii) hydroxy; (iii) C₁-C₃alkoxy; (iv) hydroxy-C₁-C₃alkyl; (v) C₁-C₃alkyl; and (vi) C₁-C₃alkoxy-C₁-C₃alkyl;
Q is selected from (i) —C(R⁷)₂-N(R⁸)-R¹; (ii) 9- or 10-membered partially saturated heteroaryl comprising at least one N heteroatom; and (iii) 4-, 5- or 6-membered saturated heterocyclic ring comprising at least one heteroatom or heteroatom group selected from N, O, S, —S(═O) and —S(═O)₂, with the proviso that at least one N heteroatom is present, wherein the heterocyclic ring is unsubstituted or substituted with one or more substituents independently selected from hydroxy, C₁-C₃alkyl, C₁-C₃alkoxy, halo and C₁-C₃alkylene forming a bridge between two ring atoms of the saturated heterocyclic ring, thus forming a bridged bicyclic structure;
R¹ is selected from (i) hydrogen; (ii) C₁-C₆alkyl which is optionally deuterated; and (iii) (CH₂)₀₋₂R₁ₐ; R₁ₐ is selected from (i) hydroxyC₁-C₄alkyl; (ii) C₁-C₃alkoxy; (iii) a 5- or 6-membered saturated heterocyclic ring comprising at least one heteroatom selected from N and O, which saturated heterocyclic ring is optionally substituted once or more than once independently with C₁-C₃alkyl; (CH₂)₀₋₁C(O)di(C₁-C₃alkyl)amino; S02C₁-C₃alkyl; C(O)C₁-C₃alkyl; or oxo; (iv) C₃-C₆cycloalkyl optionally substituted once or more than once independently with hydroxy; hydroxyC₁-C₄alkyl; C₁-C₆alkoxy; C(O)OC₁-C₃alkyl; CO₂H; SO₂C₁-C₃alkyl; haloC₁-C₃alkyl; NHR^{1b}; (CH₂)₀₋₁C(O)NR^{1c}R^{1d}; C₁-C₆alkyl; haloC₁-C₃alkoxy-C₁-C₃alkyl; halo; a 5- or 6-membered aromatic heterocyclic ring comprising at least one heteroatom selected from N, O, and S; or with two R^{1e} groups, wherein the two R^{1e} attached at the same carbon atom form together with the carbon atom to which they are attached a 5-membered saturated heterocyclic ring comprising at least one heteroatom selected from N and O, or a C₃-C₆cycloalkyl, which saturated heterocyclic ring or cycloalkyl are optionally substituted with hydroxy or oxo;
R^{1b} is selected from (i) C(O)C₁-C₃alkyl; and (ii) SO₂C₁-C₃alkyl;
R^{1c} and R^{1d} are each independently selected from (i) hydrogen; (ii) C₁-C₃alkyl; and (iii) hydroxyC₁-C₄alkyl;
R₂ is selected from (i) hydrogen; and (ii) halo;
R₃ is selected from (i) halo; (ii) haloC₁-C₃alkyl; and (iii) cyano;
R₄ is selected from (i) hydrogen; (ii) halo; and (iii) C₁-C₃alkyl;
R₅ is selected from (i) hydrogen; (ii) C₁-C₆alkoxy optionally substituted with C₃-C₆cycloalkyl; CO₂H; SO₂C₁-C₃alkyl; a 5- or 6-membered aromatic heterocyclic ring comprising at least one heteroatom selected from N, O, and S; or a 5- or 6-membered saturated heterocyclic ring comprising at least one heteroatom selected from N and O, which ring is optionally substituted with C(O)C₁-C₃alkyl;
(iii) halo; (iv) hydroxyC₁-C₆alkoxy, wherein the alkoxy is optionally deuterated; (v) haloC₁-C₆alkoxy optionally substituted with hydroxy; (vi)S-haloC₁-C₃alkyl optionally substituted with hydroxy; (vii) C₁-C₃alkoxyC₁-C₃alkoxy; (viii) NR^{5a}R^{5b}; (ix) C₁-C₃alkyl; (x) a 5- or 6-membered aromatic heterocyclic ring comprising at least one heteroatom selected from N, O, and S; and (xi) hydroxy;
R^{5a} and R^{5b} are each independently selected from (i) hydrogen; and (ii) C₁-C₃alkyl; or
R^{5a} and R^{5b} together with the nitrogen atom to which they are attached form a 5- or 6-membered saturated heterocyclic ring, which saturated heterocyclic ring optionally in addition carries a hydroxy group;
R₆ is selected from (i) hydrogen; (ii) cyano; (iii) C(O)NHR^{6a}; (iv) NHR^{6b}; and (v) C₁-C₃alkoxy substituted with NH₂ or hydroxy;
R^{6a} is selected from (i) hydrogen; (ii) C₁-C₃alkyl; (iii) C₃-C₆cycloalkyl; (iv) a 5- or 6-membered aromatic heterocyclic ring comprising at least one heteroatom selected from N, O, and S which aromatic heterocyclic ring is optionally substituted with C₁-C₃alkyl;
R^{6b} is C₁-C₃alkyl substituted with NH₂ or hydroxy;
R⁷ is each independently selected from hydrogen and C₁-C₃alkyl; and
R⁸ is hydrogen or C₁-C₃-alkyl.

In an embodiment, the compound of formula (I) or a pharmaceutically acceptable salt thereof, is of formula (Ia), (Ic), or (Id),

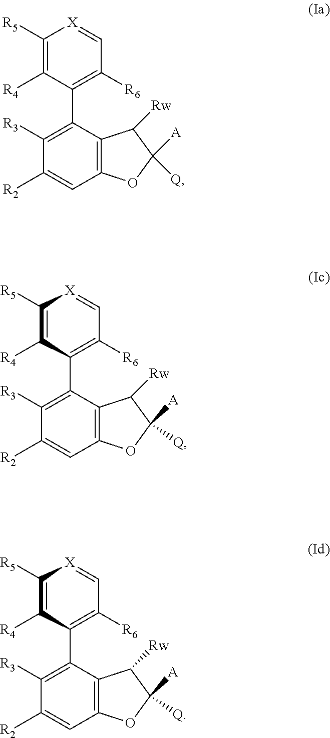

In an embodiment, X is selected from CH; and N;
A is selected from
(i) phenyl, which phenyl is optionally substituted with halo; or haloC₁-C₃alkoxy;
(ii) a 5- or 6-membered aromatic heterocyclic ring comprising at least one heteroatom selected from N, O, and S, preferably from N and S, which aromatic heterocyclic ring is optionally substituted with hydroxy; C₁-C₃alkoxy; or oxo; and
(iii) a halobenzodioxole moiety of formula

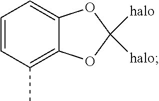

R_{w} is selected from (i) hydrogen; (ii) hydroxy; (iii) C₁-C₃alkoxy; (iv) hydroxy-C₁-C₃alkyl; (v) C₁-C₃alkyl; and (vi) C₁-C₃alkoxy-C₁-C₃alkyl;
Q is selected from (i) —C(R⁷)₂-N(R⁸)-R¹; (ii) 9- or 10-membered partially saturated heteroaryl comprising at least one N heteroatom; and (iii) 4-, 5- or 6-membered saturated heterocyclic ring comprising at least one heteroatom selected from N, O and S, with the proviso that at least one N heteroatom is present, and wherein the N is optionally present in the α-positon to the atom binding Q to the rest of the molecule, and wherein the heterocyclic ring is unsubstituted or substituted with one or more substituents independently selected from hydroxy, C₁-C₃alkyl, C₁-C₃alkoxy, halo and methylene forming a bridge between two ring atoms of the saturated heterocyclic ring, thus forming a bridged bicyclic structure;
R₁ is selected from hydrogen; C₁-C₆alkyl; and (CH₂)₀₋₂R₁ₐ wherein
R₁ₐ is selected from (i) C₁-C₃alkoxy; (ii) C₃-C₆cycloalkyl optionally substituted once or more than once independently with hydroxy; hydroxyC₁-C₄alkyl; C₁-C₄alkoxy; C(O)OC₁-C₃alkyl; CO₂H; C(O)NR¹cR^{1d}; C₁-C₅alkyl; halo; haloC₁-C₃alkoxy-C₁-C₃alkyl; SO₂C₁-C₃alkyl; haloC₁-C₃alkyl; NHR^{1b}; C(O)NR¹CR^{1d}; a 5- or 6-membered aromatic heterocyclic ring comprising at least one heteroatom selected from N, O, and S; or with two R^{1e} groups, wherein the two R^{1e} groups are attached at the same carbon atom and form together with the carbon atom to which they are attached a 5-membered saturated heterocyclic ring comprising at least one heteroatom selected from N and O, or a C₃-C₆cycloalkyl, which saturated heterocyclic ring or cycloalkyl are optionally substituted with hydroxy or oxo; (iii) a 5- or 6-membered saturated heterocyclic ring comprising at least one heteroatom selected from N and O, which saturated heterocyclic ring is optionally substituted once or more than once independently with C₁-C₃alkyl; (CH₂)₀₋₁C(O)di(C₁-Cₐalkyl)amino; SO₂C₁-C₃alkyl; C(O)C₁-C₃alkyl; or oxo;
R^{1b} is selected from C(O)C₁-C₃alkyl; and SO₂C₁-C₃alkyl;
R^{1c} and R^{1d} are each independently selected from (i) hydrogen; (ii) C₁-C₃alkyl; and (iii) hydroxyC₁-C₄alkyl,
R₂ is hydrogen or halo,
R₃ is halo; haloC₁-C₃alkyl; or cyano,
R₄ is selected from hydrogen; halo; and C₁-C₃alkyl,
R₅ is selected from (i) hydrogen; (ii) halo-C₁-C₆alkoxy optionally substituted with hydroxy;
(iii)S-haloC₁-C₃alkyl optionally substituted with hydroxy; (iv) C₁-C₃alkoxyC₁-C₃alkoxy; (v) C₁-C₆alkoxy optionally substituted with SO₂C₁-C₃alkyl, C₃-C₆cycloalkyl, CO₂H or a 5- or 6-membered saturated heterocyclic ring comprising at least one heteroatom selected from N and O, which ring is optionally substituted with C(O)C₁-C₃alkyl; (vi) C₁-C₃alkyl;
(vii) hydroxyC₁-C₆alkoxy; (viii) a 5- or 6-membered aromatic heterocyclic ring comprising at least one heteroatom selected from N, O, and S, preferably at least one N heteroatom; and (ix) hydroxy,
R₆ is cyano; C(O)NHR^{6a}; NHR^{6b}; or C₁-C₃alkoxy substituted with NH₂ or hydroxy, R^{6a} is selected from (i) hydrogen; (ii) C₁-C₃alkyl; (iii) C₃-C₆cycloalkyl; and (iv) a 5- or 6-membered aromatic heterocyclic ring comprising at least one heteroatom selected from N, O, and S, preferably at least one N heteroatom, which aromatic heterocyclic ring is optionally substituted with C₁-C₃alkyl;
R^{6b} is C₁-C₃alkyl substituted with NH₂ or hydroxy;
R⁷ is each independently selected from hydrogen and C₁-C₃alkyl, and R⁸ is hydrogen or C₁-C₃alkyl.

In an embodiment, X is selected from CH; and N;
A is phenyl, which phenyl is optionally substituted with halo; or haloC₁-C₃alkoxy;
R^{w} is selected from (i) hydrogen; (ii) C₁-C₃alkoxy; (iii) hydroxy-C₁-C₃alkyl; (iv) C₁-C₃alkyl; and (v)C₁-C₃alkoxy-C₁-C₃alkyl;
Q is selected from (i) —C(R⁷)₂-NH—R₁; and (ii) 4-, 5- or 6-membered saturated heterocyclic ring comprising one or two heteroatoms independently selected from N, O and S, with the proviso that at least one N heteroatom is present, wherein the N is present in the α-positon to the atom binding Q to the rest of the molecule, and wherein the heterocyclic ring is unsubstituted or substituted with one or more substituents independently selected from hydroxy, C₁-C₃alkyl and halo;
R_{w} is selected from (i) C₁-C₆alkyl; and (ii) Ria; wherein
R₁ₐ is selected from C₃-C₆cycloalkyl optionally substituted once or more than once independently with hydroxy; C₁-C₆alkyl; or halo;
R₂ is hydrogen or halo;
R₃ is halo;
R₄ is selected from (i) hydrogen; and (ii) halo;
R₅ is selected from halo-C₁-C₆alkoxy, hydroxy, C₁-C₆alkoxy; and hydroxyC₁-C₆alkoxy;
R₆ is C(O)NHR⁶a;
R^{6a} is selected from (i) hydrogen; and (ii) C₁-C₃alkyl;
and
R⁷ is each independently selected from hydrogen and C₁-C₃alkyl.

In an embodiment, X is selected from CH; and N;
A is phenyl, which phenyl is optionally substituted with halo; or haloC₁-C₃alkoxy, especially unsubstituted phenyl;
R_{w} is selected from (i) hydrogen; and (ii) C₁-C₃alkyl,
Q is selected from (i) —C(R⁷)₂-NH—R₁; and (ii) 4-, 5- or 6-membered saturated heterocyclic ring comprising one or two heteroatoms independently selected from N and O, with the proviso that at least one N heteroatom is present and is in the α-positon to the carbon atom binding Q to the rest of the molecule, wherein the heterocyclic ring is unsubstituted or substituted with one or more substituents independently selected from hydroxy, C₁-C₃alkyl and halo;
R₁ is selected from (i) C₁-C₆alkyl; and (ii) R₁ₐ; wherein
R₁ₐ is C₃-C₆cycloalkyl optionally substituted once or more than once independently with hydroxy; C₁-C₆alkyl; or halo;
R₂ is halo, especially fluoro;
R₃ is halo, especially chloro;
R₄ is halo, especially fluoro;
R₅ is selected from C₁-C₆alkoxy; and hydroxyC₁-C₆alkoxy;
R₆ is C(O)NHR⁶a;
R^{6a} is selected from (i) hydrogen; and (ii) C₁-C₃alkyl;
and
each R⁷ is hydrogen.

In an embodiment, the compound of formula (I) is selected from the group consisting of
(S)-(5-chloro-2,4-diphenyl-2,3-dihydrobenzofuran-2-yl)methanamine;
N1-(2-((2S,4S)-2-(Aminomethyl)-5-chloro-2-phenyl-2,3-dihydrobenzofuran-4-yl)-3-fluoro-phenyl)ethane-1,2-diamine;
2-(2-((2S,4S)-2-(Aminomethyl)-5-chloro-2-phenyl-2,3-dihydrobenzofuran-4-yl)-3-fluoro-phenoxy)ethanamine;
2-((2S,4S)-2-(aminomethyl)-5-chloro-2-phenyl-2,3-dihydrobenzofuran-4-yl)-3-fluorobenz-amide;
2-((2S,4S)-2-(Aminomethyl)-5-chloro-2-phenyl-2,3-dihydrobenzofuran-4-yl)-3-fluoro-4-meth-oxybenzamide;
2-((2S,4R)-2-(aminomethyl)-5-chloro-2-phenyl-2,3-dihydrobenzofuran-4-yl)-3-methylbenz-amide;
2-((2S,4S)-2-(aminomethyl)-5-chloro-2-phenyl-2,3-dihydrobenzofuran-4-yl)-3-chlorobenz-amide trifluoroacetate salt;
2-((2S,4S)-2-(aminomethyl)-5-chloro-2-phenyl-2,3-dihydrobenzofuran-4-yl)-4-chloro-3-fluoro-benzamide;
2-((2S,4S)-2-(aminomethyl)-5-chloro-2-phenyl-2,3-dihydrobenzofuran-4-yl)-3,4-difluoro-benzamide;
2-((2S,4S)-2-(aminomethyl)-5-chloro-2-phenyl-2,3-dihydrobenzofuran-4-yl)-3-fluoro-4-(2-hydroxyethoxy)benzamide;
2-((2S,4S)-2-(aminomethyl)-5-chloro-2-phenyl-2,3-dihydrobenzofuran-4-yl)-4-(difluoro-methoxy)-3-fluorobenzamide;
2-((2S,4S)-2-(aminomethyl)-5-chloro-2-phenyl-2,3-dihydrobenzofuran-4-yl)-3-fluoro-4-(2-methoxyethoxy)benzamide;
2-((2S,4S)-2-(aminomethyl)-5-chloro-2-phenyl-2,3-dihydrobenzofuran-4-yl)-3-fluoro-4-((S)-2-hydroxypropoxy)benzamide;
2-((2S,4S)-2-(aminomethyl)-5-chloro-2-phenyl-2,3-dihydrobenzofuran-4-yl)-3-fluoro-4-((R)-2-hydroxypropoxy)benzamide;
2-((2S,4S)-2-(aminomethyl)-5-chloro-2-phenyl-2,3-dihydrobenzofuran-4-yl)-3-fluoro-4-((R)-2-fluoropropoxy)benzamide;
2-(3-((2S,4S)-2-(aminomethyl)-5-chloro-2-phenyl-2,3-dihydrobenzofuran-4-yl)-4-carbamoyl-2-fluorophenoxy)acetic acid trifluoroacetate salt;
4-(((R)-4-acetylmorpholin-2-yl)methoxy)-2-((2S,4S)-2-(aminomethyl)-5-chloro-2-phenyl-2,3-dihydrobenzofuran-4-yl)-3-fluorobenzamide:
4-((2S,4S)-2-(Aminomethyl)-5-chloro-2-phenyl-2,3-dihydrobenzofuran-4-yl)-5-fluoro-6-meth-oxynicotinamide;
4-((2S,4S)-2-(Aminomethyl)-5-chloro-2-phenyl-2,3-dihydrobenzofuran-4-yl)-6-(difluorometh-oxy)-5-fluoronicotinamide;
2-((2S,4S)-2-(Aminomethyl)-5-chloro-2-phenyl-2,3-dihydrobenzofuran-4-yl)-3-fluoro-4-(methylamino)benzamide;
2-((2S,4S)-5-Chloro-2-((methylamino)methyl)-2-phenyl-2,3-dihydrobenzofuran-4-yl)-3-fluoro-4-methoxybenzamide;
2-((2S,4S)-2-(Aminomethyl)-5-chloro-2-phenyl-2,3-dihydrobenzofuran-4-yl)-3-fluoro-4-methoxy-N-methylbenzamide;
2-((2S,4S)-2-(aminomethyl)-5-chloro-2-phenyl-2,3-dihydrobenzofuran-4-yl)-N-cyclopropyl-3-fluoro-4-methoxybenzamide;
2-((2S,4S)-2-(aminomethyl)-5-chloro-2-phenyl-2,3-dihydrobenzofuran-4-yl)-3,4-difluoro-N-(1-methyl-1H-pyrazol-5-yl)benzamide;
2-((2S,4S)-2-(aminomethyl)-5-chloro-2-phenyl-2,3-dihydrobenzofuran-4-yl)-3-fluoro-4-methoxy-N-(pyridin-3-yl)benzamide;
2-((2S,4S)-5-Chloro-2-((methylamino)methyl)-2-phenyl-2,3-dihydrobenzofuran-4-yl)-3-fluoro-4-methoxy-N-methylbenzamide;
2-((2S,4S)-5-Chloro-2-(((trans-4-hydroxycyclohexyl)amino)methyl)-2-phenyl-2,3-dihydroben-zofuran-4-yl)-3-fluoro-4-methoxybenzamide;
2-((2S,4S)-5-chloro-2-(((cis-4-hydroxycyclohexyl)amino)methyl)-2-phenyl-2,3-dihydrobenzo-furan-4-yl)-3-fluoro-4-methoxybenzamide;
(trans)-4-((((2S,4S)-4-(6-carbamoyl-2-fluoro-3-methoxyphenyl)-5-chloro-2-phenyl-2,3-dihy-drobenzofuran-2-yl)methyl)amino)cyclohexanecarboxylic acid;
(cis)-4-((((2S,4S)-4-(6-carbamoyl-2-fluoro-3-methoxyphenyl)-5-chloro-2-phenyl-2,3-dihydro-benzofuran-2-yl)methyl)amino)cyclohexanecarboxylic acid;
2-((2R,4S)-2-(Aminomethyl)-5-chloro-2-(thiazol-4-yl)-2,3-dihydrobenzofuran-4-yl)-3-fluoro-4-methoxybenzamide;
2-((2S,4S)-2-(aminomethyl)-5-chloro-2-(2,2-difluorobenzo[d][1,3]dioxol-4-yl)-2,3-dihydroben-zofuran-4-yl)-3-fluorobenzamide;
2-((2S,4S)-2-(Aminomethyl)-5-chloro-2-(2-fluorophenyl)-2,3-dihydrobenzofuran-4-yl)-3-fluoro-4-methoxybenzamide;
2-((2S,4S)-2-(Aminomethyl)-5-chloro-6-fluoro-2-phenyl-2,3-dihydrobenzofuran-4-yl)-3-fluorobenzamide;
2-((2S,4S)-2-(aminomethyl)-5-chloro-6-fluoro-2-phenyl-2,3-dihydrobenzofuran-4-yl)-3-fluoro-4-methoxybenzamide;
2-((2S,4S)-2-(aminomethyl)-5-chloro-6-fluoro-2-phenyl-2,3-dihydrobenzofuran-4-yl)-4-(cyclo-propylmethoxy)-3-fluorobenzamide;
2-((2S,4S)-2-(aminomethyl)-5-chloro-6-fluoro-2-phenyl-2,3-dihydrobenzofuran-4-yl)-4-(difluo-romethoxy)-3-fluorobenzamide;
2-((2S,4S)-2-(aminomethyl)-5-chloro-6-fluoro-2-phenyl-2,3-dihydrobenzofuran-4-yl)-4-(1,1-difluoro-2-hydroxyethoxy)-3-fluorobenzamide;
2-((2S,4S)-2-(aminomethyl)-5-chloro-6-fluoro-2-phenyl-2,3-dihydrobenzofuran-4-yl)-3-fluoro-4-(2-methoxyethoxy)benzamide;
2-((2S,4S)-2-(aminomethyl)-5-chloro-6-fluoro-2-phenyl-2,3-dihydrobenzofuran-4-yl)-3-fluoro-4-(2-hydroxyethoxy)benzamide;
2-((2S,4R)-2-(aminomethyl)-5-chloro-6-fluoro-2-phenyl-2,3-dihydrobenzofuran-4-yl)-4-((1,1-difluoro-2-hydroxyethyl)thio)benzamide;
2-((2S,4S)-2-(aminomethyl)-5-chloro-6-fluoro-2-phenyl-2,3-dihydrobenzofuran-4-yl)-3-fluoro-4-((methylsulfonyl)methoxy)benzamide;
2-((2S,4S)-2-(aminomethyl)-5-chloro-6-fluoro-2-phenyl-2,3-dihydrobenzofuran-4-yl)-4-(3,3-difluoropropoxy)-3-fluorobenzamide;
4-((2S,4S)-2-(aminomethyl)-5-chloro-6-fluoro-2-phenyl-2,3-dihydrobenzofuran-4-yl)-5-fluoro-6-methoxynicotinamide;
4-((2S,4S)-2-(Aminomethyl)-5-chloro-6-fluoro-2-phenyl-2,3-dihydrobenzofuran-4-yl)-5-fluoro-6-(2-hydroxyethoxy)nicotinamide;
2-((2S,4S)-2-(Aminomethyl)-5-chloro-6-fluoro-2-phenyl-2,3-dihydrobenzofuran-4-yl)-3-fluoro-4-(2-hydroxyethoxy)-N-methylbenzamide;
4-((2S,4S)-2-(Aminomethyl)-5-chloro-6-fluoro-2-phenyl-2,3-dihydrobenzofuran-4-yl)-5-fluoro-6-(2-hydroxyethoxy)-N-methylnicotinamide;
2-((2S,4S)-5-Chloro-6-fluoro-2-((methylamino)methyl)-2-phenyl-2,3-dihydrobenzofuran-4-yl)-3-fluoro-4-methoxybenzamide;
4-((2S,4S)-5-Chloro-6-fluoro-2-((methylamino)methyl)-2-phenyl-2,3-dihydrobenzofuran-4-yl)-5-fluoro-6-(2-methoxyethoxy)nicotinamide;
2-((2S,4S)-5-Chloro-6-fluoro-2-((methylamino)methyl)-2-phenyl-2,3-dihydrobenzofuran-4-yl)-3-fluoro-4-(2-hydroxyethoxy)benzamide;
4-((2S,4S)-5-Chloro-6-fluoro-2-((methylamino)methyl)-2-phenyl-2,3-dihydrobenzofuran-4-yl)-5-fluoro-6-(2-hydroxyethoxy)nicotinamide;
2-((2S,4S)-5-Chloro-2-(((cyclopropylmethyl)amino)methyl)-6-fluoro-2-phenyl-2,3-dihydroben-zofuran-4-yl)-4-(difluoromethoxy)-3-fluorobenzamide;
2-((2S,4S)-5-Chloro-6-fluoro-2-((methylamino)methyl)-2-phenyl-2,3-dihydrobenzofuran-4-yl)-3-fluoro-4-(2-hydroxyethoxy)-N-methylbenzamide;
2-((2S,4S)-5-chloro-6-fluoro-2-((methylamino)methyl)-2-phenyl-2,3-dihydrobenzofuran-4-yl)-3-fluoro-4-((S)-2-hydroxypropoxy)-N-methylbenzamide;
4-((2S,4S)-5-chloro-6-fluoro-2-((methylamino)methyl)-2-phenyl-2,3-dihydrobenzofuran-4-yl)-5-fluoro-6-(2-hydroxyethoxy)-N-methylnicotinamide;
2-((2S,4S)-5-Chloro-6-fluoro-2-((((cis)-4-(methylsulfonyl)cyclohexyl)amino)methyl)-2-phenyl-2,3-dihydrobenzofuran-4-yl)-4-(difluoromethoxy)-3-fluorobenzamide;
2-((2S,4S)-5-chloro-6-fluoro-2-((((trans)-4-(methylsulfonyl)cyclohexyl)amino)methyl)-2-phenyl-2,3-dihydrobenzofuran-4-yl)-4-(difluoromethoxy)-3-fluorobenzamide;
2-((2S,4S)-5-chloro-6-fluoro-2-(((4-(fluoromethyl)-4-hydroxycyclohexyl)amino)methyl)-2-phenyl-2,3-dihydrobenzofuran-4-yl)-4-(difluoromethoxy)-3-fluorobenzamide;
2-((2S,4S)-2-(((4-acetamidocyclohexyl)amino)methyl)-5-chloro-6-fluoro-2-phenyl-2,3-dihydrobenzofuran-4-yl)-4-(difluoromethoxy)-3-fluorobenzamide;
2-((2S,4S)-5-chloro-6-fluoro-2-(((4-(methylsulfonamido)cyclohexyl)amino)methyl)-2-phenyl-2,3-dihydrobenzofuran-4-yl)-4-(difluoromethoxy)-3-fluorobenzamide;
2-((2S,4S)-5-chloro-2-(((4-(dimethylcarbamoyl)cyclohexyl)amino)methyl)-6-fluoro-2-phenyl-2,3-dihydrobenzofuran-4-yl)-4-(difluoromethoxy)-3-fluorobenzamide;
2-((2S,4S)-5-chloro-6-fluoro-2-(((2-oxo-1-azaspiro[4.5]decan-8-yl)amino)methyl)-2-phenyl-2,3-dihydrobenzofuran-4-yl)-4-(difluoromethoxy)-3-fluorobenzamide;
2-((2S,4S)-5-chloro-2-(((1-(2-(dimethylamino)-2-oxoethyl)piperidin-4-yl)amino)methyl)-6-fluoro-2-phenyl-2,3-dihydrobenzofuran-4-yl)-4-(difluoromethoxy)-3-fluorobenzamide;
2-((2S,4S)-5-chloro-6-fluoro-2-(((4-(hydroxymethyl)cyclohexyl)amino)methyl)-2-phenyl-2,3-dihydrobenzofuran-4-yl)-4-(difluoromethoxy)-3-fluorobenzamide;
2-((2S,4S)-5-chloro-6-fluoro-2-(((3-(2-hydroxypropan-2-yl)cyclobutyl)amino)methyl)-2-phenyl-2,3-dihydrobenzofuran-4-yl)-4-(difluoromethoxy)-3-fluorobenzamide;
2-((2S,4S)-5-chloro-6-fluoro-2-(((1-(methylsulfonyl)piperidin-4-yl)amino)methyl)-2-phenyl-2,3-dihydrobenzofuran-4-yl)-4-(difluoromethoxy)-3-fluorobenzamide;
2-((2S,4S)-5-chloro-6-fluoro-2-((((trans)-3-(hydroxymethyl)cyclobutyl)amino)methyl)-2-phenyl-2,3-dihydrobenzofuran-4-yl)-3-fluoro-4-(2-hydroxyethoxy)benzamide;
2-((2S,4S)-5-chloro-6-fluoro-2-((((cis)-3-(hydroxymethyl)cyclobutyl)amino)methyl)-2-phenyl-2,3-dihydrobenzofuran-4-yl)-3-fluoro-4-(2-hydroxyethoxy)benzamide;
2-((2S,4S)-5-chloro-2-((((2R,4r,6S)-2,6-dimethyltetrahydro-2H-pyran-4-yl)amino)methyl)-6-fluoro-2-phenyl-2,3-dihydrobenzofuran-4-yl)-3-fluoro-4-(2-hydroxyethoxy)benzamide;
2-((2S,4S)-5-chloro-2-((((2R,4s,6S)-2,6-dimethyltetrahydro-2H-pyran-4-yl)amino)methyl)-6-fluoro-2-phenyl-2,3-dihydrobenzofuran-4-yl)-3-fluoro-4-(2-hydroxyethoxy)benzamide;
4-((2S,4S)-5-chloro-6-fluoro-2-((((trans)-4-hydroxy-4-methylcyclohexyl)amino)methyl)-2-phenyl-2,3-dihydrobenzofuran-4-yl)-5-fluoro-6-methoxynicotinamide;
4-((2S,4S)-5-chloro-6-fluoro-2-((((cis)-4-hydroxy-4-methylcyclohexyl)amino)methyl)-2-phenyl-2,3-dihydrobenzofuran-4-yl)-5-fluoro-6-methoxynicotinamide;
2-((2S,4S)-5-chloro-6-fluoro-2-((((cis)-4-hydroxy-4-methylcyclohexyl)amino)methyl)-2-phenyl-2,3-dihydrobenzofuran-4-yl)-3-fluoro-4-(((R)-tetrahydrofuran-2-yl)methoxy)benzamide;
2-((2S,4S)-5-chloro-6-fluoro-2-((((trans)-4-hydroxy-4-methylcyclohexyl)amino)methyl)-2-phenyl-2,3-dihydrobenzofuran-4-yl)-3-fluoro-4-(((R)-tetrahydrofuran-2-yl)methoxy)benzamide;
2-((2S,4S)-5-chloro-6-fluoro-2-((((trans)-4-hydroxy-4-methylcyclohexyl)amino)methyl)-2-phenyl-2,3-dihydrobenzofuran-4-yl)-4-ethyl-3-fluorobenzamide;
2-((2S,4S)-5-chloro-6-fluoro-2-((((cis)-4-hydroxy-4-methylcyclohexyl)amino)methyl)-2-phenyl-2,3-dihydrobenzofuran-4-yl)-4-ethyl-3-fluorobenzamide;
2-((2S,4S)-5-chloro-6-fluoro-2-((((trans)-4-hydroxy-4-methylcyclohexyl)amino)methyl)-2-phenyl-2,3-dihydrobenzofuran-4-yl)-3-fluoro-4-(1H-imidazol-1-yl)benzamide;
2-((2S,4S)-5-chloro-6-fluoro-2-((((cis)-4-hydroxy-4-methylcyclohexyl)amino)methyl)-2-phenyl-2,3-dihydrobenzofuran-4-yl)-3-fluoro-4-(1H-imidazol-1-yl)benzamide;
2-((2S,4S)-2-(((1-acetylpiperidin-4-yl)amino)methyl)-5-chloro-6-fluoro-2-phenyl-2,3-dihydrobenzofuran-4-yl)-4-(difluoromethoxy)-3-fluorobenzamide;
2-((2S,4S)-5-chloro-6-fluoro-2-((((trans)-4-hydroxy-4-methylcyclohexyl)amino)methyl)-2-phenyl-2,3-dihydrobenzofuran-4-yl)-3-fluoro-4-(pyrimidin-2-ylmethoxy)benzamide;
2-((2S,4S)-5-chloro-6-fluoro-2-((((cis)-4-hydroxy-4-methylcyclohexyl)amino)methyl)-2-phenyl-2,3-dihydrobenzofuran-4-yl)-3-fluoro-4-(pyrimidin-2-ylmethoxy)benzamide;
2-((2S,4S)-5-chloro-6-fluoro-2-((((trans)-4-hydroxycyclohexyl)amino)methyl)-2-phenyl-2,3-dihydrobenzofuran-4-yl)-4-(difluoromethoxy)-3-fluorobenzamide;
2-((2S,4S)-2-((tert-butylamino)methyl)-5-chloro-6-fluoro-2-phenyl-2,3-dihydrobenzofuran-4-yl)-4-(difluoromethoxy)-3-fluorobenzamide;
2-((2S,4S)-5-chloro-6-fluoro-2-((((R)-2-hydroxypropyl)amino)methyl)-2-phenyl-2,3-dihydrobenzofuran-4-yl)-4-(difluoromethoxy)-3-fluorobenzamide:
2-((2S,4S)-5-chloro-6-fluoro-2-((((S)-1-hydroxypropan-2-yl)amino)methyl)-2-phenyl-2,3-dihydrobenzofuran-4-yl)-4-(difluoromethoxy)-3-fluorobenzamide;
2-((2S,4S)-5-chloro-6-fluoro-2-(((1-methylcyclopropyl)amino)methyl)-2-phenyl-2,3-di-hydrobenzofuran-4-yl)-4-(difluoromethoxy)-3-fluorobenzamide;
2-((2S,4S)-5-chloro-6-fluoro-2-(((2-methoxyethyl)amino)methyl)-2-phenyl-2,3-dihydro-benzofuran-4-yl)-4-(difluoromethoxy)-3-fluorobenzamide;
2-((2S,4S)-5-chloro-6-fluoro-2-(((2-(2-oxopyrrolidin-1-yl)ethyl)amino)methyl)-2-phenyl-2,3-dihydrobenzofuran-4-yl)-4-(difluoromethoxy)-3-fluorobenzamide;
2-((2S,4S)-2-((((trans)-4-(1H-tetrazol-1-yl)cyclohexyl)amino)methyl)-5-chloro-6-fluoro-2-phenyl-2,3-dihydrobenzofuran-4-yl)-4-(difluoromethoxy)-3-fluorobenzamide;
2-((2S,4S)-5-chloro-2-(((trans-3-((difluoromethoxy)methyl)cyclobutyl)amino)methyl)-6-fluoro-2-phenyl-2,3-dihydrobenzofuran-4-yl)-4-(difluoromethoxy)-3-fluorobenzamide;
2-((2S,4S)-5-chloro-6-fluoro-2-((((1S,3R,4R)-3-fluoro-4-hydroxycyclohexyl)amino)methyl)-2-phenyl-2,3-dihydrobenzofuran-4-yl)-4-(difluoromethoxy)-3-fluorobenzamide;
2-((2S,4S)-5-chloro-6-fluoro-2-((((1R,3S,4S)-3-fluoro-4-hydroxycyclohexyl)amino)methyl)-2-phenyl-2,3-dihydrobenzofuran-4-yl)-4-(difluoromethoxy)-3-fluorobenzamide;
2-((2S,4S)-5-chloro-6-fluoro-2-(((trans-3-fluorocyclobutyl)amino)methyl)-2-phenyl-2,3-di-hydrobenzofuran-4-yl)-4-(difluoromethoxy)-3-fluorobenzamide;
2-((2S,4S)-5-chloro-6-fluoro-2-((((1R,3S)-3-hydroxycyclohexyl)amino)methyl)-2-phenyl-2,3-dihydrobenzofuran-4-yl)-4-(difluoromethoxy)-3-fluorobenzamide;
2-((2S,4S)-5-chloro-6-fluoro-2-((((trans)-4-hydroxy-1-methylcyclohexyl)amino)methyl)-2-phenyl-2,3-dihydrobenzofuran-4-yl)-4-(difluoromethoxy)-3-fluorobenzamide;
2-((2S,4S)-5-Chloro-6-fluoro-2-(((6-hydroxyspiro[3.3]heptan-2-yl)amino)methyl)-2-phenyl-2,3-dihydrobenzofuran-4-yl)-4-(difluoromethoxy)-3-fluorobenzamide;
2-((2S,3S,4S)-2-(Aminomethyl)-5-chloro-3-hydroxy-2-phenyl-2,3-dihydrobenzofuran-4-yl)-3-fluoro-4-methoxybenzamide;
2-((2S,3S,4S)-2-(aminomethyl)-5-chloro-3-hydroxy-2-phenyl-2,3-dihydrobenzofuran-4-yl)-4-(difluoromethoxy)-3-fluorobenzamide;
2-((2S,3S,4S)-2-(aminomethyl)-5-chloro-3-hydroxy-2-phenyl-2,3-dihydrobenzofuran-4-yl)-3-fluoro-4-(2-methoxyethoxy)benzamide;
2-((2R,3S,4S)-2-(Aminomethyl)-5-chloro-3-hydroxy-2-(pyridin-2-yl)-2,3-dihydrobenzofuran-4-yl)-3-fluoro-4-methoxybenzamide;
2-((2R,3S,4S)-2-(Aminomethyl)-5-chloro-3-hydroxy-2-(6-methoxypyridin-2-yl)-2,3-dihydro-benzofuran-4-yl)-4-(difluoromethoxy)-3-fluorobenzamide:
2-((2S,3S,4S)-2-(Aminomethyl)-5-chloro-3-hydroxy-2-(2-methoxypyridin-3-yl)-2,3-dihydro-benzofuran-4-yl)-3-fluoro-4-methoxybenzamide;
2-((2S,3S,4S)-2-(Aminomethyl)-5-chloro-3-hydroxy-2-(2-oxo-1,2-dihydropyridin-3-yl)-2,3-dihydrobenzofuran-4-yl)-3-fluoro-4-methoxybenzamide;
2-((2S,3S,4S)-2-(Aminomethyl)-5-chloro-3-hydroxy-2-(2-(trifluoromethoxy)phenyl)-2,3-dihydrobenzofuran-4-yl)-3-fluoro-4-methoxybenzamide;
2-((2S,3S,4S)-5-chloro-3-hydroxy-2-((((trans)-4-hydroxycyclohexyl)amino)methyl)-2-phenyl-2,3-dihydrobenzofuran-4-yl)-4-(difluoromethoxy)-3-fluorobenzamide;
2-((2S,3S,4S)-5-chloro-3-hydroxy-2-((((cis)-4-hydroxycyclohexyl)amino)methyl)-2-phenyl-2,3-dihydrobenzofuran-4-yl)-4-(difluoromethoxy)-3-fluorobenzamide;
2-((2R,3S,4S)-5-Chloro-3-hydroxy-2-((((trans)-4-hydroxycyclohexyl)amino)methyl)-2-(pyridin-2-yl)-2,3-dihydrobenzofuran-4-yl)-3-fluoro-4-methoxybenzamide;
2-((2R,3S,4S)-5-chloro-3-hydroxy-2-((((cis)-4-hydroxycyclohexyl)amino)methyl)-2-(pyridin-2-yl)-2,3-dihydrobenzofuran-4-yl)-3-fluoro-4-methoxybenzamide;
2-((2S,3S,4S)-5-chloro-2-((cyclobutylamino)methyl)-3-hydroxy-2-phenyl-2,3-dihydrobenzo-furan-4-yl)-4-(difluoromethoxy)-3-fluorobenzamide;
2-((2S,3S,4S)-2-(Aminomethyl)-5-chloro-6-fluoro-3-hydroxy-2-phenyl-2,3-dihydrobenzo-furan-4-yl)-3-fluoro-4-methoxybenzamide;
2-((2S,3S,4S)-2-(Aminomethyl)-5-chloro-6-fluoro-3-hydroxy-2-phenyl-2,3-dihydrobenzo-furan-4-yl)-3-fluoro-4-(2-hydroxyethoxy)benzamide;
2-((2S,3S,4S)-5-Chloro-2-((cyclobutylamino)methyl)-6-fluoro-3-hydroxy-2-phenyl-2,3-di-hydrobenzofuran-4-yl)-3-fluoro-4-(2-hydroxyethoxy)benzamide;
2-((2S,3S,4S)-5-Chloro-6-fluoro-3-hydroxy-2-((((trans)-4-hydroxycyclohexyl)amino)methyl)-2-phenyl-2,3-dihydrobenzofuran-4-yl)-4-(difluoromethoxy)-3-fluorobenzamide;
2-((2S,3S,4S)-5-chloro-6-fluoro-3-hydroxy-2-((((cis)-4-hydroxycyclohexyl)amino)methyl)-2-phenyl-2,3-dihydrobenzofuran-4-yl)-4-(difluoromethoxy)-3-fluorobenzamide;
2-((2S,3S,4S)-2-(Aminomethyl)-5-chloro-6-fluoro-3-methoxy-2-phenyl-2,3-dihydrobenzo-furan-4-yl)-3-fluoro-4-(2-hydroxyethoxy)benzamide;
2-((2S,3S,4S)-5-Chloro-6-fluoro-2-((((trans)-4-hydroxycyclohexyl)amino)methyl)-3-methoxy-2-phenyl-2,3-dihydrobenzofuran-4-yl)-4-(difluoromethoxy)-3-fluorobenzamide;
2-((2S,3S,4S)-5-chloro-6-fluoro-2-((((cis)-4-hydroxycyclohexyl)amino)methyl)-3-methoxy-2-phenyl-2,3-dihydrobenzofuran-4-yl)-4-(difluoromethoxy)-3-fluorobenzamide;
2-((2S,3S,4S)-2-(aminomethyl)-5-chloro-3-methyl-2-phenyl-2,3-dihydrobenzofuran-4-yl)-4-(difluoromethoxy)-3-fluorobenzamide:
2-((2S,3S,4S)-5-chloro-2-((((cis)-4-hydroxy-4-methylcyclohexyl)amino)methyl)-3-methyl-2-phenyl-2,3-dihydrobenzofuran-4-yl)-4-(difluoromethoxy)-3-fluorobenzamide;
2-((2S,3S,4S)-5-chloro-2-((((trans)-4-hydroxy-4-methylcyclohexyl)amino)methyl)-3-methyl-2-phenyl-2,3-dihydrobenzofuran-4-yl)-4-(difluoromethoxy)-3-fluorobenzamide;
2-((2S,3S,4S)-2-(Aminomethyl)-5-chloro-6-fluoro-3-methyl-2-phenyl-2,3-dihydrobenzofuran-4-yl)-4-(difluoromethoxy)-3-fluorobenzamide;
2-((2S,3S,4S)-2-(aminomethyl)-5-chloro-6-fluoro-3-methyl-2-phenyl-2,3-dihydrobenzofuran-4-yl)-3-fluoro-4-(2-methoxyethoxy)benzamide;
2-((2S,3S,4S)-2-(aminomethyl)-5-chloro-6-fluoro-3-methyl-2-phenyl-2,3-dihydrobenzofuran-4-yl)-3-fluoro-4-(2-hydroxyethoxy)benzamide;
4-((2S,3S,4S)-2-(aminomethyl)-5-chloro-6-fluoro-3-methyl-2-phenyl-2,3-dihydrobenzofuran-4-yl)-5-fluoro-6-(2-methoxyethoxy)nicotinamide;
2-((2R,3S,4S)-2-(aminomethyl)-5-chloro-6-fluoro-3-methyl-2-(pyridin-2-yl)-2,3-dihydro-benzofuran-4-yl)-3-fluoro-4-methoxybenzamide;
2-((2S,3S,4S)-5-Chloro-6-fluoro-3-methyl-2-((methylamino)methyl)-2-phenyl-2,3-dihydro-benzofuran-4-yl)-3-fluoro-4-(2-hydroxyethoxy)benzamide;
2-((2R,3S,4S)-5-chloro-6-fluoro-3-methyl-2-((methylamino)methyl)-2-(pyridin-2-yl)-2,3-di-hydrobenzofuran-4-yl)-3-fluoro-4-(2-methoxyethoxy)benzamide;
2-((2R,3S,4S)-5-chloro-6-fluoro-3-methyl-2-((methylamino)methyl)-2-(pyridin-2-yl)-2,3-di-hydrobenzofuran-4-yl)-3-fluoro-4-(2-hydroxyethoxy)benzamide;
2-((2S,3S,4S)-5-Chloro-6-fluoro-3-methyl-2-((methylamino)methyl)-2-phenyl-2,3-dihydro-benzofuran-4-yl)-3-fluoro-4-(2-hydroxyethoxy)-N-methylbenzamide;
2-((2S,3S,4S)-5-chloro-6-fluoro-2-((((trans)-4-hydroxy-4-methylcyclohexyl)amino)methyl)-3-methyl-2-phenyl-2,3-dihydrobenzofuran-4-yl)-3-fluoro-4-((S)-2-hydroxypropoxy)benzamide;
2-((2S,3S,4S)-5-chloro-6-fluoro-2-((((cis)-4-hydroxy-4-methylcyclohexyl)amino)methyl)-3-methyl-2-phenyl-2,3-dihydrobenzofuran-4-yl)-3-fluoro-4-((S)-2-hydroxypropoxy)benzamide;
2-((2S,3S,4S)-5-chloro-6-fluoro-2-(((trans-4-hydroxycyclohexyl)amino)methyl)-3-methyl-2-phenyl-2,3-dihydrobenzofuran-4-yl)-4-(difluoromethoxy)-3-fluorobenzamide;
2-((2S,3S,4S)-5-chloro-6-fluoro-2-((((cis)-4-hydroxycyclohexyl)amino)methyl)-3-methyl-2-phenyl-2,3-dihydrobenzofuran-4-yl)-4-(difluoromethoxy)-3-fluorobenzamide;
2-((2S,3S,4S)-5-chloro-6-fluoro-2-((((trans)-4-hydroxy-4-methylcyclohexyl)amino)methyl)-3-methyl-2-phenyl-2,3-dihydrobenzofuran-4-yl)-3-fluoro-4-(2-methoxyethoxy)benzamide;
2-((2S,3S,4S)-5-chloro-6-fluoro-2-((((cis)-4-hydroxy-4-methylcyclohexyl)amino)methyl)-3-methyl-2-phenyl-2,3-dihydrobenzofuran-4-yl)-3-fluoro-4-(2-methoxyethoxy)benzamide:
2-((2S,3S,4S)-5-chloro-6-fluoro-2-(((trans-4-hydroxy-4-methylcyclohexyl)amino)methyl)-3-methyl-2-phenyl-2,3-dihydrobenzofuran-4-yl)-3-fluoro-4-(2-hydroxyethoxy)benzamide;
2-((2S,3S,4S)-5-chloro-6-fluoro-2-(((cis-4-hydroxy-4-methylcyclohexyl)amino)methyl)-3-methyl-2-phenyl-2,3-dihydrobenzofuran-4-yl)-3-fluoro-4-(2-hydroxyethoxy)benzamide;
2-((2S,3S,4S)-5-chloro-2-((cyclobutylamino)methyl)-6-fluoro-3-methyl-2-phenyl-2,3-di-hydrobenzofuran-4-yl)-3-fluoro-4-(2-hydroxyethoxy)benzamide;
4-((2S,3S,4S)-5-Chloro-6-fluoro-2-((((trans-4-hydroxy-4-methylcyclohexyl)amino)methyl)-3-methyl-2-phenyl-2,3-dihydrobenzofuran-4-yl)-5-fluoro-6-(2-hydroxyethoxy)nicotinamide;
2-((2S,3S,4S)-5-Chloro-6-fluoro-2-((((trans)-4-hydroxy-4-methylcyclohexyl)amino)methyl)-3-methyl-2-phenyl-2,3-dihydrobenzofuran-4-yl)-3-fluoro-4-((S)-2-hydroxypropoxy)benzonitrile;
2-((2S,3S,4S)-5-Chloro-6-fluoro-2-(((trans-4-hydroxy-4-methylcyclohexyl)amino)methyl)-3-methyl-2-phenyl-2,3-dihydrobenzofuran-4-yl)-3-fluoro-4-(2-hydroxyethoxy)-N-methylbenz-amide;
2-((2S,3S,4S)-5-Chloro-6-fluoro-3-methyl-2-((methylamino)methyl)-2-phenyl-2,3-dihydro-benzofuran-4-yl)-3-fluoro-4-((S)-2-hydroxypropoxy)-N-methylbenzamide;
2-((2S,3S,4S)-2-(Aminomethyl)-5-chloro-6-fluoro-3-methyl-2-phenyl-2,3-dihydrobenzofuran-4-yl)-3-fluoro-4-((S)-2-hydroxypropoxy)benzamide;
2-(2-((2S,3S,4S)-2-(Aminomethyl)-5-chloro-6-fluoro-3-methyl-2-phenyl-2,3-dihydrobenzo-furan-4-yl)-3,4-difluorophenoxy)ethan-1-ol;
2-((2R,3S,4S)-5-Chloro-6-fluoro-2-(6-hydroxypyridin-2-yl)-3-methyl-2-((methylamino)methyl)-2,3-dihydrobenzofuran-4-yl)-3-fluoro-4-methoxybenzamide;
2-((2R,3S,4S)-5-Chloro-6-fluoro-2-((((trans)-4-hydroxy-4-methylcyclohexyl)amino)methyl)-3-methyl-2-(pyridin-2-yl)-2,3-dihydrobenzofuran-4-yl)-3-fluoro-4-(2-methoxyethoxy)benzamide;
2-((2S,3S,4S)-5-Chloro-6-fluoro-2-((((trans)-4-hydroxy-4-methylcyclohexyl)amino)methyl)-3-methyl-2-(pyridin-3-yl)-2,3-dihydrobenzofuran-4-yl)-3-fluoro-4-methoxybenzamide;
2-((2S,4R)-2-(Aminomethyl)-2-phenyl-5-(trifluoromethyl)-2,3-dihydrobenzofuran-4-yl)-4-methoxybenzamide;
2-((2S,4R)-5-Cyano-2-((((trans)-4-hydroxy-4-methylcyclohexyl)amino)methyl)-2-phenyl-2,3-dihydrobenzofuran-4-yl)-3-fluoro-4-methoxy-N-methylbenzamide;
(S)-2-((((trans)-4-Hydroxycyclohexyl)amino)methyl)-2,4-diphenyl-2,3-dihydrobenzofuran-5-carbonitrile;
2-((2S,4S)-2-(Aminomethyl)-5-chloro-2-phenyl-2,3-dihydrofuro[2,3-b]pyridin-4-yl)-3-fluoro-benzamide:
2-(2-(Aminomethyl)-6-chloro-2-phenyl-2,3-dihydrobenzofuran-7-yl)-3-fluoro-4-methoxy-benzamide;
2-(2-(Aminomethyl)-5-chloro-2-phenylbenzo[d][1,3]dioxol-4-yl)-3-fluorobenzamide;
2-((2S,4S)-2-(aminomethyl)-5-chloro-6-fluoro-2-phenylindolin-4-yl)-3-fluorobenzamide;
2-((2S,4S)-5-Chloro-2-((cyclohexylamino)methyl)-2-phenyl-2,3-dihydrobenzofuran-4-yl)-4-(difluoromethoxy)-3-fluorobenzamide;
2-((2S,3S,4S)-5-Chloro-6-fluoro-3-hydroxy-2-((((cis)-4-methoxycyclohexyl)amino)methyl)-2-phenyl-2,3-dihydrobenzofuran-4-yl)-4-(difluoromethoxy)-3-fluorobenzamide;
2-((2S,3S,4S)-5-chloro-6-fluoro-3-hydroxy-2-((((trans)-4-methoxycyclohexyl)amino)methyl)-2-phenyl-2,3-dihydrobenzofuran-4-yl)-4-(difluoromethoxy)-3-fluorobenzamide;
Methyl (cis)-4-((((2S,4S)-4-(6-carbamoyl-2,3-difluorophenyl)-5-chloro-2-phenyl-2,3-di-hydrobenzofuran-2-yl)methyl)amino)cyclohexane-1-carboxylate;
Methyl (trans)-4-((((2S,4S)-4-(6-carbamoyl-2,3-difluorophenyl)-5-chloro-2-phenyl-2,3-di-hydrobenzofuran-2-yl)methyl)amino)cyclohexane-1-carboxylate;
2-((2S,4S)-2-((((Trans)-4-carbamoylcyclohexyl)amino)methyl)-5-chloro-2-phenyl-2,3-di-hydrobenzofuran-4-yl)-3-fluorobenzamide;
2-((2S,4S)-2-((((cis)-4-carbamoylcyclohexyl)amino)methyl)-5-chloro-2-phenyl-2,3-dihydro-benzofuran-4-yl)-3-fluorobenzamide;
2-((2S,4S)-5-Chloro-2-((((trans)-4-(methylcarbamoyl)cyclohexyl)amino)methyl)-2-phenyl-2,3-dihydrobenzofuran-4-yl)-3-fluorobenzamide;
2-((2S,4S)-5-chloro-2-((((cis)-4-(methylcarbamoyl)cyclohexyl)amino)methyl)-2-phenyl-2,3-dihydrobenzofuran-4-yl)-3-fluorobenzamide;
2-((2S,4S)-5-Chloro-2-((((cis)-3-(difluoromethyl)cyclobutyl)amino)methyl)-6-fluoro-2-phenyl-2,3-dihydrobenzofuran-4-yl)-3-fluoro-4-(2-hydroxyethoxy)benzamide;
2-((2S,4S)-5-chloro-2-((((trans)-3-(difluoromethyl)cyclobutyl)amino)methyl)-6-fluoro-2-phenyl-2,3-dihydrobenzofuran-4-yl)-3-fluoro-4-(2-hydroxyethoxy)benzamide;
2-((2S,4S)-2-(Aminomethyl)-5-chloro-6-fluoro-2-phenyl-2,3-dihydrobenzofuran-4-yl)-3-fluoro-4-(2-hydroxyethoxy-1,1,2,2-d4)-N-methylbenzamide;
2-((2S,4S)-5-Chloro-6-fluoro-2-(((methyl-d3)amino)methyl)-2-phenyl-2,3-dihydrobenzofuran-4-yl)-4-(difluoromethoxy)-3-fluorobenzamide;
2-((2S,4S)-5-Chloro-6-fluoro-2-(((methyl-d3)amino)methyl)-2-phenyl-2,3-dihydrobenzofuran-4-yl)-3-fluoro-4-(2-hydroxyethoxy-1,1,2,2-d4)benzamide;
2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide,
4-((2S,3S,4S)-5-Chloro-6-fluoro-3-methyl-2-((methylamino)methyl)-2-phenyl-2,3-dihydrobenzofuran-4-yl)-5-fluoro-6-(2-hydroxyethoxy)-N-methylnicotinamide
2-((2S,3R,4S)-5-Chloro-6-fluoro-3-(methoxymethyl)-2-((methylamino)methyl)-2-phenyl-2,3-dihydrobenzofuran-4-yl)-3-fluoro-4-methoxybenzamide,
2-((2S,3R,4S)-5-Chloro-6-fluoro-3-(hydroxymethyl)-2-((methylamino)methyl)-2-phenyl-2,3-dihydrobenzofuran-4-yl)-3-fluoro-4-methoxybenzamide,
2-((2S,3S,4S)-5-Chloro-6-fluoro-2-((((trans)-4-hydroxy-4-methylcyclohexyl)amino) methyl)-3-methyl-2-phenyl-2,3-dihydrobenzofuran-4-yl)-3-fluoro-4-(2-hydroxy-2-methylpropoxy)benzamide,
2-((2S,4S)-2-(Azetidin-2-yl)-5-chloro-6-fluoro-2-phenyl-2,3-dihydrobenzofuran-4-yl)-3-fluoro-4-(2-hydroxyethoxy)-N-methylbenzamide,
2-((2S,3S,4S)-2-(Azetidin-2-yl)-5-chloro-6-fluoro-3-methyl-2-phenyl-2,3-dihydrobenzofuran-4-yl)-3-fluoro-4-((S)-2-hydroxypropoxy)-N-methylbenzamide,
2-((2S,3S,4S)-2-(Azetidin-2-yl)-5-chloro-6-fluoro-3-methyl-2-phenyl-2,3-dihydrobenzofuran-4-yl)-3-fluoro-4-((S)-2-hydroxypropoxy)-N-methylbenzamide,
(2-((2S,4S)-5-Chloro-6-fluoro-2-phenyl-2-((S)-pyrrolidin-2-yl)-2,3-dihydrobenzofuran-4-yl)-3-fluoro-4-(2-hydroxyethoxy)benzamide,
2-((2S,4S)-5-Chloro-6-fluoro-2-phenyl-2-((S)-pyrrolidin-2-yl)-2,3-dihydrobenzofuran-4-yl)-3-fluoro-4-((S)-2-hydroxypropoxy)benzamide,
2-((2S,4S)-5-Chloro-6-fluoro-2-phenyl-2-((S)-pyrrolidin-2-yl)-2,3-dihydrobenzofuran-4-yl)-3-fluoro-4-(2-hydroxyethoxy)-N-methylbenzamide,
2-((2S,4S)-5-Chloro-6-fluoro-2-phenyl-2-((S)-pyrrolidin-2-yl)-2,3-dihydrobenzofuran-4-yl)-3-fluoro-4-((S)-2-hydroxypropoxy)-N-methylbenzamide,
4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide,
2-((4-((2S,4S)-5-Chloro-6-fluoro-2-phenyl-2-((S)-pyrrolidin-2-yl)-2,3-dihydrobenzofuran-4-yl)-3-fluoro-5-(methylcarbamoyl)pyridin-2-yl)oxy)acetic acid,
4-((2S,4S)-5-chloro-6-fluoro-2-phenyl-2-((S)-pyrrolidin-2-yl)-2,3-dihydrobenzofuran-4-yl)-5-fluoro-6-hydroxy-N-methylnicotinamide,
2-((2S,4S)-5-Chloro-6-fluoro-2-(4-hydroxypyrrolidin-2-yl)-2-phenyl-2,3-dihydrobenzofuran-4-yl)-4-(difluoromethoxy)-3-fluorobenzamide,
2-((2S,4S)-5-Chloro-6-fluoro-2-(4-hydroxy-4-methylpyrrolidin-2-yl)-2-phenyl-2,3-dihydrobenzofuran-4-yl)-3-fluoro-4-((S)-2-hydroxypropoxy)benzamide,
(2S,4R)-2-((S)-5-chloro-6-fluoro-2,4-diphenyl-2,3-dihydrobenzofuran-2-yl)-4-fluoropyrrolidine,
2-((2S,4S)-5-Chloro-6-fluoro-2-phenyl-2-((S)-piperidin-2-yl)-2,3-dihydrobenzofuran-4-yl)-3-fluoro-4-(2-hydroxyethoxy)-N-methylbenzamide,
(3-((S)-5-Chloro-6-fluoro-2,4-diphenyl-2,3-dihydrobenzofuran-2-yl)morpholine,
2-((2S,4S)-5-Chloro-6-fluoro-2-(morpholin-3-yl)-2-phenyl-2,3-dihydrobenzofuran-4-yl)-3-fluoro-4-methoxybenzamide,
2-((2S,3S,4S)-5-chloro-6-fluoro-3-hydroxy-2-phenyl-2-(pyrrolidin-2-yl)-2,3-dihydrobenzofuran-4-yl)-3-fluoro-4-methoxybenzamide,
2-((2S,3S,4S)-5-chloro-6-fluoro-3-methoxy-2-phenyl-2-(pyrrolidin-2-yl)-2,3-dihydrobenzofuran-4-yl)-3-fluoro-4-methoxybenzamide,
2-((2S,3S,4S)-5-chloro-6-fluoro-3-methoxy-2-phenyl-2-(pyrrolidin-2-yl)-2,3-dihydrobenzofuran-4-yl)-3-fluoro-4-(2-hydroxyethoxy)benzamide,
2-((2S,3S,4S)-5-chloro-6-fluoro-3-methyl-2-phenyl-2-(pyrrolidin-2-yl)-2,3-dihydrobenzofuran-4-yl)-3-fluoro-4-((S)-2-hydroxypropoxy)benzamide,
2-((2S,4S)-2-(1-Aminoethyl)-5-chloro-2-phenyl-2,3-dihydrobenzofuran-4-yl)-3-fluoro-4-methoxybenzamide,
2-((2S,3S,4S)-2-(1-Aminoethyl)-5-chloro-6-fluoro-3-methyl-2-phenyl-2,3-dihydrobenzofuran-4-yl)-3-fluoro-4-(2-methoxyethoxy)benzamide,
2-((2S,4S)-5-Chloro-6-fluoro-2-phenyl-2-((S)-pyrrolidin-2-yl)indolin-4-yl)-3-fluoro-4-(2-hydroxyethoxy)benzamide,
2-((2S,4S)-5-Chloro-6-fluoro-2-phenyl-2-((S)-pyrrolidin-2-yl)indolin-4-yl)-3-fluoro-4-methoxybenzamide,
2-((2S,4S)-5-Chloro-6-fluoro-2-phenyl-2-((S)-pyrrolidin-2-yl)indolin-4-yl)-3-fluoro-4-((S)-2-hydroxypropoxy)-N-methylbenzamide,
4-((2S,4S)-5-Chloro-6-fluoro-2-phenyl-2-((S)-pyrrolidin-2-yl)indolin-4-yl)-5-fluoro-6-(2-hydroxyethoxy)-N-methylnicotinamide,
2-((2S,4S)-5-Chloro-6-fluoro-2-phenyl-2-((S)-pyrrolidin-2-yl)-2,3-dihydrobenzofuran-4-yl)-3-fluoro-4-methoxybenzamide;
2-((2S,4S)-5-Chloro-6-fluoro-2-phenyl-2-((S)-pyrrolidin-2-yl)-2,3-dihydrobenzofuran-4-yl)-3-fluoro-4-methoxy-N-methylbenzamide; and
2-((2S,3S,4S)-2-(Aminomethyl)-5-chloro-6-fluoro-3-methyl-2-phenyl-2,3-dihydrobenzofuran-4-yl)-3-fluoro-4-methoxybenzamide, or a pharmaceutically acceptable salt thereof.

In an embodiment, the compound of formula (I) is

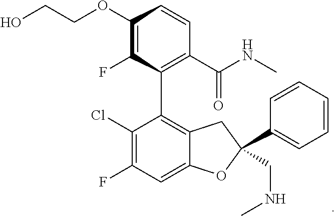

In an embodiment, the compound of formula (I)

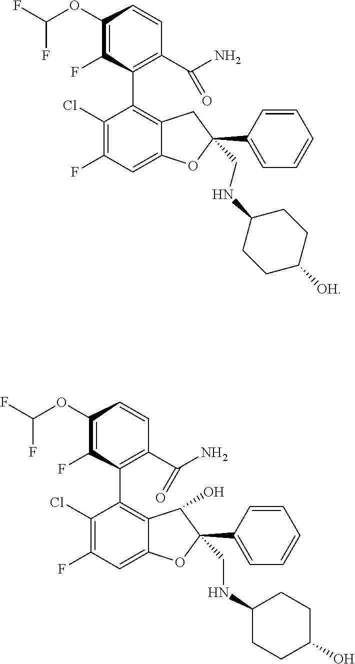

In an embodiment, the compound of formula (I) is

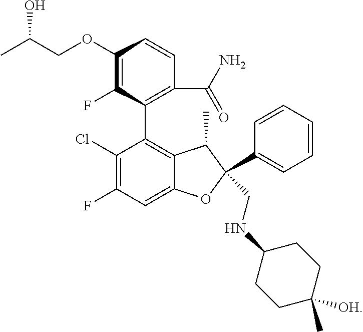

In an embodiment, the compound of formula (I) is

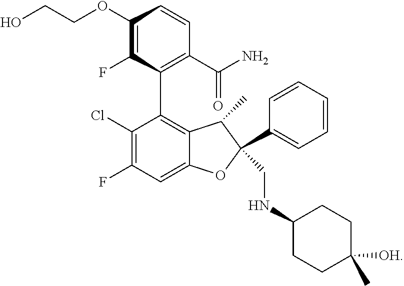

In an embodiment, the compound of formula (I) is

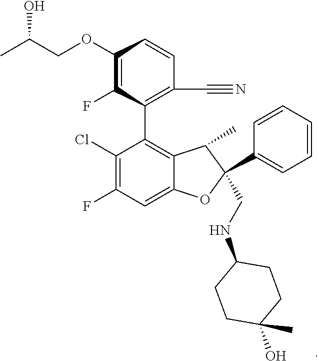

In an embodiment, the compound of formula (I) is

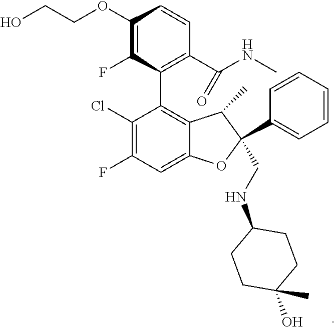

In an embodiment, the compound of formula (I) is

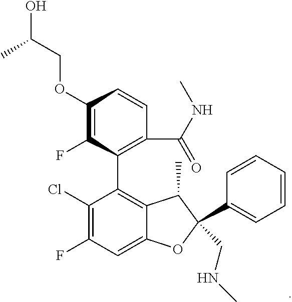

In an embodiment, the compound of formula (I) is

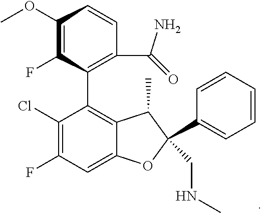

In an embodiment, the compound of formula (I) is

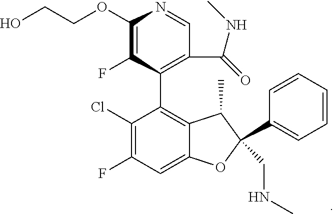

In an embodiment, the compound of formula (I) is

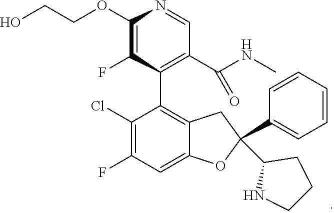

In an embodiment, the compound of formula (I) is

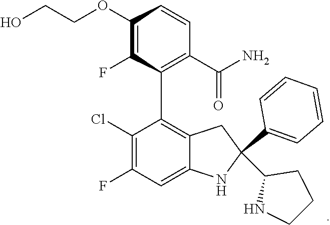

In an embodiment, the compound of formula (I) is (2P)-2-{(2S)-5-Chloro-6-fluoro-2-[(methylamino)methyl]-2-phenyl-2,3-dihydro-1-benzofuran-4-yl}-3-fluoro-4-(2-hydroxyethoxy)-N-methylbenzamide, and has the following structure:

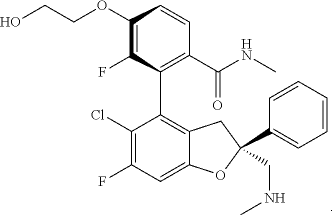

In an embodiment, the compound of formula (I) is (2P)-2-[(2S)-5-Chloro-6-fluoro-2-({[(1r,4S)-4-hydroxycyclohexyl]amino}methyl)-2-phenyl-2,3-dihydro-1-benzofuran-4-yl]-4-(difluoromethoxy)-3-fluorobenzamide, and has the following 10 structure:

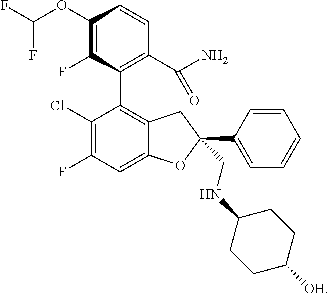

In an embodiment, the compound of formula (I) is (2P)-2-[(2S,3S)-5-Chloro-6-fluoro-3-hydroxy-2-({[(1r,4S)-4-hydroxycyclohexyl]amino}methyl)-2-phenyl-2,3-dihydro-1-benzofuran-4-yl]-4-(difluoromethoxy)-3-fluorobenzamide, and has the following structure:

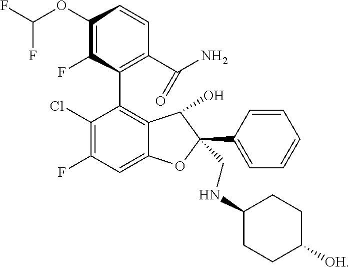

In an embodiment, the compound of formula (I) is (2P)-2-[(2S,3S)-5-Chloro-6-fluoro-2-({[(1r,4S)-4-hydroxy-4-methylcyclohexyl]amino}methyl)-3-methyl-2-phenyl-2,3-dihydro-1-benzofuran-4-yl]-3-fluoro-4-[(2S)-2-hydroxypropoxy]benzamide, and has the following structure:

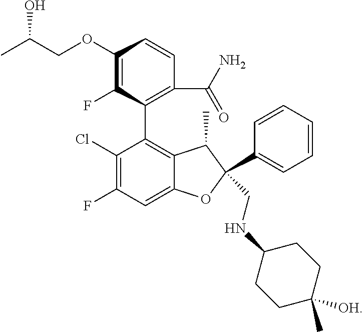

In an embodiment, the compound of formula (I) is (2P)-2-[(2S,3S)-5-Chloro-6-fluoro-2-({[(1r,4S)-4-hydroxy-4-methylcyclohexyl]amino}methyl)-3-methyl-2-phenyl-2,3-dihydro-1-benzofuran-4-yl]-3-fluoro-4-(2-hydroxyethoxy)benzamide, and has the following structure:

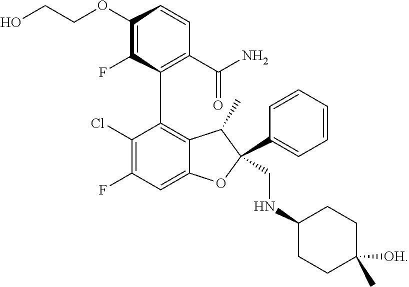

In an embodiment, the compound of formula (I) is (2P)-2-[(2S,3S)-5-Chloro-6-fluoro-2-({[(1r,4S)-4-hydroxy-4-methylcyclohexyl]amino}methyl)-3-methyl-2-phenyl-2,3-dihydro-1-benzofuran-4-yl]-3-fluoro-4-[(2S)-2-hydroxypropoxy]benzonitrile, and has the following structure:

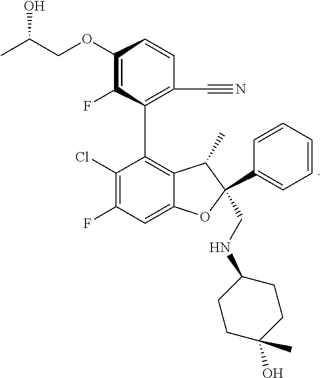

In an embodiment, the compound of formula (I) is (2P)-2-[(2S,3S)-5-Chloro-6-fluoro-2-({[(1r,4S)-4-hydroxy-4-methylcyclohexyl]amino}methyl)-3-methyl-2-phenyl-2,3-dihydro-1-benzofuran-4-yl]-3-fluoro-4-(2-hydroxyethoxy)-N-methylbenzamide, and has the following structure:

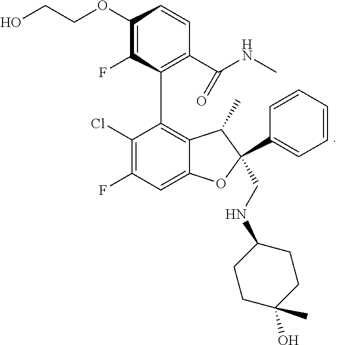

In an embodiment, the compound of formula (I) is (2P)-2-{(2S,3S)-5-Chloro-6-fluoro-3-methyl-2-[(methylamino)methyl]-2-phenyl-2,3-dihydro-1-benzofuran-4-yl}-3-fluoro-4-[(2S)-2-hydroxypropoxy]-N-methylbenzamide, and has the following structure:

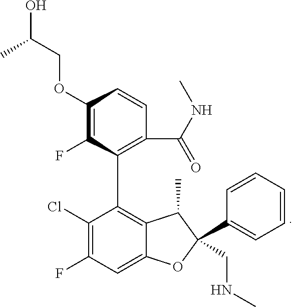

In an embodiment, the compound of formula (I) is (2P)-2-{(2S,3S)-5-Chloro-6-fluoro-3-methyl-2-[(methylamino)methyl]-2-phenyl-2,3-dihydro-1-benzofuran-4-yl}-3-fluoro-4-methoxybenzamide, and has the following structure:

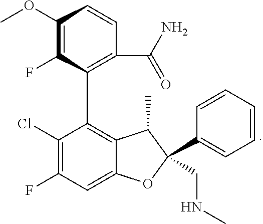

In an embodiment, the compound of formula (I) is (4P)-4-{(2S,3S)-5-Chloro-6-fluoro-3-methyl-2-[(methylamino)methyl]-2-phenyl-2,3-dihydro-1-benzofuran-4-yl}-5-fluoro-6-(2-hydroxyethoxy)-N-methylpyridine-3-carboxamide, and has the following structure:

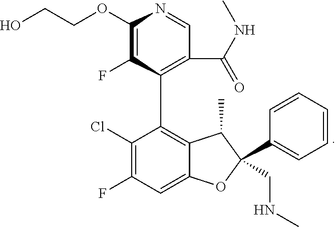

In an embodiment, the compound of formula (I) is (4P)-4-{(2S)-5-Chloro-6-fluoro-2-phenyl-2-[(2S)-pyrrolidin-2-yl]-2,3-dihydro-1-benzofuran-4-yl}-5-fluoro-6-(2-hydroxyethoxy)-N-methylpyridine-3-carboxamide, and has the following structure:

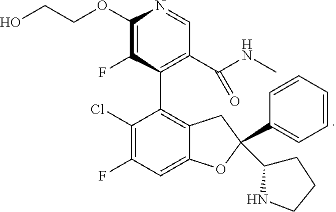

In an embodiment, the compound of formula (I) is (2P)-2-{(2S)-5-Chloro-6-fluoro-2-phenyl-2-[(2S)-pyrrolidin-2-yl]-2,3-dihydro-1H-indol-4-yl}-3-fluoro-4-(2-hydroxyethoxy)benzamide, and has the following structure:

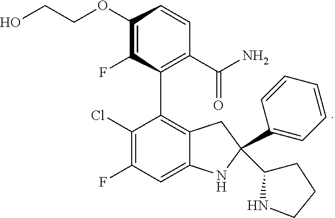

Herein, the term Compound F refers to OH

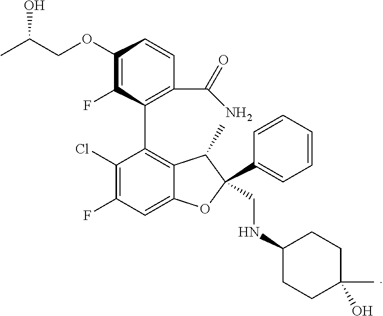

Herein, the term Compound G refers to

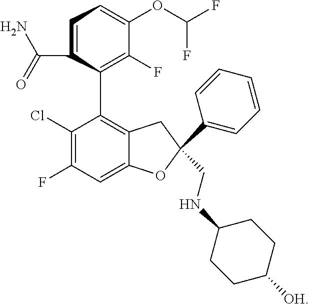

Herein, the term Compound D refers to

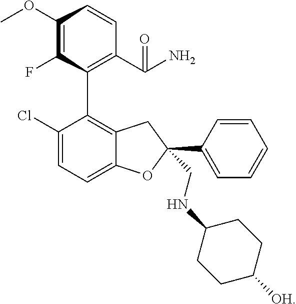

Herein, the term Compound E refers to

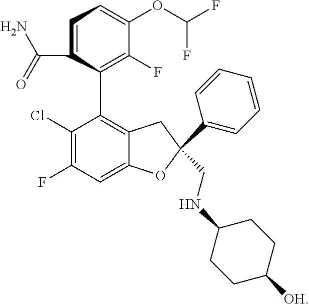

Herein, the term Compound A refers to

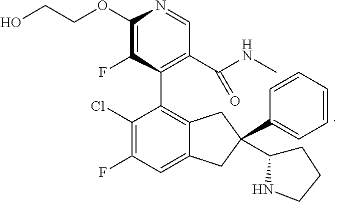

Herein, the term Compound H refers to

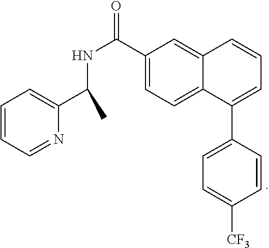 [SMILES: C[C@H](NC(=O)c1ccc2c(-c3ccc(C(F)(F)F)cc3)cccc2c1)c1ccccn1]

Compound H is also known as VT-104, CAS #2417718-25-1. The synthesis of Compound H is described in WO2020/097389.

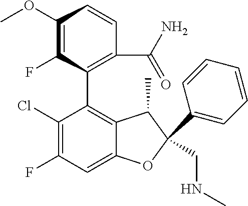

In an embodiment, the TEAD inhibitor is selected from the group consisting of Compound A, Compound B, Compound F, Compound G, Compound D, Compound E, Compound H, VT3989 (Vivace Therapeutics) and TEAD inhibitors selected from those disclosed in WO201753706, WO2017064277, WO2017058716, WO2018185266, WO2018204532, WO2019040380, WO2019113236, WO2019171268, WO2019222431, WO2019232216, WO2020097389, WO202014734, WO2020051099, WO2020243415, WO2020243423, WO2021097110, WO2021102204, WO2021133896 and WO2022087008, and pharmaceutically acceptable salts thereof.

Each of WO201753706, WO2017064277, WO2017058716, WO2018185266, WO2018204532, WO2019040380, WO2019113236, WO2019171268, WO2019222431, WO2019232216, WO2020097389, WO202014734, WO2020051099, WO2020243415, WO2020243423, WO2021097110, WO2021102204, WO2021133896 and WO2022087008, and the TEAD inhibitors disclosed therein are incorporated by reference.

In an embodiment, TEAD inhibitor is selected from the group consisting of Compound A, Compound B, Compound F, Compound G, Compound D, Compound E, Compound H, VT3989 (Vivace Therapeutics), 3-methyl-3-(5-(2-((4-(trifluoromethyl)phenyl)amino)pyridin-3-yl)-1,3,4-oxadiazol-2-yl)pyrrolidin-2-one, (R)-3-methyl-3-(5-(2-((4-(trifluoromethyl)phenyl)amino)pyridin-3-yl)-1,3,4-oxadiazol-2-yl)pyrrolidin-2-one, (S)-3-methyl-3-(5-(2-((4-(trifluoromethyl)phenyl)amino)pyridin-3-yl)-1,3,4-oxadiazol-2-yl)pyrrolidin-2-one, (R)-N-(1-hydroxypropan-2-yl)-5-(4-(trifluoromethyl)phenoxy)-2-naphthamide, N-(2-hydroxy-1-(pyridin-2-yl)ethyl)-5-(4-(trifluoromethyl)phenoxy)-2-naphthamide, (S)-N-(1-(pyridin-2-yl)ethyl)-5-(4-(trifluoromethyl)phenoxy)-2-naphthamide, N-(3-hydroxy-1-(pyridin-2-yl)propyl)-5-(4-(trifluoromethyl)phenoxy)-2-naphthamide, (S)-N-(3-hydroxy-1-(pyridin-2-yl)propyl)-5-(4-(trifluoromethyl)phenoxy)-2-naphthamide, (R)-N-(4-hydroxybutan-2-yl)-5-(4-(trifluoromethyl)phenoxy)-2-naphthamide, (S)-N-(4-hydroxybutan-2-yl)-5-(4-(trifluoromethyl)phenoxy)-2-naphthamide, (S)-N-(2-hydroxy-1-(pyridin-2-yl)ethyl)-5-(4-(trifluoromethyl)phenoxy)-2-naphthamide, N-(1,5-dihydroxypentan-3-yl)-5-(4-(trifluoromethyl)phenoxy)-2-naphthamide, and pharmaceutically acceptable salts thereof.

In an alternative embodiment, the TEAD inhibitor is

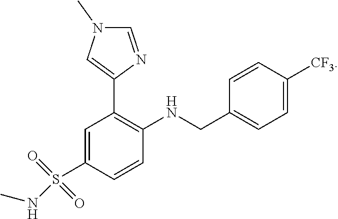 [SMILES: CNS(=O)(=O)c1ccc(NCc2ccc(C(F)(F)F)cc2)c(-c2cn(C)cn2)c1]

This inhibitor is disclosed in WO2022/159986 and WO2020/243415.

Some particularly preferred combinations include the following combinations. According to a further aspect of the invention there is hereby provided a method of treating cancer in a patient in need thereof comprising administering any of the following combinations to said patient. According to a further aspect of the invention there is hereby provided any of the compounds listed in the following combinations for use in the treatment of cancer, wherein said treatment further comprises administration of the other combination partner(s).

### Combinations

VT3989 (Vivace Therapeutics) or a pharmaceutically acceptable salt thereof and a cMET inhibitor (e.g. capmatinib or tepotinib, e.g. capmatinib) or a pharmaceutically acceptable salt thereof
VT3989 (Vivace Therapeutics) or a pharmaceutically acceptable salt thereof and a MAPK pathway inhibitor or a pharmaceutically acceptable salt thereof
VT3989 (Vivace Therapeutics) or a pharmaceutically acceptable salt thereof and a KRAS G12/G13 inhibitor or a pharmaceutically acceptable salt thereof (e.g. a KRAS inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable saltthereof selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas& Otsuka Pharmas), X-37-SHP2 (X-37),

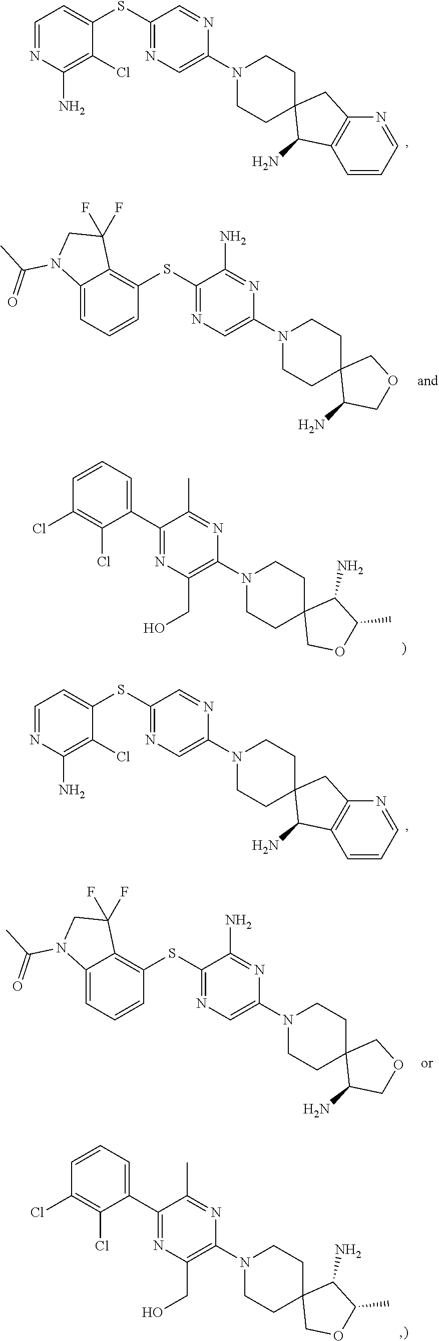

VT3989 (Vivace Therapeutics) or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
VT3989 (Vivace Therapeutics) or a pharmaceutically acceptable salt thereof and an EGFR inhibitor or a pharmaceutically acceptable salt thereof (e.g. erlotinib, osimertinib, neratinib, gefitinib, cetuximab, panitumumab, lapatinib, dacomitinib, necitumumab, nazartinib, LTT462 or vandetanib, e.g. LTT462 or erlotinib)
VT3989 (Vivace Therapeutics) or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
VT3989 (Vivace Therapeutics) or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
VT3989 (Vivace Therapeutics) or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
VT3989 (Vivace Therapeutics) or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin))
VT3989 (Vivace Therapeutics) or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
VT3989 (Vivace Therapeutics) or a pharmaceutically acceptable salt thereof and a Raf inhibitor or a pharmaceutically acceptable salt thereof (e.g. belvarafenib, naporafenib, Encorafenib, vemurafenib or dabrafenib)
VT3989 (Vivace Therapeutics) or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
VT3989 (Vivace Therapeutics) or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)

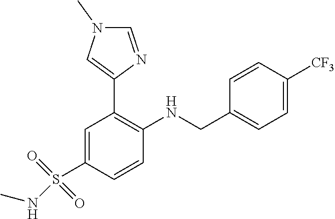 [SMILES: CNS(=O)(=O)c1ccc(NCc2ccc(C(F)(F)F)cc2)c(-c2cn(C)cn2)c1]

or a pharmaceutically acceptable salt thereof and a cMET inhibitor (e.g. capmatinib or tepotinib, e.g. capmatinib) or a pharmaceutically acceptable salt thereof

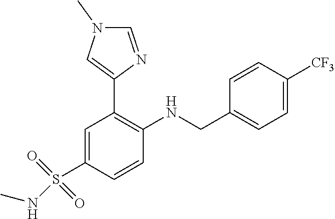 [SMILES: CNS(=O)(=O)c1ccc(NCc2ccc(C(F)(F)F)cc2)c(-c2cn(C)cn2)c1]

H or a pharmaceutically acceptable salt thereof and a MAPK pathway inhibitor or a pharmaceutically acceptable salt thereof

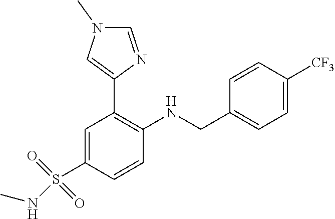 [SMILES: CNS(=O)(=O)c1ccc(NCc2ccc(C(F)(F)F)cc2)c(-c2cn(C)cn2)c1]

H or a pharmaceutically acceptable salt thereof and a KRAS G12/G13 inhibitor or a pharmaceutically acceptable salt thereof (e.g. a KRAS inhibitor or

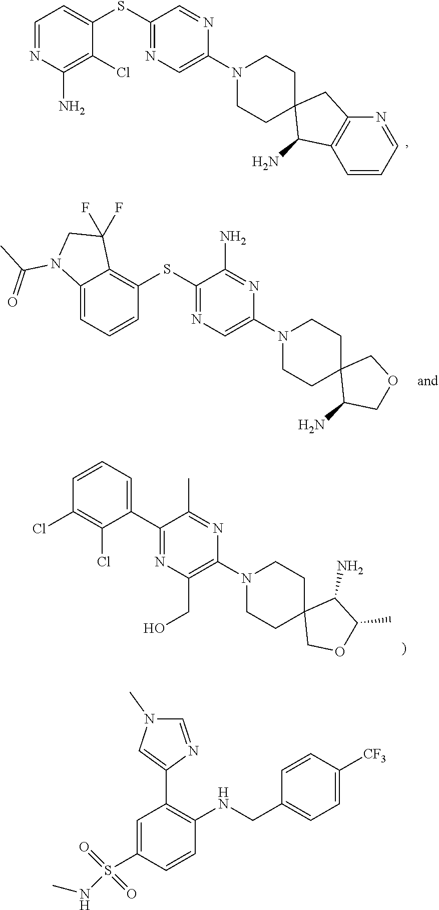

or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

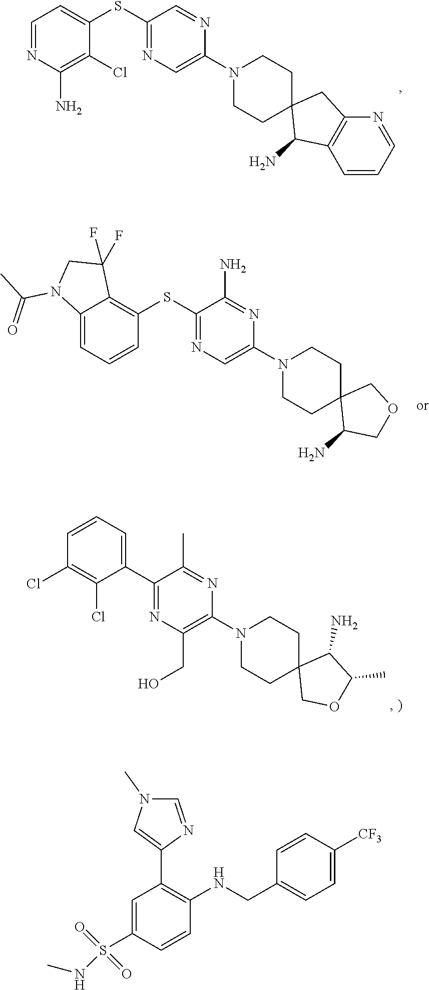

or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)

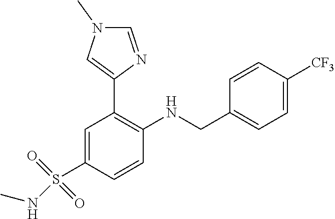 [SMILES: CNS(=O)(=O)c1ccc(NCc2ccc(C(F)(F)F)cc2)c(-c2cn(C)cn2)c1]

or a pharmaceutically acceptable salt thereof and an EGFR inhibitor or a pharmaceutically acceptable salt thereof (e.g. erlotinib, osimertinib, neratinib, gefitinib, cetuximab, panitumumab, lapatinib, dacomitinib, necitumumab, nazartinib, LTT462 or vandetanib, e.g. LTT462 or erlotinib)

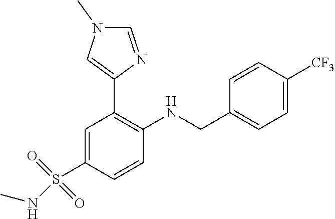 [SMILES: CNS(=O)(=O)c1ccc(NCc2ccc(C(F)(F)F)cc2)c(-c2cn(C)cn2)c1]

or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)

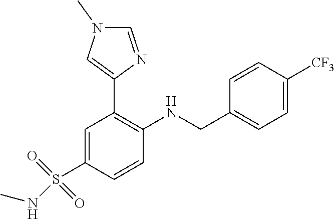 [SMILES: CNS(=O)(=O)c1ccc(NCc2ccc(C(F)(F)F)cc2)c(-c2cn(C)cn2)c1]

or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)

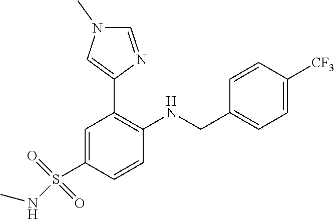 [SMILES: CNS(=O)(=O)c1ccc(NCc2ccc(C(F)(F)F)cc2)c(-c2cn(C)cn2)c1]

or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)

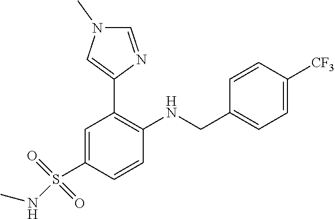 [SMILES: CNS(=O)(=O)c1ccc(NCc2ccc(C(F)(F)F)cc2)c(-c2cn(C)cn2)c1]

or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828 milademetan or hdm201 (also known as siremadlin))

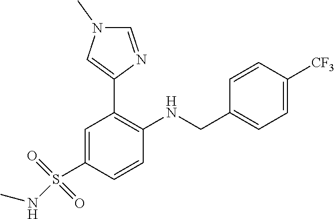 [SMILES: CNS(=O)(=O)c1ccc(NCc2ccc(C(F)(F)F)cc2)c(-c2cn(C)cn2)c1]

or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)

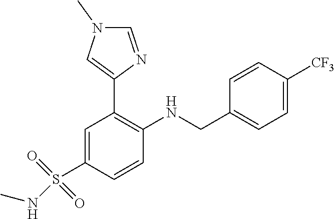 [SMILES: CNS(=O)(=O)c1ccc(NCc2ccc(C(F)(F)F)cc2)c(-c2cn(C)cn2)c1]

or a pharmaceutically acceptable salt thereof and a Raf inhibitor or a pharmaceutically acceptable salt thereof (e.g. belvarafenib, naporafenib, Encorafenib, vemurafenib or dabrafenib)

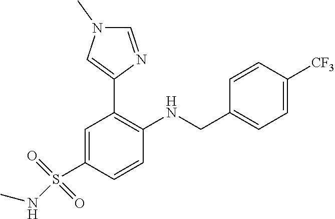 [SMILES: CNS(=O)(=O)c1ccc(NCc2ccc(C(F)(F)F)cc2)c(-c2cn(C)cn2)c1]

or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)

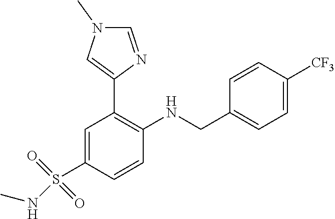 [SMILES: CNS(=O)(=O)c1ccc(NCc2ccc(C(F)(F)F)cc2)c(-c2cn(C)cn2)c1]

or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Compound B or a pharmaceutically acceptable salt thereof and a cMET inhibitor or a pharmaceutically acceptable salt thereof (e.g. capmatinib or tepotinib, e.g. capmatinib)
Compound B or a pharmaceutically acceptable salt thereof and a MAPK pathway inhibitor or a pharmaceutically acceptable salt thereof
Compound B or a pharmaceutically acceptable salt thereof (e.g. a KRAS G12/G13 inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

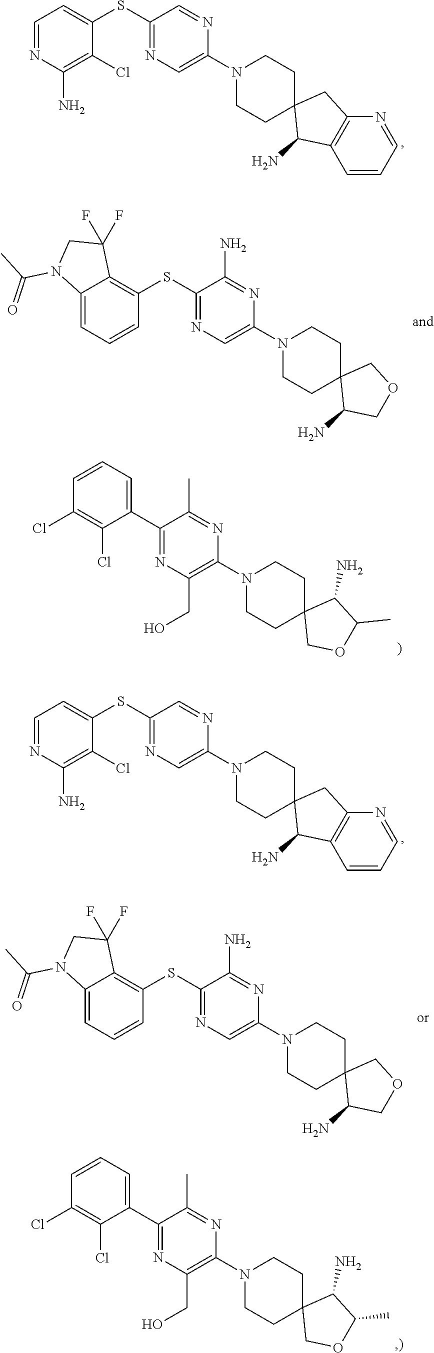

Compound B or a pharmaceutically acceptable salt thereof and a KRAS G121G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
Compound B or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
Compound B or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
Compound B or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Compound B or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Compound B or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
Compound B or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
Compound B or a pharmaceutically acceptable salt thereof and a Raf inhibitor or a pharmaceutically acceptable salt thereof (e.g. belvarafenib, naporafenib, Encorafenib, vemurafenib or dabrafenib)
Compound B or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Compound B or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Compound A or a pharmaceutically acceptable salt thereof and a cMET inhibitor or a pharmaceutically acceptable salt thereof (e.g. capmatinib or tepotinib, e.g. capmatinib)
Compound A or a pharmaceutically acceptable salt thereof and a MAPK pathway inhibitor or a pharmaceutically acceptable salt thereof
Compound A or a pharmaceutically acceptable salt thereof (e.g. a KRAS G12/G13 inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

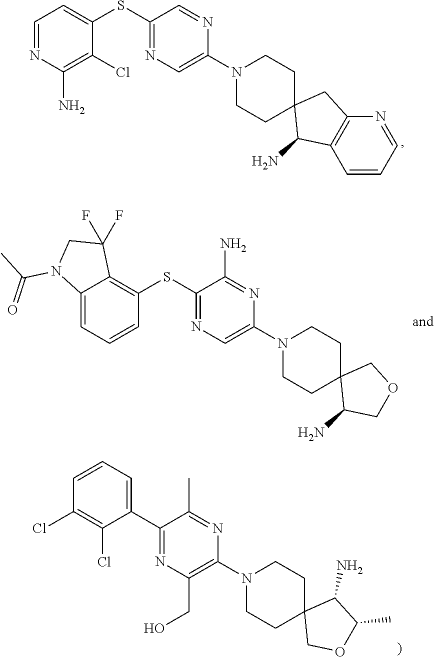 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

Compound A or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

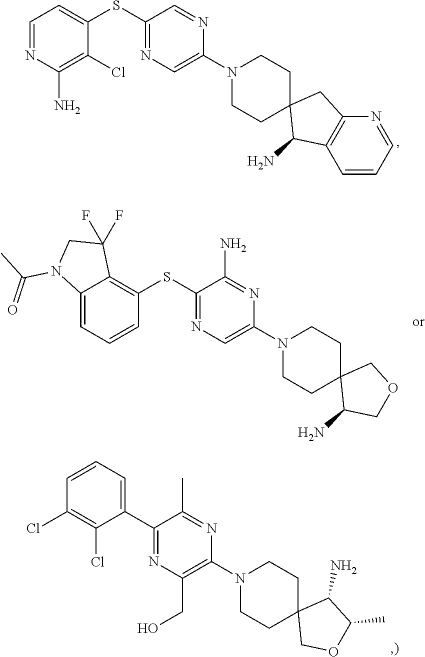 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

Compound A or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
Compound A or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
Compound A or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
Compound A or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Compound A or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Compound A or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
Compound A or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
Compound A or a pharmaceutically acceptable salt thereof and a Raf inhibitor or a pharmaceutically acceptable salt thereof (e.g. belvarafenib, naporafenib, Encorafenib, vemurafenib or dabrafenib)
Compound A or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Compound A or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Compound D or a pharmaceutically acceptable salt thereof and a cMET inhibitor or a pharmaceutically acceptable salt thereof (e.g. capmatinib or tepotinib, e.g. capmatinib)
Compound D or a pharmaceutically acceptable salt thereof and a MAPK pathway inhibitor or a pharmaceutically acceptable salt thereof
Compound D or a pharmaceutically acceptable salt thereof (e.g. a KRAS G12/G13 inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 37),

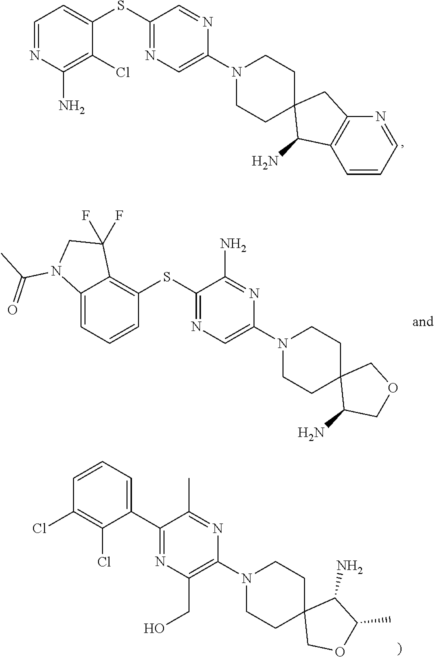 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

Compound D or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

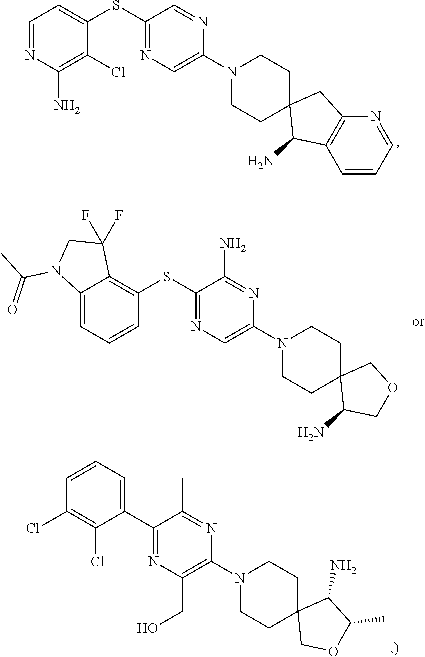 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

Compound D or a pharmaceutically acceptable salt thereof and a KRAS G121G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
Compound D or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
Compound D or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
Compound D or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Compound D or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Compound D or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
Compound D or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
Compound D or a pharmaceutically acceptable salt thereof and a Raf inhibitor or a pharmaceutically acceptable salt thereof (e.g. belvarafenib, naporafenib, Encorafenib, vemurafenib or dabrafenib)
Compound D or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Compound D or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Compound E or a pharmaceutically acceptable salt thereof and a cMET inhibitor or a pharmaceutically acceptable salt thereof (e.g. capmatinib or tepotinib, e.g. capmatinib)
Compound E or a pharmaceutically acceptable salt thereof and a MAPK pathway inhibitor or a pharmaceutically acceptable salt thereof
Compound E or a pharmaceutically acceptable salt thereof (e.g. a KRAS G12/G13 inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB

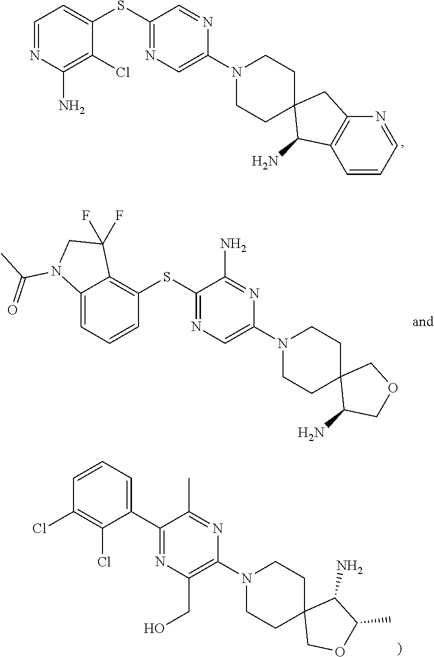 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

Compound E or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

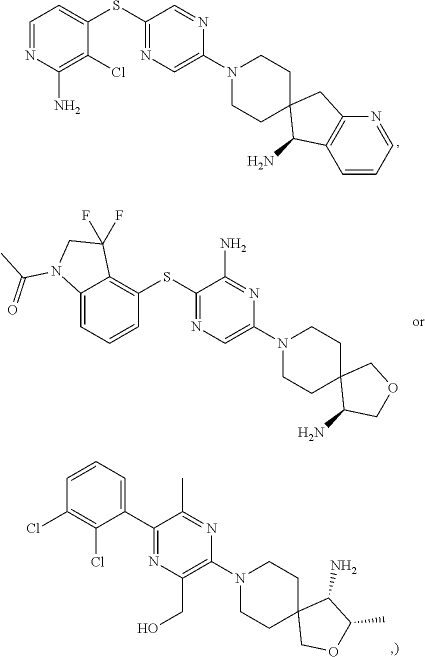 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

Compound E or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
Compound E or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
Compound E or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
Compound E or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Compound E or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Compound E or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
Compound E or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
Compound E or a pharmaceutically acceptable salt thereof and a Raf inhibitor or a pharmaceutically acceptable salt thereof (e.g. belvarafenib, naporafenib, Encorafenib, vemurafenib or dabrafenib)
Compound E or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Compound E or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Compound F (2-((2S,3S)-5-chloro-6-fluoro-2-((((1 r,4S)-4-hydroxy-4-methylcyclohexyl)amino)methyl)-3-methyl-2-phenyl-2,3-dihydrobenzofuran-4-yl)-3-fluoro-4-((S)-2-hydroxypropoxy)benzamide) or a pharmaceutically acceptable salt thereof and a cMET inhibitor or a pharmaceutically acceptable salt thereof (e.g. capmatinib or tepotinib, e.g. capmatinib)
Compound F or a pharmaceutically acceptable salt thereof and a MAPK pathway inhibitor or a pharmaceutically acceptable salt thereof

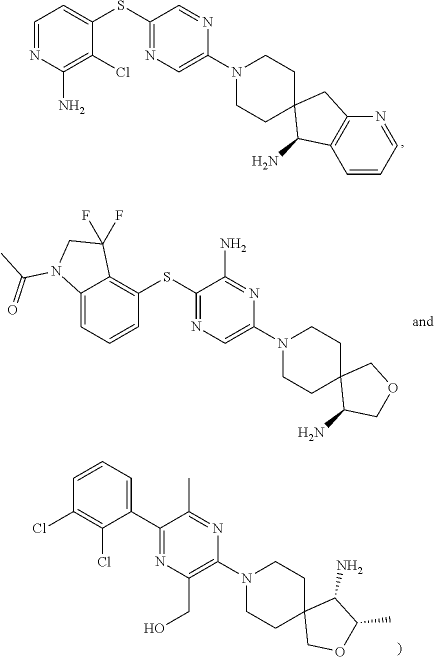 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

Compound F or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

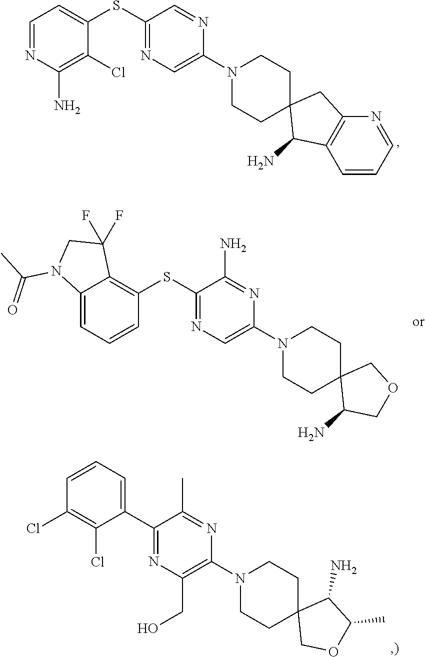 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

Compound F or a pharmaceutically acceptable salt thereof and a KRAS G121G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
Compound F or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
Compound F or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
Compound F or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Compound F or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Compound F or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
Compound F or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
Compound F or a pharmaceutically acceptable salt thereof and a Raf inhibitor or a pharmaceutically acceptable salt thereof (e.g. belvarafenib, naporafenib, Encorafenib, vemurafenib or dabrafenib)
Compound F or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Compound F or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Compound G or a pharmaceutically acceptable salt thereof and a cMET inhibitor or a pharmaceutically acceptable salt thereof (e.g. capmatinib or tepotinib, e.g. capmatinib)
Compound G or a pharmaceutically acceptable salt thereof and a MAPK pathway inhibitor or a pharmaceutically acceptable salt thereof
Compound G or a pharmaceutically acceptable salt thereof (e.g. a KRAS G12/G13 inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

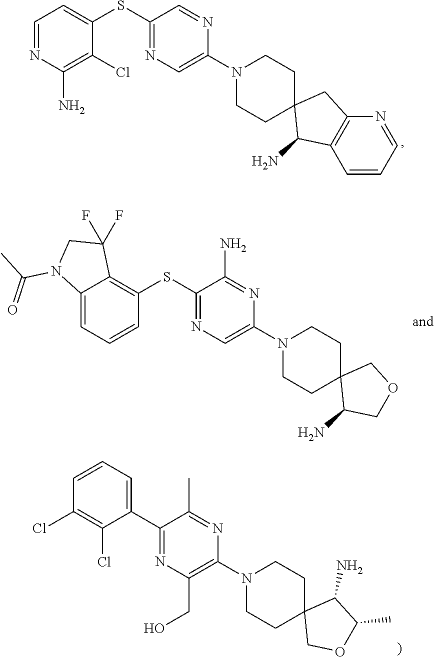 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

Compound G or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

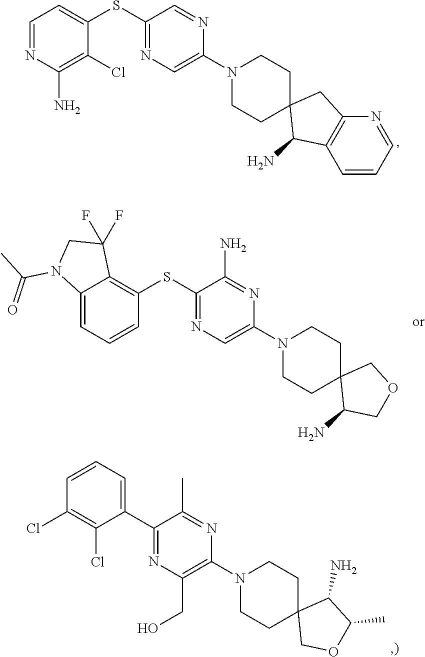 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

Compound G or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
Compound G or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
Compound G or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
Compound G or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Compound G or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Compound G or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
Compound G or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
Compound G or a pharmaceutically acceptable salt thereof and a Raf inhibitor or a pharmaceutically acceptable salt thereof (e.g. belvarafenib, naporafenib, Encorafenib, vemurafenib or dabrafenib)
Compound G or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Compound G or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a cMET inhibitor or a pharmaceutically acceptable salt thereof (e.g. capmatinib or tepotinib, e.g. capmatinib)
3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a MAPK pathway inhibitor or a pharmaceutically acceptable salt thereof
3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a KRAS G12/G13 inhibitor or a pharmaceutically acceptable salt thereof (e.g. a KRAS G12C inhibitor or a pharmaceutically acceptable salt thereof or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

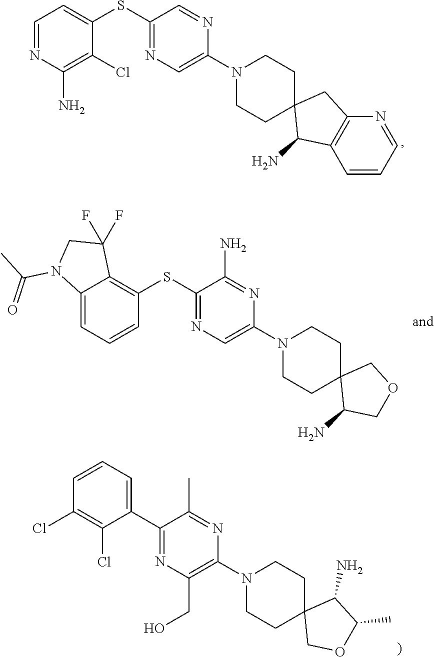 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

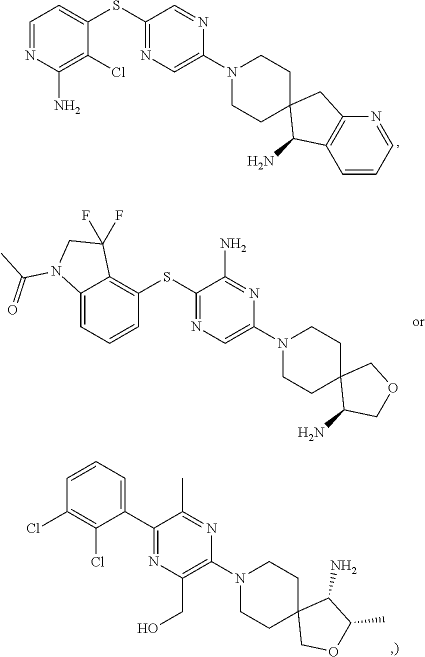 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

3-methyl-3-(5-(2-((4-(trifluoromethyl)phenyl)amino)pyridin-3-yl)-1,3,4-oxadiazol-2-yl)pyrrolidin-2-one and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a Raf inhibitor or a pharmaceutically acceptable salt thereof (e.g. belvarafenib, naporafenib, Encorafenib, vemurafenib or dabrafenib)
3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(R)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a cMET inhibitor or a pharmaceutically acceptable salt thereof (e.g. capmatinib or tepotinib, e.g. capmatinib)
(R)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a MAPK pathway inhibitor or a pharmaceutically acceptable salt thereof
(R)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), ASTX (Taiho & Otsuka Pharmas), X-37-SHP2 (X-37),

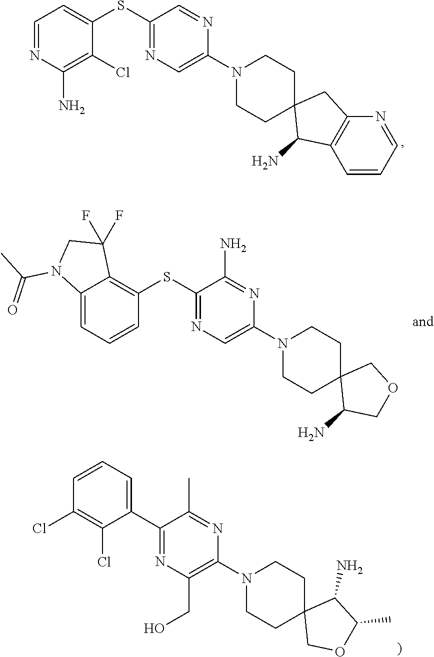 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(R)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

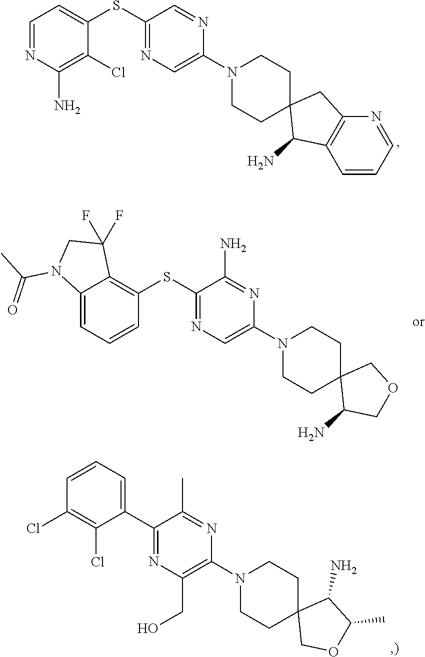 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(R)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a KRAS G121G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(R)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(R)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(R)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
(R)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(R)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
(R)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a Raf inhibitor or a pharmaceutically acceptable salt thereof (e.g. belvarafenib, naporafenib, Encorafenib, vemurafenib or dabrafenib)
(R)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
(S)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a cMET inhibitor or a pharmaceutically acceptable salt thereof (e.g. capmatinib or tepotinib, e.g. capmatinib)
(S)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a MAPK pathway inhibitor or a pharmaceutically acceptable salt thereof
(S)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), ASTX (Taiho & Otsuka Pharmas), X-37-SHP2 (X-37),

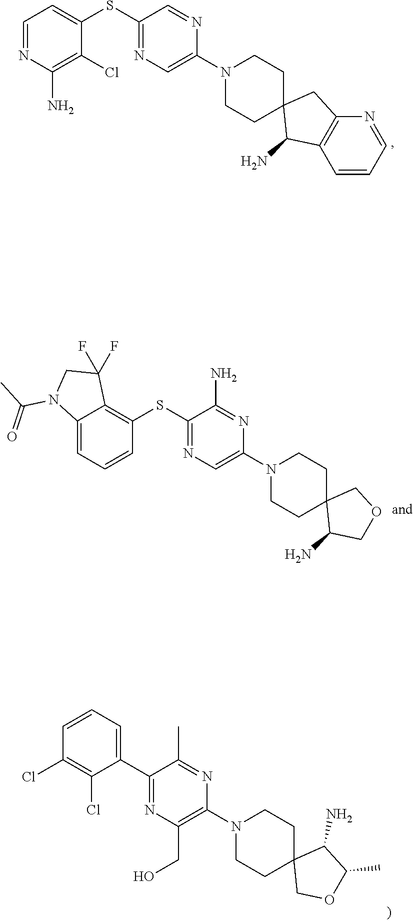 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(S)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

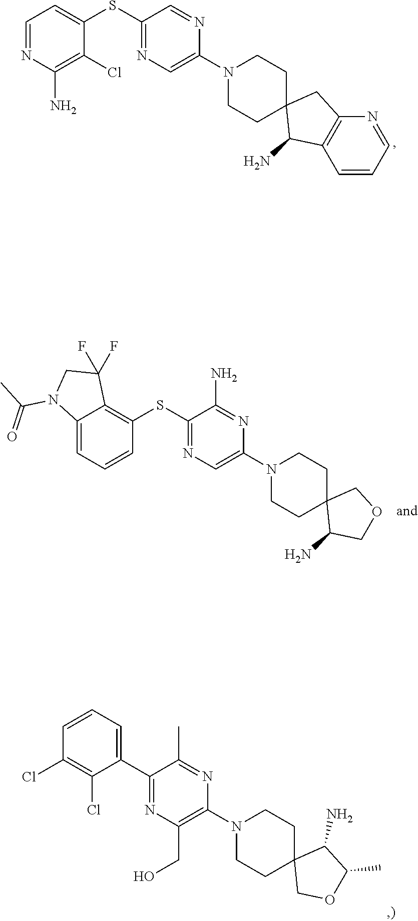 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(S)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a KRAS G121G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(S)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(S)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(S)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
(S)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(S)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
(S)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a Raf inhibitor or a pharmaceutically acceptable salt thereof (e.g. belvarafenib, naporafenib, Encorafenib, vemurafenib or dabrafenib)
(S)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-3-methyl-3-(5-(2-((4-(trifluoromethyl)phenyl)amino)pyridin-3-yl)-1,3,4-oxadiazol-2-yl)pyrrolidin-2-one or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(R)-N-(1-hydroxypropan-2-yl)-5-(4-(trifluoromethyl)phenoxy)-2-naphthamide or a pharmaceutically acceptable salt thereof and a cMET inhibitor or a pharmaceutically acceptable salt thereof (e.g. capmatinib or tepotinib, e.g. capmatinib)
(R)-N-(1-hydroxypropan-2-yl)-5-(4-(trifluoromethyl)phenoxy)-2-naphthamide or a pharmaceutically acceptable salt thereof and a MAPK pathway inhibitor or a pharmaceutically acceptable salt thereof
(R)-N-(1-hydroxypropan-2-yl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), ASTX (Taiho & Otsuka Pharmas), X-37-SHP2 (X-37),

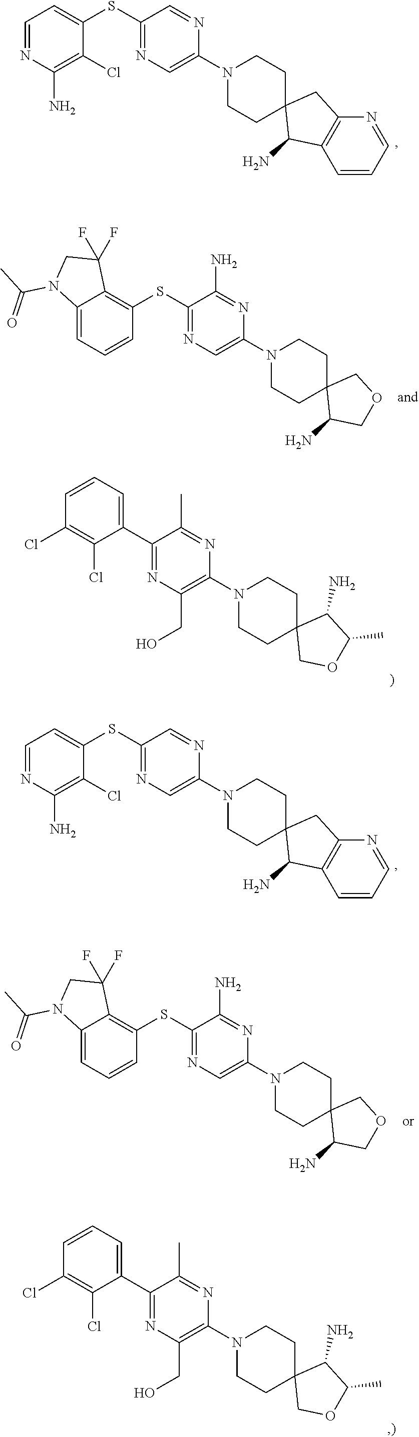

(R)-N-(1-hydroxypropan-2-yl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(R)-N-(1-hydroxypropan-2-yl)-5-(4-(trifluoromethyl)phenoxy)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(R)-N-(1-hydroxypropan-2-yl)-5-(4-(trifluoromethyl)phenoxy)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(R)-N-(1-hydroxypropan-2-yl)-5-(4-(trifluoromethyl)phenoxy)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-N-(1-hydroxypropan-2-yl)-5-(4-(trifluoromethyl)phenoxy)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(R)-N-(1-hydroxypropan-2-yl)-5-(4-(trifluoromethyl)phenoxy)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(R)-N-(1-hydroxypropan-2-yl)-5-(4-(trifluoromethyl)phenoxy)-2-naphthamide or a pharmaceutically acceptable salt thereof and a CDK4/6 inhibitor or a pharmaceutically acceptable salt thereof (e.g. a CDK416 inhibitor or a pharmaceutically acceptable salt thereof selected from the group consisting of palbociclib, ribociclib and abemaciclib)
(R)-N-(1-hydroxypropan-2-yl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(R)-N-(1-hydroxypropan-2-yl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting
(R)-N-(1-hydroxypropan-2-yl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(2-hydroxy-1-(pyridin-2-yl)ethyl)-5-(4-(trifluoromethyl)phenoxy)-2-naphthamide or a pharmaceutically acceptable salt thereof and a cMET inhibitor or a pharmaceutically acceptable salt thereof (e.g. capmatinib or tepotinib, e.g. capmatinib)
N-(2-hydroxy-1-(pyridin-2-yl)ethyl)-5-(4-(trifluoromethyl)phenoxy)-2-naphthamide or a pharmaceutically acceptable salt thereof and a MAPK pathway inhibitor or a pharmaceutically acceptable salt thereof
N-(2-hydroxy-1-(pyridin-2-yl)ethyl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), ASTX (Taiho & Otsuka Pharmas), X-37-SHP2 (X-37),

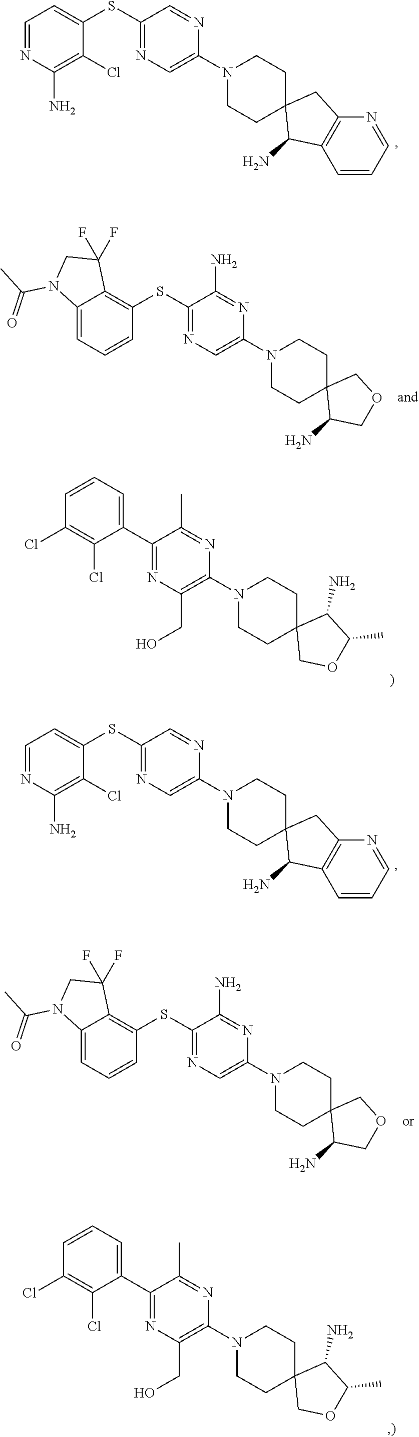

N-(2-hydroxy-1-(pyridin-2-yl)ethyl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(N-(2-hydroxy-1-(pyridin-2-yl)ethyl)-5-(4-(trifluoromethyl)phenoxy)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-(2-hydroxy-1-(pyridin-2-yl)ethyl)-5-(4-(trifluoromethyl)phenoxy)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-(2-hydroxy-1-(pyridin-2-yl)ethyl)-5-(4-(trifluoromethyl)phenoxy)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(2-hydroxy-1-(pyridin-2-yl)ethyl)-5-(4-(trifluoromethyl)phenoxy)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(2-hydroxy-1-(pyridin-2-yl)ethyl)-5-(4-(trifluoromethyl)phenoxy)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-(2-hydroxy-1-(pyridin-2-yl)ethyl)-5-(4-(trifluoromethyl)phenoxy)-2-naphthamide or a pharmaceutically acceptable salt thereof and a CDK4/6 inhibitor or a pharmaceutically acceptable salt thereof (e.g. a CDK416 inhibitor or a pharmaceutically acceptable salt thereof selected from the group consisting of palbociclib, ribociclib and abemaciclib)
N-(2-hydroxy-1-(pyridin-2-yl)ethyl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-(2-hydroxy-1-(pyridin-2-yl)ethyl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(2-hydroxy-1-(pyridin-2-yl)ethyl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(S)-N-(1-(pyridin-2-yl)ethyl)-5-(4-(trifluoromethyl)phenoxy)-2-naphthamide or a pharmaceutically acceptable salt thereof and a cMET inhibitor or a pharmaceutically acceptable salt thereof (e.g. capmatinib or tepotinib, e.g. capmatinib)
(S)-N-(1-(pyridin-2-yl)ethyl)-5-(4-(trifluoromethyl)phenoxy)-2-naphthamide or a pharmaceutically acceptable salt thereof and a MAPK pathway inhibitor or a pharmaceutically acceptable salt thereof
(S)-N-(1-(pyridin-2-yl)ethyl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), ASTX (Taiho & Otsuka Pharmas), X-37-SHP2 (X-37),

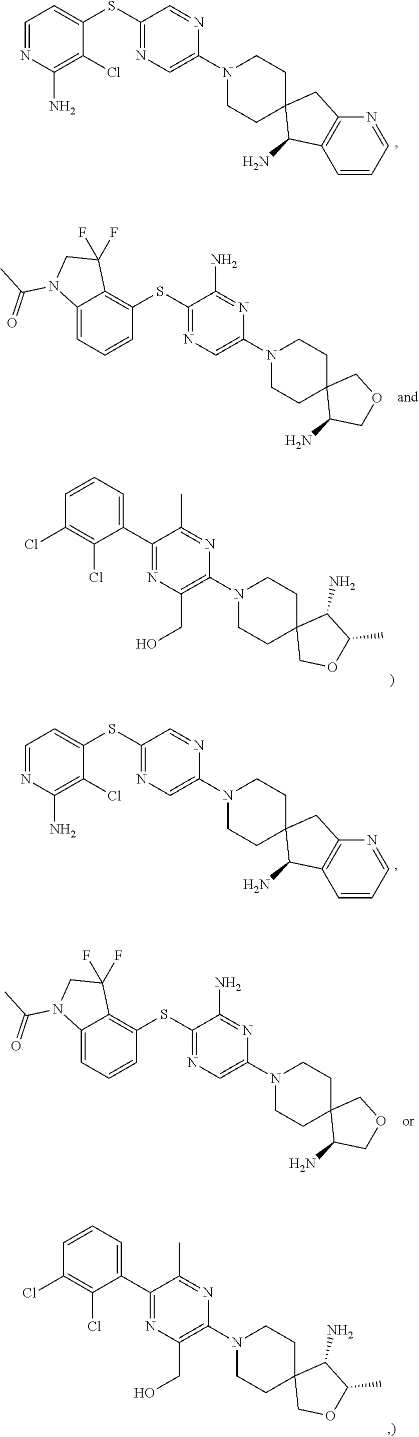

(S)-N-(1-(pyridin-2-yl)ethyl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(S)-N-(1-(pyridin-2-yl)ethyl)-5-(4-(trifluoromethyl)phenoxy)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(S)-N-(1-(pyridin-2-yl)ethyl)-5-(4-(trifluoromethyl)phenoxy)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(S)-N-(1-(pyridin-2-yl)ethyl)-5-(4-(trifluoromethyl)phenoxy)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-N-(1-(pyridin-2-yl)ethyl)-5-(4-(trifluoromethyl)phenoxy)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(S)-N-(1-(pyridin-2-yl)ethyl)-5-(4-(trifluoromethyl)phenoxy)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(S)-N-(1-(pyridin-2-yl)ethyl)-5-(4-(trifluoromethyl)phenoxy)-2-naphthamide or a pharmaceutically acceptable salt thereof and a CDK4/6 inhibitor or a pharmaceutically acceptable salt thereof (e.g. a CDK416 inhibitor or a pharmaceutically acceptable salt thereof selected from the group consisting of palbociclib, ribociclib and abemaciclib)
(S)-N-(1-(pyridin-2-yl)ethyl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(S)-N-(1-(pyridin-2-yl)ethyl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting
(S)-N-(1-(pyridin-2-yl)ethyl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(3-hydroxy-1-(pyridin-2-yl)propyl)-5-(4-(trifluoromethyl)phenoxy)-2-naphthamide or a pharmaceutically acceptable salt thereof and a cMET inhibitor or a pharmaceutically acceptable salt thereof (e.g. capmatinib or tepotinib, e.g. capmatinib)
N-(3-hydroxy-1-(pyridin-2-yl)propyl)-5-(4-(trifluoromethyl)phenoxy)-2-naphthamide or a pharmaceutically acceptable salt thereof and a MAPK pathway inhibitor or a pharmaceutically acceptable salt thereof
N-(3-hydroxy-1-(pyridin-2-yl)propyl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), ASTX (Taiho & Otsuka Pharmas), X-37-SHP2 (X-37),

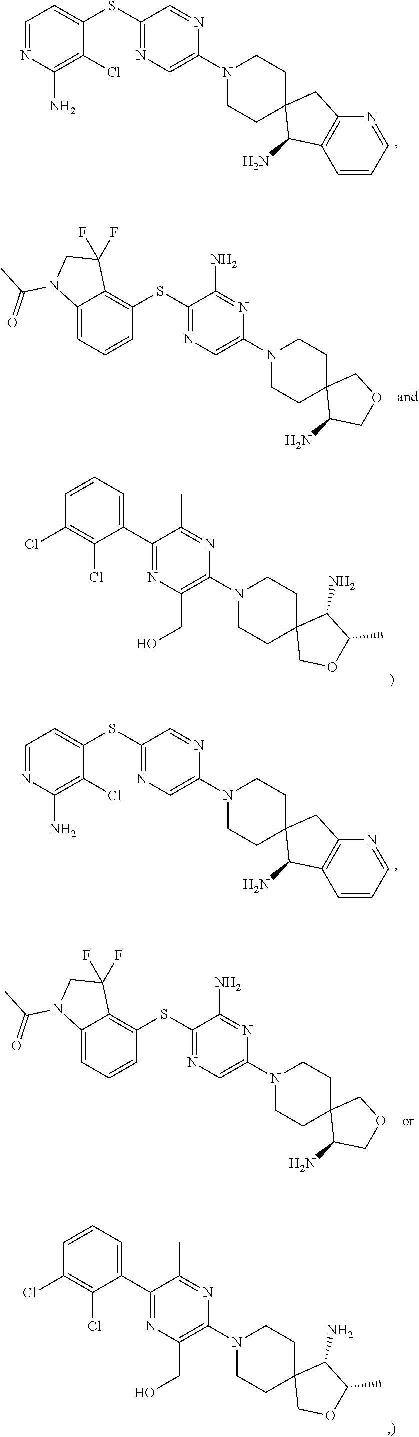

N-(3-hydroxy-1-(pyridin-2-yl)propyl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
N-(3-hydroxy-1-(pyridin-2-yl)propyl)-5-(4-(trifluoromethyl)phenoxy)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-(3-hydroxy-1-(pyridin-2-yl)propyl)-5-(4-(trifluoromethyl)phenoxy)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-(3-hydroxy-1-(pyridin-2-yl)propyl)-5-(4-(trifluoromethyl)phenoxy)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(3-hydroxy-1-(pyridin-2-yl)propyl)-5-(4-(trifluoromethyl)phenoxy)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(3-hydroxy-1-(pyridin-2-yl)propyl)-5-(4-(trifluoromethyl)phenoxy)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-(3-hydroxy-1-(pyridin-2-yl)propyl)-5-(4-(trifluoromethyl)phenoxy)-2-naphthamide or a pharmaceutically acceptable salt thereof and a CDK4/6 inhibitor or a pharmaceutically acceptable salt thereof (e.g. a CDK416 inhibitor or a pharmaceutically acceptable salt thereof selected from the group consisting of palbociclib, ribociclib and abemaciclib)
N-(3-hydroxy-1-(pyridin-2-yl)propyl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-(3-hydroxy-1-(pyridin-2-yl)propyl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(3-hydroxy-1-(pyridin-2-yl)propyl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(S)-N-(3-hydroxy-1-(pyridin-2-yl)propyl)-5-(4-(trifluoromethyl)phenoxy)-2-naphthamide or a pharmaceutically acceptable salt thereof and a cMET inhibitor or a pharmaceutically acceptable salt thereof (e.g. capmatinib or tepotinib, e.g. capmatinib)
(S)-N-(3-hydroxy-1-(pyridin-2-yl)propyl)-5-(4-(trifluoromethyl)phenoxy)-2-naphthamide or a pharmaceutically acceptable salt thereof and a MAPK pathway inhibitor or a pharmaceutically acceptable salt thereof
(S)-N-(3-hydroxy-1-(pyridin-2-yl)propyl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), ASTX (Taiho & Otsuka Pharmas), X-37-SHP2 (X-37),

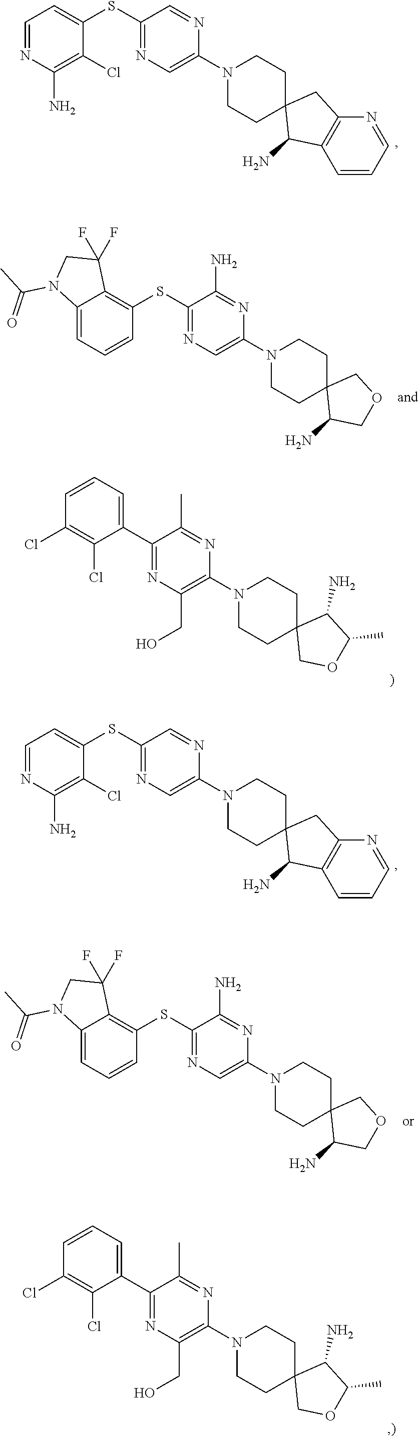

(S)-N-(3-hydroxy-1-(pyridin-2-yl)propyl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(S)-N-(3-hydroxy-1-(pyridin-2-yl)propyl)-5-(4-(trifluoromethyl)phenoxy)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(S)-N-(3-hydroxy-1-(pyridin-2-yl)propyl)-5-(4-(trifluoromethyl)phenoxy)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(S)-N-(3-hydroxy-1-(pyridin-2-yl)propyl)-5-(4-(trifluoromethyl)phenoxy)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-N-(3-hydroxy-1-(pyridin-2-yl)propyl)-5-(4-(trifluoromethyl)phenoxy)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(S)-N-(3-hydroxy-1-(pyridin-2-yl)propyl)-5-(4-(trifluoromethyl)phenoxy)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(S)-N-(3-hydroxy-1-(pyridin-2-yl)propyl)-5-(4-(trifluoromethyl)phenoxy)-2-naphthamide or a pharmaceutically acceptable salt thereof and a CDK4/6 inhibitor or a pharmaceutically acceptable salt thereof (e.g. a CDK416 inhibitor or a pharmaceutically acceptable salt thereof selected from the group consisting of palbociclib, ribociclib and abemaciclib)
(S)-N-(3-hydroxy-1-(pyridin-2-yl)propyl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(S)-N-(3-hydroxy-1-(pyridin-2-yl)propyl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting
(S)-N-(3-hydroxy-1-(pyridin-2-yl)propyl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor (e.g. ulixertinib, GDC-0994, KO-947, Vtx-11e, SCH-772984, MK2853, LY3214996, MK08353, LTT462 or BVD-523, e.g. LTT462)
(R)-N-(4-hydroxybutan-2-yl)-5-(4-(trifluoromethyl)phenoxy)-2-naphthamide or a pharmaceutically acceptable salt thereof and a cMET inhibitor or a pharmaceutically acceptable salt thereof (e.g. capmatinib or tepotinib, e.g. capmatinib)
(R)-N-(4-hydroxybutan-2-yl)-5-(4-(trifluoromethyl)phenoxy)-2-naphthamide or a pharmaceutically acceptable salt thereof and a MAPK pathway inhibitor or a pharmaceutically acceptable salt thereof
(R)-N-(4-hydroxybutan-2-yl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), ASTX (Taiho & Otsuka Pharmas), X-37-SHP2 (X-37),

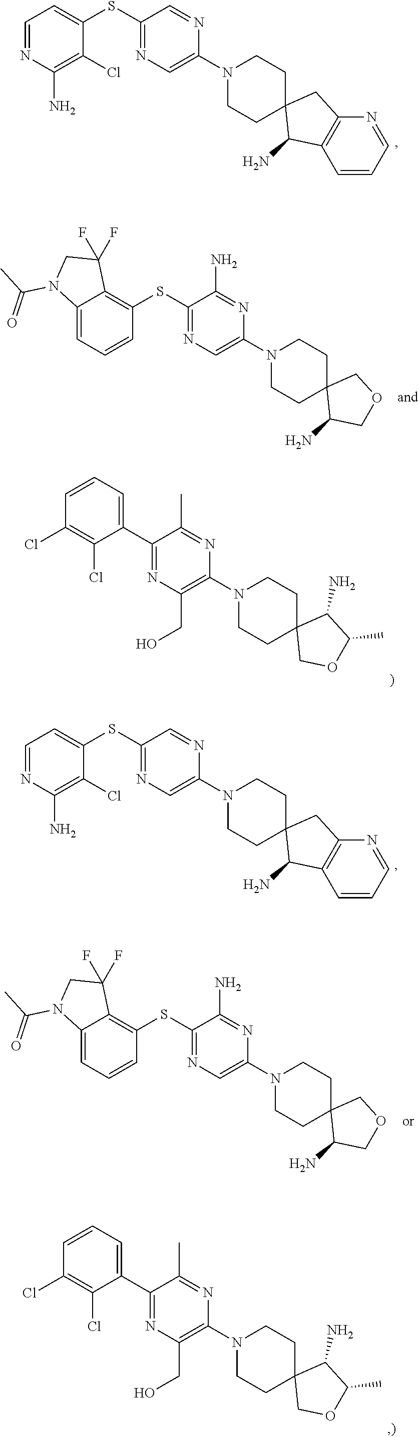

(R)-N-(4-hydroxybutan-2-yl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(R)-N-(4-hydroxybutan-2-yl)-5-(4-(trifluoromethyl)phenoxy)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(R)-N-(4-hydroxybutan-2-yl)-5-(4-(trifluoromethyl)phenoxy)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(R)-N-(4-hydroxybutan-2-yl)-5-(4-(trifluoromethyl)phenoxy)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-N-(4-hydroxybutan-2-yl)-5-(4-(trifluoromethyl)phenoxy)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(R)-N-(4-hydroxybutan-2-yl)-5-(4-(trifluoromethyl)phenoxy)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(R)-N-(4-hydroxybutan-2-yl)-5-(4-(trifluoromethyl)phenoxy)-2-naphthamide or a pharmaceutically acceptable salt thereof and a CDK4/6 inhibitor or a pharmaceutically acceptable salt thereof (e.g. a CDK416 inhibitor or a pharmaceutically acceptable salt thereof selected from the group consisting of palbociclib, ribociclib and abemaciclib)
(R)-N-(4-hydroxybutan-2-yl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(R)-N-(4-hydroxybutan-2-yl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting
(R)-N-(4-hydroxybutan-2-yl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(S)-N-(4-hydroxybutan-2-yl)-5-(4-(trifluoromethyl)phenoxy)-2-naphthamide or a pharmaceutically acceptable salt thereof and a cMET inhibitor or a pharmaceutically acceptable salt thereof (e.g. capmatinib or tepotinib, e.g. capmatinib)
(S)-N-(4-hydroxybutan-2-yl)-5-(4-(trifluoromethyl)phenoxy)-2-naphthamide or a pharmaceutically acceptable salt thereof and a MAPK pathway inhibitor or a pharmaceutically acceptable salt thereof
(S)-N-(4-hydroxybutan-2-yl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), ASTX (Taiho & Otsuka Pharmas), X-37-SHP2 (X-37),

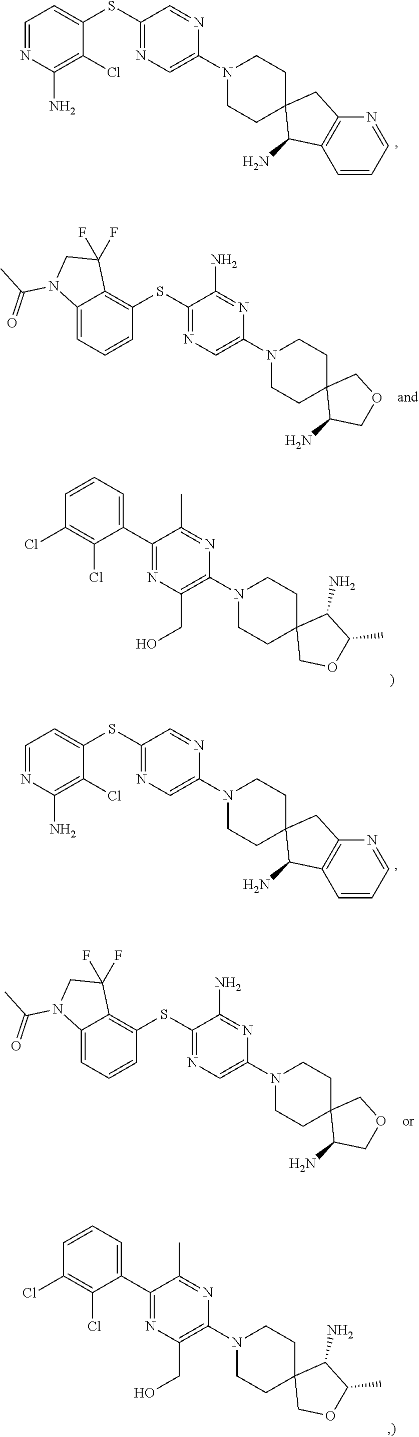

(S)-N-(4-hydroxybutan-2-yl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(S)-N-(4-hydroxybutan-2-yl)-5-(4-(trifluoromethyl)phenoxy)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(S)-N-(4-hydroxybutan-2-yl)-5-(4-(trifluoromethyl)phenoxy)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(S)-N-(4-hydroxybutan-2-yl)-5-(4-(trifluoromethyl)phenoxy)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-N-(4-hydroxybutan-2-yl)-5-(4-(trifluoromethyl)phenoxy)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(S)-N-(4-hydroxybutan-2-yl)-5-(4-(trifluoromethyl)phenoxy)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(S)-N-(4-hydroxybutan-2-yl)-5-(4-(trifluoromethyl)phenoxy)-2-naphthamide or a pharmaceutically acceptable salt thereof and a CDK4/6 inhibitor or a pharmaceutically acceptable salt thereof (e.g. a CDK416 inhibitor or a pharmaceutically acceptable salt thereof selected from the group consisting of palbociclib, ribociclib and abemaciclib)
(S)-N-(4-hydroxybutan-2-yl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(S)-N-(4-hydroxybutan-2-yl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting
(S)-N-(4-hydroxybutan-2-yl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(S)-N-(2-hydroxy-1-(pyridin-2-yl)ethyl)-5-(4-(trifluoromethyl)phenoxy)-2-naphthamide or a pharmaceutically acceptable salt thereof and a cMET inhibitor or a pharmaceutically acceptable salt thereof (e.g. capmatinib or tepotinib, e.g. capmatinib)
(S)-N-(2-hydroxy-1-(pyridin-2-yl)ethyl)-5-(4-(trifluoromethyl)phenoxy)-2-naphthamide or a pharmaceutically acceptable salt thereof and a MAPK pathway inhibitor or a pharmaceutically acceptable salt thereof
(S)-N-(2-hydroxy-1-(pyridin-2-yl)ethyl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), ASTX (Taiho & Otsuka Pharmas), X-37-SHP2 (X-37),

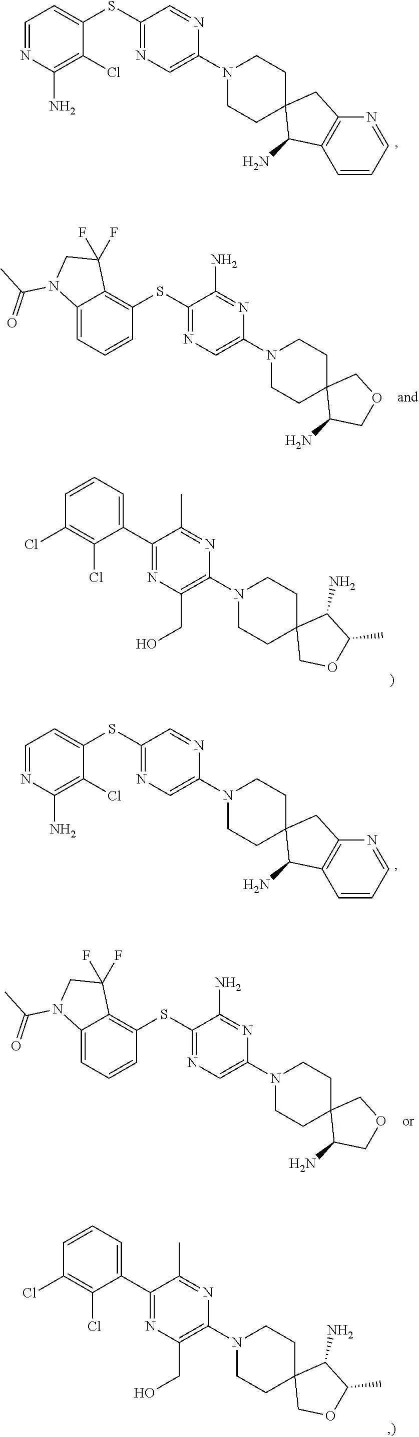

(S)-N-(2-hydroxy-1-(pyridin-2-yl)ethyl)-5-(4-(trifluoromethyl)phenoxy)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(S)-N-(2-hydroxy-1-(pyridin-2-yl)ethyl)-5-(4-(trifluoromethyl)phenoxy)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(S)-N-(2-hydroxy-1-(pyridin-2-yl)ethyl)-5-(4-(trifluoromethyl)phenoxy)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(S)-N-(2-hydroxy-1-(pyridin-2-yl)ethyl)-5-(4-(trifluoromethyl)phenoxy)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-N-(2-hydroxy-1-(pyridin-2-yl)ethyl)-5-(4-(trifluoromethyl)phenoxy)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(S)-N-(2-hydroxy-1-(pyridin-2-yl)ethyl)-5-(4-(trifluoromethyl)phenoxy)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(S)-N-(2-hydroxy-1-(pyridin-2-yl)ethyl)-5-(4-(trifluoromethyl)phenoxy)-2-naphthamide or a pharmaceutically acceptable salt thereof and a CDK4/6 inhibitor naphthamide or a pharmaceutically acceptable salt thereof (e.g. a CDK4/6 inhibitor naphthamide or a pharmaceutically acceptable salt thereof selected from the group consisting of palbociclib, ribociclib and abemaciclib)
(S)-N-(2-hydroxy-1-(pyridin-2-yl)ethyl)-5-(4-(trifluoromethyl)phenoxy)-2-naphthamide naphthamide or a pharmaceutically acceptable salt thereof and a Raf inhibitor naphthamide or a pharmaceutically acceptable salt thereof (e.g. belvarafenib, naporafenib, Encorafenib, vemurafenib or dabrafenib)
(S)-N-(2-hydroxy-1-(pyridin-2-yl)ethyl)-5-(4-(trifluoromethyl)phenoxy)-2-naphthamide naphthamide or a pharmaceutically acceptable salt thereof and a Raf inhibitor naphthamide or a pharmaceutically acceptable salt thereof (e.g. belvarafenib, naporafenib, Encorafenib, vemurafenib or dabrafenib) and a MEK inhibitor naphthamide or a pharmaceutically acceptable salt thereof (e.g. a MEK inhibitor naphthamide or a pharmaceutically acceptable salt thereof selected from the group consisting of trametinib, binimetinib, selumetinib, pimasertib, PD-0325901, and cobimetinib, e.g. trametinib)
(S)-N-(2-hydroxy-1-(pyridin-2-yl)ethyl)-5-(4-(trifluoromethyl)phenoxy)-2-naphthamide naphthamide or a pharmaceutically acceptable salt thereof and a Raf inhibitor naphthamide or a pharmaceutically acceptable salt thereof (e.g. belvarafenib, naporafenib, Encorafenib, vemurafenib or dabrafenib) and an ERK inhibitor naphthamide or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(1,5-dihydroxypentan-3-yl)-5-(4-(trifluoromethyl)phenoxy)-2-naphthamide naphthamide or a pharmaceutically acceptable salt thereof and a cMET inhibitor naphthamide or a pharmaceutically acceptable salt thereof (e.g. capmatinib or tepotinib, e.g. capmatinib)
N-(1,5-dihydroxypentan-3-yl)-5-(4-(trifluoromethyl)phenoxy)-2-naphthamide naphthamide or a pharmaceutically acceptable salt thereof and a MAPK pathway inhibitor naphthamide or a pharmaceutically acceptable salt thereof
N-(1,5-dihydroxypentan-3-yl)-5-(4-(trifluoromethyl)phenoxy)-2-naphthamide naphthamide or a pharmaceutically acceptable salt thereof and a KRAS G12/G13 inhibitor naphthamide or a pharmaceutically acceptable salt thereof (e.g. a KRAS G12C inhibitor naphthamide or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor naphthamide or a pharmaceutically acceptable salt thereof (e.g. a SHP2 inhibitor naphthamide or a pharmaceutically acceptable salt thereof selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

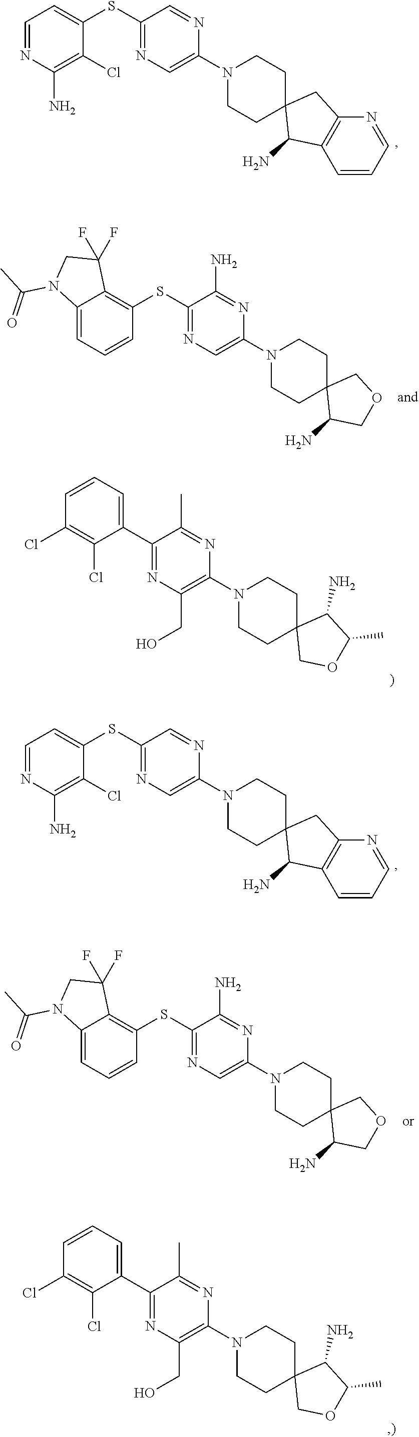

N-(1,5-dihydroxypentan-3-yl)-5-(4-(trifluoromethyl)phenoxy)-2-naphthamide naphthamide or a pharmaceutically acceptable salt thereof and a KRAS G12/G 13 inhibitor naphtha mide or a pharmaceutically acceptable salt thereof (e.g. a KRAS G12C inhibitor naphthamide or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
N-(1,5-dihydroxypentan-3-yl)-5-(4-(trifluoromethyl)phenoxy)-2-naphthamide naphthamide or a pharmaceutically acceptable salt thereof and an EGFR inhibitor naphthamide or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-(1,5-dihydroxypentan-3-yl)-5-(4-(trifluoromethyl)phenoxy)-2-naphthamide naphthamide or a pharmaceutically acceptable salt thereof and a PI3K inhibitor naphthamide or a pharmaceutically acceptable salt thereof (e.g. AMG511, QAU421, buparlisib, Idelalisib, Copanlisib, Duvelisib, Alpelisib or Umbralisib e.g. Alpelisib)
N-(1,5-dihydroxypentan-3-yl)-5-(4-(trifluoromethyl)phenoxy)-2-naphthamide naphthamide or a pharmaceutically acceptable salt thereof and a MEK inhibitor naphthamide or a pharmaceutically acceptable salt thereof (e.g. a MEK inhibitor naphthamide or a pharmaceutically acceptable salt thereof selected from the group consisting of trametinib, binimetinib, selumetinib, pimasertib, PD-0325901, and cobimetinib, e.g. trametinib)
N-(1,5-dihydroxypentan-3-yl)-5-(4-(trifluoromethyl)phenoxy)-2-naphthamide naphthamide or a pharmaceutically acceptable salt thereof and an ERK inhibitor naphthamide or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(1,5-dihydroxypentan-3-yl)-5-(4-(trifluoromethyl)phenoxy)-2-naphthamide naphthamide or a pharmaceutically acceptable salt thereof and an MDM2 inhibitor naphthamide or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin))
N-(1,5-dihydroxypentan-3-yl)-5-(4-(trifluoromethyl)phenoxy)-2-naphthamide naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
N-(1,5-dihydroxypentan-3-yl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-(1,5-dihydroxypentan-3-yl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(1,5-dihydroxypentan-3-yl)-5-(4-(trifluoromethyl)phenoxy)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-isopropyl-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
N-isopropyl-5-(4-(trifluoromethyl)phenyl)-2-naphthamide or a pharmaceutically acceptable salt thereof and a MAPK pathway inhibitor or a pharmaceutically acceptable salt thereof
N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

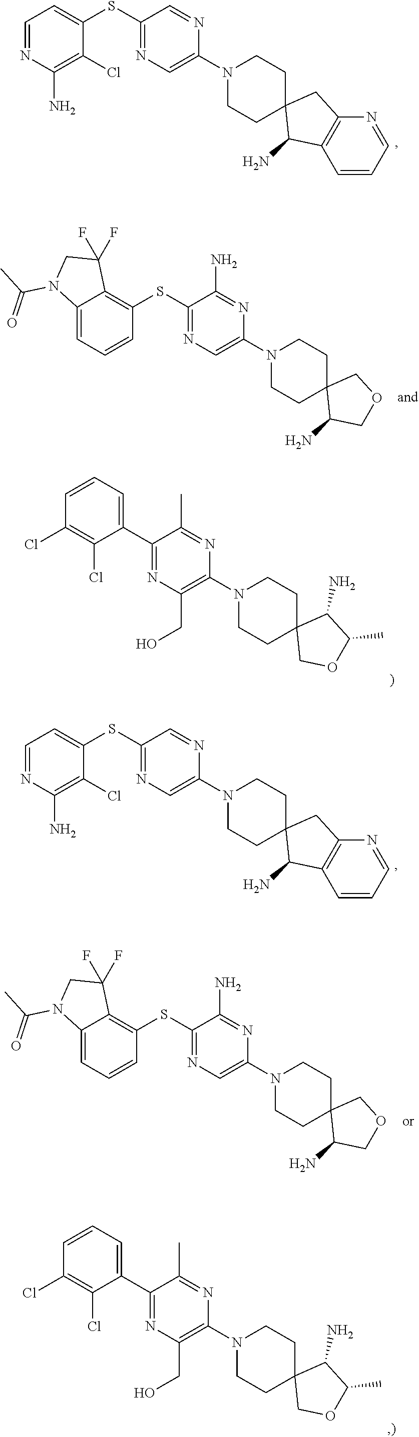

N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
N-isopropyl-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-isopropyl-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-isopropyl-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-isopropyl-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-isopropyl-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-isopropyl-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
8-bromo-N-isopropyl-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
8-bromo-N-isopropyl-5-(4-(trifluoromethyl)phenyl)-2-naphthamide or a pharmaceutically acceptable salt thereof and a MAPK pathway inhibitor or a pharmaceutically acceptable salt thereof
8-bromo-N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

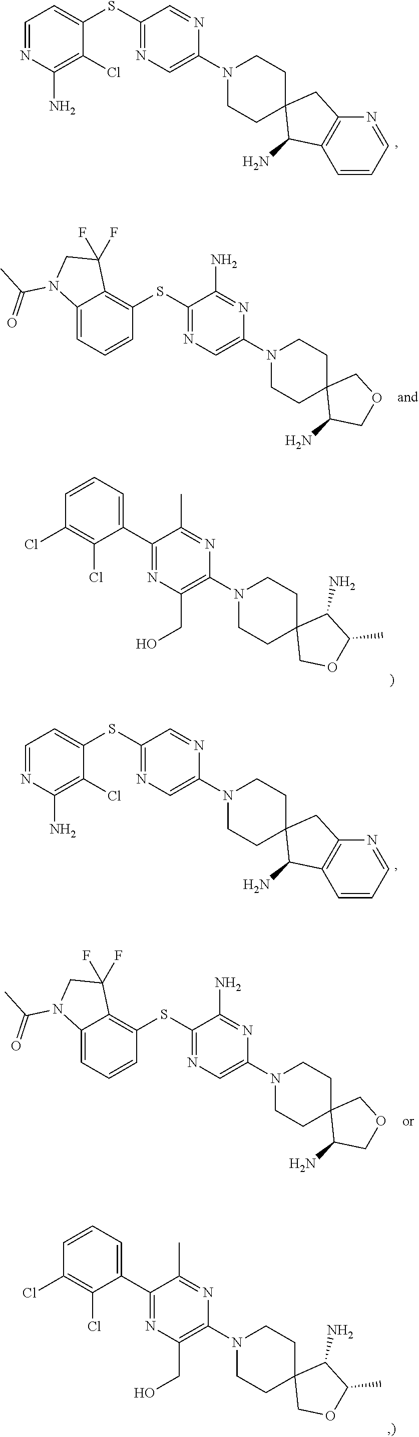

8-bromo-N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
8-bromo-N-isopropyl-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
8-bromo-N-isopropyl-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
8-bromo-N-isopropyl-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
8-bromo-N-isopropyl-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
8-bromo-N-isopropyl-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
8-bromo-N-isopropyl-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
8-bromo-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
8-bromo-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
8-bromo-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
8-amino-N-isopropyl-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
8-amino-N-isopropyl-5-(4-(trifluoromethyl)phenyl)-2-naphthamide or a pharmaceutically acceptable salt thereof and a MAPK pathway inhibitor or a pharmaceutically acceptable salt thereof
8-amino-N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

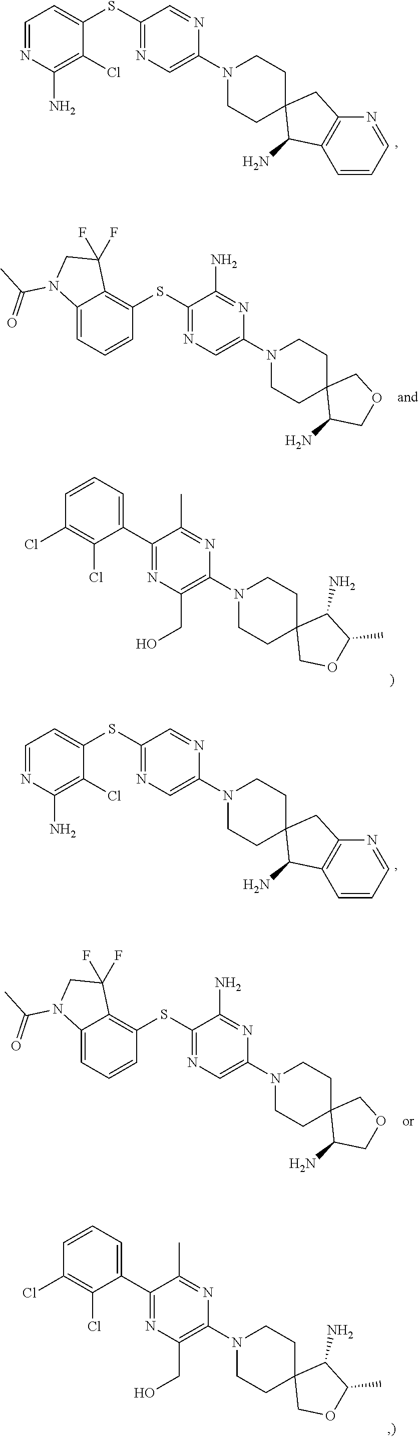

8-amino-N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
8-amino-N-isopropyl-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
8-amino-N-isopropyl-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
8-amino-N-isopropyl-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
8-amino-N-isopropyl-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
8-amino-N-isopropyl-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
8-amino-N-isopropyl-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
8-amino-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
8-amino-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
8-amino-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-isopropyl-8-methyl-5-[4-(trifluoromethyl)phenyl]naphthalene-2-carboxamide or a pharmaceutically acceptable salt thereof and a cMET inhibitor or a pharmaceutically acceptable salt thereof (e.g. capmatinib or tepotinib, e.g. capmatinib)
N-isopropyl-8-methyl-5-[4-(trifluoromethyl)phenyl]naphthalene-2-carboxamide or a pharmaceutically acceptable salt thereof and a MAPK pathway inhibitor or a pharmaceutically acceptable salt thereof
N-isopropyl-8-methyl-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), ASTX (Taiho & Otsuka Pharmas), X-37-SHP2 (X-37),

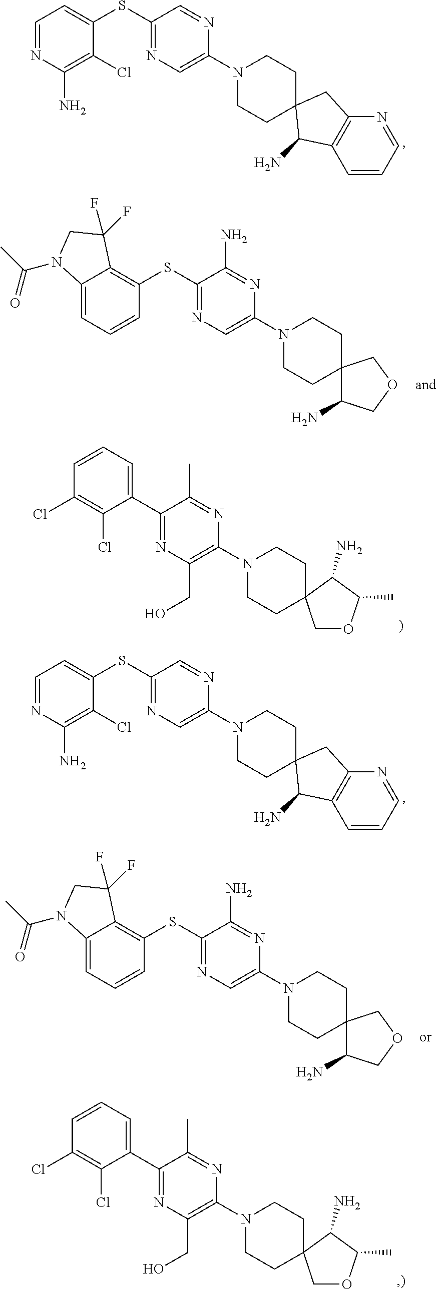

N-isopropyl-8-methyl-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitorora pharmaceutically acceptable salt thereof selected from the group consisting of TNO155, JAB3068 (Jacobio), JAB3312 (Jacobio), (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS
ASTX (Taiho & Otsuka Pharmas), X-37-SHP2 (X-37),

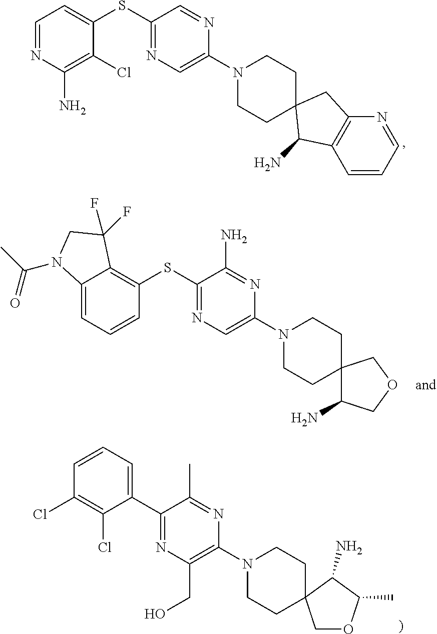 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

N-isopropyl-8-methyl-5-[4-(trifluoromethyl)phenyl]naphthalene-2-carboxamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

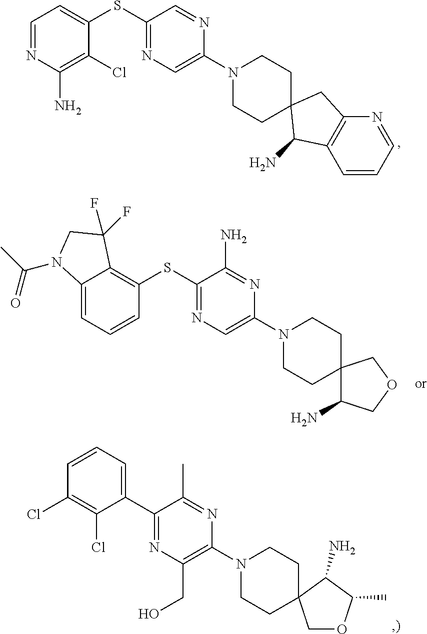 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

N-isopropyl-8-methyl-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982. B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
N-isopropyl-8-methyl-5-[4-(trifluoromethyl)phenyl]naphthalene-2-carboxamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-isopropyl-8-methyl-5-[4-(trifluoromethyl)phenyl]naphthalene-2-carboxamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-isopropyl-8-methyl-5-[4-(trifluoromethyl)phenyl]naphthalene-2-carboxamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-isopropyl-8-methyl-5-[4-(trifluoromethyl)phenyl]naphthalene-2-carboxamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
N-isopropyl-8-methyl-5-[4-(trifluoromethyl)phenyl]naphthalene-2-carboxamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-isopropyl-8-methyl-5-[4-(trifluoromethyl)phenyl]naphthalene-2-carboxamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
N-isopropyl-8-methyl-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-isopropyl-8-methyl-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-isopropyl-8-methyl-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
8-ethyl-N-isopropyl-5-[4-(trifluoromethyl)phenyl]naphthalene-2-carboxamide or a pharmaceutically acceptable salt thereof and a cMET inhibitor or a pharmaceutically acceptable salt thereof (e.g. capmatinib or tepotinib, e.g. capmatinib)
8-ethyl-N-isopropyl-5-[4-(trifluoromethyl)phenyl]naphthalene-2-carboxamide or a pharmaceutically acceptable salt thereof and a MAPK pathway inhibitor or a pharmaceutically acceptable salt thereof
8-ethyl-N-isopropyl-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), ASTX (Taiho & Otsuka Pharmas), X-37-SHP2 (X-37),

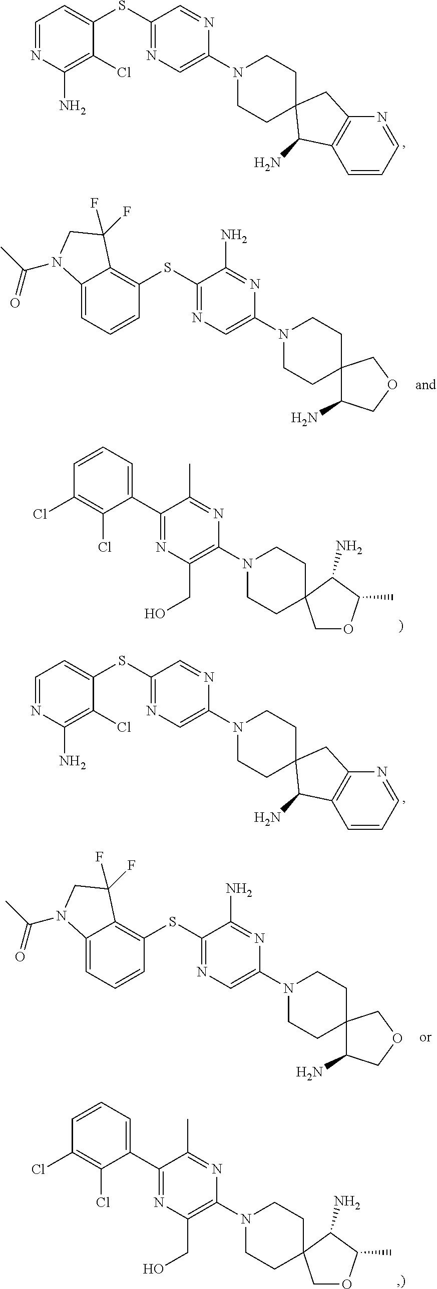

8-ethyl-N-isopropyl-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
8-ethyl-N-isopropyl-5-[4-(trifluoromethyl)phenyl]naphthalene-2-carboxamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
8-ethyl-N-isopropyl-5-[4-(trifluoromethyl)phenyl]naphthalene-2-carboxamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
8-ethyl-N-isopropyl-5-[4-(trifluoromethyl)phenyl]naphthalene-2-carboxamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
8-ethyl-N-isopropyl-5-[4-(trifluoromethyl)phenyl]naphthalene-2-carboxamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
8-ethyl-N-isopropyl-5-[4-(trifluoromethyl)phenyl]naphthalene-2-carboxamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
8-ethyl-N-isopropyl-5-[4-(trifluoromethyl)phenyl]naphthalene-2-carboxamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
8-ethyl-N-isopropyl-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib)
8-ethyl-N-isopropyl-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
8-ethyl-N-isopropyl-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
8-cyclopropyl-N-isopropyl-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
8-cyclopropyl-N-isopropyl-5-(4-(trifluoromethyl)phenyl)-2-naphthamide or a pharmaceutically acceptable salt thereof and a MAPK pathway inhibitor or a pharmaceutically acceptable salt thereof
8-cyclopropyl-N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), ASTX (Taiho & Otsuka Pharmas), X-37-SHP2 (X-37),

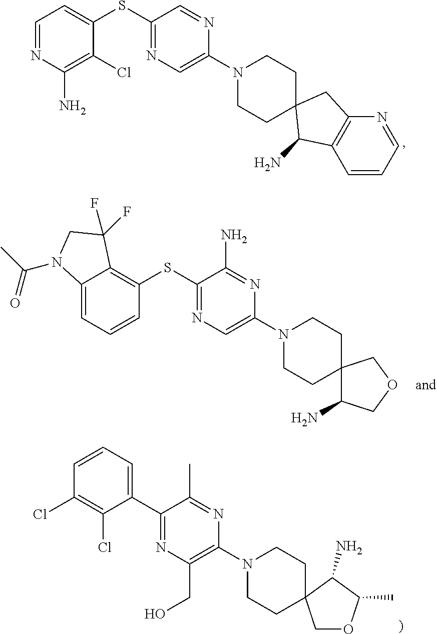 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

8-cyclopropyl-N-isopropyl-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

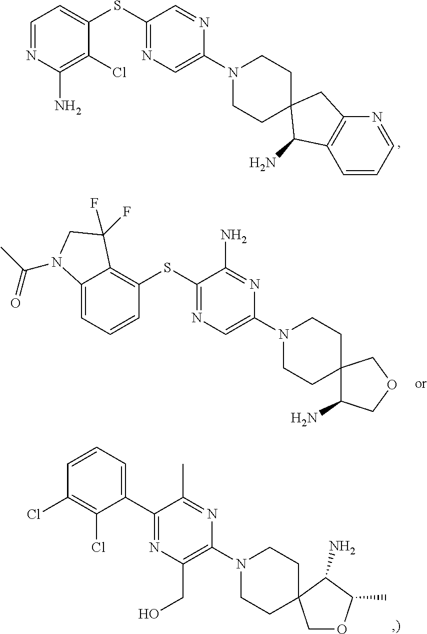 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

8-cyclopropyl-N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
8-cyclopropyl-N-isopropyl-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
8-cyclopropyl-N-isopropyl-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
8-cyclopropyl-N-isopropyl-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
8-cyclopropyl-N-isopropyl-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
8-cyclopropyl-N-isopropyl-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
8-cyclopropyl-N-isopropyl-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
8-cyclopropyl-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
8-cyclopropyl-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
8-cyclopropyl-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
8-amino-7-bromo-N-isopropyl-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
8-amino-7-bromo-N-isopropyl-5-(4-(trifluoromethyl)phenyl)-2-naphthamide or a pharmaceutically acceptable salt thereof and a MAPK pathway inhibitor or a pharmaceutically acceptable salt thereof
8-amino-7-bromo-N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), ASTX (Taiho & Otsuka Pharmas), X-37-SHP2 (X-37),

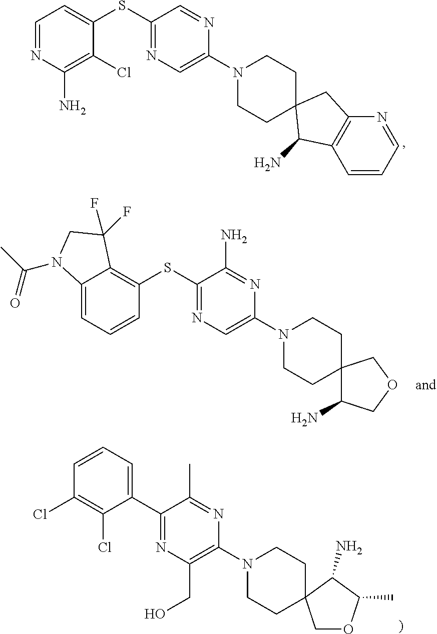 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

8-amino-7-bromo-N-isopropyl-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

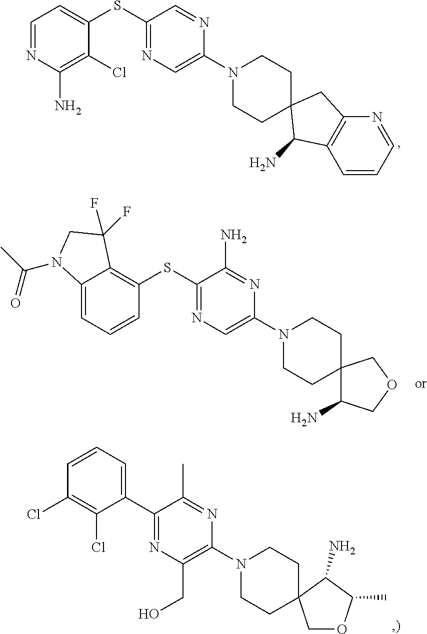 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

8-amino-7-bromo-N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
8-amino-7-bromo-N-isopropyl-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
8-amino-7-bromo-N-isopropyl-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
8-amino-7-bromo-N-isopropyl-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
8-amino-7-bromo-N-isopropyl-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
8-amino-7-bromo-N-isopropyl-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
8-amino-7-bromo-N-isopropyl-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
8-amino-7-bromo-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
8-amino-7-bromo-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
8-amino-7-bromo-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
7-bromo-N-isopropyl-5-[4-(trifluoromethyl)phenyl]naphthalene-2-carboxamide or a pharmaceutically acceptable salt thereof and a cMET inhibitor or a pharmaceutically acceptable salt thereof (e.g. capmatinib or tepotinib, e.g. capmatinib)
7-bromo-N-isopropyl-5-[4-(trifluoromethyl)phenyl]naphthalene-2-carboxamide or a pharmaceutically acceptable salt thereof and a MAPK pathway inhibitor or a pharmaceutically acceptable salt thereof
7-bromo-N-isopropyl-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), ASTX (Taiho & Otsuka Pharmas), X-37-SHP2 (X-37),

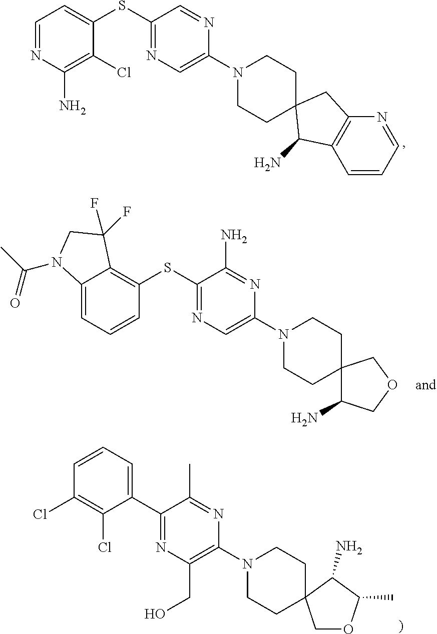 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

7-bromo-N-isopropyl-5-[4-(trifluoromethyl)phenyl]naphthalene-2-carboxamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

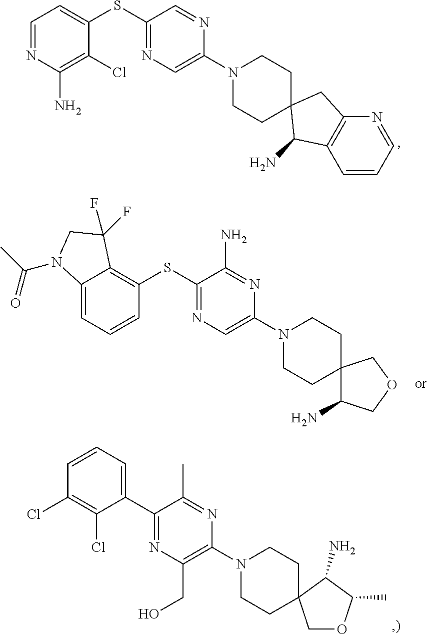 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

7-bromo-N-isopropyl-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
7-bromo-N-isopropyl-5-[4-(trifluoromethyl)phenyl]naphthalene-2-carboxamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
7-bromo-N-isopropyl-5-[4-(trifluoromethyl)phenyl]naphthalene-2-carboxamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
7-bromo-N-isopropyl-5-[4-(trifluoromethyl)phenyl]naphthalene-2-carboxamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
7-bromo-N-isopropyl-5-[4-(trifluoromethyl)phenyl]naphthalene-2-carboxamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
7-bromo-N-isopropyl-5-[4-(trifluoromethyl)phenyl]naphthalene-2-carboxamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
7-bromo-N-isopropyl-5-[4-(trifluoromethyl)phenyl]naphthalene-2-carboxamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
7-bromo-N-isopropyl-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib)
7-bromo-N-isopropyl-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
7-bromo-N-isopropyl-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
8-amino-7-chloro-N-isopropyl-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
8-amino-7-chloro-N-isopropyl-5-(4-(trifluoromethyl)phenyl)-2-naphthamide or a pharmaceutically acceptable salt thereof and a MAPK pathway inhibitor or a pharmaceutically acceptable salt thereof
8-amino-7-chloro-N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), ASTX (Taiho & Otsuka Pharmas), X-37-SHP2 (X-37),

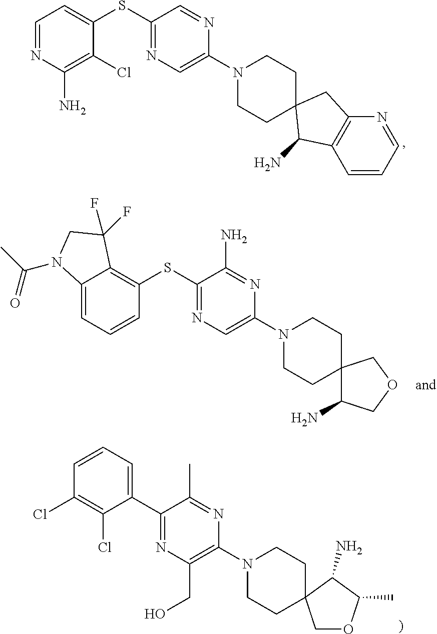 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

8-amino-7-chloro-N-isopropyl-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

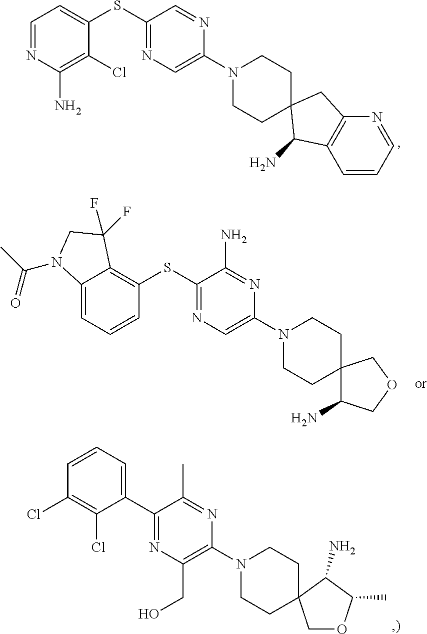 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

8-amino-7-chloro-N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
8-amino-7-chloro-N-isopropyl-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
8-amino-7-chloro-N-isopropyl-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
8-amino-7-chloro-N-isopropyl-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
8-amino-7-chloro-N-isopropyl-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
8-amino-7-chloro-N-isopropyl-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
8-amino-7-chloro-N-isopropyl-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
8-amino-7-chloro-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
8-amino-7-chloro-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting
8-amino-7-chloro-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
7-chloro-N-isopropyl-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
7-chloro-N-isopropyl-5-(4-(trifluoromethyl)phenyl)-2-naphthamide or a pharmaceutically acceptable salt thereof and a MAPK pathway inhibitor or a pharmaceutically acceptable salt thereof
7-chloro-N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor or a pharmaceutically acceptable salt thereof (e.g. a SHP2 inhibitor or a pharmaceutically acceptable salt thereof selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

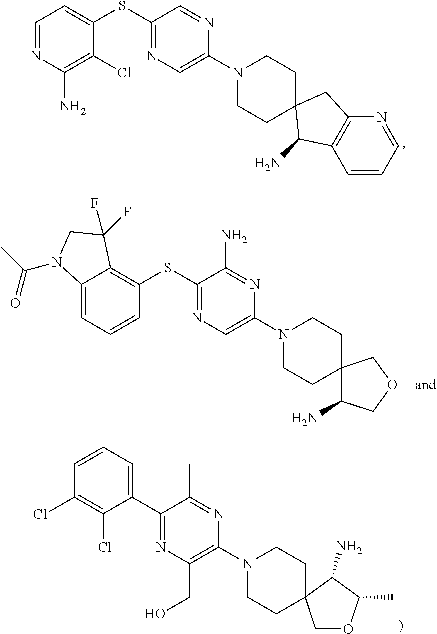 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

7-chloro-N-isopropyl-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

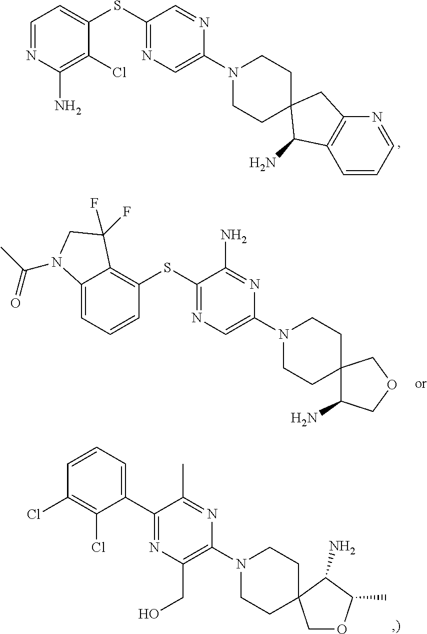 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

7-chloro-N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
7-chloro-N-isopropyl-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
7-chloro-N-isopropyl-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
7-chloro-N-isopropyl-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
7-chloro-N-isopropyl-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
7-chloro-N-isopropyl-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
7-chloro-N-isopropyl-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
7-chloro-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
7-chloro-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
7-chloro-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-isopropyl-8-methoxy-5-[4-(trifluoromethyl)phenyl]naphthalene-2-carboxamide or a pharmaceutically acceptable salt thereof and a cMET inhibitor or a pharmaceutically acceptable salt thereof (e.g. capmatinib or tepotinib, e.g. capmatinib)
N-isopropyl-8-methoxy-5-[4-(trifluoromethyl)phenyl]naphthalene-2-carboxamide or a pharmaceutically acceptable salt thereof and a MAPK pathway inhibitor or a pharmaceutically acceptable salt thereof
N-isopropyl-8-methoxy-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 ASTX (Taiho & Otsuka Pharmas), X-37-SHP2 (X-37),

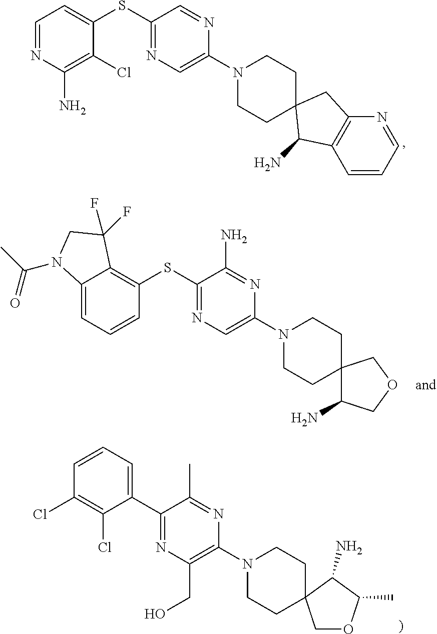 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

N-isopropyl-8-methoxy-5-[4-(trifluoromethyl)phenyl]naphthalene-2-carboxamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

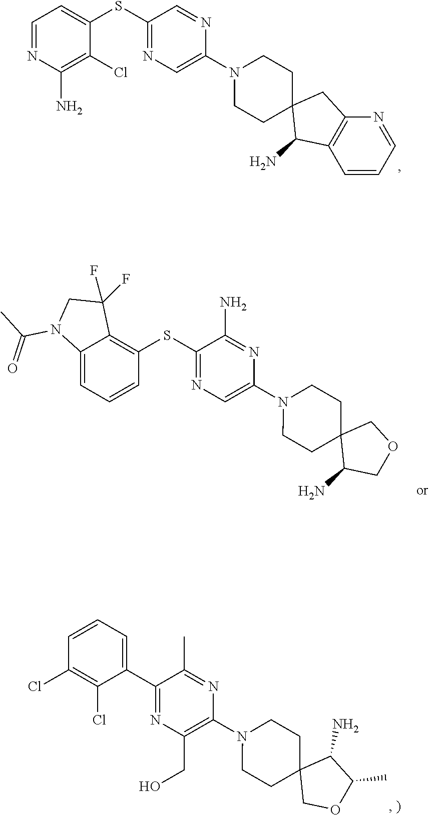 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-isopropyl-8-methoxy-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
N-isopropyl-8-methoxy-5-[4-(trifluoromethyl)phenyl]naphthalene-2-carboxamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-isopropyl-8-methoxy-5-[4-(trifluoromethyl)phenyl]naphthalene-2-carboxamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-isopropyl-8-methoxy-5-[4-(trifluoromethyl)phenyl]naphthalene-2-carboxamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-isopropyl-8-methoxy-5-[4-(trifluoromethyl)phenyl]naphthalene-2-carboxamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
N-isopropyl-8-methoxy-5-[4-(trifluoromethyl)phenyl]naphthalene-2-carboxamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-isopropyl-8-methoxy-5-[4-(trifluoromethyl)phenyl]naphthalene-2-carboxamide or a pharmaceutically acceptable salt thereof and a CDK4/6 inhibitor or a pharmaceutically acceptable salt thereof (e.g. a CDK416 inhibitor or a pharmaceutically acceptable salt thereof selected from the group consisting of palbociclib, ribociclib and abemaciclib)
N-isopropyl-8-methoxy-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-isopropyl-8-methoxy-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-isopropyl-8-methoxy-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-isopropyl-7-methoxy-5-[4-(trifluoromethyl)phenyl]naphthalene-2-carboxamide or a pharmaceutically acceptable salt thereof and a cMET inhibitor or a pharmaceutically acceptable salt thereof (e.g. capmatinib or tepotinib, e.g. capmatinib)
N-isopropyl-7-methoxy-5-[4-(trifluoromethyl)phenyl]naphthalene-2-carboxamide or a pharmaceutically acceptable salt thereof and a MAPK pathway inhibitor or a pharmaceutically acceptable salt thereof
N-isopropyl-7-methoxy-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

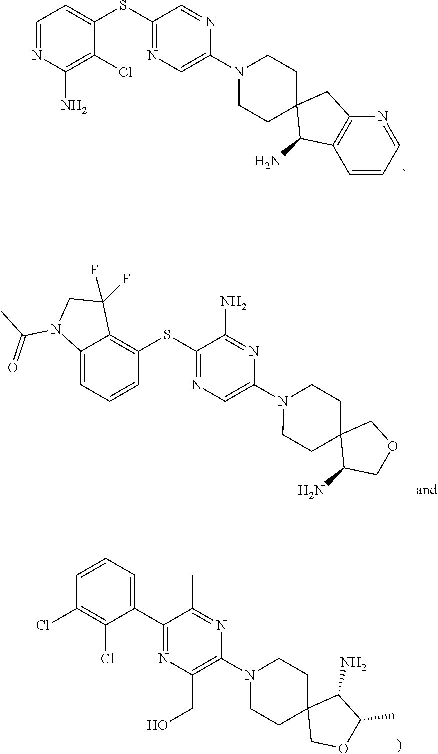 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-isopropyl-7-methoxy-5-[4-(trifluoromethyl)phenyl]naphthalene-2-carboxamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

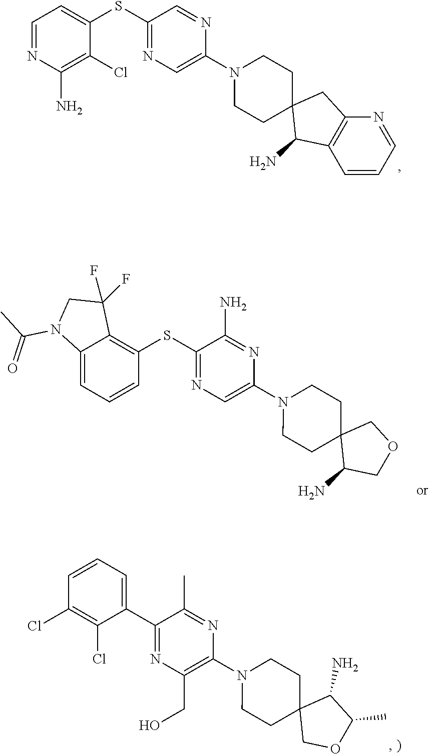 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-isopropyl-7-methoxy-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
N-isopropyl-7-methoxy-5-[4-(trifluoromethyl)phenyl]naphthalene-2-carboxamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-isopropyl-7-methoxy-5-[4-(trifluoromethyl)phenyl]naphthalene-2-carboxamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-isopropyl-7-methoxy-5-[4-(trifluoromethyl)phenyl]naphthalene-2-carboxamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-isopropyl-7-methoxy-5-[4-(trifluoromethyl)phenyl]naphthalene-2-carboxamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
N-isopropyl-7-methoxy-5-[4-(trifluoromethyl)phenyl]naphthalene-2-carboxamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-isopropyl-7-methoxy-5-[4-(trifluoromethyl)phenyl]naphthalene-2-carboxamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
N-isopropyl-7-methoxy-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-isopropyl-7-methoxy-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-isopropyl-7-methoxy-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-isopropyl-7-methyl-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
N-isopropyl-7-methyl-5-(4-(trifluoromethyl)phenyl)-2-naphthamide or a pharmaceutically acceptable salt thereof and a MAPK pathway inhibitor or a pharmaceutically acceptable salt thereof
N-isopropyl-7-methyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

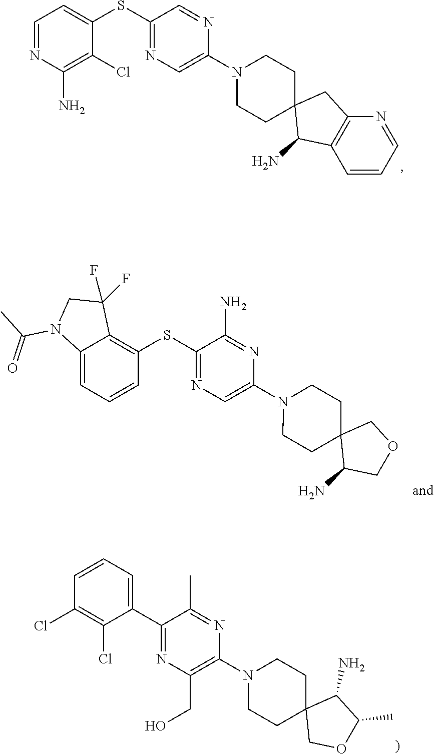 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-isopropyl-7-methyl-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

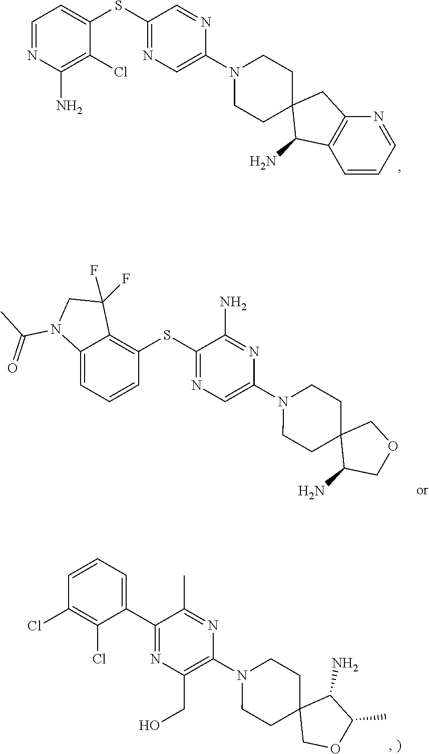 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-isopropyl-7-methyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12/G13 inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
N-isopropyl-7-methyl-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-isopropyl-7-methyl-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-isopropyl-7-methyl-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-isopropyl-7-methyl-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-isopropyl-7-methyl-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-isopropyl-7-methyl-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
N-isopropyl-7-methyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-isopropyl-7-methyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-isopropyl-7-methyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
7-cyclopropyl-N-isopropyl-5-[4-(trifluoromethyl)phenyl]naphthalene-2-carboxamide or a pharmaceutically acceptable salt thereof and a cMET inhibitor or a pharmaceutically acceptable salt thereof (e.g. capmatinib or tepotinib, e.g. capmatinib)
7-cyclopropyl-N-isopropyl-5-[4-(trifluoromethyl)phenyl]naphthalene-2-carboxamide or a pharmaceutically acceptable salt thereof and a MAPK pathway inhibitor or a pharmaceutically acceptable salt thereof
7-cyclopropyl-N-isopropyl-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

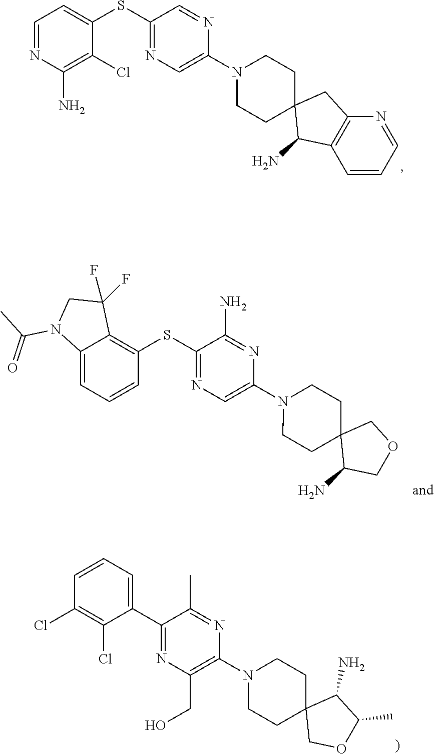 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

7-cyclopropyl-N-isopropyl-5-[4-(trifluoromethyl)phenyl]naphthalene-2-carboxamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), & Otsuka Pharmas) and X-37-SHP2 (X-37),

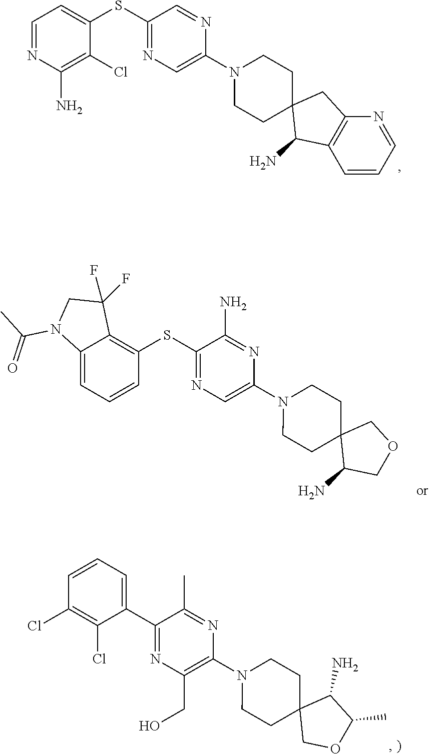 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

7-cyclopropyl-N-isopropyl-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
7-cyclopropyl-N-isopropyl-5-[4-(trifluoromethyl)phenyl]naphthalene-2-carboxamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
7-cyclopropyl-N-isopropyl-5-[4-(trifluoromethyl)phenyl]naphthalene-2-carboxamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
7-cyclopropyl-N-isopropyl-5-[4-(trifluoromethyl)phenyl]naphthalene-2-carboxamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
7-cyclopropyl-N-isopropyl-5-[4-(trifluoromethyl)phenyl]naphthalene-2-carboxamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
7-cyclopropyl-N-isopropyl-5-[4-(trifluoromethyl)phenyl]naphthalene-2-carboxamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
7-cyclopropyl-N-isopropyl-5-[4-(trifluoromethyl)phenyl]naphthalene-2-carboxamide or a pharmaceutically acceptable salt thereof and a CDK4/6 inhibitor or a pharmaceutically acceptable salt thereof (e.g. a CDK416 inhibitor or a pharmaceutically acceptable salt thereof selected from the group consisting of palbociclib, ribociclib and abemaciclib)
7-cyclopropyl-N-isopropyl-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib)
7-cyclopropyl-N-isopropyl-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
7-cyclopropyl-N-isopropyl-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
7-amino-N-isopropyl-5-[4-(trifluoromethyl)phenyl]naphthalene-2-carboxamide or a pharmaceutically acceptable salt thereof and a cMET inhibitor or a pharmaceutically acceptable salt thereof (e.g. capmatinib or tepotinib, e.g. capmatinib)
7-amino-N-isopropyl-5-[4-(trifluoromethyl)phenyl]naphthalene-2-carboxamide or a pharmaceutically acceptable salt thereof and a MAPK pathway inhibitor or a pharmaceutically acceptable salt thereof
7-amino-N-isopropyl-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

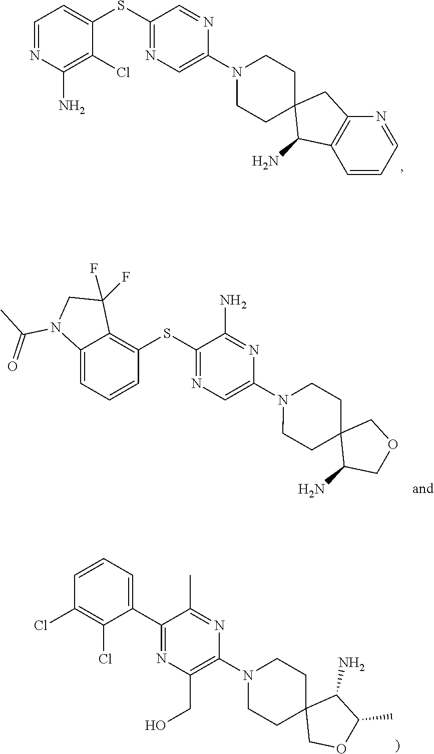 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

7-amino-N-isopropyl-5-[4-(trifluoromethyl)phenyl]naphthalene-2-carboxamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

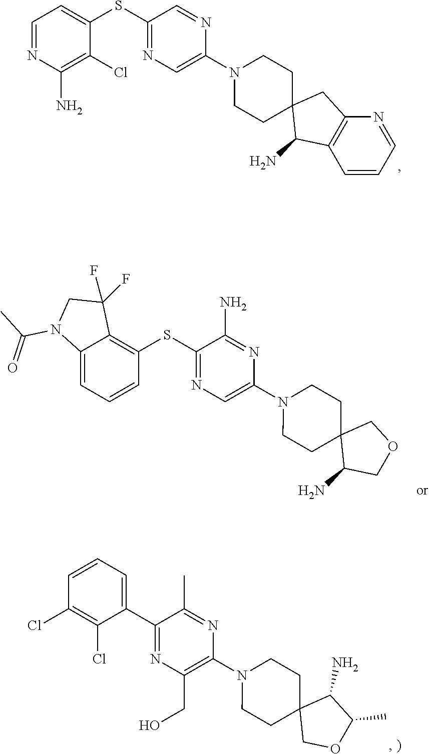 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

7-amino-N-isopropyl-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
7-amino-N-isopropyl-5-[4-(trifluoromethyl)phenyl]naphthalene-2-carboxamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
7-amino-N-isopropyl-5-[4-(trifluoromethyl)phenyl]naphthalene-2-carboxamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
7-amino-N-isopropyl-5-[4-(trifluoromethyl)phenyl]naphthalene-2-carboxamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
7-amino-N-isopropyl-5-[4-(trifluoromethyl)phenyl]naphthalene-2-carboxamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
7-amino-N-isopropyl-5-[4-(trifluoromethyl)phenyl]naphthalene-2-carboxamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
7-amino-N-isopropyl-5-[4-(trifluoromethyl)phenyl]naphthalene-2-carboxamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
7-amino-N-isopropyl-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib)
7-amino-N-isopropyl-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
7-amino-N-isopropyl-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
7-ethyl-N-isopropyl-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
7-ethyl-N-isopropyl-5-(4-(trifluoromethyl)phenyl)-2-naphthamide or a pharmaceutically acceptable salt thereof and a MAPK pathway inhibitor or a pharmaceutically acceptable salt thereof
7-ethyl-N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

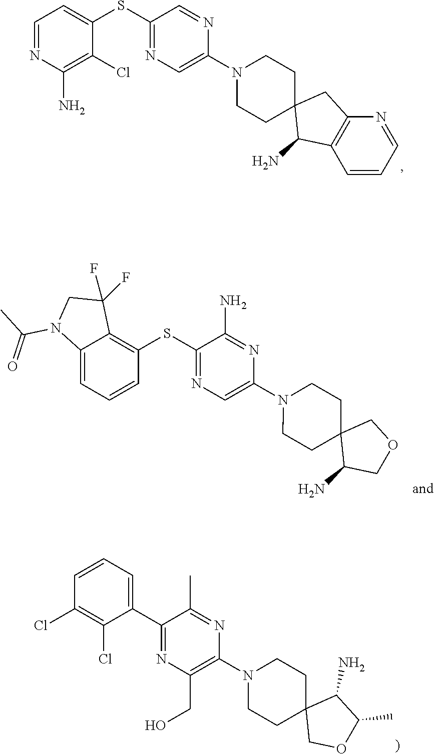 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

7-ethyl-N-isopropyl-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

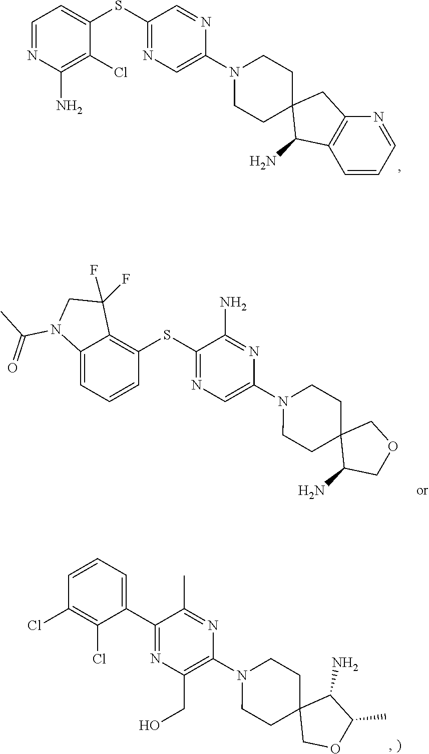 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

7-ethyl-N-isopropyl-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
7-ethyl-N-isopropyl-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
7-ethyl-N-isopropyl-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
7-ethyl-N-isopropyl-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
7-ethyl-N-isopropyl-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
7-ethyl-N-isopropyl-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
7-ethyl-N-isopropyl-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
7-ethyl-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
7-ethyl-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
7-ethyl-N-isopropyl-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(1,3-dihydroxypropan-2-yl)-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
N-(1,3-dihydroxypropan-2-yl)-5-(4-(trifluoromethyl)phenyl)-2-naphthamide or a pharmaceutically acceptable salt thereof and a MAPK pathway inhibitor or a pharmaceutically acceptable salt thereof
N-(1,3-dihydroxypropan-2-yl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 ASTX (Taiho & Otsuka Pharmas), X-37-SHP2 (X-37),

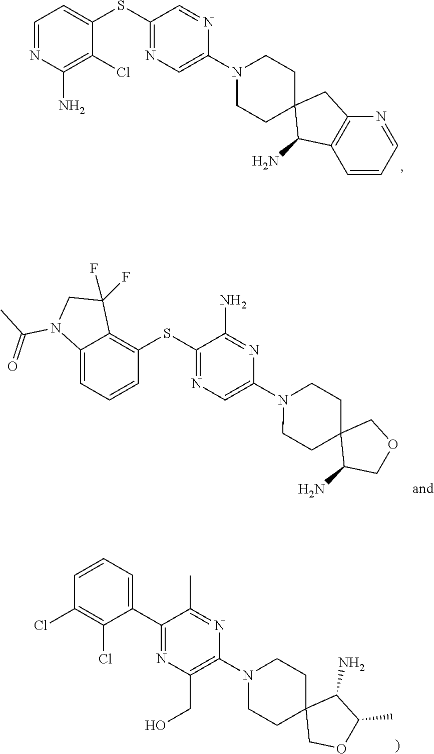 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-(1,3-dihydroxypropan-2-yl)-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

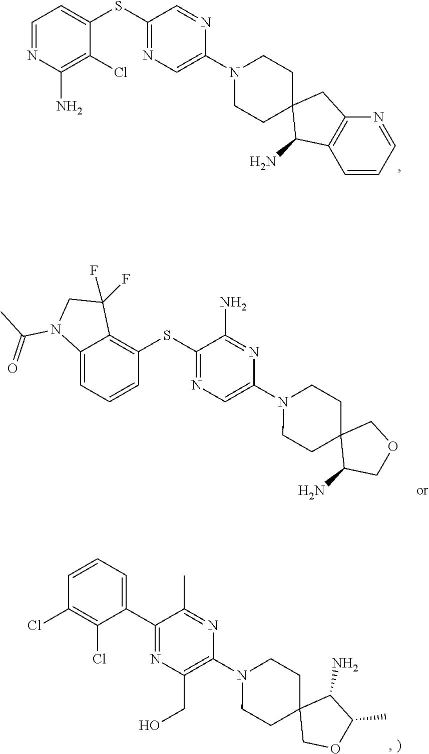 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-(1,3-dihydroxypropan-2-yl-5-(trifuromethyl)phenyl)-2-naphthamide or a pharmaceutically acceptable salt thereof and a KRAS G12/G13 inhibitor or a pharmaceutically acceptable salt thereof (e.g. a KRAS G12/G13 inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12/G13, SF KRAS G121G13, RMC032, JAB-21822, AST-KRAS G12/G13, MRTX1257, AZ KRAS G12/G13, NYU-12VC1, RMC6291 and Compound C, e.g. Compound C, Sotorasib or Adagrasib)
N-(1,3-dihydroxypropan-2-yl)-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-(1,3-dihydroxypropan-2-yl)-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-(1,3-dihydroxypropan-2-yl)-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(1,3-dihydroxypropan-2-yl)-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
N-(1,3-dihydroxypropan-2-yl)-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-(1,3-dihydroxypropan-2-yl)-5-(4-(trifluoromethyl)phenyl)-2-naphthamide or a pharmaceutically acceptable salt thereof and a CDK4/6 inhibitor or a pharmaceutically acceptable salt thereof (e.g. a CDK416 inhibitor or a pharmaceutically acceptable salt thereof selected from the group consisting of palbociclib, ribociclib and abemaciclib)
N-(1,3-dihydroxypropan-2-yl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-(1,3-dihydroxypropan-2-yl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(1,3-dihydroxypropan-2-yl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(R)-N-(1-aminopropan-2-yl)-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
(R)-N-(1-aminopropan-2-yl)-5-(4-(trifluoromethyl)phenyl)-2-naphthamide or a pharmaceutically acceptable salt thereof and a MAPK pathway inhibitor or a pharmaceutically acceptable salt thereof
(R)-N-(1-aminopropan-2-yl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

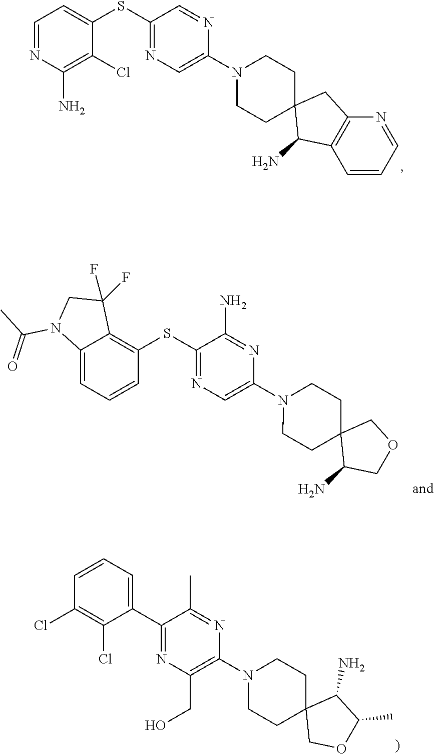 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(R)-N-(1-aminopropan-2-yl)-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

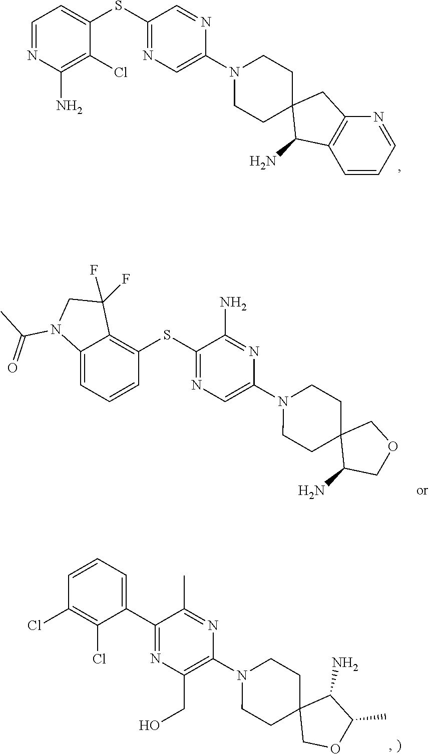 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(R)-N-(1-aminopropan-2-yl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(R)-N-(1-aminopropan-2-yl)-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(R)-N-(1-aminopropan-2-yl)-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(R)-N-(1-aminopropan-2-yl)-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-N-(1-aminopropan-2-yl)-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
(R)-N-(1-aminopropan-2-yl)-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(R)-N-(1-aminopropan-2-yl)-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
(R)-N-(1-aminopropan-2-yl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(R)-N-(1-aminopropan-2-yl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-N-(1-aminopropan-2-yl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(S)-N-(1-hydroxypropan-2-yl)-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
(S)-N-(1-hydroxypropan-2-yl)-5-(4-(trifluoromethyl)phenyl)-2-naphthamide or a pharmaceutically acceptable salt thereof and a MAPK pathway inhibitor or a pharmaceutically acceptable salt thereof
(S)-N-(1-hydroxypropan-2-yl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

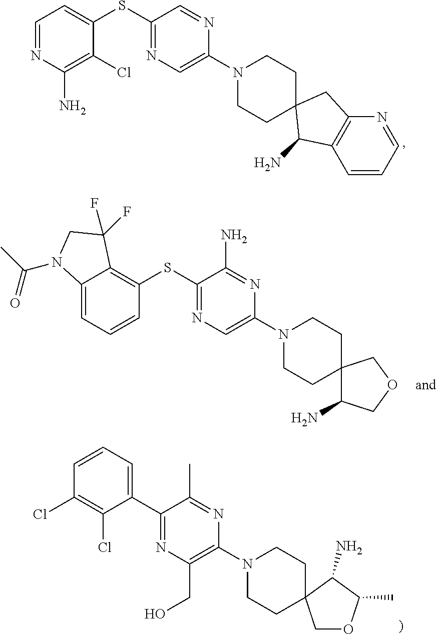 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

(S)-N-(1-hydroxypropan-2-yl)-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

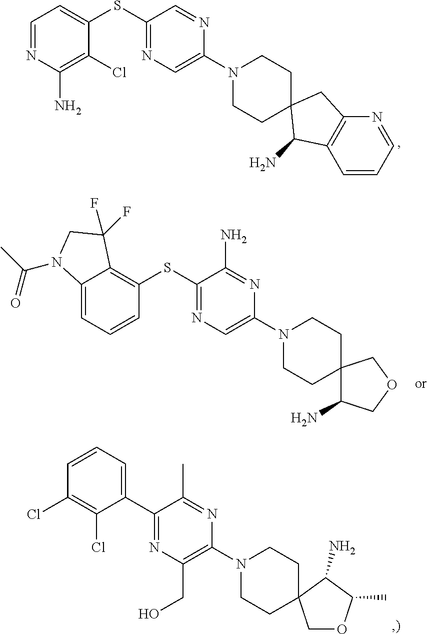 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

(S-N-(1-hydroxypropan-2-yl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(S)-N-(1-hydroxypropan-2-yl)-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(S)-N-(1-hydroxypropan-2-yl)-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(S)-N-(1-hydroxypropan-2-yl)-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-N-(1-hydroxypropan-2-yl)-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
(S)-N-(1-hydroxypropan-2-yl)-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(S)-N-(1-hydroxypropan-2-yl)-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
(S)-N-(1-hydroxypropan-2-yl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(S)-N-(1-hydroxypropan-2-yl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-N-(1-hydroxypropan-2-yl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(R)-N-(1-hydroxypropan-2-yl)-5-(4-trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
(R)-N-(1-hydroxypropan-2-yl)-5-(4-trifluoromethyl)phenyl)-2-naphthamide or a pharmaceutically acceptable salt thereof and a MAPK pathway inhibitor or a pharmaceutically acceptable salt thereof
(R)-N-(1-hydroxypropan-2-yl)-5-(4-trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

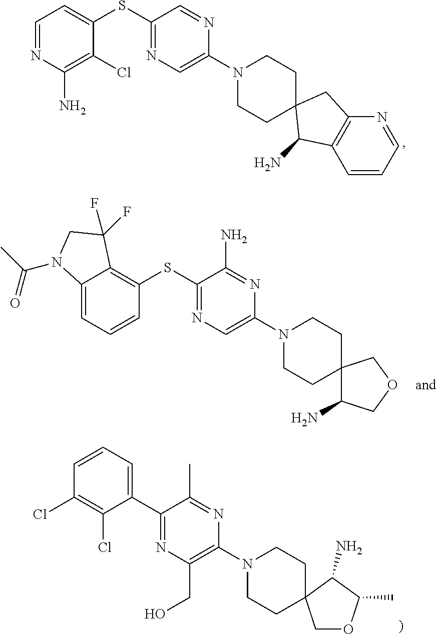 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

(R)-N-(1-hydroxypropan-2-yl)-5-(4-trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

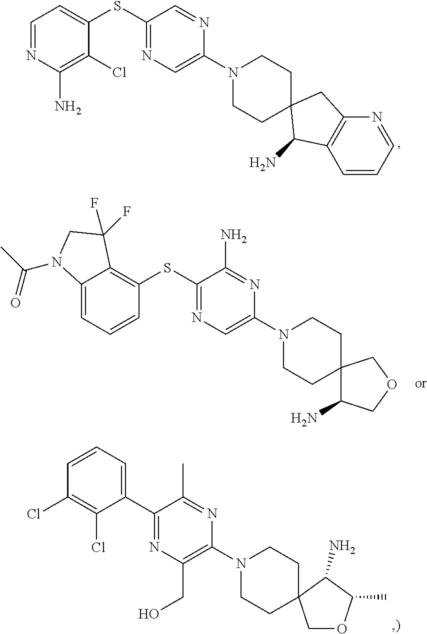 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

(R)-N-(1-hydroxypropan-2-yl)-5-(4-trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(R)-N-(1-hydroxypropan-2-yl)-5-(4-trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(R)-N-(1-hydroxypropan-2-yl)-5-(4-trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(R)-N-(1-hydroxypropan-2-yl)-5-(4-trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-N-(1-hydroxypropan-2-yl)-5-(4-trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
(R)-N-(1-hydroxypropan-2-yl)-5-(4-trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(R)-N-(1-hydroxypropan-2-yl)-5-(4-trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
(R)-N-(1-hydroxypropan-2-yl)-5-(4-trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(R)-N-(1-hydroxypropan-2-yl)-5-(4-trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-N-(1-hydroxypropan-2-yl)-5-(4-trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(2-hydroxyethyl)-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
N-(2-hydroxyethyl)-5-(4-(trifluoromethyl)phenyl)-2-naphthamide or a pharmaceutically acceptable salt thereof and a MAPK pathway inhibitor or a pharmaceutically acceptable salt thereof
N-(2-hydroxyethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

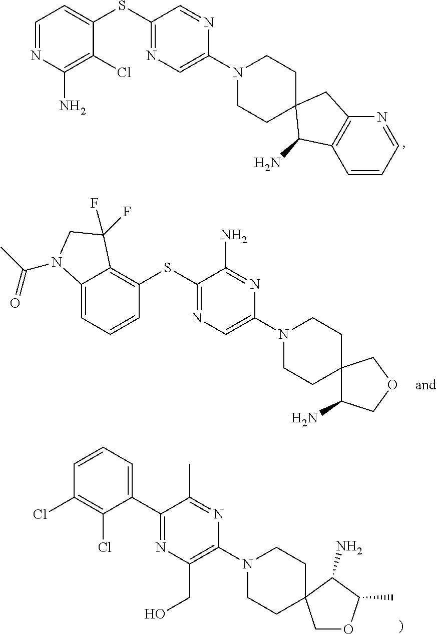 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

N-(2-hydroxyethyl)-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

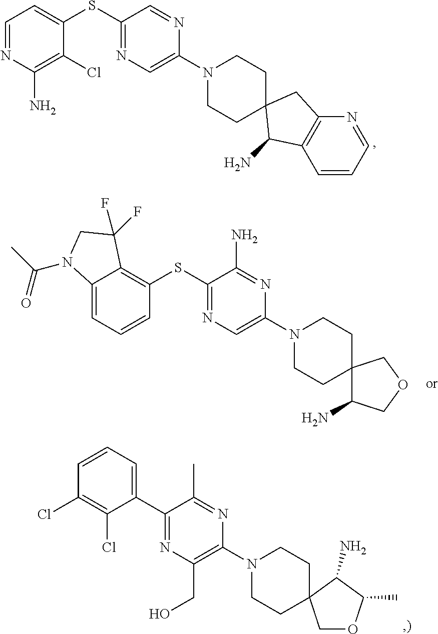 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

N-(2-hydroxyethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12/G13 inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
N-(2-hydroxyethyl)-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-(2-hydroxyethyl)-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-(2-hydroxyethyl)-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(2-hydroxyethyl)-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(2-hydroxyethyl)-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-(2-hydroxyethyl)-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
N-(2-hydroxyethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-(2-hydroxyethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(2-hydroxyethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(S)-N-(1-(pyridin-2-yl)ethyl)-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
(S)-N-(1-(pyridin-2-yl)ethyl)-5-(4-(trifluoromethyl)phenyl)-2-naphthamide or a pharmaceutically acceptable salt thereof and a MAPK pathway inhibitor or a pharmaceutically acceptable salt thereof
(S)-N-(1-(pyridin-2-yl)ethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

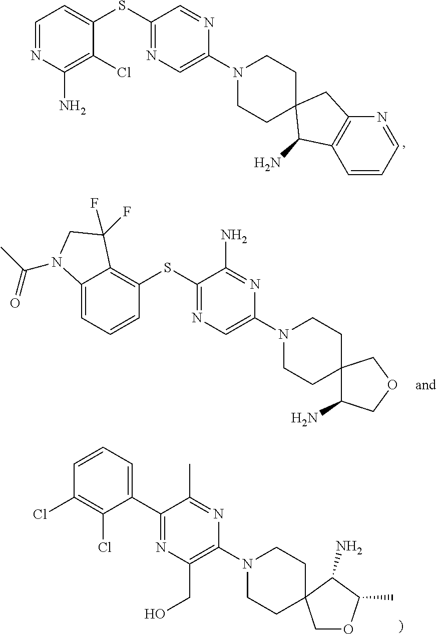 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

(S)-N-(1-(pyridin-2-yl)ethyl)-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), & Otsuka Pharmas) and X-37-SHP2 (X-37),

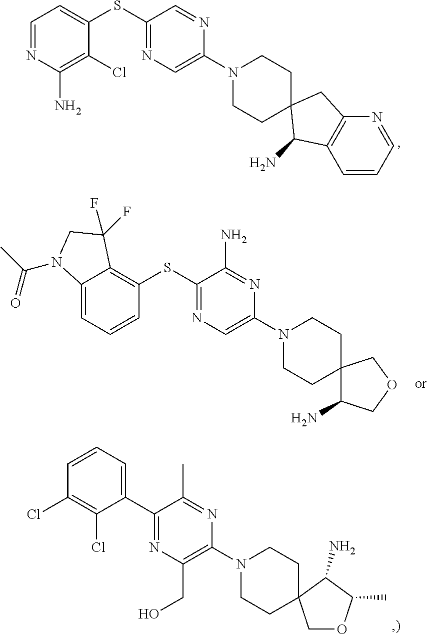 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

(S)-N-(1-(pyridin-2-yl)ethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(S)-N-(1-(pyridin-2-yl)ethyl)-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(S)-N-(1-(pyridin-2-yl)ethyl)-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(S)-N-(1-(pyridin-2-yl)ethyl)-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-N-(1-(pyridin-2-yl)ethyl)-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
(S)-N-(1-(pyridin-2-yl)ethyl)-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(S)-N-(1-(pyridin-2-yl)ethyl)-5-(4-(trifluoromethyl)phenyl)-2-naphthamide or a pharmaceutically acceptable salt thereof and a CDK4/6 inhibitor or a pharmaceutically acceptable salt thereof (e.g. a CDK416 inhibitor or a pharmaceutically acceptable salt thereof selected from the group consisting of palbociclib, ribociclib and abemaciclib)
(S)-N-(1-(pyridin-2-yl)ethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(S)-N-(1-(pyridin-2-yl)ethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-N-(1-(pyridin-2-yl)ethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(2-hydroxy-1-(pyridin-2-yl)ethyl)-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
N-(2-hydroxy-1-(pyridin-2-yl)ethyl)-5-(4-(trifluoromethyl)phenyl)-2-naphthamide or a pharmaceutically acceptable salt thereof and a MAPK pathway inhibitor or a pharmaceutically acceptable salt thereof
N-(2-hydroxy-1-(pyridin-2-yl)ethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

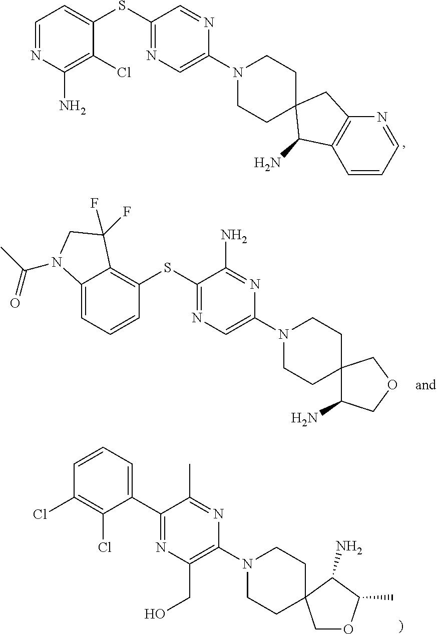 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

N-(2-hydroxy-1-(pyridin-2-yl)ethyl)-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

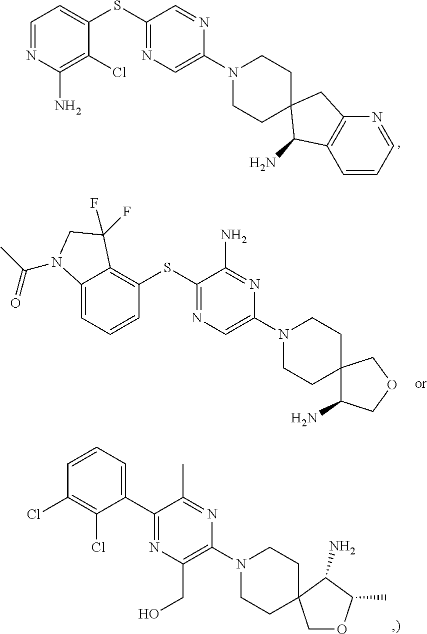 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

N-(2-hydroxy-1-(pyridin-2-yl)ethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
N-(2-hydroxy-1-(pyridin-2-yl)ethyl)-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-(2-hydroxy-1-(pyridin-2-yl)ethyl)-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-(2-hydroxy-1-(pyridin-2-yl)ethyl)-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(2-hydroxy-1-(pyridin-2-yl)ethyl)-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
N-(2-hydroxy-1-(pyridin-2-yl)ethyl)-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-(2-hydroxy-1-(pyridin-2-yl)ethyl)-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
N-(2-hydroxy-1-(pyridin-2-yl)ethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-(2-hydroxy-1-(pyridin-2-yl)ethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(2-hydroxy-1-(pyridin-2-yl)ethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(R)-N-(1-6-aminopyridin-2-yl)ethyl)-5-(4-(trifluoromethyl)phenyl)-2-napthamide or a pharmaceutically acceptable salt thereof and a cMET inhibitor or a pharmaceutically acceptable salt thereof (e.g. capmatinib or tepotinib, e.g. capmatinib)
(R)-N-(1-6-aminopyridin-2-yl)ethyl)-5-(4-(trifluoromethyl)phenyl)-2-napthamide or a pharmaceutically acceptable salt thereof and a MAPK pathway inhibitor or a pharmaceutically acceptable salt thereof
(R)-N-(1-6-aminopyridin-2-yl)ethyl)-5-(4-(trifluoromethyl)phenyl)-2-napthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), ASTX (Taiho & Otsuka Pharmas), X-37-SHP2 (X-37),

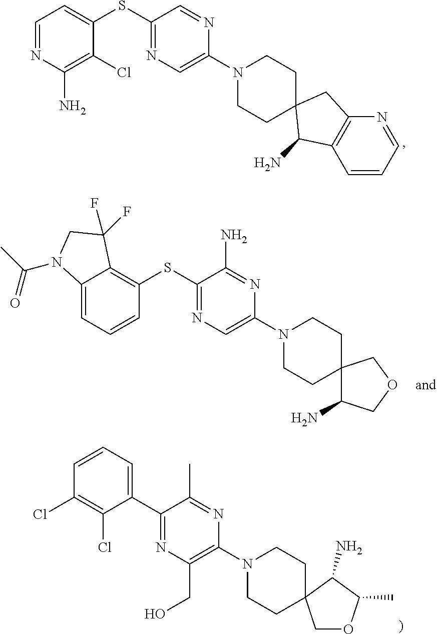 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

(R)-N-(1-6-aminopyridin-2-yl)ethyl)-5-(4-(trifluoromethyl)phenyl)-2-napthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

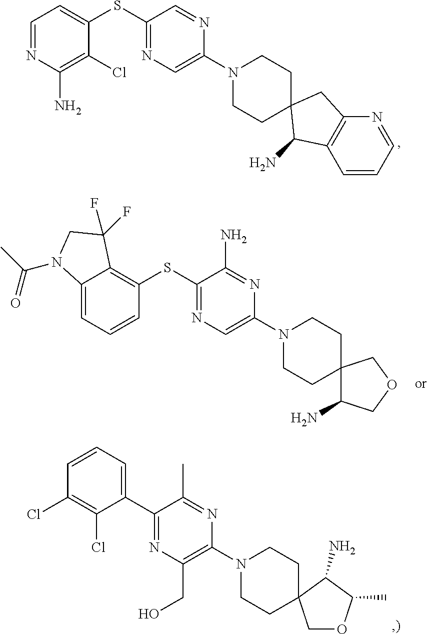 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

(R)-N-(1-6-aminopyridin-2-yl)ethyl)-5-(4-(trifluoromethyl)phenyl)-2-napthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(R)-N-(1-6-aminopyridin-2-yl)ethyl)-5-(4-(trifluoromethyl)phenyl)-2-napthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(R)-N-(1-6-aminopyridin-2-yl)ethyl)-5-(4-(trifluoromethyl)phenyl)-2-napthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(R)-N-(1-6-aminopyridin-2-yl)ethyl)-5-(4-(trifluoromethyl)phenyl)-2-napthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-N-(1-6-aminopyridin-2-yl)ethyl)-5-(4-(trifluoromethyl)phenyl)-2-napthamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
(R)-N-(1-6-aminopyridin-2-yl)ethyl)-5-(4-(trifluoromethyl)phenyl)-2-napthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(R)-N-(1-6-aminopyridin-2-yl)ethyl)-5-(4-(trifluoromethyl)phenyl)-2-napthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
(R)-N-(1-6-aminopyridin-2-yl)ethyl)-5-(4-(trifluoromethyl)phenyl)-2-napthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(R)-N-(1-6-aminopyridin-2-yl)ethyl)-5-(4-(trifluoromethyl)phenyl)-2-napthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-N-(1-6-aminopyridin-2-yl)ethyl)-5-(4-(trifluoromethyl)phenyl)-2-napthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-[(1R)-2-hydroxy-1-(2-pyridyl)ethyl]-5-[4-(trifluoromethyl)phenyl]naphthalene-2-carboxamide or a pharmaceutically acceptable salt thereof and a cMET inhibitor or a pharmaceutically acceptable salt thereof (e.g. capmatinib or tepotinib, e.g. capmatinib)
N-[(1R)-2-hydroxy-1-(2-pyridyl)ethyl]-5-[4-(trifluoromethyl)phenyl]naphthalene-2-carboxamide or a pharmaceutically acceptable salt thereof and a MAPK pathway inhibitor or a pharmaceutically acceptable salt thereof
N-[(1R)-2-hydroxy-1-(2-pyridyl)ethyl]-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), ASTX (Taiho & Otsuka Pharmas), X-37-SHP2 (X-37),

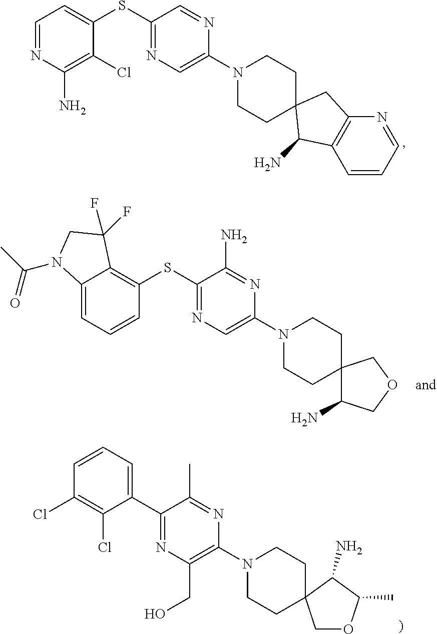 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

N-[(1R)-2-hydroxy-1-(2-pyridyl)ethyl]-5-[4-(trifluoromethyl)phenyl]naphthalene-2-carboxamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

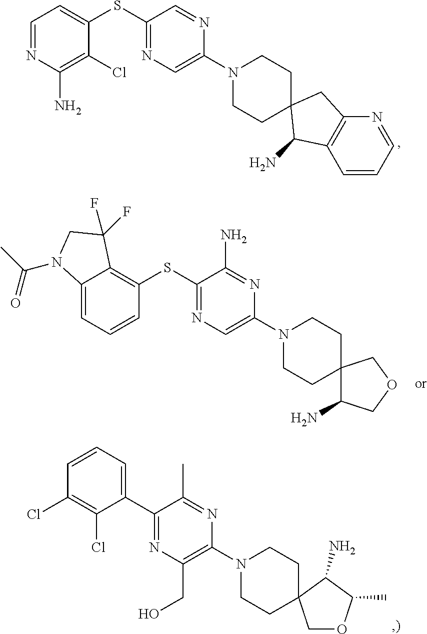 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

N-[(1R)-2-hydroxy-1-(2-pyridyl)ethyl]-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
N-[(1R)-2-hydroxy-1-(2-pyridyl)ethyl]-5-[4-(trifluoromethyl)phenyl]naphthalene-2-carboxamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-[(1R)-2-hydroxy-1-(2-pyridyl)ethyl]-5-[4-(trifluoromethyl)phenyl]naphthalene-2-carboxamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-[(1R)-2-hydroxy-1-(2-pyridyl)ethyl]-5-[4-(trifluoromethyl)phenyl]naphthalene-2-carboxamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-[(1R)-2-hydroxy-1-(2-pyridyl)ethyl]-5-[4-(trifluoromethyl)phenyl]naphthalene-2-carboxamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-[(1R)-2-hydroxy-1-(2-pyridyl)ethyl]-5-[4-(trifluoromethyl)phenyl]naphthalene-2-carboxamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-[(1R)-2-hydroxy-1-(2-pyridyl)ethyl]-5-[4-(trifluoromethyl)phenyl]naphthalene-2-carboxamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
N-[(1R)-2-hydroxy-1-(2-pyridyl)ethyl]-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-[(1R)-2-hydroxy-1-(2-pyridyl)ethyl]-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-[(1R)-2-hydroxy-1-(2-pyridyl)ethyl]-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(S)-N-(3-hydroxy-1-(pyridin-2-yl)propyl)-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
(S)-N-(3-hydroxy-1-(pyridin-2-yl)propyl)-5-(4-(trifluoromethyl)phenyl)-2-naphthamide or a pharmaceutically acceptable salt thereof and a MAPK pathway inhibitor or a pharmaceutically acceptable salt thereof
(S)-N-(3-hydroxy-1-(pyridin-2-yl)propyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 ASTX (Taiho & Otsuka Pharmas), X-37-SHP2 (X-37),

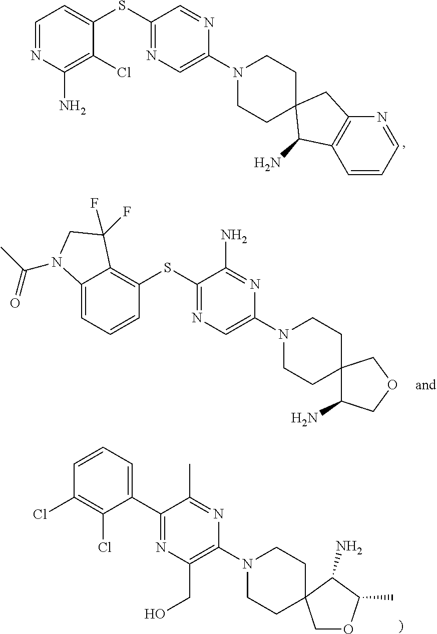 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

(S)-N-(3-hydroxy-1-(pyridin-2-yl)propyl)-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

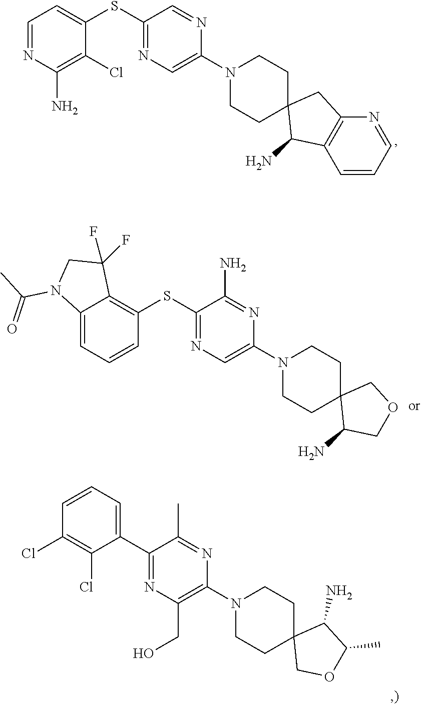 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(S)-N-(3-hydroxy-1-(pyridin-2-yl)propyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(S)-N-(3-hydroxy-1-(pyridin-2-yl)propyl)-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(S)-N-(3-hydroxy-1-(pyridin-2-yl)propyl)-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(S)-N-(3-hydroxy-1-(pyridin-2-yl)propyl)-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-N-(3-hydroxy-1-(pyridin-2-yl)propyl)-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
(S)-N-(3-hydroxy-1-(pyridin-2-yl)propyl)-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(S)-N-(3-hydroxy-1-(pyridin-2-yl)propyl)-5-(4-(trifluoromethyl)phenyl)-2-naphthamide or a pharmaceutically acceptable salt thereof and a CDK4/6 inhibitor or a pharmaceutically acceptable salt thereof (e.g. a CDK416 inhibitor or a pharmaceutically acceptable salt thereof selected from the group consisting of palbociclib, ribociclib and abemaciclib)
(S)-N-(3-hydroxy-1-(pyridin-2-yl)propyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(S)-N-(3-hydroxy-1-(pyridin-2-yl)propyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-N-(3-hydroxy-1-(pyridin-2-yl)propyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-[(1S)-3-hydroxy-1-methyl-propyl]-5-[4-(trifluoromethyl)phenyl]naphthalene-2-carboxamide or a pharmaceutically acceptable salt thereof and a cMET inhibitor or a pharmaceutically acceptable salt thereof (e.g. capmatinib or tepotinib, e.g. capmatinib)
N-[(1S)-3-hydroxy-1-methyl-propyl]-5-[4-(trifluoromethyl)phenyl]naphthalene-2-carboxamide or a pharmaceutically acceptable salt thereof and a MAPK pathway inhibitor or a pharmaceutically acceptable salt thereof
N-[(1S)-3-hydroxy-1-methyl-propyl]-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

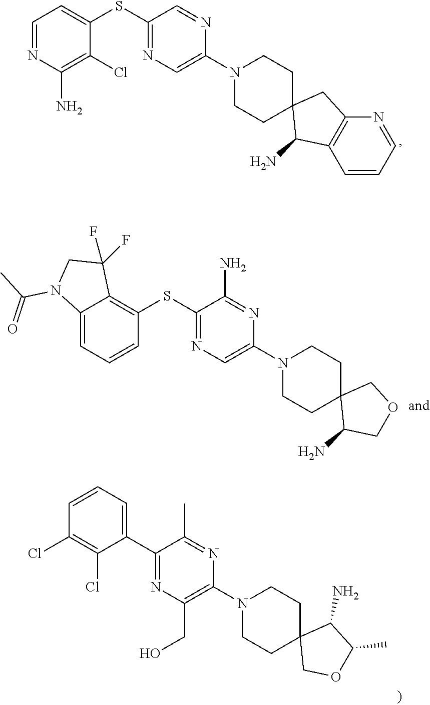 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-[(1S)-3-hydroxy-1-methyl-propyl]-5-[4-(trifluoromethyl)phenyl]naphthalene-2-carboxamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

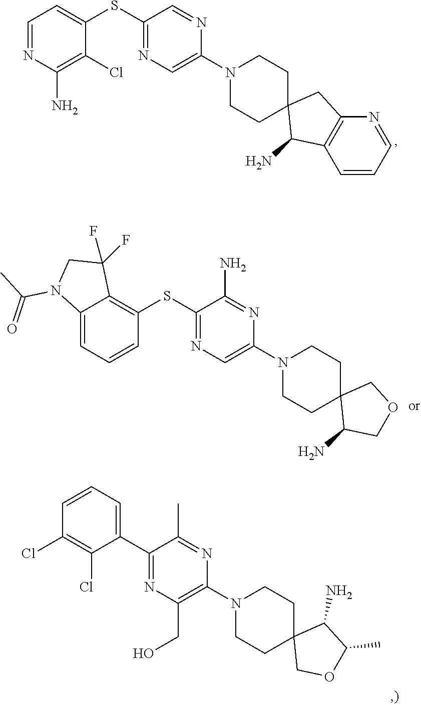 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-[(1S)-3-hydroxy-1-methyl-propyl]-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
N-[(1S)-3-hydroxy-1-methyl-propyl]-5-[4-(trifluoromethyl)phenyl]naphthalene-2-carboxamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-[(1S)-3-hydroxy-1-methyl-propyl]-5-[4-(trifluoromethyl)phenyl]naphthalene-2-carboxamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-[(1S)-3-hydroxy-1-methyl-propyl]-5-[4-(trifluoromethyl)phenyl]naphthalene-2-carboxamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-[(1S)-3-hydroxy-1-methyl-propyl]-5-[4-(trifluoromethyl)phenyl]naphthalene-2-carboxamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-[(1S)-3-hydroxy-1-methyl-propyl]-5-[4-(trifluoromethyl)phenyl]naphthalene-2-carboxamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-[(1S)-3-hydroxy-1-methyl-propyl]-5-[4-(trifluoromethyl)phenyl]naphthalene-2-carboxamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
N-[(1S)-3-hydroxy-1-methyl-propyl]-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-[(1S)-3-hydroxy-1-methyl-propyl]-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-[(1S)-3-hydroxy-1-methyl-propyl]-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-[(1R)-3-hydroxy-1-methyl-propyl]-5-[4-(trifluoromethyl)phenyl]naphthalene-2-carboxamide or a pharmaceutically acceptable salt thereof and a cMET inhibitor or a pharmaceutically acceptable salt thereof (e.g. capmatinib or tepotinib, e.g. capmatinib)
N-[(1R)-3-hydroxy-1-methyl-propyl]-5-[4-(trifluoromethyl)phenyl]naphthalene-2-carboxamide or a pharmaceutically acceptable salt thereof and a MAPK pathway inhibitor or a pharmaceutically acceptable salt thereof
N-[(1R)-3-hydroxy-1-methyl-propyl]-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

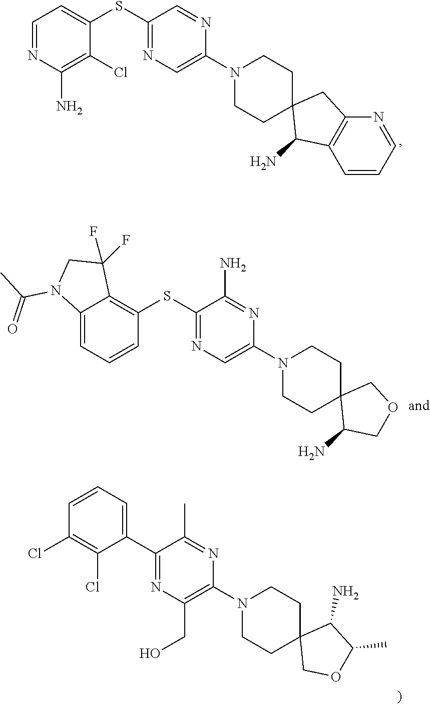 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-[(1R)-3-hydroxy-1-methyl-propyl]-5-[4-(trifluoromethyl)phenyl]naphthalene-2-carboxamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

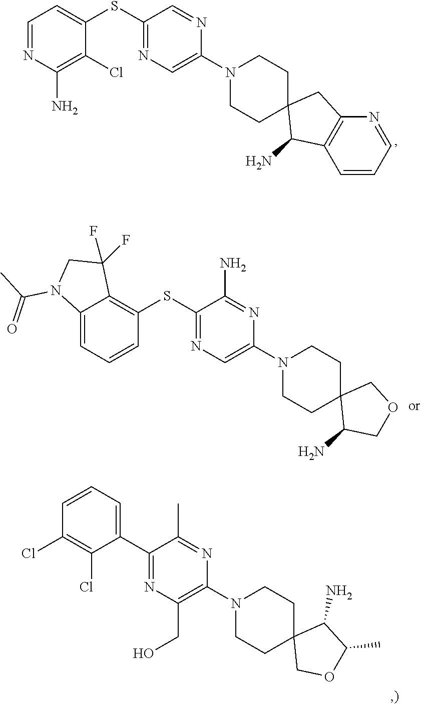 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-[(1R)-3-hydroxy-1-methyl-propyl]-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
N-[(1R)-3-hydroxy-1-methyl-propyl]-5-[4-(trifluoromethyl)phenyl]naphthalene-2-carboxamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-[(1R)-3-hydroxy-1-methyl-propyl]-5-[4-(trifluoromethyl)phenyl]naphthalene-2-carboxamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-[(1R)-3-hydroxy-1-methyl-propyl]-5-[4-(trifluoromethyl)phenyl]naphthalene-2-carboxamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-[(1R)-3-hydroxy-1-methyl-propyl]-5-[4-(trifluoromethyl)phenyl]naphthalene-2-carboxamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-[(1R)-3-hydroxy-1-methyl-propyl]-5-[4-(trifluoromethyl)phenyl]naphthalene-2-carboxamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-[(1R)-3-hydroxy-1-methyl-propyl]-5-[4-(trifluoromethyl)phenyl]naphthalene-2-carboxamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
N-[(1R)-3-hydroxy-1-methyl-propyl]-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-[(1R)-3-hydroxy-1-methyl-propyl]-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-[(1R)-3-hydroxy-1-methyl-propyl]-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(S)-N-(4-aminobutan-2-yl)-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
(S)-N-(4-aminobutan-2-yl)-5-(4-(trifluoromethyl)phenyl)-2-naphthamide or a pharmaceutically acceptable salt thereof and a MAPK pathway inhibitor or a pharmaceutically acceptable salt thereof
(S)-N-(4-aminobutan-2-yl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), (InnoCare), HBI2376 (HUYA Bioscience), ETS001 (Shanghai ETERN Biopharma), TAS-ASTX (Taiho & Otsuka Pharmas), X-37-SHP2 (X-37),

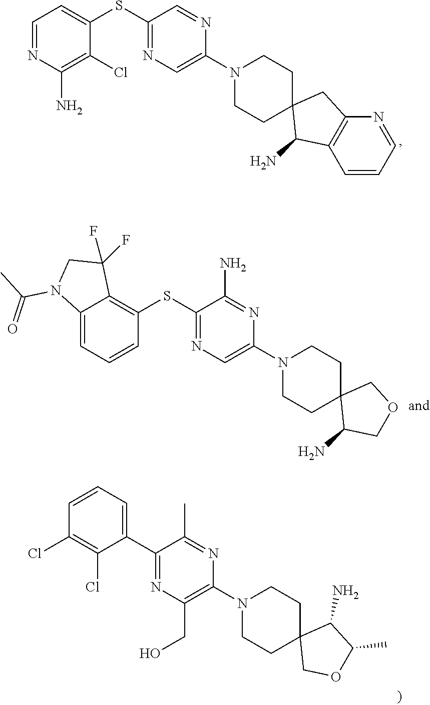 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(S)-N-(4-aminobutan-2-yl)-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

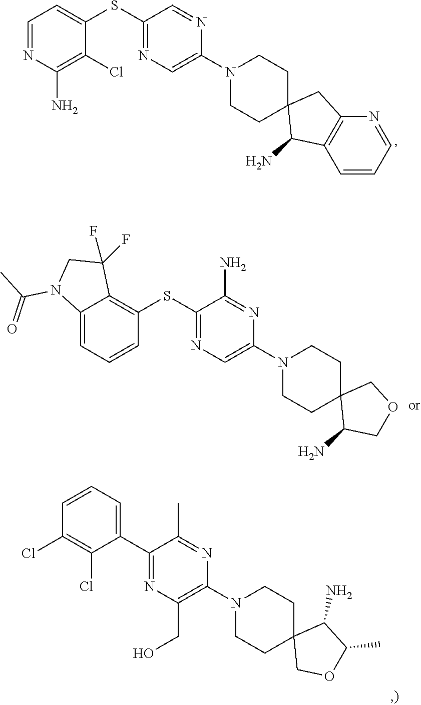 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(S)-N-(4-aminobutan-2-yl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(S)-N-(4-aminobutan-2-yl)-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(S)-N-(4-aminobutan-2-yl)-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(S)-N-(4-aminobutan-2-yl)-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-N-(4-aminobutan-2-yl)-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(S)-N-(4-aminobutan-2-yl)-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(S)-N-(4-aminobutan-2-yl)-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
(S)-N-(4-aminobutan-2-yl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(S)-N-(4-aminobutan-2-yl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting
(S)-N-(4-aminobutan-2-yl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-[3-hydroxy-1-(2-pyridyl)propyl]-5-[4-(trifluoromethyl)phenyl]naphthalene-2-carboxamide or a pharmaceutically acceptable salt thereof and a cMET inhibitor or a pharmaceutically acceptable salt thereof (e.g. capmatinib or tepotinib, e.g. capmatinib)
N-[3-hydroxy-1-(2-pyridyl)propyl]-5-[4-(trifluoromethyl)phenyl]naphthalene-2-carboxamide or a pharmaceutically acceptable salt thereof and a MAPK pathway inhibitor or a pharmaceutically acceptable salt thereof
N-[3-hydroxy-1-(2-pyridyl)propyl]-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), ASTX (Taiho & Otsuka Pharmas), X-37-SHP2 (X-37),

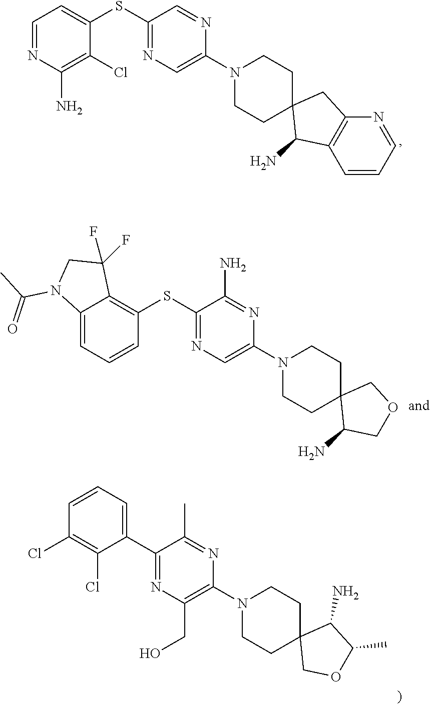 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-[3-hydroxy-1-(2-pyridyl)propyl]-5-[4-(trifluoromethyl)phenyl]naphthalene-2-carboxamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

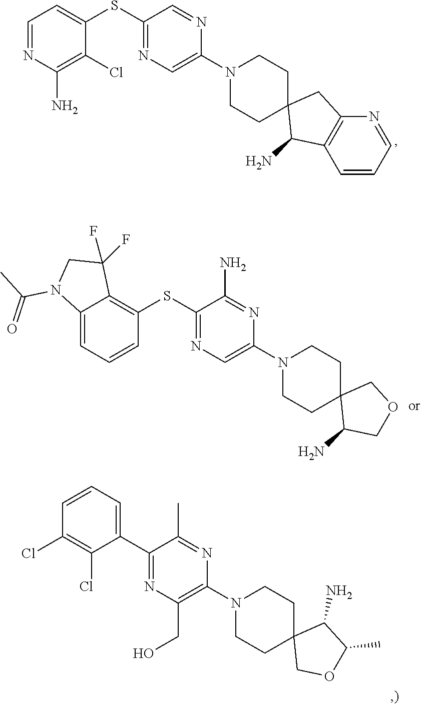 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-[3-hydroxy-1-(2-pyridyl)propyl]-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
N-[3-hydroxy-1-(2-pyridyl)propyl]-5-[4-(trifluoromethyl)phenyl]naphthalene-2-carboxamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-[3-hydroxy-1-(2-pyridyl)propyl]-5-[4-(trifluoromethyl)phenyl]naphthalene-2-carboxamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-[3-hydroxy-1-(2-pyridyl)propyl]-5-[4-(trifluoromethyl)phenyl]naphthalene-2-carboxamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-[3-hydroxy-1-(2-pyridyl)propyl]-5-[4-(trifluoromethyl)phenyl]naphthalene-2-carboxamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-[3-hydroxy-1-(2-pyridyl)propyl]-5-[4-(trifluoromethyl)phenyl]naphthalene-2-carboxamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-[3-hydroxy-1-(2-pyridyl)propyl]-5-[4-(trifluoromethyl)phenyl]naphthalene-2-carboxamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
N-[3-hydroxy-1-(2-pyridyl)propyl]-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-[3-hydroxy-1-(2-pyridyl)propyl]-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-[3-hydroxy-1-(2-pyridyl)propyl]-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(R)-N-(1-(azetidin-3-yl)ethyl)-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
(R)-N-(1-(azetidin-3-yl)ethyl)-5-(4-(trifluoromethyl)phenyl)-2-naphthamide or a pharmaceutically acceptable salt thereof and a MAPK pathway inhibitor or a pharmaceutically acceptable salt thereof
(R)-N-(1-(azetidin-3-yl)ethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

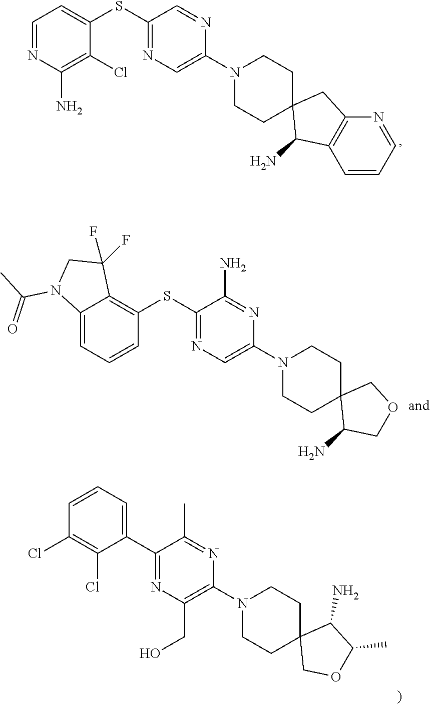 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(R)-N-(1-(azetidin-3-yl)ethyl)-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

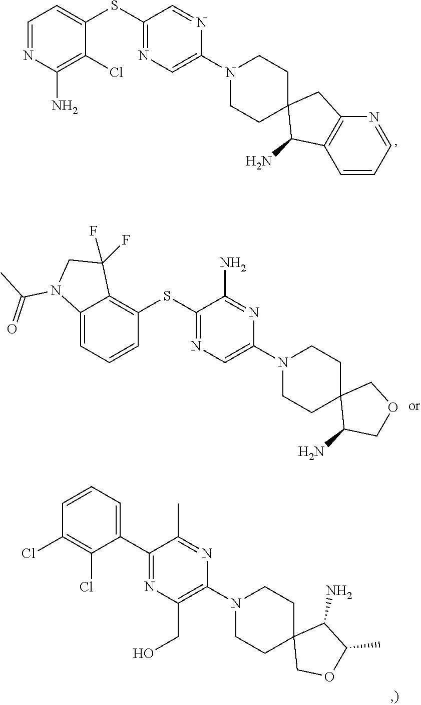 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(R)-N-(1-(azetidin-3-yl)ethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(R)-N-(1-(azetidin-3-yl)ethyl)-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(R)-N-(1-(azetidin-3-yl)ethyl)-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(R)-N-(1-(azetidin-3-yl)ethyl)-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-N-(1-(azetidin-3-yl)ethyl)-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
(R)-N-(1-(azetidin-3-yl)ethyl)-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(R)-N-(1-(azetidin-3-yl)ethyl)-5-(4-(trifluoromethyl)phenyl)-2-naphthamide or a pharmaceutically acceptable salt thereof and a CDK4/6 inhibitor or a pharmaceutically acceptable salt thereof (e.g. a CDK416 inhibitor or a pharmaceutically acceptable salt thereof selected from the group consisting of palbociclib, ribociclib and abemaciclib)
(R)-N-(1-(azetidin-3-yl)ethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(R)-N-(1-(azetidin-3-yl)ethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-N-(1-(azetidin-3-yl)ethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(R)-N-[1-(2-amino-3-pyridyl)ethyl]-5-[4-(trifluoromethyl)phenyl]naphthalene-2-carboxamide or a pharmaceutically acceptable salt thereof and a cMET inhibitor or a pharmaceutically acceptable salt thereof (e.g. capmatinib or tepotinib, e.g. capmatinib)
(R)-N-[1-(2-amino-3-pyridyl)ethyl]-5-[4-(trifluoromethyl)phenyl]naphthalene-2-carboxamide or a pharmaceutically acceptable salt thereof and a MAPK pathway inhibitor or a pharmaceutically acceptable salt thereof
(R)-N-[1-(2-amino-3-pyridyl)ethyl]-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

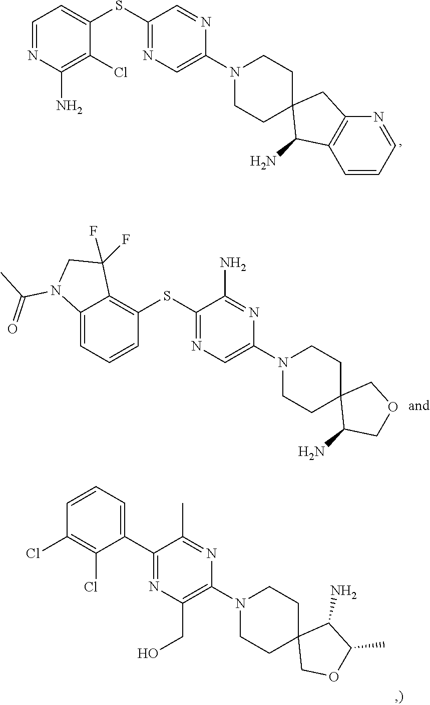 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(R)-N-[1-(2-amino-3-pyridyl)ethyl]-5-[4-(trifluoromethyl)phenyl]naphthalene-2-carboxamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

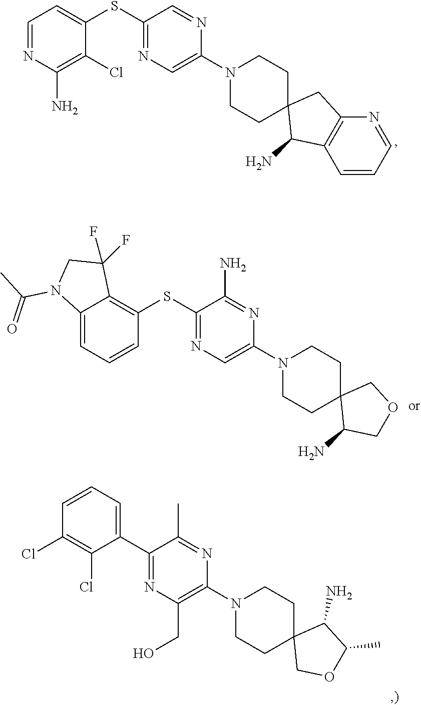 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(R)-N-[1-(2-amino-3-pyridyl)ethyl]-5-[4-(trifluoromethyl)phenyl]naphthalene-2-carboxamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(R)-N-[1-(2-amino-3-pyridyl)ethyl]-5-[4-(trifluoromethyl)phenyl]naphthalene-2-carboxamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(R)-N-[1-(2-amino-3-pyridyl)ethyl]-5-[4-(trifluoromethyl)phenyl]naphthalene-2-carboxamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(R)-N-[1-(2-amino-3-pyridyl)ethyl]-5-[4-(trifluoromethyl)phenyl]naphthalene-2-carboxamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-N-[1-(2-amino-3-pyridyl)ethyl]-5-[4-(trifluoromethyl)phenyl]naphthalene-2-carboxamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(R)-N-[1-(2-amino-3-pyridyl)ethyl]-5-[4-(trifluoromethyl)phenyl]naphthalene-2-carboxamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(R)-N-[1-(2-amino-3-pyridyl)ethyl]-5-[4-(trifluoromethyl)phenyl]naphthalene-2-carboxamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
(R)-N-[1-(2-amino-3-pyridyl)ethyl]-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib)
(R)-N-[1-(2-amino-3-pyridyl)ethyl]-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-N-[1-(2-amino-3-pyridyl)ethyl]-5-[4-(trifluoromethyl)phenyl]naphthalene-2-carboxamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(pyridin-4-ylmethyl)-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
N-(pyridin-4-ylmethyl)-5-(4-(trifluoromethyl)phenyl)-2-naphthamide or a pharmaceutically acceptable salt thereof and a MAPK pathway inhibitor or a pharmaceutically acceptable salt thereof
N-(pyridin-4-ylmethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

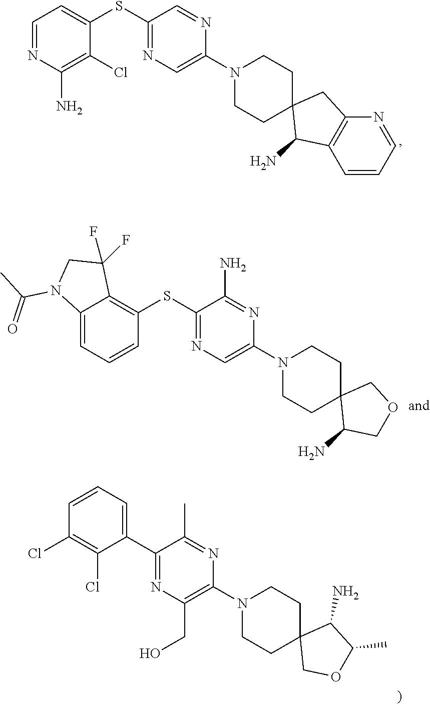 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-(pyridin-4-ylmethyl)-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

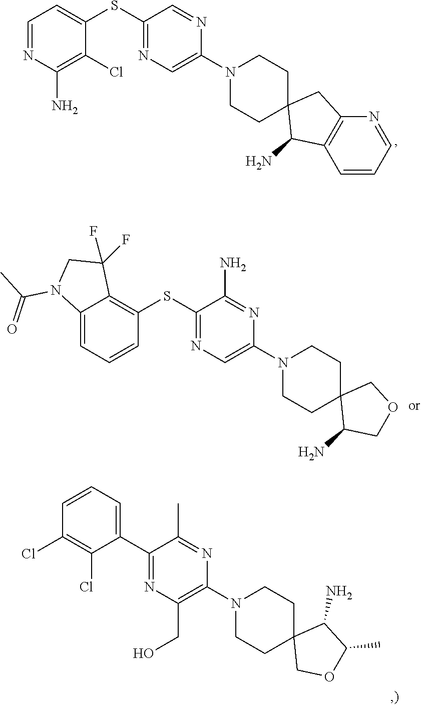 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-(pyridin-4-ylmethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
N-(pyridin-4-ylmethyl)-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-(pyridin-4-ylmethyl)-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-(pyridin-4-ylmethyl)-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(pyridin-4-ylmethyl)-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(pyridin-4-ylmethyl)-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-(pyridin-4-ylmethyl)-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
N-(pyridin-4-ylmethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-(pyridin-4-ylmethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(pyridin-4-ylmethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(pyridin-2-ylmethyl)-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
N-(pyridin-2-ylmethyl)-5-(4-(trifluoromethyl)phenyl)-2-naphthamide or a pharmaceutically acceptable salt thereof and a MAPK pathway inhibitor or a pharmaceutically acceptable salt thereof
N-(pyridin-2-ylmethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

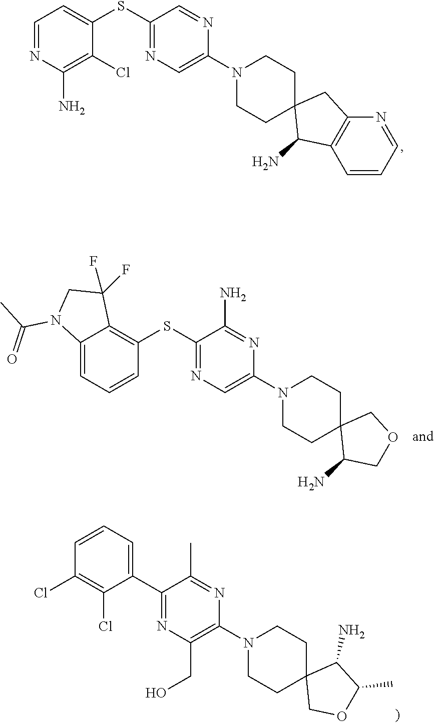 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-(pyridin-2-ylmethyl)-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

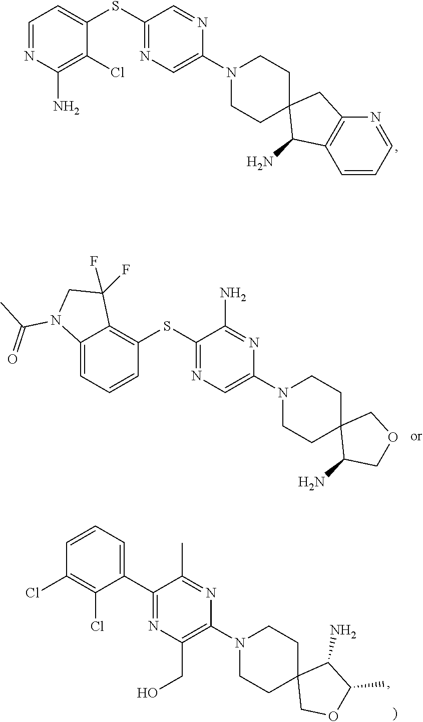 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

N-(pyridin-2-ylmethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adaarasib)
N-(pyridin-2-ylmethyl)-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
N-(pyridin-2-ylmethyl)-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-(pyridin-2-ylmethyl)-5-(4-(trifluoromethyl)phenyl)-2-naphthamide or a pharmaceutically acceptable salt thereof and a MEK inhibitor or a pharmaceutically acceptable salt thereof (e.g. a MEK inhibitoror a pharmaceutically acceptable salt thereof selected from the group consisting of trametinib, binimetinib, selumetinib, pimasertib, PD-0325901, and cobimetinib, e.g. trametinib)
N-(pyridin-2-ylmethyl)-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(pyridin-2-ylmethyl)-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-(pyridin-2-ylmethyl)-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
N-(pyridin-2-ylmethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-(pyridin-2-ylmethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(pyridin-2-ylmethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(2-Hydroxy-1-(pyridin-2-yl)ethyl)-7-methoxy-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
N-(2-Hydroxy-1-(pyridin-2-yl)ethyl)-7-methoxy-5-(4-(trifluoromethyl)phenyl)-2-naphthamide or a pharmaceutically acceptable salt thereof and a MAPK pathway inhibitor or a pharmaceutically acceptable salt thereof
N-(2-Hydroxy-1-(pyridin-2-yl)ethyl)-7-methoxy-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), ASTX (Taiho & Otsuka Pharmas), X-37-SHP2 (X-37),

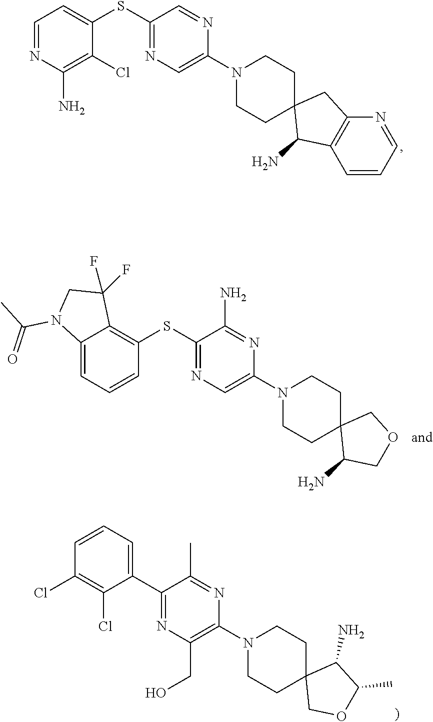 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

N-(2-Hydroxy-1-(pyridin-2-yl)ethyl)-7-methoxy-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

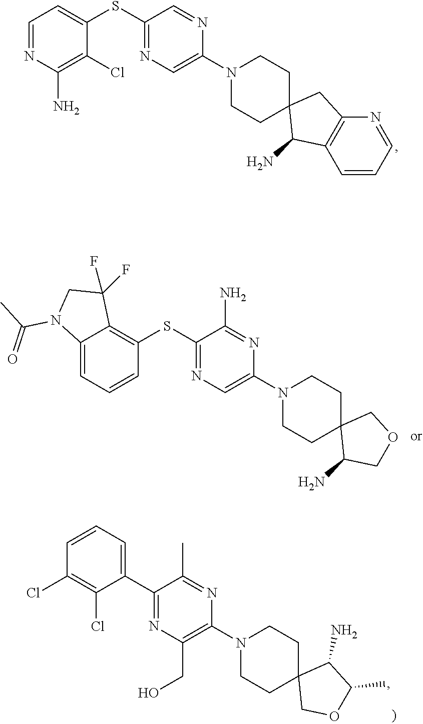 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

N-(2-Hydroxy-1-(pyridin-2-yl)ethyl)-7-methoxy-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
N-(2-Hydroxy-1-(pyri din-2-yl)ethyl)-7-methoxy-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib. e.d. LTT462 or erlotinib)
N-(2-Hydroxy-1-(pyridin-2-yl)ethyl)-7-methoxy-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
N-(2-Hydroxy-1-(pyridin-2-yl)ethyl)-7-methoxy-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(2-Hydroxy-1-(pyridin-2-yl)ethyl)-7-methoxy-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
N-(2-Hydroxy-1-(pyridin-2-yl)ethyl)-7-methoxy-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
N-(2-Hydroxy-1-(pyridin-2-yl)ethyl)-7-methoxy-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
N-(2-Hydroxy-1-(pyridin-2-yl)ethyl)-7-methoxy-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
N-(2-Hydroxy-1-(pyrdin-2-yl)ethyl)-7-methoxy-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
N-(2-Hydroxy-1-(pyridin-2-yl)ethyl)-7-methoxy-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(S)-7-Methoxy-N-(1-(pyridin-2-yl)ethyl)-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
(S)-7-Methoxy-N-(1-(pyridin-2-yl)ethyl)-5-(4-(trifluoromethyl)phenyl)-2-naphthamide or a pharmaceutically acceptable salt thereof and a MAPK pathway inhibitor or a pharmaceutically acceptable salt thereof
(S)-7-Methoxy-N-(1-(pyridin-2-yl)ethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 ASTX (Taiho & Otsuka Pharmas), X-37-SHP2 (X-37),

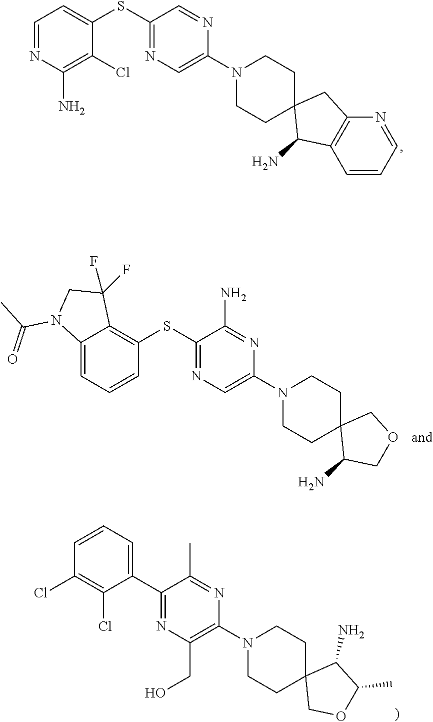 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(S)-7-Methoxy-N-(1-(pyridin-2-yl)ethyl)-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

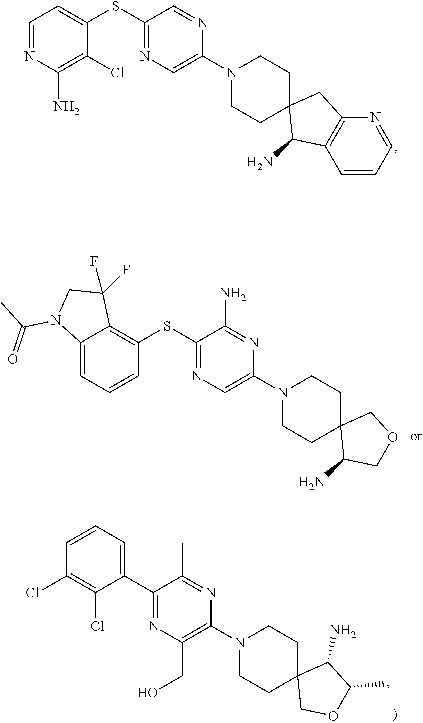 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

(S)-7-Methoxy-N-(1-(pyridin-2-yl)ethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(S)-7-Methoxy-N-(1-(pyridin-2-yl)ethyl)-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(S)-7-Methoxy-N-(1-(pyridin-2-yl)ethyl)-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(S)-7-Methoxy-N-(1-(pyridin-2-yl)ethyl)-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-7-Methoxy-N-(1-(pyridin-2-yl)ethyl)-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
(S)-7-Methoxy-N-(1-(pyridin-2-yl)ethyl)-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(S)-7-Methoxy-N-(1-(pyridin-2-yl)ethyl)-5-(4-(trifluoromethyl)phenyl)-2-naphthamide or a pharmaceutically acceptable salt thereof and a CDK4/6 inhibitor or a pharmaceutically acceptable salt thereof (e.g. a CDK416 inhibitor or a pharmaceutically acceptable salt thereof selected from the group consisting of palbociclib, ribociclib and abemaciclib)
(S)-7-Methoxy-N-(1-(pyridin-2-yl)ethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(S)-7-Methoxy-N-(1-(pyridin-2-yl)ethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-7-Methoxy-N-(1-(pyridin-2-yl)ethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(R)-7-Methoxy-N-(1-(pyridin-2-yl)ethyl)-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
(R)-7-Methoxy-N-(1-(pyridin-2-yl)ethyl)-5-(4-(trifluoromethyl)phenyl)-2-naphthamide or a pharmaceutically acceptable salt thereof and a MAPK pathway inhibitor or a pharmaceutically acceptable salt thereof
(R)-7-Methoxy-N-(1-(pyridin-2-yl)ethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

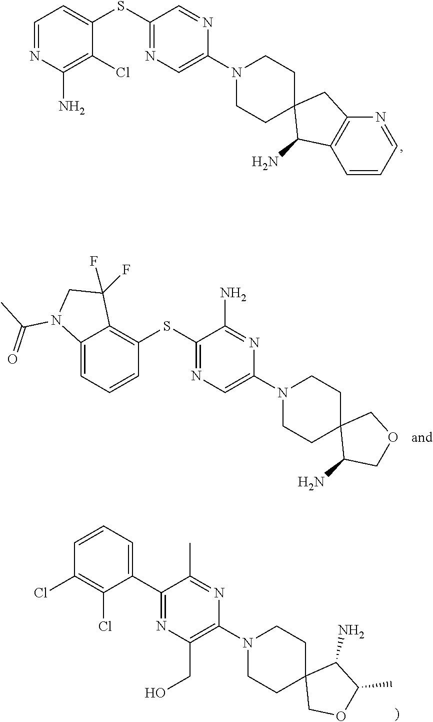 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(R)-7-Methoxy-N-(1-(pyridin-2-yl)ethyl)-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

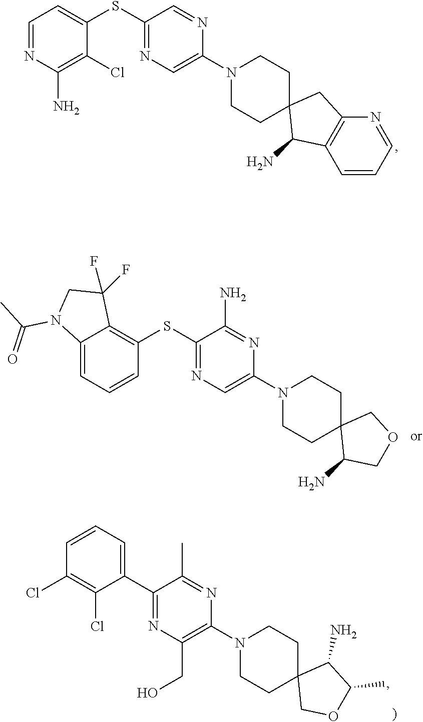 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

(R)-7-Methoxy-N-(1-(pyridin-2-yl)ethyl)-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(R)-7-Methoxy-N-(1-(pyridin-2-yl)ethyl)-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(R)-7-Methoxy-N-(1-(pyridin-2-yl)ethyl)-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(R)-7-Methoxy-N-(1-(pyridin-2-yl)ethyl)-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-7-Methoxy-N-(1-(pyridin-2-yl)ethyl)-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
(R)-7-Methoxy-N-(1-(pyridin-2-yl)ethyl)-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(R)-7-Methoxy-N-(1-(pyridin-2-yl)ethyl)-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
(R)-7-Methoxy-N-(1-(pyridin-2-yl)ethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(R)-7-Methoxy-N-(1-(pyridin-2-yl)ethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(R)-7-Methoxy-N-(1-(pyridin-2-yl)ethyl)-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
(S)-N-(1-Hydroxypropan-2-yl)-7-methoxy-5-(4-(trifluoromethyl)phenyl)-2-naphthamide or a pharmaceutically acceptable salt thereof and a cMET inhibitor or a pharmaceutically acceptable salt thereof (e.g. capmatinib or tepotinib, e.g. capmatinib)
(S)-N-(1-Hydroxypropan-2-yl)-7-methoxy-5-(4-(trifluoromethyl)phenyl)-2-naphthamide or a pharmaceutically acceptable salt thereof and a MAPK pathway inhibitor or a pharmaceutically acceptable salt thereof
(S)-N-(1-Hydroxypropan-2-yl)-7-methoxy-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

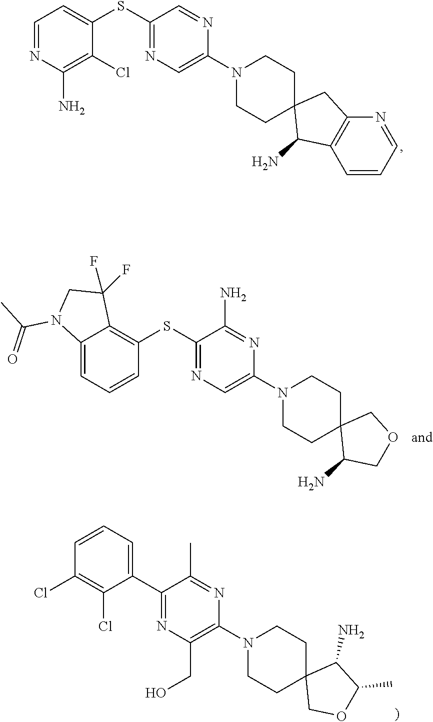 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

(S)-N-(1-Hydroxypropan-2-yl)-7-methoxy-5-(4-(trifluoromethyl)phenyl)-2-naphthamide or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

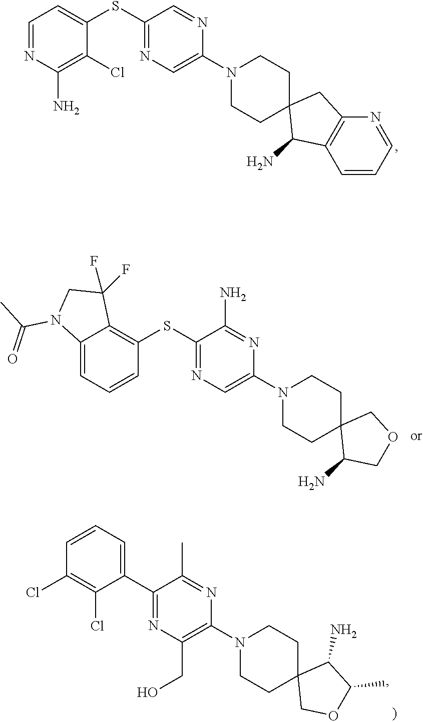 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

(S)-N-(1-Hydroxypropan-2-yl)-7-methoxy-5-(4-(trifluoromethyl)phenyl)-2-naphthamide or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
(S)-N-(1-Hydroxypropan-2-yl)-7-methoxy-5-(4-(trifluoromethyl)phenyl)-2-naphthamide or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
(S)-N-(1-Hydroxypropan-2-yl)-7-methoxy-5-(4-(trifluoromethyl)phenyl)-2-naphthamide or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
(S)-N-(1-Hydroxypropan-2-yl)-7-methoxy-5-(4-(trifluoromethyl)phenyl)-2-naphthamide or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-N-(1-Hydroxypropan-2-yl)-7-methoxy-5-(4-(trifluoromethyl)phenyl)-2-naphthamide or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
(S)-N-(1-Hydroxypropan-2-yl)-7-methoxy-5-(4-(trifluoromethyl)phenyl)-2-naphthamide or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
(S)-N-(1-Hydroxypropan-2-yl)-7-methoxy-5-(4-(trifluoromethyl)phenyl)-2-naphthamide or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
(S)-N-(1-Hydroxypropan-2-yl)-7-methoxy-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib)
(S)-N-(1-Hydroxypropan-2-yl)-7-methoxy-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
(S)-N-(1-Hydroxypropan-2-yl)-7-methoxy-5-(4-(trifluoromethyl)phenyl)-2-naphthamide or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Any one of Compound Nos. 2, 3, 5, 14, 15, 17, 22, 25, 26, 29, 41, 42, 44, 45, 47, 49, 51, 52, 54, 73, 75, 76, 77, 79-81, 83, 85-87, 96, 102, 108, 113-116, 120-124, 129, 130, 132, 135, 140, 146, 149, 152, 155, 158, 161, 162, 164,166, 168, 171, 174, 178-180, 182, 184, 187, 190, 191, 197, 198, 201-203 of WO2019/222431 or a pharmaceutically acceptable salt thereof and a cMET inhibitor or a pharmaceutically acceptable salt thereof (e.g. capmatinib or tepotinib, e.g. capmatinib)
Any one of Compound Nos. 2, 3, 5, 14, 15, 17, 22, 25, 26, 29, 41, 42, 44, 45, 47, 49, 51, 52, 54, 73, 75, 76, 77, 79-81, 83, 85-87, 96, 102, 108, 113-116, 120-124, 129, 130, 132, 135, 140, 146, 149, 152, 155, 158, 161, 162, 164, 166, 168, 171, 174, 178-180, 182, 184, 187, 190, 191, 197, 198, 201-203 of WO2019/222431 or a pharmaceutically acceptable salt thereof and a MAPK pathway inhibitor or a pharmaceutically acceptable salt thereof
Any one of Compound Nos. 2, 3, 5, 14, 15, 17, 22, 25, 26, 29, 41, 42, 44, 45, 47, 49, 51, 52, 54, 73, 75, 76, 77, 79-81, 83, 85-87, 96, 102, 108, 113-116, 120-124, 129, 130, 132, 135, 140, 146, 149, 152, 155, 158, 161, 162, 164, 166, 168, 171, 174, 178-180, 182, 184, 187, 190, 191, 197, 198, 201-203 of WO2019/222431 or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

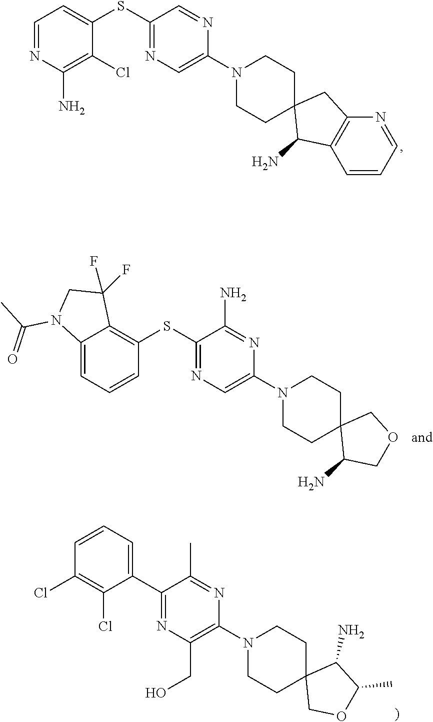 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

Any one of Compound Nos. 2, 3, 5, 14, 15, 17, 22, 25, 26, 29, 41, 42, 44, 45, 47, 49, 51, 52, 54, 73, 75, 76, 77, 79-81, 83, 85-87, 96, 102, 108, 113-116, 120-124, 129, 130, 132, 135, 140, 146, 149, 152, 155, 158, 161, 162, 164, 166, 168, 171, 174, 178-180, 182, 184, 187, 190, 191, 197, 198, 201-203 of WO2019/222431 or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

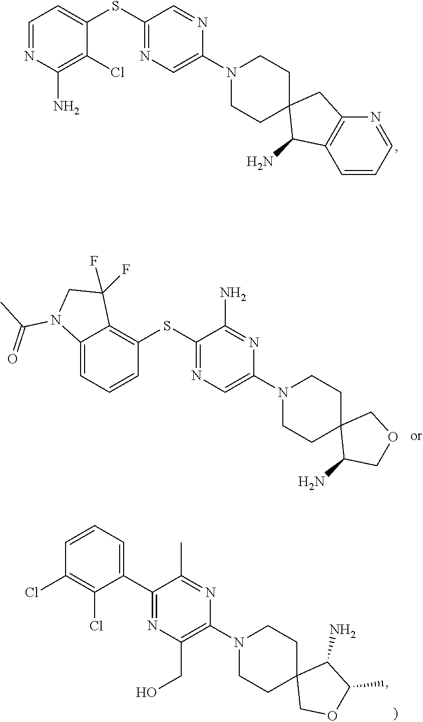 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

Any one of Compound Nos. 2, 3, 5, 14, 15, 17, 22, 25, 26, 29, 41, 42, 44, 45, 47, 49, 51, 52, 54, 73, 75, 76, 77, 79-81, 83, 85-87, 96, 102, 108, 113-116, 120-124, 129, 130, 132, 135, 140, 146, 149, 152, 155, 158, 161, 162, 164,166, 168, 171, 174, 178-180, 182, 184, 187, 190, 191, 197, 198, 201-203 of WO2019/222431 or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
Any one of Compound Nos. 2, 3, 5, 14, 15, 17, 22, 25, 26, 29, 41, 42, 44, 45, 47, 49, 51, 52, 54, 73, 75, 76, 77, 79-81, 83, 85-87, 96, 102, 108, 113-116, 120-124, 129, 130, 132, 135, 140, 146, 149, 152, 155, 158, 161, 162, 164,166, 168, 171, 174, 178-180, 182, 184, 187, 190, 191, 197, 198, 201-203 of WO2019/222431 or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
Any one of Compound Nos. 2, 3, 5, 14, 15, 17, 22, 25, 26, 29, 41, 42, 44, 45, 47, 49, 51, 52, 54, 73, 75, 76, 77, 79-81, 83, 85-87, 96, 102, 108, 113-116, 120-124, 129, 130, 132, 135, 140, 146, 149, 152, 155, 158, 161, 162, 164,166, 168, 171, 174, 178-180, 182, 184, 187, 190, 191, 197, 198, 201-203 of WO2019/222431 or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
Any one of Compound Nos. 2, 3, 5, 14, 15, 17, 22, 25, 26, 29, 41, 42, 44, 45, 47, 49, 51, 52, 54, 73, 75, 76, 77, 79-81, 83, 85-87, 96, 102, 108, 113-116, 120-124, 129, 130, 132, 135, 140, 146, 149, 152, 155, 158, 161, 162, 164,166, 168, 171, 174, 178-180, 182, 184, 187, 190, 191, 197, 198, 201-203 of WO2019/222431 or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Any one of Compound Nos. 2, 3, 5, 14, 15, 17, 22, 25, 26, 29, 41, 42, 44, 45, 47, 49, 51, 52, 54, 73, 75, 76, 77, 79-81, 83, 85-87, 96, 102, 108, 113-116, 120-124, 129, 130, 132, 135, 140, 146, 149, 152, 155, 158, 161, 162, 164,166, 168, 171, 174, 178-180, 182, 184, 187, 190, 191, 197, 198, 201-203 of WO2019/222431 or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Any one of Compound Nos. 2, 3, 5, 14, 15, 17, 22, 25, 26, 29, 41, 42, 44, 45, 47, 49, 51, 52, 54, 73, 75, 76, 77, 79-81, 83, 85-87, 96, 102, 108, 113-116, 120-124, 129, 130, 132, 135, 140, 146, 149, 152, 155, 158, 161, 162, 164,166, 168, 171, 174, 178-180, 182, 184, 187, 190, 191, 197, 198, 201-203 of WO2019/222431 or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
Any one of Compound Nos. 2, 3, 5, 14, 15, 17, 22, 25, 26, 29, 41, 42, 44, 45, 47, 49, 51, 52, 54, 73, 75, 76, 77, 79-81, 83, 85-87, 96, 102, 108, 113-116, 120-124, 129, 130, 132, 135, 140, 146, 149, 152, 155, 158, 161, 162, 164,166, 168, 171, 174, 178-180, 182, 184, 187, 190, 191, 197, 198, 201-203 of WO2019/222431 or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
Any one of Compound Nos. 2, 3, 5, 14, 15, 17, 22, 25, 26, 29, 41, 42, 44, 45, 47, 49, 51, 52, 54, 73, 75, 76, 77, 79-81, 83, 85-87, 96, 102, 108, 113-116, 120-124, 129, 130, 132, 135, 140, 146, 149, 152, 155, 158, 161, 162, 164,166, 168, 171, 174, 178-180, 182, 184, 187, 190, 191, 197, 198, 201-203 of WO20191222431 or a pharmaceutically acceptable salt thereof and a Raf inhibitor or a pharmaceutically acceptable salt thereof (e.g. belvarafenib, naporafenib, Encorafenib, vemurafenib or dabrafenib)
Any one of Compound Nos. 2, 3, 5, 14, 15, 17, 22, 25, 26, 29, 41, 42, 44, 45, 47, 49, 51, 52, 54, 73, 75, 76, 77, 79-81, 83, 85-87, 96, 102, 108, 113-116, 120-124, 129, 130, 132, 135, 140, 146, 149, 152, 155, 158, 161, 162, 164,166, 168, 171, 174, 178-180, 182, 184, 187, 190, 191, 197, 198, 201-203 of WO2019/222431 or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Any one of Compound Nos. 2, 3, 5, 14, 15, 17, 22, 25, 26, 29, 41, 42, 44, 45, 47, 49, 51, 52, 54, 73, 75, 76, 77, 79-81, 83, 85-87, 96, 102, 108, 113-116, 120-124, 129, 130, 132, 135, 140, 146, 149, 152, 155, 158, 161, 162, 164,166, 168, 171, 174, 178-180, 182, 184, 187, 190, 191, 197, 198, 201-203 of WO2019/222431 or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Any one of Compound Nos. 1-8, 10, 11, 15, 21-23, 25, 27, 28, 30 33, 34, 36-43, 45, 46, 49-59 and 61 (e.g. any one of Compounds 1, 3, 7, 8, 10, 11, 15, 25, 28, 30, 33, 34, 36, 37-43, 45, 46, 49-59 and 61) of WO2019/113236 or a pharmaceutically acceptable salt thereof and a cMET inhibitor or a pharmaceutically acceptable salt thereof (e.g. capmatinib or tepotinib, e.g. capmatinib)
Any one of Compound Nos. 1-8, 10, 11, 15, 21-23, 25, 27, 28, 30 33, 34, 36-43, 45, 46, 49-59 and 61 (e.g. any one of Compounds 1, 3, 7, 8, 10, 11, 15, 25, 28, 30, 33, 34, 36, 37-43, 45, 46, 49-59 and 61) of WO2019/113236 or a pharmaceutically acceptable salt thereof and a MAPK pathway inhibitor or a pharmaceutically acceptable salt thereof
Any one of Compound Nos. 1-8, 10, 11, 15, 21-23, 25, 27, 28, 30 33, 34, 36-43, 45, 46, 49-59 and 61 (e.g. any one of Compounds 1, 3, 7, 8, 10, 11, 15, 25, 28, 30, 33, 34, 36, 37-43, 45, 46, 49-59 and 61) of WO2019/113236 or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

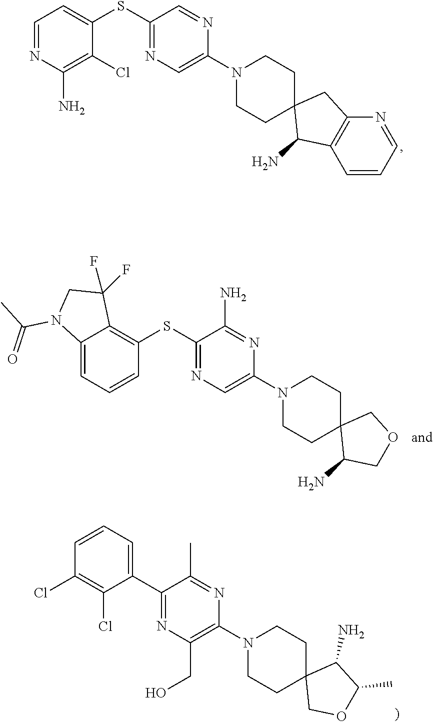 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

Any one of Compound Nos. 1-8, 10, 11, 15, 21-23, 25, 27, 28, 30 33, 34, 36-43, 45, 46, 49-59 and 61 (e.g. any one of Compounds 1, 3, 7, 8, 10, 11, 15, 25, 28, 30, 33, 34, 36, 37-43, 45, 46, 49-59 and 61) of WO2019/113236 or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

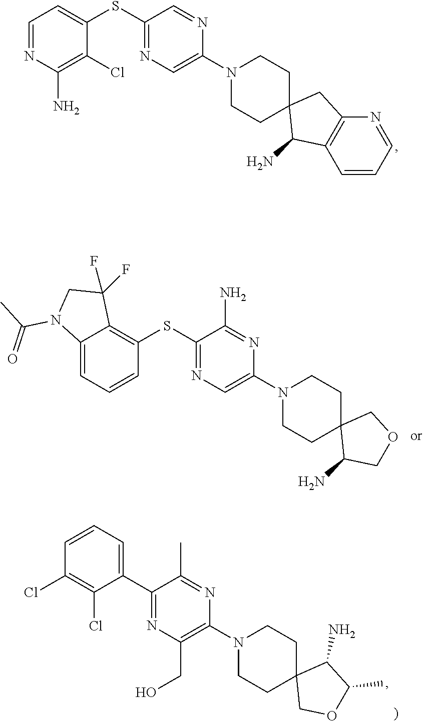 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

Any one of Compound Nos. 1-8, 10, 11, 15, 21-23, 25, 27, 28, 30 33, 34, 36-43, 45, 46, 49-59 and 61 (e.g. any one of Compounds 1, 3, 7, 8, 10, 11, 15, 25, 28, 30, 33, 34, 36, 37-43, 45, 46, 49-59 and 61) of WO2019/113236 or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
Any one of Compound Nos. 1-8, 10, 11, 15, 21-23, 25, 27, 28, 30 33, 34, 36-43, 45, 46, 49-59 and 61 (e.g. any one of Compounds 1, 3, 7, 8, 10, 11, 15, 25, 28, 30, 33, 34, 36, 37-43, 45, 46, 49-59 and 61) of WO2019/113236 or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
Any one of Compound Nos. 1-8, 10, 11, 15, 21-23, 25, 27, 28, 30 33, 34, 36-43, 45, 46, 49-59 and 61 (e.g. any one of Compounds 1, 3, 7, 8, 10, 11, 15, 25, 28, 30, 33, 34, 36, 37-43, 45, 46, 49-59 and 61) of WO2019/113236 or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
Any one of Compound Nos. 1-8, 10, 11, 15, 21-23, 25, 27, 28, 30 33, 34, 36-43, 45, 46, 49-59 and 61 (e.g. any one of Compounds 1, 3, 7, 8, 10, 11, 15, 25, 28, 30, 33, 34, 36, 37-43, 45, 46, 49-59 and 61) of WO2019/113236 or a pharmaceutically acceptable salt thereof and a MEK inhibitor or a pharmaceutically acceptable salt thereof (e.g. a MEK inhibitor or a pharmaceutically acceptable salt thereof selected from the group consisting
Any one of Compound Nos. 1-8, 10, 11, 15, 21-23, 25, 27, 28, 30 33, 34, 36-43, 45, 46, 49-59 and 61 (e.g. any one of Compounds 1, 3, 7, 8, 10, 11, 15, 25, 28, 30, 33, 34, 36, 37-43, 45, 46, 49-59 and 61) of WO2019/113236 or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Any one of Compound Nos. 1-8, 10, 11, 15, 21-23, 25, 27, 28, 30 33, 34, 36-43, 45, 46, 49-59 and 61 (e.g. any one of Compounds 1, 3, 7, 8, 10, 11, 15, 25, 28, 30, 33, 34, 36, 37-43, 45, 46, 49-59 and 61) of WO2019/113236 or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
Any one of Compound Nos. 1-8, 10, 11, 15, 21-23, 25, 27, 28, 30 33, 34, 36-43, 45, 46, 49-59 and 61 (e.g. any one of Compounds 1, 3, 7, 8, 10, 11, 15, 25, 28, 30, 33, 34, 36, 37-43, 45, 46, 49-59 and 61) of WO2019/113236 or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
Any one of Compound Nos. 1-8, 10, 11, 15, 21-23, 25, 27, 28, 30 33, 34, 36-43, 45, 46, 49-59 and 61 (e.g. any one of Compounds 1, 3, 7, 8, 10, 11, 15, 25, 28, 30, 33, 34, 36, 37-43, 45, 46, 49-59 and 61) of WO2019/113236 or a pharmaceutically acceptable salt thereof and a Raf inhibitor or a pharmaceutically acceptable salt thereof (e.g. belvarafenib, naporafenib, Encorafenib, vemurafenib or dabrafenib)
Any one of Compound Nos. 1-8, 10, 11, 15, 21-23, 25, 27, 28, 30 33, 34, 36-43, 45, 46, 49-59 and 61 of WO2019/113236 (e.g. any one of Compounds 1, 3, 7, 8, 10, 11, 15, 25, 28, 30, 33, 34, 36, 37-43, 45, 46, 49-59 and 61) or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Any one of Compound Nos. 1-8, 10, 11, 15, 21-23, 25, 27, 28, 30 33, 34, 36-43, 45, 46, 49-59 and 61 (e.g. any one of Compounds 1, 3, 7, 8, 10, 11, 15, 25, 28, 30, 33, 34, 36, 37-43, 45, 46, 49-59 and 61) of WO2019/113236 or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Any one of Compound Nos. 3, 14, 15, 17, 19-22, 26, 29, 40, 45, 48, 56-58, 64, 67, 72, 73, 79, 81, 82, 87, 88, 90, 92, 94, 95, 103, 105, 110-113, 116, 131, 136, 137, 139-145, 147-149, 150 and 151 (e.g. any one of Compounds 90, 92, 94, 95, 103, 110, 116, 131, 136, 137, 139-145, 147-149) of WO2019/040380 or a pharmaceutically acceptable salt thereof and a cMET inhibitor or a pharmaceutically acceptable salt thereof (e.g. capmatinib or tepotinib, e.g. capmatinib)
Any one of Compound Nos. 3, 14, 15, 17, 19-22, 26, 29, 40, 45, 48, 56-58, 64, 67, 72, 73, 79, 81, 82, 87, 88, 90, 92, 94, 95, 103, 105, 110-113, 116, 131, 136, 137, 139-145, 147-149, 150 and 151 (e.g. any one of Compounds 90, 92, 94, 95, 103, 110, 116, 131, 136, 137, 139-145, 147-149) of WO2019/040380 or a pharmaceutically acceptable salt thereof and a MAPK pathway inhibitor or a pharmaceutically acceptable salt thereof
Any one of Compound Nos. 3, 14, 15, 17, 19-22, 26, 29, 40, 45, 48, 56-58, 64, 67, 72, 73, 79, 81, 82, 87, 88, 90, 92, 94, 95, 103, 105, 110-113, 116, 131, 136, 137, 139-145, 147-149, 150 and 151 (e.g. any one of Compounds 90, 92, 94, 95, 103, 110, 116, 131, 136, 137, 139-145, 147-149) of WO2019/040380 or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

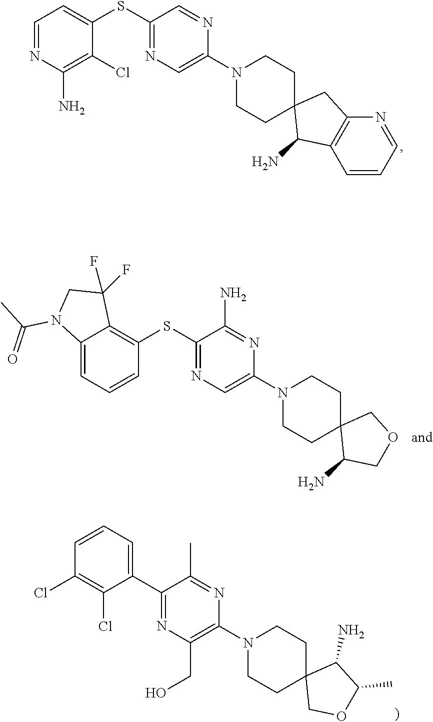 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; Cc1nc(N2CCC3(CC2)CO[C@@H](C)[C@H]3N)c(CO)nc1-c1cccc(Cl)c1Cl]

Any one of Compound Nos. 3, 14, 15, 17, 19-22, 26, 29, 40, 45, 48, 56-58, 64, 67, 72, 73, 79, 81, 82, 87, 88, 90, 92, 94, 95, 103, 105, 110-113, 116, 131, 136, 137, 139-145, 147-149, 150 and 151 (e.g. any one of Compounds 90, 92, 94, 95, 103, 110, 116, 131, 136, 137, 139-145, 147-149) of WO2019/040380 or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

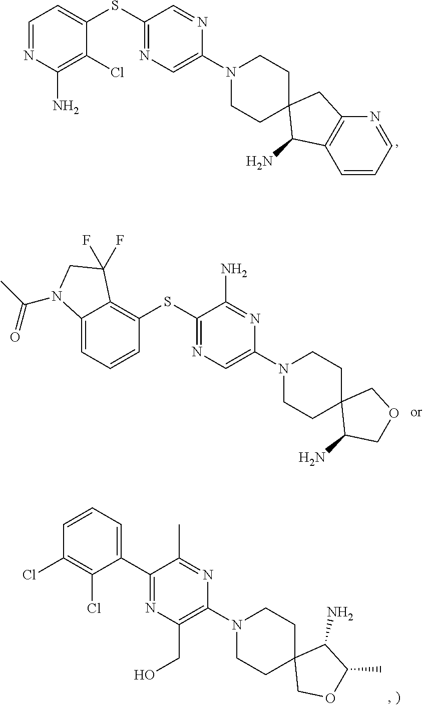 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

Any one of Compound Nos. 3, 14, 15, 17, 19-22, 26, 29, 40, 45, 48, 56-58, 64, 67, 72, 73, 79, 81, 82, 87, 88, 90, 92, 94, 95, 103, 105, 110-113, 116, 131, 136, 137, 139-145, 147-149, 150 and 151 (e.g. any one of Compounds 90, 92, 94, 95, 103, 110, 116, 131, 136, 137, 139-145, 147-149) of WO2019/040380 or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
Any one of Compound Nos. 3, 14, 15, 17, 19-22, 26, 29, 40, 45, 48, 56-58, 64, 67, 72, 73, 79, 81, 82, 87, 88, 90, 92, 94, 95, 103, 105, 110-113, 116, 131, 136, 137, 139-145, 147-149, 150 and 151 (e.g. any one of Compounds 90, 92, 94, 95, 103, 110, 116, 131, 136, 137, 139-145, 147-149) of WO2019/040380 or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
Any one of Compound Nos. 3, 14, 15, 17, 19-22, 26, 29, 40, 45, 48, 56-58, 64, 67, 72, 73, 79, 81, 82, 87, 88, 90, 92, 94, 95, 103, 105, 110-113, 116, 131, 136, 137, 139-145, 147-149, 150 and 151 (e.g. any one of Compounds 90, 92, 94, 95, 103, 110, 116, 131, 136, 137, 139-145, 147-149) of WO2019/040380 or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
Any one of Compound Nos. 3, 14, 15, 17, 19-22, 26, 29, 40, 45, 48, 56-58, 64, 67, 72, 73, 79, 81, 82, 87, 88, 90, 92, 94, 95, 103, 105, 110-113, 116, 131, 136, 137, 139-145, 147-149, 150 and 151 (e.g. any one of Compounds 90, 92, 94, 95, 103, 110, 116, 131, 136, 137, 139-145, 147-149) of WO2019/040380 or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Any one of Compound Nos. 3, 14, 15, 17, 19-22, 26, 29, 40, 45, 48, 56-58, 64, 67, 72, 73, 79, 81, 82, 87, 88, 90, 92, 94, 95, 103, 105, 110-113, 116, 131, 136, 137, 139-145, 147-149, 150 and 151 (e.g. any one of Compounds 90, 92, 94, 95, 103, 110, 116, 131, 136, 137, 139-145, 147-149) of WO2019/040380 or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Any one of Compound Nos. 3, 14, 15, 17, 19-22, 26, 29, 40, 45, 48, 56-58, 64, 67, 72, 73, 79, 81, 82, 87, 88, 90, 92, 94, 95, 103, 105, 110-113, 116, 131, 136, 137, 139-145, 147-149, 150 and 151 (e.g. any one of Compounds 90, 92, 94, 95, 103, 110, 116, 131, 136, 137, 139-145, 147-149) of WO2019/040380 or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
Any one of Compound Nos. 3, 14, 15, 17, 19-22, 26, 29, 40, 45, 48, 56-58, 64, 67, 72, 73, 79, 81, 82, 87, 88, 90, 92, 94, 95, 103, 105, 110-113, 116, 131, 136, 137, 139-145, 147-149, 150 and 151 (e.g. any one of Compounds 90, 92, 94, 95, 103, 110, 116, 131, 136, 137, 139-145, 147-149) of WO2019/040380 or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
Any one of Compound Nos. 3, 14, 15, 17, 19-22, 26, 29, 40, 45, 48, 56-58, 64, 67, 72, 73, 79, 81, 82, 87, 88, 90, 92, 94, 95, 103, 105, 110-113, 116, 131, 136, 137, 139-145, 147-149, 150 and 151 (e.g. any one of Compounds 90, 92, 94, 95, 103, 110, 116, 131, 136, 137, 139-145, 147-149) of WO2019/040380 or a pharmaceutically acceptable salt thereof and a Raf inhibitor or a pharmaceutically acceptable salt thereof (e.g. belvarafenib, naporafenib, Encorafenib, vemurafenib or dabrafenib)
Any one of Compound Nos. 3, 14, 15, 17, 19-22, 26, 29, 40, 45, 48, 56-58, 64, 67, 72, 73, 79, 81, 82, 87, 88, 90, 92, 94, 95, 103, 105, 110-113, 116, 131, 136, 137, 139-145, 147-149, 150 and 151 (e.g. any one of Compounds 90, 92, 94, 95, 103, 110, 116, 131, 136, 137, 139-145, 147-149) of WO2019/040380 or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Any one of Compound Nos. 3, 14, 15, 17, 19-22, 26, 29, 40, 45, 48, 56-58, 64, 67, 72, 73, 79, 81, 82, 87, 88, 90, 92, 94, 95, 103, 105, 110-113, 116, 131, 136, 137, 139-145, 147-149, 150 and 151 (e.g. any one of Compounds 90, 92, 94, 95, 103, 110, 116, 131, 136, 137, 139-145, 147-149) of WO2019/040380 or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Any one of Compound Nos. 11, 23, 42, 60, 77, 98, 102, 105, 106, 108, 114, 116, 119, 123-125, 127, 129, 131, 139, 151, 152, 169, 173, 175, 181, 186-188, 191-195, 201, 204, 210, 212, 218, 238-244, 246, 254-256, 260, 263 and 264 of WO2018/204532 or a pharmaceutically acceptable salt thereof and a cMET inhibitor or a pharmaceutically acceptable salt thereof (e.g. capmatinib or tepotinib, e.g. capmatinib)
Any one of Compound Nos. 11, 23, 42, 60, 77, 98, 102, 105, 106, 108, 114, 116, 119, 123-125, 127, 129, 131, 139, 151, 152, 169, 173, 175, 181, 186-188, 191-195, 201, 204, 210, 212, 218, 238-244, 246, 254-256, 260, 263 and 264 of WO2018/204532 or a pharmaceutically acceptable salt thereof and a MAPK pathway inhibitor or a pharmaceutically acceptable salt thereof
Any one of Compound Nos. 11, 23, 42, 60, 77, 98, 102, 105, 106, 108, 114, 116, 119, 123-125, 127, 129, 131, 139, 151, 152, 169, 173, 175, 181, 186-188, 191-195, 201, 204, 210, 212, 218, 238-244, 246, 254-256, 260, 263 and 264 of WO2018/204532 or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 ASTX (Taiho & Otsuka Pharmas), X-37-SHP2 (X-37),

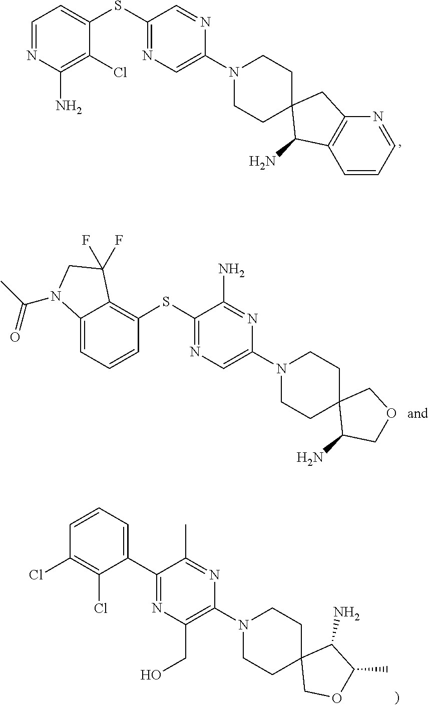 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

Any one of Compound Nos. 11, 23, 42, 60, 77, 98, 102, 105, 106, 108, 114, 116, 119, 123-125, 127, 129, 131, 139, 151, 152, 169, 173, 175, 181, 186-188, 191-195, 201, 204, 210, 212, 218, 238-244, 246, 254-256, 260, 263 and 264 of WO2018/204532 or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

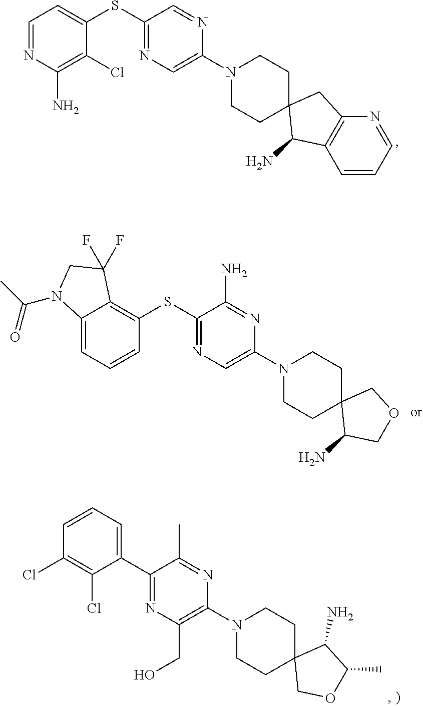 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

Any one of Compound Nos. 11, 23, 42, 60, 77, 98, 102, 105, 106, 108, 114, 116, 119, 123-125, 127, 129, 131, 139, 151, 152, 169, 173, 175, 181, 186-188, 191-195, 201, 204, 210, 212, 218, 238-244, 246, 254-256, 260, 263 and 264 of WO2018/204532 or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
Any one of Compound Nos. 11, 23, 42, 60, 77, 98, 102, 105, 106, 108, 114, 116, 119, 123-125, 127, 129, 131, 139, 151, 152, 169, 173, 175, 181, 186-188, 191-195, 201, 204, 210, 212, 218, 238-244, 246, 254-256, 260, 263 and 264 of WO2018/204532 or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
Any one of Compound Nos. 11, 23, 42, 60, 77, 98, 102, 105, 106, 108, 114, 116, 119, 123-125, 127, 129, 131, 139, 151, 152, 169, 173, 175, 181, 186-188, 191-195, 201, 204, 210, 212, 218, 238-244, 246, 254-256, 260, 263 and 264 of WO2018/204532 or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
Any one of Compound Nos. 11, 23, 42, 60, 77, 98, 102, 105, 106, 108, 114, 116, 119, 123-125, 127, 129, 131, 139, 151, 152, 169, 173, 175, 181, 186-188, 191-195, 201, 204, 210, 212, 218, 238-244, 246, 254-256, 260, 263 and 264 of WO2018/204532 or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Any one of Compound Nos. 11, 23, 42, 60, 77, 98, 102, 105, 106, 108, 114, 116, 119, 123-125, 127, 129, 131, 139, 151, 152, 169, 173, 175, 181, 186-188, 191-195, 201, 204, 210, 212, 218, 238-244, 246, 254-256, 260, 263 and 264 of WO2018/204532 or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
Any one of Compound Nos. 11, 23, 42, 60, 77, 98, 102, 105, 106, 108, 114, 116, 119, 123-125, 127, 129, 131, 139, 151, 152, 169, 173, 175, 181, 186-188, 191-195, 201, 204, 210, 212, 218, 238-244, 246, 254-256, 260, 263 and 264 of WO2018/204532 or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
Any one of Compound Nos. 11, 23, 42, 60, 77, 98, 102, 105, 106, 108, 114, 116, 119, 123-125, 127, 129, 131, 139, 151, 152, 169, 173, 175, 181, 186-188, 191-195, 201, 204, 210, 212, 218, 238-244, 246, 254-256, 260, 263 and 264 of WO2018/204532 or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
Any one of Compound Nos. 11, 23, 42, 60, 77, 98, 102, 105, 106, 108, 114, 116, 119, 123-125, 127, 129, 131, 139, 151, 152, 169, 173, 175, 181, 186-188, 191-195, 201, 204, 210, 212, 218, 238-244, 246, 254-256, 260, 263 and 264 of WO2018/204532 or a pharmaceutically acceptable salt thereof and a Raf inhibitor or a pharmaceutically acceptable salt thereof (e.g. belvarafenib, naporafenib, Encorafenib, vemurafenib or dabrafenib)
Any one of Compound Nos. 11, 23, 42, 60, 77, 98, 102, 105, 106, 108, 114, 116, 119, 123-125, 127, 129, 131, 139, 151, 152, 169, 173, 175, 181, 186-188, 191-195, 201, 204, 210, 212, 218, 238-244, 246, 254-256, 260, 263 and 264 of WO2018/204532 or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Any one of Compound Nos. 11, 23, 42, 60, 77, 98, 102, 105, 106, 108, 114, 116, 119, 123-125, 127, 129, 131, 139, 151, 152, 169, 173, 175, 181, 186-188, 191-195, 201, 204, 210, 212, 218, 238-244, 246, 254-256, 260, 263 and 264 of WO2018/204532 or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Any one of Compound Nos. 1-58, preferably any one of compounds 54, 55, 56A, 56B, 57A, 57B and 58, preferably compound 58 of WO2020/051099 or a pharmaceutically acceptable salt thereof and a cMET inhibitor or a pharmaceutically acceptable salt thereof (e.g. capmatinib or tepotinib, e.g. capmatinib)
Any one of Compound Nos. 1-58, preferably any one of compounds 54, 55, 56A, 56B, 57A, 57B and 58, preferably compound 58 of WO2020/051099 or a pharmaceutically acceptable salt thereof and a MAPK pathway inhibitor or a pharmaceutically acceptable salt thereof
Any one of Compound Nos. 1-58, preferably any one of compounds 54, 55, 56A, 56B, 57A, 57B and 58, preferably compound 58 of WO2020/051099 or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37), h and

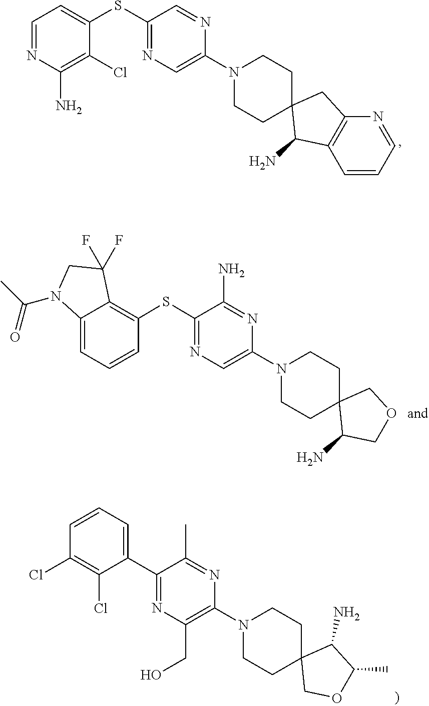 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

Any one of Compound Nos. 1-58, preferably any one of compounds 54, 55, 56A, 56B, 57A, 57B and 58, preferably compound 58 of WO2020/051099 or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma) TAS-ASTX (Taiho & Otsuka Pharmas) and X-37-SHP2 (X-37),

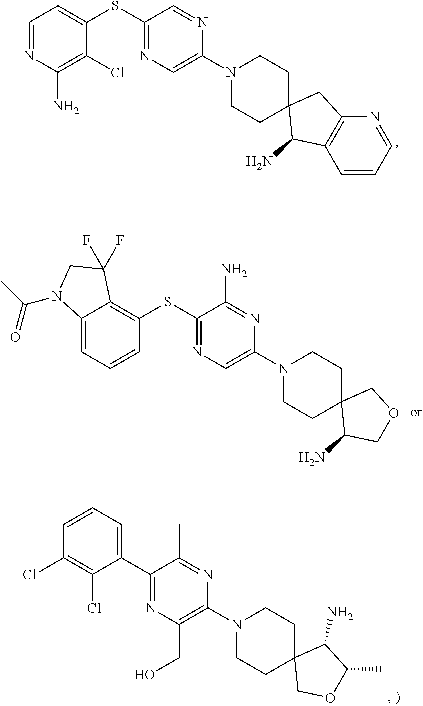 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

Any one of Compound Nos. 1-58, preferably any one of compounds 54, 55, 56A, 56B, 57A, 57B and 58, preferably compound 58 of WO2020/051099 or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
Any one of Compound Nos. 1-58, preferably any one of compounds 54, 55, 56A, 56B, 57A, 57B and 58, preferably compound 58 of WO2020/051099 or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
Any one of Compound Nos. 1-58, preferably any one of compounds 54, 55, 56A, 56B, 57A, 57B and 58, preferably compound 58 of WO2020/051099 or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
Any one of Compound Nos. 1-58, preferably any one of compounds 54, 55, 56A, 56B, 57A, 57B and 58, preferably compound 58 of WO2020/051099 or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Any one of Compound Nos. 1-58, preferably any one of compounds 54, 55, 56A, 56B, 57A, 57B and 58, preferably compound 58 of WO2020/051099 or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
Any one of Compound Nos. 1-58, preferably any one of compounds 54, 55, 56A, 56B, 57A, 57B and 58, preferably compound 58 of WO2020/051099 or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
Any one of Compound Nos. 1-58, preferably any one of compounds 54, 55, 56A, 56B, 57A, 57B and 58, preferably compound 58 of WO2020/051099 or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
Any one of Compound Nos. 1-58, preferably any one of compounds 54, 55, 56A, 56B, 57A, 57B and 58, preferably compound 58 of WO2020/051099 or a pharmaceutically acceptable salt thereof and a Raf inhibitor or a pharmaceutically acceptable salt thereof (e.g. belvarafenib, naporafenib, Encorafenib, vemurafenib or dabrafenib)
Any one of Compound Nos. 1-58, preferably any one of compounds 54, 55, 56A, 56B, 57A, 57B and 58, preferably compound 58 of WO2020/051099 or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Any one of Compound Nos. 1-58, preferably any one of compounds 54, 55, 56A, 56B, 57A, 57B and 58, preferably compound 58 of WO2020/051099 or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Any one of the Compound selected from Table 1, Table 2 and Isomer2 of I-12, for example any one of I-27, I-30, I-31, I-35, I-36, I-40, I-44, I-45, I-64, I-69, I-70, I-71, Isomer 1 of I-77, Isomer 2 of I-170, Isomer 2 of 1-175, Isomer 2 of I-177, Isomer 2 of 1-180, Isomer 1 of I-182, Isomer 1 of 1-186, Isomer 1 of 1-188, Isomer 1 of 1-189, 208, 208, 211, 220, 222, 231, Isomer 2 of 1-334, Isomer 1 of 1-334, Isomer 1 of 1-341, Isomer 1 of 1-343 or Isomer 2 of I-352, for example Isomer 1 of 1-186 or Isomer 2 of 1-12 of WO2020/243323 or a pharmaceutically acceptable salt thereof and a cMET inhibitor or a pharmaceutically acceptable salt thereof (e.g. capmatinib or tepotinib, e.g. capmatinib)
Any one of the Compound selected from Table 1, Table 2 and Isomer2 of 1-12, for example any one of 1-27, I-30, I-31, I-35, I-36, I-40, I-44, I-45, I-64, I-69, I-70, I-71, Isomer 1 of 1-77, Isomer 2 of I-170, Isomer 2 of 1-175, Isomer 2 of I-177, Isomer 2 of 1-180, Isomer 1 of I-182, Isomer 1 of 1-186, Isomer 1 of 1-188, Isomer 1 of 1-189, 208, 208, 211, 220, 222, 231, Isomer 2 of 1-334, Isomer 1 of 1-334, Isomer 1 of 1-341, Isomer 1 of 1-343 or Isomer 2 of I-352, for example Isomer 1 of 1-186 or Isomer 2 of 1-12 of WO2020/243323 or a pharmaceutically acceptable salt thereof and a MAPK pathway inhibitor or a pharmaceutically acceptable salt thereof
Any one of the Compound selected from Table 1, Table 2 and Isomer2 of 1-12, forexample any one of 1-27, I-30, I-31, I-35, I-36, I-40, I-44, I-45, I-64, I-69, I-70, I-71, Isomer 1 of 1-77, Isomer 2 of I-170, Isomer 2 of 1-175, Isomer 2 of I-177, Isomer 2 of 1-180, Isomer 1 of I-182, Isomer 1 of 1-186, Isomer 1 of 1-188, Isomer 1 of 1-189, 208, 208, 211, 220, 222, 231, Isomer 2 of 1-334, Isomer 1 of 1-334, Isomer 1 of 1-341, Isomer 1 of 1-343 or Isomer 2 of I-352, for example Isomer 1 of 1-186 or Isomer 2 of 1-12 of WO2020/243323 or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

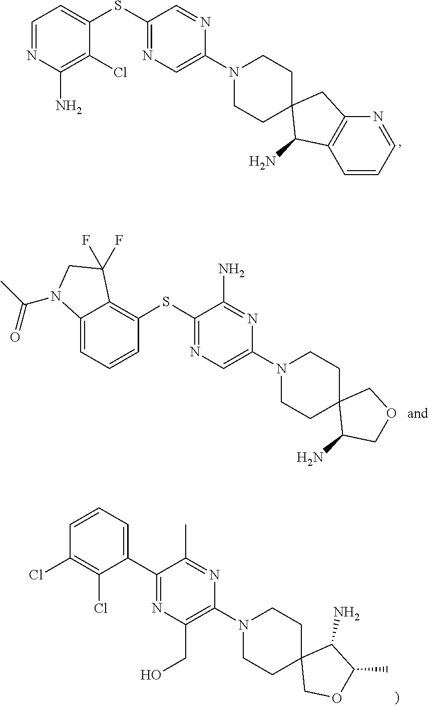 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

Any one of the Compound selected from Table 1, Table 2 and Isomer2 of 1-12, for example any one of 1-27, I-30, I-31, I-35, I-36, I-40, I-44, I-45, I-64, I-69, I-70, I-71, Isomer 1 of 1-77, Isomer 2 of 1-170, Isomer 2 of 1-175, Isomer 2 of I-177, Isomer 2 of 1-180, Isomer 1 of I-182, Isomer 1 of 1-186, Isomer 1 of 1-188, Isomer 1 of 1-189, 208, 208, 211, 220, 222, 231, Isomer 2 of 1-334, Isomer 1 of 1-334, Isomer 1 of 1-341, Isomer 1 of 1-343 or Isomer 2 of I-352, for example Isomer 1 of 1-186 or Isomer 2 of 1-12 of WO2020/243323 or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37),

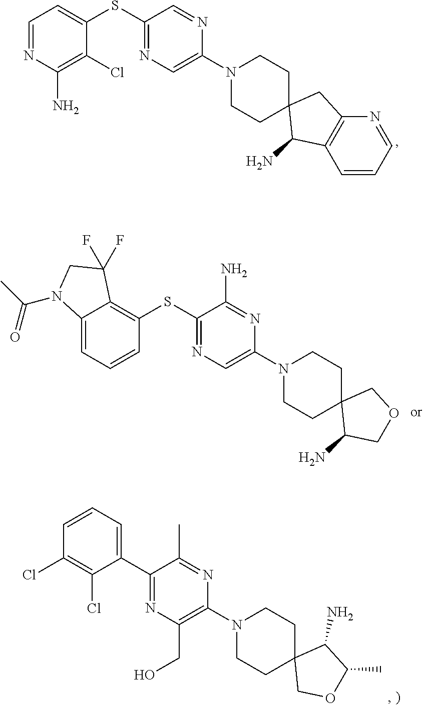 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

Any one of the Compound selected from Table 1, Table 2 and Isomer2 of 1-12, for example any one of 1-27, I-30, I-31, I-35, I-36,1-40, I-44, I-45, I-64, I-69, I-70, I-71, Isomer 1 of 1-77, Isomer 2 of I-170, Isomer 2 of 1-175, Isomer 2 of I-177, Isomer 2 of 1-180, Isomer 1 of I-182, Isomer 1 of 1-186, Isomer 1 of 1-188, Isomer 1 of 1-189, 208, 208, 211, 220, 222, 231, Isomer 2 of 1-334, Isomer 1 of 1-334, Isomer 1 of 1-341, Isomer 1 of 1-343 or Isomer 2 of I-352, for example Isomer 1 of 1-186 or Isomer 2 of 1-12 of WO2020/243323 or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
Any one of the Compound selected from Table 1, Table 2 and Isomer2 of 1-12, for example any one of 1-27, I-30, I-31, I-35, I-36, I-40, I-44, I-45, I-64, I-69, I-70, I-71, Isomer 1 of 1-77, Isomer 2 of I-170, Isomer 2 of 1-175, Isomer 2 of I-177, Isomer 2 of 1-180, Isomer 1 of I-182, Isomer 1 of 1-186, Isomer 1 of 1-188, Isomer 1 of 1-189, 208, 208, 211, 220, 222, 231, Isomer 2 of 1-334, Isomer 1 of 1-334, Isomer 1 of 1-341, Isomer 1 of 1-343 or Isomer 2 of I-352, for example Isomer 1 of 1-186 or Isomer 2 of 1-12 of WO2020/243323 or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
Any one of the Compound selected from Table 1, Table 2 and Isomer2 of 1-12, for example any one of 1-27, I-30, I-31, I-35, I-36, I-40, I-44, I-45, I-64, I-69, I-70, I-71, Isomer 1 of 1-77, Isomer 2 of I-170, Isomer 2 of 1-175, Isomer 2 of I-177, Isomer 2 of 1-180, Isomer 1 of I-182, Isomer 1 of 1-186, Isomer 1 of 1-188, Isomer 1 of 1-189, 208, 208, 211, 220, 222, 231, Isomer 2 of 1-334, Isomer 1 of 1-334, Isomer 1 of 1-341, Isomer 1 of 1-343 or Isomer 2 of I-352, for example Isomer 1 of 1-186 or Isomer 2 of 1-12 of WO2020/243323 or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
Any one of the Compound selected from Table 1, Table 2 and Isomer2 of 1-12, forexample any one of 1-27, I-30, I-31, I-35, I-36,1-40, I-44, I-45, I-64, I-69, I-70, I-71, Isomer 1 of 1-77, Isomer 2 of I-170, Isomer 2 of 1-175, Isomer 2 of I-177, Isomer 2 of 1-180, Isomer 1 of I-182, Isomer 1 of 1-186, Isomer 1 of 1-188, Isomer 1 of 1-189, 208, 208, 211, 220, 222, 231, Isomer 2 of 1-334, Isomer 1 of 1-334, Isomer 1 of 1-341, Isomer 1 of 1-343 or Isomer 2 of I-352, for example Isomer 1 of 1-186 or Isomer 2 of 1-12 of WO2020/243323 or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Any one of the Compound selected from Table 1, Table 2 and Isomer2 of 1-12, forexample any one of 1-27, I-30, I-31, I-35, I-36, I-40, I-44, I-45, I-64, I-69, I-70, I-71, Isomer 1 of 1-77, Isomer 2 of I-170, Isomer 2 of 1-175, Isomer 2 of I-177, Isomer 2 of 1-180, Isomer 1 of I-182, Isomer 1 of 1-186, Isomer 1 of 1-188, Isomer 1 of 1-189, 208, 208, 211, 220, 222, 231, Isomer 2 of 1-334, Isomer 1 of 1-334, Isomer 1 of 1-341, Isomer 1 of 1-343 or Isomer 2 of I-352, for example Isomer 1 of 1-186 or Isomer 2 of 1-12 of WO2020/243323 or a pharmaceutically acceptable salt thereof and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)
Any one of the Compound selected from Table 1, Table 2 and Isomer2 of 1-12, forexample any one of 1-27, I-30, I-31, I-35, I-36, I-40, I-44, I-45, I-64, I-69, I-70, I-71, Isomer 1 of 1-77, Isomer 2 of I-170, Isomer 2 of 1-175, Isomer 2 of I-177, Isomer 2 of 1-180, Isomer 1 of I-182, Isomer 1 of 1-186, Isomer 1 of 1-188, Isomer 1 of 1-189, 208, 208, 211, 220, 222, 231, Isomer 2 of I-334, Isomer 1 of I-334, Isomer 1 of 1-341, Isomer 1 of 1-343 or Isomer 2 of I-352, for example Isomer 1 of 1-186 or Isomer 2 of 1-12 of WO2020/243323 or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
Any one of the Compound selected from Table 1, Table 2 and Isomer2 of 1-12, for example any one of 1-27, I-30, I-31, I-35, I-36, I-40, I-44, I-45, I-64, I-69, I-70, I-71, Isomer 1 of 1-77, Isomer 2 of I-170, Isomer 2 of 1-175, Isomer 2 of I-177, Isomer 2 of 1-180, Isomer 1 of I-182, Isomer 1 of 1-186, Isomer 1 of 1-188, Isomer 1 of 1-189, 208, 208, 211, 220, 222, 231, Isomer 2 of 1-334, Isomer 1 of 1-334, Isomer 1 of 1-341, Isomer 1 of 1-343 or Isomer 2 of I-352, for example Isomer 1 of 1-186 or Isomer 2 of 1-12 of WO2020/243323 or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
Any one of the Compound selected from Table 1, Table 2 and Isomer2 of 1-12, forexample any one of 1-27, I-30, I-31, I-35, I-36, I-40, I-44, I-45, I-64, I-69, I-70, I-71, Isomer 1 of 1-77, Isomer 2 of I-170, Isomer 2 of 1-175, Isomer 2 of I-177, Isomer 2 of 1-180, Isomer 1 of I-182, Isomer 1 of 1-186, Isomer 1 of 1-188, Isomer 1 of 1-189, 208, 208, 211, 220, 222, 231, Isomer 2 of 1-334, Isomer 1 of 1-334, Isomer 1 of 1-341, Isomer 1 of 1-343 or Isomer 2 of I-352, for example Isomer 1 of 1-186 or Isomer 2 of 1-12 of WO2020/243323 or a pharmaceutically acceptable salt thereof and a Raf inhibitor or a pharmaceutically acceptable salt thereof (e.g. belvarafenib, naporafenib, Encorafenib, vemurafenib or dabrafenib)
Any one of the Compound selected from Table 1, Table 2 and Isomer2 of 1-12, for example any one of 1-27, I-30, I-31, I-35, I-36, I-40, I-44, I-45, I-64, I-69, I-70, I-71, Isomer 1 of 1-77, Isomer 2 of I-170, Isomer 2 of 1-175, Isomer 2 of I-177, Isomer 2 of 1-180, Isomer 1 of I-182, Isomer 1 of 1-186, Isomer 1 of 1-188, Isomer 1 of 1-189, 208, 208, 211, 220, 222, 231, Isomer 2 of 1-334, Isomer 1 of 1-334, Isomer 1 of 1-341, Isomer 1 of 1-343 or Isomer 2 of I-352, for example Isomer 1 of 1-186 or Isomer 2 of 1-12 of WO2020/243323 or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Any one of the Compound selected from Table 1, Table 2 and Isomer2 of 1-12, for example any one of 1-27, I-30, I-31, I-35, I-36, I-40, I-44, I-45, I-64, I-69, I-70, I-71, Isomer 1 of 1-77, Isomer 2 of I-170, Isomer 2 of 1-175, Isomer 2 of I-177, Isomer 2 of 1-180, Isomer 1 of I-182, Isomer 1 of 1-186, Isomer 1 of 1-188, Isomer 1 of 1-189, 208, 208, 211, 220, 222, 231, Isomer 2 of 1-334, Isomer 1 of 1-334, Isomer 1 of 1-341, Isomer 1 of 1-343 or Isomer 2 of I-352, for example Isomer 1 of 1-186 or Isomer 2 of 1-12 of WO2020/243323 or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Any one of the Compound selected from Table 1 or Table 2, for example any one of 1-27, 1-30, I-31, I-32, I-33, I-36, I-37, I-38, I-51, I-54, I-59, I-65, I-67, I-68, I-74, I-75, I-80, I-81, I-84 and 1-85 of WO2020/243315 or a pharmaceutically acceptable salt thereof and a cMET inhibitor or a pharmaceutically acceptable salt thereof (e.g. capmatinib or tepotinib, e.g. capmatinib)
Any one of the Compound selected from Table 1 or Table 2, for example any one of 1-27, 1-30, I-31, I-32, I-33, I-36, I-37, I-38, I-51, I-54, I-59, I-65, I-67, I-68, I-74, I-75, I-80, I-81, I-84 and 1-85 of WO2020/243315 or a pharmaceutically acceptable salt thereof and a MAPK pathway inhibitor or a pharmaceutically acceptable salt thereof
Any one of the Compound selected from Table 1 or Table 2, for example any one of 1-27, 1-30, I-31, I-32, I-33, I-36, I-37, I-38, I-51, I-54, I-59, I-65, I-67, I-68, I-74, I-75, I-80, I-81, I-84 and 1-85 of WO2020/243315 or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib) and a SHP2 inhibitor (e.g. a SHP2 inhibitor selected from the group consisting of TN0155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas), X-37-SHP2 (X-37),

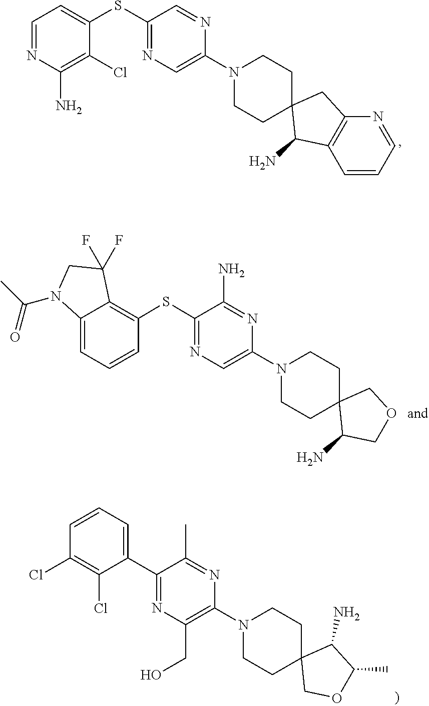 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "and" ; Cc1nc(N2CCC3(CC2)CO[C@@H](C)[C@H]3N)c(CO)nc1-c1cccc(Cl)c1Cl]

Any one of the Compound selected from Table 1 or Table 2, for example any one of 1-27, 1-30, I-31, I-32, I-33, I-36, I-37, I-38, I-51, I-54, I-59, I-65, I-67, I-68, I-74, I-75, I-80, I-81, I-84 and 1-85 of WO2020/243315 or a pharmaceutically acceptable salt thereof and a SHP2 inhibitor or a pharmaceutically acceptable salt thereof (e.g. TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX_(Taiho & Otsuka Pharmas) and X-37-SHP2 (X-37),

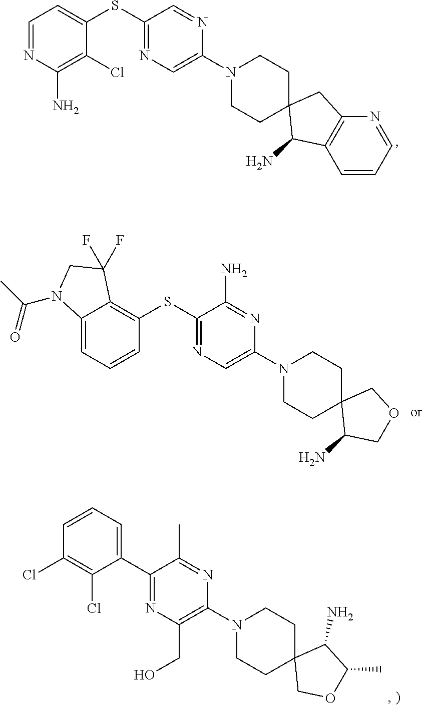 [SMILES: Nc1nccc(Sc2cnc(N3CCC4(CC3)Cc3ncccc3[C@H]4N)cn2)c1Cl ; CC(=O)N1CC(F)(F)c2c(Sc3ncc(N4CCC5(CC4)COC[C@H]5N)nc3N)cccc21 ; "or" ; Cc1nc(N2CCC3(CC2)CO[C@@H](C)[C@H]3N)c(CO)nc1-c1cccc(Cl)c1Cl]

Any one of the Compound selected from Table 1 or Table 2, for example any one of I-27, 1-30, I-31, I-32, I-33, I-36, I-37, I-38, I-51, I-54, I-59, I-65, I-67, I-68, I-74, I-75, I-80, I-81, I-84 and 1-85 of WO2020/243315 or a pharmaceutically acceptable salt thereof and a KRAS G12/G13 inhibitor or a pharmaceutically acceptable salt thereof (e.g. a KRAS G12C inhibitor or a pharmaceutically acceptable salt thereof selected from the group consisting of Sotorasib, Adagrasib, LY349446, D-1553, B11701963, GDC6036, JNJ74699157, X-Chem KRAS, LY3537982, B11823911, AS KRAS G12C, SF KRAS G12C, RMC032, JAB-21822, AST-KRAS G12C, MRTX1257, AZ KRAS G12C, NYU-12VC1, RMC6291 and Compound C, e.g. Compound C, Sotorasib or Adagrasib)
Any one of the Compound selected from Table 1 or Table 2, for example any one of 1-27, 1-30, I-31, I-32, I-33, I-36, I-37, I-38, I-51, I-54, I-59, I-65, I-67, I-68, I-74, I-75, I-80, I-81, I-84 and 1-85 of WO2020/243315 or a pharmaceutically acceptable salt thereof and an EGFR inhibitor or a pharmaceutically acceptable salt thereof (e.g. erlotinib, osimertinib, neratinib, gefnitib, cetuximab, panitumumab, lapatinib, dacomitinib, necitumumab, nazartinib, LTT462 or vandetanib, e.g. LTT462 or erlotinib)
Any one of the Compound selected from Table 1 or Table 2, for example any one of 1-27, 1-30, I-31, I-32, I-33, I-36, I-37, I-38, I-51, I-54, I-59, I-65, I-67, I-68, I-74, I-75, I-80, I-81, I-84 and 1-85 of WO2020/243315 or a pharmaceutically acceptable salt thereof and a PI3K inhibitor or a pharmaceutically acceptable salt thereof (e.g. AMG511, QAU421, buparlisib, Idelalisib, Copanlisib, Duvelisib, Alpelisib or Umbralisib e.g. Alpelisib)
Any one of the Compound selected from Table 1 or Table 2, for example any one of 1-27, 1-30, I-31, I-32, I-33, I-36, I-37, I-38, I-51, I-54, I-59, I-65, I-67, I-68, I-74, I-75, I-80, I-81, I-84 and 1-85 of WO2020/243315 or a pharmaceutically acceptable salt thereof and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Any one of the Compound selected from Table 1 or Table 2, for example any one of 1-27, 1-30, I-31, I-32, I-33, I-36, I-37, I-38, I-51, I-54, I-59, I-65, I-67, I-68, I-74, I-75, I-80, I-81, I-84 and 1-85 of WO2020/243315 or a pharmaceutically acceptable salt thereof and an ERK inhibitor or a pharmaceutically acceptable salt thereof (e.g. ulixertinib, GDC-0994, KO-947, Vtx-11e, SCH-772984, MK2853, LY3214996, MK08353, LTT462 or BVD-523, e.g. LTT462)
Any one of the Compound selected from Table 1 or Table 2, for example any one of 1-27, 1-30, I-31, I-32, I-33, I-36, I-37, I-38, I-51, I-54, I-59, I-65, I-67, I-68, I-74, I-75, I-80, I-81, I-84 and 1-85 of WO2020/243315 or a pharmaceutically acceptable salt thereof and an MDM2 inhibitor or a pharmaceutically acceptable salt thereof (e.g. nutlin-3a, idasanutlin (also known as RG7388), RG7112, AMG-232 (also known as KRT-232), APG-115, BI-907828, milademetan or hdm201 (also known as siremadlin)
Any one of the Compound selected from Table 1 or Table 2, for example any one of 1-27, 1-30, I-31, I-32, I-33, I-36, I-37, I-38, I-51, I-54, I-59, I-65, I-67, I-68, I-74, I-75, I-80, I-81, I-84 and 1-85 of WO2020/243315 or a pharmaceutically acceptable salt thereof and a CDK4/6 inhibitor or a pharmaceutically acceptable salt thereof (e.g. a CDK4/6 inhibitor or a pharmaceutically acceptable salt thereof selected from the group consisting of palbociclib, ribociclib and abemaciclib)
Any one of the Compound selected from Table 1 or Table 2, for example any one of 1-27, 1-30, I-31, I-32, I-33, I-36, I-37, I-38, I-51, I-54, I-59, I-65, I-67, I-68, I-74, I-75, I-80, I-81, I-84 and 1-85 of WO2020/243315 or a pharmaceutically acceptable salt thereof and a Raf inhibitor or a pharmaceutically acceptable salt thereof (e.g. belvarafenib, naporafenib, Encorafenib, vemurafenib or dabrafenib)
Any one of the Compound selected from Table 1 or Table 2, for example any one of 1-27, 1-30, I-31, I-32, I-33, I-36, I-37, I-38, I-51, I-54, I-59, I-65, I-67, I-68, I-74, I-75, I-80, I-81, I-84 and 1-85 of WO2020/243315 or a pharmaceutically acceptable salt thereof and a Raf inhibitor or a pharmaceutically acceptable salt thereof (e.g. belvarafenib, naporafenib, Encorafenib, vemurafenib or dabrafenib) and a MEK inhibitor or a pharmaceutically acceptable salt thereof (e.g. a MEK inhibitor or a pharmaceutically acceptable salt thereof selected from the group consisting of trametinib, binimetinib, selumetinib, pimasertib, PD-0325901, and cobimetinib, e.g. trametinib)
Any one of the Compound selected from Table 1 or Table 2, for example any one of 1-27, 1-30, I-31, I-32, I-33, I-36, I-37, I-38, I-51, I-54, I-59, I-65, I-67, I-68, I-74, I-75, I-80, I-81, I-84 and 1-85 of WO2020/243315 or a pharmaceutically acceptable salt thereof and a Raf inhibitor or a pharmaceutically acceptable salt thereof (e.g. belvarafenib, naporafenib, Encorafenib, vemurafenib or dabrafenib) and an ERK inhibitor or a pharmaceutically acceptable salt thereof (e.g. ulixertinib, GDC-0994, KO-947, Vtx-11 e, SCH-772984, MK2853, LY3214996, MK08353, LTT462 or BVD-523, e.g. LTT462)

In an embodiment, the TEAD inhibitor is selected from any one of the compounds disclosed in Table 1 of WO2020/243423. In an embodiment, the TEAD inhibitor is selected from any one of the Examples disclosed in Table 1 of WO2020/243415, e.g.

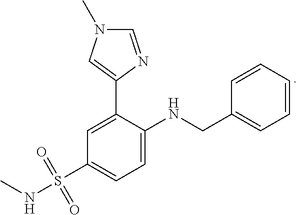 [SMILES: CNS(=O)(=O)c1ccc(NCc2ccccc2)c(-c2cn(C)cn2)c1]

Also disclosed herein are any of the above exemplified combinations for use in the treatment of cancer.

Also disclosed herein is the first named combination partner of any of the above exemplified combinations for use in the treatment of cancer, wherein the treatment further comprises administration of the second named (and third named where present) combination partner (e.g. where the combination is Compound B and trametinib disclosed is Compound B for use in the treatment of cancer wherein the treatment further comprises administration of trametinib).

Also disclosed herein is the second named combination partner of any of the above exemplified combinations for use in the treatment of cancer, wherein the treatment further comprises administration of the first named (and third named where present) combination partner (e.g. where the combination is Compound B and trametinib disclosed is trametinib for use in the treatment of cancer wherein the treatment further comprises administration of Compound B).

Also disclosed herein is the third named combination partner of any of the above exemplified triple combinations for use in the treatment of cancer, wherein the treatment further comprises administration of the first named and second named combination partners.

Also disclosed herein is a method of treating cancer in a subject in need thereof, wherein the method comprises administering to the subject a therapeutically effective amount of the first named combination partner in combination with the second named combination partner (and the third named combination partner where present).

The term “cMET inhibitor” as used herein includes, but is not limited to, the group consisting of Tivantinib, Foretinib, Cabozantinib, Crizotinib, capmatinib, AMG337, Voltinib, BMS777607, glesatinib, tepotinib, onartuzumab, rilotummumab, ficlatuzumab, emibetuzumab and any pharmaceutically acceptable salts thereof. In an embodiment, the cMET inhibitor is selected from the group consisting of cabozantinib, crizotinib, capmatinib, and any pharmaceutically acceptable salts thereof. In an embodiment, the cMET inhibitor is capmatinib, or a pharmaceutically acceptable salt thereof. Capmatinib has the following formula:

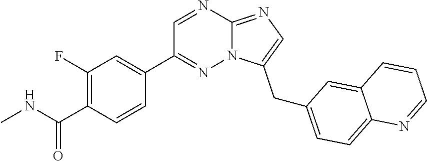 [SMILES: CNC(=O)c1ccc(-c2cnc3ncc(Cc4ccc5ncccc5c4)n3n2)cc1F]

and is also referred to as 2-fluoro-N-methyl-4-[7-quinolin-6-yl-methyl)-imidazo[1,2-b][1,2,4]triazin-2yl]benzamide or “INC280”. Capmatinib can be synthesized according to methods disclosed in, e.g., U.S. Pat. Nos. 7,767,675 and 8,420,645, which are hereby incorporated by reference in their entireties. U.S. Pat. No. 8,420,645 also discloses salts and solvated (e.g., hydrated) forms of capmatinib that may be used in the combinations of the present invention. For example, in some embodiments, capmatinib is a dihydrochloric acid salt. In some embodiments, capmatinib is a monohydrate of the dihydrochloric acid salt (i.e., 2-Fluoro-N-methyl-4-[7-(quinolin-6-ylmethyl)imidazo[1,2-b][1,2,4]triazin-2-yl]benzamide-hydrogen chloride-water (1/2/1)).

The term “KRAS G12/G13 inhibitor” as used herein includes, but is not limited to KRAS G12 C inhibitors. Preferably the KRAS G12/G13 inhibitor is a KRAS G12C inhibitor. The term KRAS G12 C inhibitor includes, but is not limited to the group consisting of 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one, (Compound C), sotorasib (Amgen), adagrasib (Mirati), D-1553 (InventisBio), B11701963 (Boehringer), GDC6036 (Roche), JNJ74699157 (J&J), X-Chem KRAS (X-Chem), LY3537982 (Lilly), B11823911 (Boehringer), AS KRAS G12C (Ascentage Pharma), SF KRAS G12C (Sanofi), RMC032 (Revolution Medicine), JAB-21822 (Jacobio Pharmaceuticals), AST-KRAS G12C (Allist Pharmaceuticals), AZ KRAS G12C (Astra Zeneca), NYU-12VC1 (New York University), and RMC6291 (Revolution Medicines). In an embodiment the KRAS G12C inhibitor is selected from the group consisting of Compound C, sotorasib and adagrasib. In an embodiment, the KRAS G12 inhibitor is Compound C. Compound C is also known as “JDQ443” or “NVP-JDQ443” and is described in Example 1a of PCT application WO2021/124222, published 24 Jun. 2021.

The term “KRAS G12/G13 inhibitor” as used herein also includes, but is not limited to, a compound selected from 1-(4-(6-chloro-8-fluoro-7-(3-hydroxy-5-vinylphenyl)quinazolin-4-yl)piperazin-1-yl)prop-2-en-1-one-,-methane (1/2); (S)-1-(4-(6-chloro-8-fluoro-7-(2-fluoro-6-hydroxyphenyl)quinazolin-4-yl)piperazin-1-yl)prop-2-en-1-one; and 2-((S)-1-acryloyl-4-(2-(((S)-1-methylpyrrolidin-2-yl)methoxy)-7-(naphthalen-1-yl)-5,6,7,8-tetrahydropyrido[3,4-d]pyrimidin-4-yl)piperazin-2-yl)acetonitrile and any one of the compounds detailed in WO2013/155223, WO2014/143659, WO2014/152588, WO2014/160200, WO2015/054572, WO2016/044772, WO2016/049524, WO2016164675, WO2016168540, WO2017/058805, WO2017015562, WO2017058728, WO2017058768, WO2017058792, WO2017058805, WO2017058807, WO2017058902, WO2017058915, WO2017087528, WO2017100546, WO2017/201161, WO2018/064510, WO2018/068017, WO2018/119183, WO2018/217651, WO2018/140512, WO2018/140513, WO2018/140514, WO2018/140598, WO2018/140599, WO2018/140600, WO2018/143315, WO2018/206539, WO2018/218069, WO2018/218070, WO2018/218071, WO2019/051291, WO2019/099524, WO2019/110751, WO2019/141250, WO2019/150305, WO2019/155399, WO2019/213516, WO2019/213526, WO2019/215203, WO2019/217307 and WO2019/217691, WO2019/232419, WO20201028706, WO2020/047192, EP3628664, WO2020081282, WO2020085504, WO2020/085493, WO2020/097537, WO2020/106640, WO2020/113071, WO2020/146613, WO2020/156285, WO2020/181110, WO2020/178282, WO2020/216190, WO2020/236940, WO2020/233592, WO2020/238791, WO2020/239077, WO2020/239123, WO2020/259513, WO2020/259573, WO2020/259432, WO2021/000885, WO2021/023154, WO2021/027943, WO2021/027911, CN112390796, WO2021/037018, CN112430234, CN112442029, WO2021/043322, WO2021/055728.

The term “SHP2 inhibitor” as used herein includes, but is not limited to, compounds described in WO2015/107493, WO2015/107494, WO2015/107495, WO2016/203406, WO2016/203404, WO2016/203405, WO2017/216706, WO2017/156397, WO2020/063760, WO2018/172984, WO2017/211303, WO21/061706, WO2019/183367, WO2019/183364, WO2019/165073, WO2019/067843, WO2018/218133, WO2018/081091, WO2018/057884, WO2020/247643, WO2020/076723, WO2019/199792, WO2019/118909, WO2019/075265, WO2019/051084, WO2018/136265, WO2018/136264, WO2018/013597, WO2020/033828, WO2019/213318, WO2019/158019, WO2021/088945, WO2020/081848, WO21/018287, WO2020/094018, WO2021/033153, WO2020/022323, WO2020/177653, WO2021/073439, WO2020/156243, WO2020/156242, WO2021/147879, WO2020/061101, WO2019/233810, WO2021/110796, WO2020/073949, WO2020/061103, WO2021/115286, WO2021/119525, WO2021/121397, WO2021/143680, WO2021/143823, WO2021/1148010, WO2020/249079, WO2020/033286, WO2021/061515, WO2019/182960, WO2020/094104, WO2020/210384, WO2020/181283, WO2021/043077, WO2021/028362, WO2020/259679, WO2020/108590 & WO2019/051469; TNO155, JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37). In an embodiment, the SHP2 inhibitor is selected from the group consisting of TNO155, JAB3068 (Jacobio), JAB3312 (Jacobio) and RMC4630 (Revolution Medicines). In an embodiment, the SHP2 inhbitor is TNO155.

The term “EGFR inhibitor” as used herein includes, but is not limited to, the group consisting of erlotinib, osimertinib, neratinib, gefnitib, cetuximab, panitumumab, lapatinib, dacomitinib, necitumumab, nazartinib, LTT462 and vandetanib. In an embodiment, the EGFR inhibitor is selected from the group consisting of cetuximab, panitumuab, erlotinib, gefitinib, osimertinib, nazartinib and LTT462. In an embodiment, the EGFR inhibitor is LTT462 or erlotinib.

The term “PI3K inhibitor” as used herein includes, but is not limited to, the group consisting of AMG511, buparlisib, Idelalisib, Copanlisib, Duvelisib, Alpelisib, QAU421 ((S)-N¹-(5-(2-(tert-butyl)pyrimidin-4-yl)-4-methylthiazol-2-yl)pyrrolidine-1,2-dicarboxamide—a close analog of Alpelisib) and Umbralisib. In an embodiment, the PI3K inhibitor is Alpelisib or QAU421).

The term “ERK inhibitor” as used herein includes, but is not limited to, the group consisting of ulixertinib, GDC-0994, KO-947, Vtx-11e, SCH-772984, MK2853, LY3214996, MK08353, LTT462 and BVD-523. In an embodiment, the ERK inhibitor is selected from the group consisting of ulixertinib, LTT462, GDC=0994, KO-947, Vtx-11e, SCH-722894, MK2853 and LY3214996. In an embodiment, the ERK inhibitor is LTT462 or ulixertinib. In an embodiment, the ERK inhibitor is LTT462.

The term “MEK inhibitor” as used herein includes, but is not limited to, the group consisting of trametinib, binimetinib, selumetinib, pimasertib, PD-0325901, and cobimetinib. In an embodiment the MEK inhibitor is trametinib.

The term “CDK4/6 inhibitor”, as used herein includes, but is not limited to ribociclib, palbociclib and abemaciclib. In an embodiment, the CDK4/6 inhibitor is ribociclib (also known as LEE011).

The term “Raf inhibitor”, as used herein includes, but is not limited to belvarafenib, naporafenib vemurafenib, Encorafenib and dabrafenib. In an embodiment, the Raf inhibitor is naporafenib or dabrafenib.

The term “Mdm2 inhibitor”, as used herein refers to any compound inhibiting the HDM2/p53 (Mdm2/p53) interaction association. HDM2 (Human homolog of murine double minute 2) is a negative regulator of p53. Mdm2 inhibitors are useful in pharmaceutical compositions for human orveterinary use where inhibition of Mdm2/p53 association is indicated, e.g., in the treatment of tumors and/or cancerous cell growth. The term “Mdm2 inhibitor”, as used herein includes nutlin-3a, idasanutlin (also known as RG7388), RG7112, AMG-232 (also known as KRT-232), APG-115, BI-907828, milademetan and hdm201 (also known as siremadlin). In an emnodiment, the Mdm2 inhibitor is selcted from AMG-232 and HDM201. In an embodiment, the Mdm2 inhibitor is HDM201.

The term “MAPK pathway” or “MAP Kinase pathway” as herein, and also known as the MPK/ERK pathway and the Ras-Raf-MEK-ERK pathway is well understood by the skilled person, and refers to a chain of proteins in thecell that communicates a signal from a receptor of the cell to the DNA present in the nuclues of the cell. The “MAP Kinase pathway” includes, but is not limited to, EGFR, GRB2, SOS, RAS, RAF and MEK.

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. The terms “C₁-C₃alkyl” and “C₁-C₄alkyl” are to be construed accordingly. Examples of C₁-C₆alkyl include, but are not limited to, methyl, ethyl, n-propyl, 1-methylethyl (iso-propyl), n-butyl, n-pentyl, 1,1-dimethylethyl (t-butyl) and hexyl.

In general, unless otherwise indicated herein or otherwise clearly contradicted by context, for substituents comprising two or more subgroups, the last named group is the radical attachment point, for example, “alkylaryl” means a monovalent radical of the formula alkyl-aryl-, while “arylalkyl” means a monovalent radical of the formula aryl-alkyl-.

As used herein, the term “hydroxyC₁-C₄alkyl” refers to a radical of formula —Rₐ—OH, wherein Rₐ is C₁-C₄alkyl as defined above. Examples of hydroxyC₁-C₄alkyl include, but are not limited to, hydroxy-methyl, 2-hydroxy-ethyl, 2-hydroxy-propyl and 3-hydroxy-propyl.

As used herein, the term “hydroxyC₁-C₃alkyl” refers to a radical of formula —Rₐ—OH, wherein Rₐ is C₁-C₃alkyl as defined above. Examples of hydroxyC₁-C₃alkyl include, but are not limited to, hydroxy-methyl, 2-hydroxy-ethyl, 2-hydroxy-propyl and 3-hydroxy-propyl.

As used herein, the term “C₃-C₆cycloalkyl” refers to a saturated monocyclic hydrocarbon group of 3-6 carbon atoms. Examples of C₃-C₆cycloalkyl include cyclopropyl, cyclobutyl, cyclopentyl and cyclohexyl.

As used herein, the term “C₁-C₆alkoxy” refers to a radical of the formula —ORₐ where Rₐ is a C₁-C₆alkyl radical as generally defined above. The terms “C₁-C₃alkoxy” and “C₁-C₄alkoxy” are to be construed accordingly. Examples of C₁-C₆alkoxy include, but are not limited to, methoxy, ethoxy, propoxy, isopropoxy, butoxy, isobutoxy, pentoxy, and hexoxy.

As used herein, the term “C₁-C₃-alkoxy-C₁-C₃alkyl” refers to a C₁-C₃-alkyl radical as defined above, wherein one of the hydrogen atoms of the C₁-C₃-alkyl radical is replaced by C₁-C₃-alkoxy. “Halogen” or “halo” refers to fluoro, chloro, bromo or iodo. Preferably, halo is fluoro, chloro or bromo. More preferably, halo is fluoro or chloro.

The term “oxo” refers to the radical ═O.

The term “sulfonyl” refers to the radical —S(═O)₂—.

The term “amino” refers to the radical —NH₂.

The term “NHR^{1b}” refers to the radical —N(H)R^{1b}. Similarly, a term such as “NR^{5a}R^{5b}” refers to the radical—N(R^{5a})R^{5b}.

As used herein, the term “halogenC₁-C₃alkyl” or “haloC₁-C₃alkyl” refers to a C₁-C₃alkyl radical, as defined above, substituted with one or more halo radicals, as defined above. Examples of halogenC₁-C₃alkyl include, but are not limited to, trifluoromethyl, difluoromethyl, fluoromethyl, trichloromethyl, 1,1-difluoroethyl, 2,2-difluoroethyl, 2,2,2-trifluoroethyl, 2-fluoropropyl, 3,3-difluoropropyl and 1-fluoromethyl-2-fluoroethyl.

As used herein, the term “haloC₁-C₆alkoxy” refers to C₁-C₆alkoxy as defined above, wherein at least one of the hydrogen atoms of the C₁-C₆alkoxy radical is substituted with a halo radical, as defined above. The term “haloC₁-C₃alkoxy” is to be construed accordingly. Examples of haloC₁-C₆alkoxy include, but are not limited to, trifluoromethoxy, difluoromethoxy, trifluoroethoxy, 2-fluoropropoxy, 3,3-difluoropropoxy.

As used herein, the term “hydroxyC₁-C₆alkoxy” refers to a C₁-C₆alkoxy radical as defined above, wherein at least one of the hydrogen atoms of the C₁-C₆alkoxy radical is replaced by OH. The term “hydroxyC₁-C₃alkoxy is to be construed accordingly. Examples of hydroxyC₁-C₆alkoxy include, but are not limited to, hydroxymethoxy, hydroxyethoxy, 2-hydroxypropoxy.

As used herein, the term “C₁-C₃alkoxyC₁-C₃alkoxy” refers to a C₁-C₃alkoxy radical as defined above, wherein one of the hydrogen atoms of the C₁-C₃alkoxy radical is replaced by —O—C₁-C₃alkyl. An example of C₁-C₃alkoxyC₁-C₃alkoxy includes, but is not limited to, 2-methoxyethoxy.

As used herein, the term “haloC₁-C₃alkoxy-C₁-C₃alkyl” refers to a C₁-C₃alkyl radical as defined above, wherein one of the hydrogen atoms of the C₁-C₃alkyl radical is replaced by haloC₁-C₃alkoxy as defined above. Examples of haloC₁-C₃alkoxy-C₁-C₃alkyl include, but are not limited to (difluoromethoxy)methyl (i.e. CHF₂—O—CH₂—).

As used herein, the term “C(O)NR¹CR^{1d}” refers to a radical of the formula —Rₐ1-N(Rₐ₂)₂ where Rai is a carbonyl radical and each Rₐ₂ is a R^{1c} or a R^{1d} radical, each of which may be the same or different, as defined herein.

As used herein, the term “C(O)di(C₁-C₃alkyl)amino” refers to a radical of the formula —Rₐ₁—N(Rₐ₂)₂ where Rₐ₁ is a carbonyl radical and each Rₐ₂ is a C₁-C₃alkyl as defined herein, and each may be the same or different.

As used herein, the term “C(O)C₁-C₃alkyl” refers to a radical of the formula —Rₐ₁—C₁-C₃alkyl where Rₐ₁ is a carbonyl radical and C₁-C₃alkyl is as defined above.

As used herein, the term “C(O)NHR⁶a” refers to a radical of the formula —Rₐ₁—N(H)-R^{6a} where Rₐ₁ is a carbonyl radical and R^{6a} is as defined herein.

As used herein, the term “S-haloC₁-C₃alkyl” refers to a radical of the formula —S-haloC₁-C₃alkyl where haloC₁-C₃alkyl is as defined above.

As used herein, the term “C(O)OC₁-C₃alkyl” refers to a radical of the formula —Rₐ1-O-C₁-C₃alkyl where Rₐ₁ is a carbonyl radical and C₁-C₃alkyl is as defined above.

As used herein, the term “SO₂C₁-C₃alkyl” refers to a radical of the formula —S(═O)₂—Rₐ2 where Rₐ2 is a C₁-C₃alkyl as defined above.

The term “C₁-C₃alkylene” as used herein refers to a straight or branched hydrocarbon chain bivalent radical consisting solely of carbon and hydrogen atoms, containing no unsaturation, and having from one to three carbon atoms. In embodiments whereby the 4-, 5- or 6-membered saturated heterocyclic ring of Q (or Q₁) is substituted with a C₁-C₃alkylene forming a bridge between two ring atoms of the saturated heterocyclic ring, thus forming a bridged bicyclic structure, the C₁-C₃alkylene is preferably propylene (—CH₂—CH₂—CH₂—), ethylene (—CH₂—CH₂—) or methylene (—CH₂—).

The term “(CH₂)₀₋₂R₁ₐ” refers to a radical of the formula —(CH₂)₀₋₂R₁ₐ, i.e., the radical R₁ₐ is attached to the rest of the molecule via a bond, a methylene linker or an ethylene linker.

The term “(CH₂)₀₋₁C(O)di(C₁-C₃alkyl)amino” refers to a radical of the formula —(CH₂)₀₋₁-Rₐ₃ and Rₐ₃ is a C(O)di(C₁-C₃alkyl)amino radical as defined above.

The term (CH₂)₀₋₁C(O)NR¹cR^{1d} refers to a radical of the formula —(CH₂)₀₋₁C(O)NR^{1c}R^{1d}.

As used herein, the term “5- or 6-membered saturated heterocyclic ring comprising at least one heteroatom selected from N and O” refers to a monocyclic ring and includes, but is not limited to, piperazinyl, piperidyl, pyrrolidinyl, tetrahydrofuryl, tetrahydropyranyl, dioxanyl and morpholinyl. Preferably this term includes piperidyl, pyrrolidinyl, tetrahydrofuryl, tetrahydropyranyl and morpholinyl. The terms “5-membered saturated heterocyclic ring comprising at least one heteroatom selected from N and O” and “6-membered saturated heterocyclic ring comprising at least one heteroatom selected from N and O” are to be construed accordingly.

As used herein, the term “4-, 5- or 6-membered saturated heterocyclic ring comprising at least one heteroatom or heteroatom group selected from N, O, S, —S(═O) and —S(═O)₂” refers to a monocyclic ring and includes, but is not limited to, azetidinyl, pyrrolidinyl, piperidinyl, piperazinyl, morpholinyl, thiomorpholinyl, S-oxo-thiomorpholinyl or S,S-dioxothiomorpholinyl. For the avoidance of doubt, in certain embodiments whereby the N is present in the α-positon to the atom binding Q to the rest of the molecule, this may be represented by the following formula

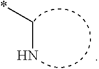 [SMILES: *CN]

As used herein, the term “5- or 6-membered aromatic heterocyclic ring comprising at least one heteroatom selected from N, O, or S, preferably from N or S” refers to a monocyclic aromatic ring. Examples of this term include but are not limited to oxazolyl, isozaolyl, pyrimidinyl, pyridazinyl, tetrazolyl, pyrazinyl, triazolyl, imidazolyl, pyrazolyl, pyridinyl and thiazolyl.

As used herein, the term “5- or 6-membered aromatic heterocyclic ring comprising at least one heteroatom selected from N, O, and S” refers to an aromatic monocyclic ring and includes, but is not limited to, pyrimidinyl, pyridazinyl, tetrazolyl, pyrazinyl, triazolyl, imidazolyl, pyrazolyl, pyridinyl, oxazolyl, and thiazolyl. The point of attachment to the imidazolyl ring is preferably to the nitrogen atom of the imidazolyl ring.

As used herein, the term “5- or 6-membered aromatic heterocyclic ring comprising at least one heteroatom selected from N and S” refers to a monocyclic aromatic ring and includes, but is not limited to, pyrimidinyl, pyridazinyl, tetrazolyl, pyrazinyl, triazolyl, imidazolyl, pyrazolyl, pyridinyl and thiazolyl.

As used herein, the term “6-membered aromatic heterocyclic ring comprising at least one N heteroatom” refers to a monocyclic aromatic ring and includes, but is not limited to, pyrimidinyl, pyridazinyl, pyrazinyl and pyridinyl.

As used herein, the term “5-membered aromatic heterocyclic ring comprising at least one N heteroatom” (where N may also be NH) refers to a monocyclic aromatic ring and includes, but is not limited to, tetrazolyl, triazolyl, imidazolyl, pyrazolyl.

As used herein, the term “5- or 6-membered aromatic heterocyclic ring comprising at least one N heteroatom” refers to a monocyclic aromatic ring and includes, but is not limited to, pyrimidinyl, pyridazinyl, tetrazolyl, pyrazinyl, triazolyl, imidazolyl, pyrazolyl and pyridinyl.

As used herein, the aromatic heterocyclic ring in the substituent defined as “5- or 6-membered aromatic heterocyclic ring comprising at least one heteroatom selected from N, O, or S, preferably from N or S” imay be optionally substituted with hydroxy; C₁-C₃alkoxy; or oxo.

It will be understood that substitution of said aromatic heterocycle with oxo is meant to include 5- or 6-membered rings in which an aromatic tautomer exists, as for example in the 1H-pyridin-2-one system (see for example Example 92).

As used herein, the term “5- or 6-membered saturated heterocyclic ring” in relation to the embodiments where R^{5a} and R^{5b} together with the N atom (where N may also be NH) to which they are attached form said ring, includes as examples, but is not limited to, an azetidinyl ring, a pyrrolidine ring, or a piperidine ring.

As used herein, the term “9- or 10-membered partially saturated heteroaryl comprising at least one N heteroatom” refers to a partially saturated aromatic bicyclic heterocyclic ring system whereby a 5- or 6-membered heterocyclic ring containing one N heteroatom, is fused with a benzene ring or a heteroaromatic ring. In certain embodiments whereby the N is present in the α-positon to the atom binding Q to the rest of the molecule, this may be represented by the following formula

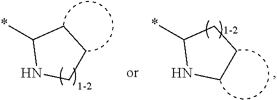

whereby the dashed ring represents the benzo or heteroaryl ring. Representative examples are indolinyl, isoindolinyl, tetrahydroquinolinyl, tetrahydroisoquinolinyl, and the like. Preferably, it is

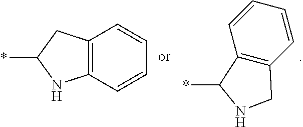

As used herein, the term “optionally substituted” includes unsubstituted or substituted.

As used herein, the term “more than once” includes 2, 3, 4, 5, or 6 times. Preferably, it includes 2 or 3 times.

As used herein, the term “more than one” includes 2, 3, 4, 5, or 6. Preferably, it includes 2 or 3.

As used herein, the term “at least one heteroatom” includes 1, 2, 3, 4 or 5, preferably 1, 2, 3 or 4, more preferably 1 or 2 heteroatoms.

The use of any and all examples, or exemplary language (e.g. “such as” or “preferably”) provided herein is intended merely to better illuminate the invention and does not pose a limitation on the scope of the invention otherwise claimed.

As used herein, the term nitrogen protecting group (PG) in a compound of formula (IV) and subformulae thereof refers to a group that should protect the functional groups concerned against unwanted secondary reactions, such as acylations, etherifications, esterifications, oxidations, solvolysis and similar reactions. It may be removed under deprotection conditions. Depending on the protecting group employed, the skilled person would know how to remove the protecting group to obtain the free amine NH₂ group by reference to known procedures. These include reference to organic chemistry textbooks and literature procedures such as J. F. W. McOmie, “Protective Groups in Organic Chemistry”, Plenum Press, London and New York 1973; T. W. Greene and P. G. M. Wuts, “Greene's Protective Groups in Organic Synthesis”, Fourth Edition, Wiley, New York 2007; in “The Peptides”; Volume 3 (editors: E. Gross and J. Meienhofer), Academic Press, London and New York 1981, and in “Methoden der organischen Chemie” (Methods of Organic Chemistry), Houben Weyl, 4th edition, Volume 15/I, Georg Thieme Verlag, Stuttgart 1974 and later editions thereof.

Preferred nitrogen protecting groups generally comprise: C₁-C₆alkyl (e.g. tert-butyl), preferably C₁-C₄alkyl, more preferably C₁-C₂alkyl, most preferably C₁alkyl which is mono-, di- or tri-substituted with trialkylsilyl-C₁-C₇alkoxy (eg. trimethylsilyethoxy), aryl, preferably phenyl, or a heterocyclic group (e.g. benzyl, cumyl, benzhydryl, pyrrolidinyl, trityl, pyrrolidinylmethyl, 1-methyl-1,1-dimethylbenzyl, (phenyl)methylbenzene) wherein the aryl ring or the heterocyclic group is unsubstituted or substituted with one or more, e.g. two orthree, residues, e.g. selected from the group consisting of C₁-C₇alkyl, hydroxy, C₁-C₇alkoxy (e.g. para-methoxy benzyl (PMB)), C₂-Cₐ-alkanoyl-oxy, halogen, nitro, cyano, and CF₃, aryl-C₁-C₂-alkoxycarbonyl (preferably phenyl-C₁-C₂-alkoxycarbonyl (eg. benzyloxycarbonyl (Cbz), benzyloxymethyl (BOM), pivaloyloxymethyl (POM)), C₁-C₁₀-alkenyloxycarbonyl, C₁-C₆alkylcarbonyl (eg. acetyl or pivaloyl), C₆-C₁₀-arylcarbonyl; C₁-C₆-alkoxycarbonyl (eg. tertbutoxycarbonyl (Boc), methylcarbonyl, trichloroethoxycarbonyl (Troc), pivaloyl (Piv), allyloxycarbonyl), C₆-C₁₀-arylC₁-C₆-alkoxycarbonyl (e.g. 9-fluorenylmethyloxycarbonyl (Fmoc)), allyl or cinnamyl, sulfonyl or sulfenyl, succinimidyl group, silyl groups (e.g. triarylsilyl, trialkylsilyl, triethylsilyl (TES), trimethylsilylethoxymethyl (SEM), trimethylsilyl (TMS), triisopropylsilyl or tertbutyldimethylsilyl).

According to the invention, the preferred protecting group (PG) can be selected from the group comprising tert-butyloxycarbonyl (Boc), benzyloxycarbonyl (Cbz), para-methoxy benzyl (PMB), methyloxycarbonyl and benzyl. The protecting group (PG) is preferably tert-butyloxycarbonyl (Boc).

The term “phenyl” refers to a radical of the formula —C₆H5.

The term “halobenzodioxole” refers to a 1,3-benzodioxole radical of the formula

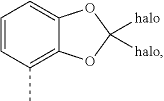

wherein halo is as defined above. Preferably, both halo groups are fluoro.

The term “stereoisomer” or “stereoisomers” refer to compounds, which have identical chemical constitution, but differ with regard to the arrangement of the atoms or groups in space.

The term “diastereoisomer” or “diastereomer” refers to stereoisomers not related as mirror images. Diastereoisomers are characterized by differences in physical properties, and by some differences in chemical behaviour. Mixtures of diastereomers may separate under analytical procedures such as chromatography or crystallisation.

The term “enantiomer” refers to one of a pair of molecular entities which are mirror images of each other and non-superimposable.

The term “enantiomeric mixture” refers to an enantiomerically enriched mixture, a composition that comprises a greater proportion or percentage of one of the enantiomers of the compounds of the invention, in relation to the other enantiomer, or a racemate.

The term “diastereomeric mixture” refers to a diastereomerically enriched mixture or a mixture of diastereoisomers of equal proportion.

The term “diastereomerically enriched” refers to a composition that comprises a greater proportion or percentage of one of the diastereomers of the compounds of the invention, in relation to the other diastereoisomer(s).

The term “atropisomer” refers to a stereoisomer resulting from restricted rotation about single bonds where the rotation barrier is high enough to permit isolation of the isomeric species.

Typically, rotation about the single bond in the molecule is prevented, or greatly slowed, as a result of steric interactions with other parts of the molecule and the substituents at both ends of the single bond are asymmetrical, resulting in a stereogenic unit termed a “chiral axis”.

As used herein, the term “YAP” refers to yes-associated protein, also known as YAP1 or YAP65.

Whenever YAP is mentioned herein it can also refer to the YAP/TAZ complex.

As used herein, the term “YAP/TAZ-TEAD” refers to the complex of YAP/TAZ with TEAD transcription factor.

As used herein, the term “NF2/LATS1/LATS2” refers to “NF2”, “LATS1”, or “LATS2” or any combinations thereof.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis- (Z)- or trans- (E)- form.

Accordingly, as used herein a compound present in any of the combinations or methods disclosed herein can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the present invention or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds of the present invention or racemic intermediates can also be resolved by chiral chromatography, e.g., high pressure liquid chromatography (HPLC) using a chiral adsorbent.

Synthesis of Compounds A, B, D, E, F and G and related TEAD inhibitors was originally described in PCT/IB2021/052136 (WO2021/186324), the contents of which are incorporated by reference.

### COMBINATION EXAMPLES

### Method

For each of the cell lines in Examples 11-38, cells were dispensed into tissue culture treated 384-well plates (Greiner #781098) in a final volume of 25 uL per well. Cells were allowed to adhere and begin growth for twenty-four hours. On plate was counted prior treatment (=Day 1), and the other plate was treated with compounds or DMSO using a HP D300 digital dispenser.

After seventy-two hours the medium was refreshed by supplementing 25 μl per well of culture medium containing the corresponding compounds or DMSO. All treatments were done in triplicates.

Seven days after treatment initiation, cell growth was determined using CellTiter-Glo® (Promega #G7573), which measures the amount of ATP in the well. Plates were equilibrated to room temperature for approximately thirty minutes and one volume of CellTiter-Glo® Reagent equal to the volume of cell culture medium was added. Cell lysis was induced for two minutes on an orbital shaker, the plates were incubated at room temperature for ten minutes, and luminescence was recorded.

Cells were treated with the indicated final concentrations of compounds. Dose response curves were derived using XLfit dose response one site, model 205. Reported is the percentage of growth inhibition versus DMSO (percentage GI) after subtracting the reads of Day 1.

### Example 1: Anti-Tumor Efficacy of a TEAD Inhibitor in Combination with a KRAS G12C Inhibitor in H2122 and 2094-HX Mouse Xenograft Models

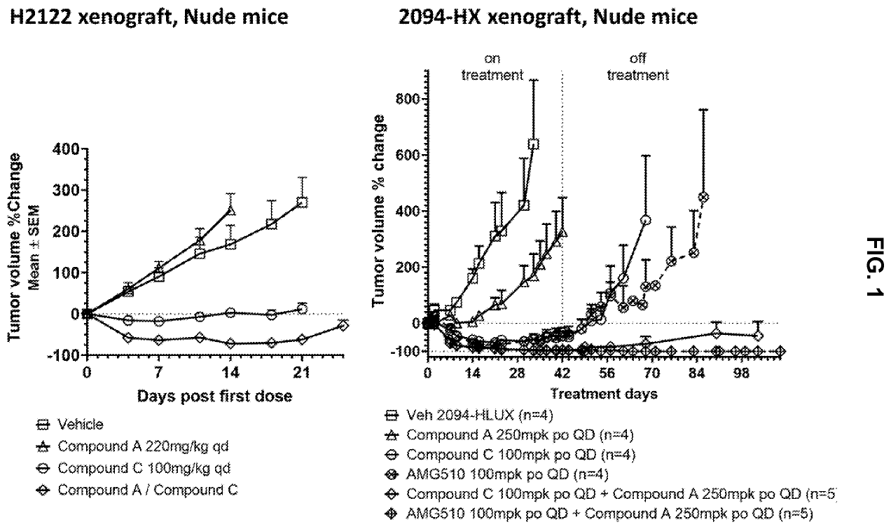

Compound C, and AMG510 (Sotorasib) from Amgen are potent, selective and irreversible inhibitors of mutant KRAS G12C protein. In these studies (FIG. 1), the anti-tumor activity and tolerability of TEAD inhibitor Compound A, Compound C and Sotorasib (AMG510) KRAS G12C inhibitors, and their combination in two mouse xenograft models were evaluated. H2122 is a human lung carcinoma cell line and 2094-HX is a Patient-Derived-Xenograft (PDX) lung carcinoma model. Both cancer models are mutant on KRAS and bear a G12C mutation. A treatment with single agent Compound A at the concentration shown does not induce stasis or tumor regression in H2122 tumor models. An anti-tumor efficacy is observed however with Compound C or AMG510 KRAS G12C inhibitors that, administered as single agents, induce stasis in H2122 and a tumor regression in the PDX model during treatment. In both experiments, deeper tumor responses are observed with the combination of Compound A with a KRAS G12C inhibitor. In particular, complete tumor regressions are observed in the combination group of the 2094-HX study. Interestingly, more than 11 weeks after the treatments are stopped, most 2094-HX tumors treated with combinations remained undetected. Body weight remained in the normal range during the course of these experiments, tolerability was good.

### Example 2: Combination of a TEAD Inhibitor with a KRAS G12C Inhibitor in Mutant Colorectal PDX Cancer Models

(KRAS) G12C mutation occurs in about 4% of colorectal cancers (CRCs). Recently, KRAS G12C was identified to be a potential drug target and predictor of response to the KRAS G12C inhibitors. However response rate and overall survival benefit remains limited upon such treatments. Here we assess the potential benefit of a combination of the KRASG12C inhibitor Compound C with a YAP/TAZ-TEAD inhibitor Compound A. Compound A potently disrupts the protein-protein interaction between YAP1/WWTR1 and all four TEAD isoforms, thereby abolishing the transcriptional activity of the complex. It is known from in vitro assays and un-shown preclinical experiments that such a YAP/TAZ-TEAD inhibitor is not efficacious in CRC tumor models as a single agent.

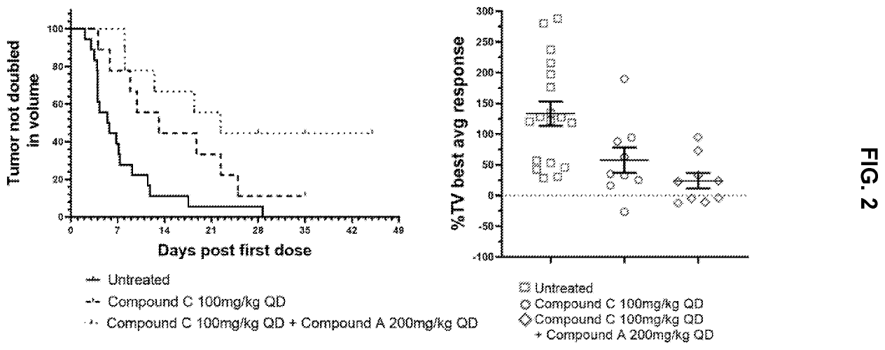

The activities of the Compound A and/or Compound C drugs were assessed in patient-derived xenograft (PDX) models implanted in Nude mice, using a MCT (Mouse Clinical Trial) scheme (FIG. 2). These PDX models originated from 9 different CRC patients. Nude mice were implanted with the same PDX model. After randomization, one mouse was treated daily with Compound C, another one with Compound C+Compound A. The two other mice were untreated, as controls. When mice bearing xenografts models were treated daily with 100 mg/kg of Compound C, significant tumor responses were observed. The anti-tumor effect was more prominent, with a tumor growth reduced by 2.4 folds, with the combination Compound C+Compound A. See FIG. 2.

### Example 3: Anti-Tumor Efficacy of a TEAD Inhibitor in Combination with a SHP2 Inhibitor in Mesothelioma and Lung Cancer Mouse Xenograft Models

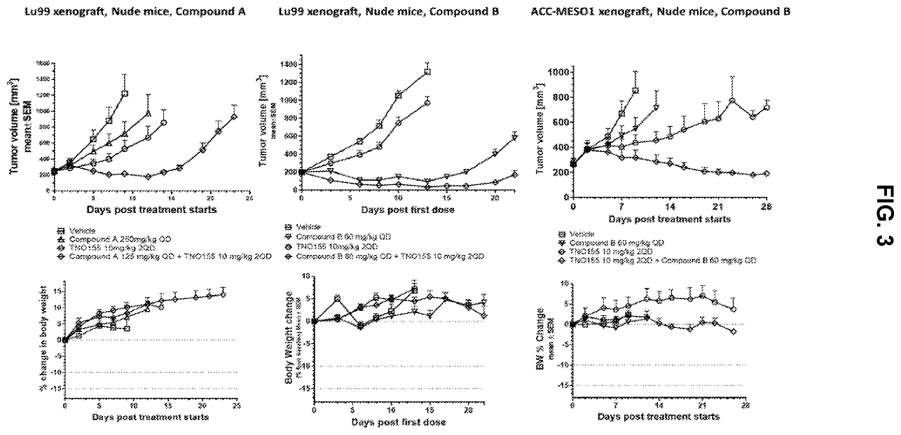

TNO155 is a first-in-class allosteric inhibitor of wild-type SHP2 that prevents the transduction of signalling from activated RTKs to the downstream RAS/MAPK pathway. The combination of a TEAD inhibitor and a SHP2 inhibitor (TNO155) in thoracic malignant tumors is assessed (FIG. 3). A heterozygous KRAS G12C lung cancer xenograft model, Lu99, and a malignant pleural mesothelioma cancer xenograft model, ACC-MESO1, are used in efficacy studies in mice. While a mild to good anti-tumor response with single agents was observed, the combinations led to better outcome with observed tumor stasis or regression. All treatments are well tolerated and no body weight loss is observed. These results demonstrate that combination of SHP2 and TEAD inhibitors are beneficial in countering these malignant tumors.

### Example 4: Triple Combination of a TEAD Inhibitor, a SHP2 Inhibitor and a KRAS G12C Inhibitor in a Lu99 Lung Cancer Xenograft Model

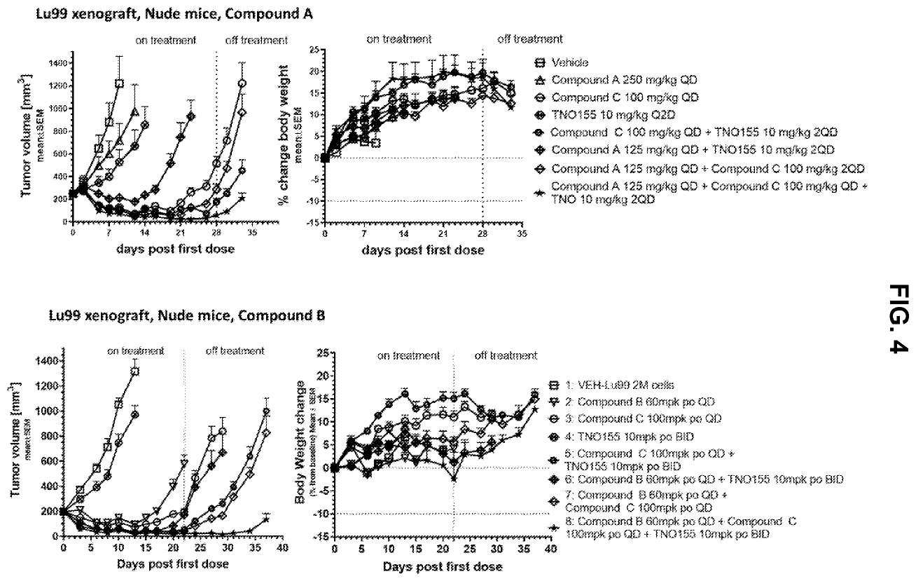

The combination of i) a TEAD inhibitor, ii) a SHP2 inhibitor, and iii) a KRAS G12C inhibitortwo by two and as a triple-combination therapy is assessed (FIG. 4). The heterozygous KRAS G12C lung cancer xenograft model, Lu99, is used in efficacy studies in Nude mice. Compound A and Compound B TEADs inhibitors are assessed in two separate studies in treatments of 28 and 22 days, respectively. The animals were monitored further after treatment was stopped to observe tumor regrowth. In both experiments, the triple combination regimen showed complete regression. Body weight remained in the normal range during the course of these experiments, tolerability was good.

### Example 5

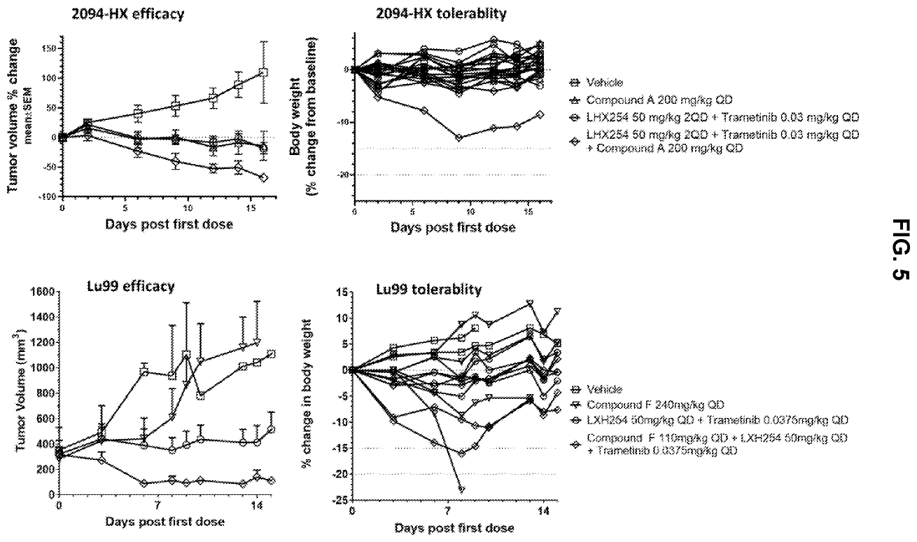

The combination of the pan-Raf inhibitor LHX254, and the MEK1/2 inhibitor Trametinib has been tested clinically in MAPK mutated cancers. The triple combination of a TEAD inhibitor (Compound F) with LHX254 and Trametinib is assessed herein (FIG. 5) for a potential beneficial anti-tumor effect on lung cancer tumors. Two KRAS mutant lung cancer xenograft models, Lu99 and 2094-HX are used in efficacy studies in mice. Whilst tumor stasis with LHX254/Trametinib therapy was observed, the triple combination with a TEAD inhibitor led to better outcome with observed tumor regression. All treatments are generally tolerated and only moderate body weight loss is observed with the triple combination. These results demonstrate a potential therapeutic benefit of adding YAP/TAZ-TEADs inhibitors to LHX254 and Trametinib treatments to counter malignant tumors.

### Example 6

The combination of the pan-Raf inhibitor LHX254, and the ERK1/2 activity LTT462 inhibitor has been tested clinically in MAPK mutated cancers.

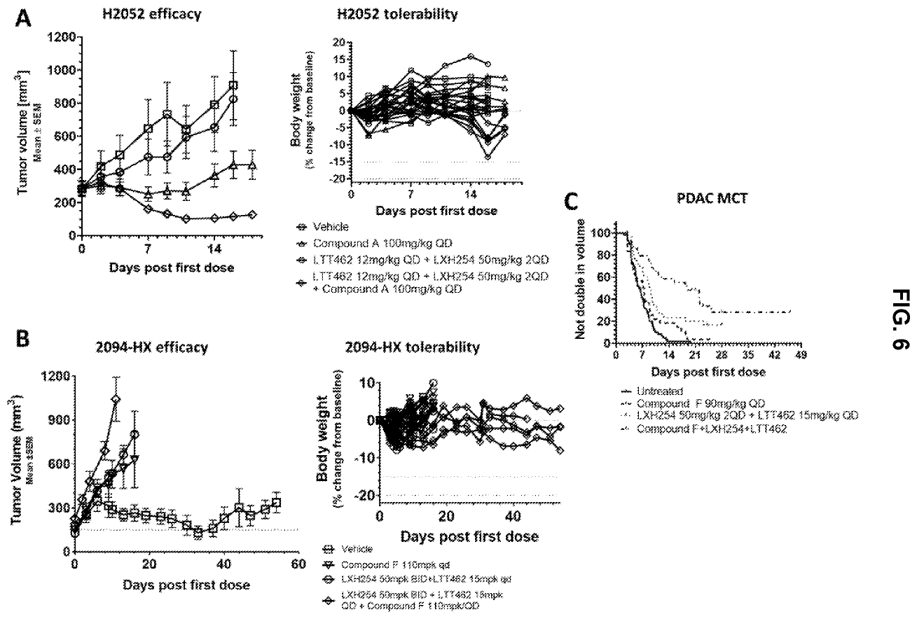

The combination of TEAD inhibitor with LHX254/LTT462 therapy is assessed herein (FIG. 6) for a potential beneficial anti-tumor effect on lung cancer tumors.

NCI-H2052 malignant pleural mesothelioma cancer xenograft model is used in a pharmacology study in mice. A mild (Compound A) to poor (LHX254/LTT462) anti-tumor response with simple targeted therapies was observed, However, the combination of these three agents led to better outcome with observed tumor regression (FIG. 6-A) in the KRAS G12C lung cancer xenograft mouse models 2094-HX. Targeted therapies Compound A or LHX254/LTT462 do not provide much anti-tumor effect. In contrast, the triple combination leads to a sustained tumor stasis (FIG. 6-B).

A PDAC mouse clinical trial (MCT) was performed (FIG. 6-C) with Compound F (2-((2S,3S)-5-chloro-6-fluoro-2-((((1r,4S)-4-hydroxy-4-methylcyclohexyl)amino)methyl)-3-methyl-2-phenyl-2,3-dihydrobenzofuran-4-yl)-3-fluoro-4-((S)-2-hydroxypropoxy)benzamide) at a moderate dose of 90 mg/kg daily and the combination of the MAPKi LHX254/LTT462. Even though these pancreatic tumors are generally difficult to treat, with the triple combination, there were 11 out of 23 responders (5 tumor stasis and 6 tumor regressions). And other PDX models considered non-responders generally displayed a reduced tumor progression. Overall these results demonstrate a potential therapeutic benefit of adding TEAD inhibitors to LHX254/LTT462 treatments to counter malignant tumors.

### Example 7

The BRAFV600E mutation is found in 8-10% of metastatic colorectal cancer (CRC) patients and it is recognized as a poor prognostic factor with a median overall survival inferior to 20 months (Cancers (Basel). 2021 January; 13(1): 137.).

One of the strategies is the targeting of MAPK pathway by applying Dabrafenib BRAF mutant inhibitor with concomitant MAPK inhibitors (i.e. MEK, RAF, and/or ERK inhibitors).

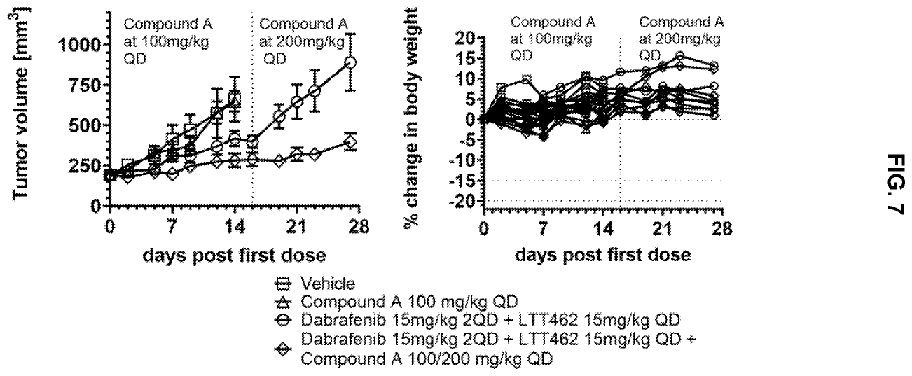

The triple combination of a TEAD inhibitor with Dabrafenib and LTT462 is assessed herein for a potential beneficial anti-tumor effect on lung cancer tumors (FIG. 7), using the HT-29 CRC xenograft model in an efficacy study in mice.

While little tumor response was observed in response to Dabrafenib/LTT462 combination therapy, the triple combination with a TEAD inhibitor led to better outcome with prolonged tumor stasis. Compound A was used at 100 mg/kg for 2 weeks and, since tolerability was acceptable, the dose was increased to 200 mg/kg for 2 additional weeks. These treatments are well tolerated and no body weight loss was observed. These results demonstrate a potential therapeutic benefit of adding YAP/TAZ-TEADs inhibitors to Dabrafenib/LTT462 treatments to counter malignant tumors.

### Example 8

The BRAFV600E mutation is found in 8-10% of metastatic colorectal cancer (CRC) patients and it is recognized as a poor prognostic factor with a median overall survival inferior to 20 months (Cancers (Basel). 2021 January; 13(1): 137.). One of the strategies in the art is the targeting of MAPK pathway by applying either Dabrafenib BRAF mutant inhibitor with concomitant MAPK inhibitors (i.e. MEK, RAF, and/or ERK inhibitors). It is herein theorised that additional inhibition of TEADs transcription factors may provide an interesting therapy to improve the anti-tumor response.

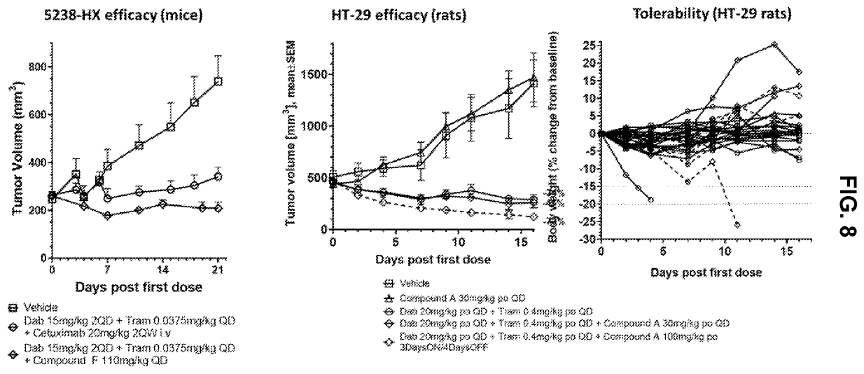

The triple combination of the TEAD inhibitor with Dabrafenib/Trametinib therapy is assessed for a potential beneficial anti-tumor effect on colorectal cancer tumors (FIG. 8). The 5238-HX PDX murine model and the HT-29 rat xenograft model are used in pharmacology studies.

Anti-tumor efficacy was evaluated on implanted xenograft from the PDX model 5238-HX. The triplet therapy of Dabrafenib, Trametinib, and an anti-EGFR antibody such as Cetuximab is one of the currently preferred combination in CRC (Ann Oncol. 2016; 27 (suppl_6):4550.). Herein, this triplet combination is compared to the Dabrafeib, Trametinib and TEAD inhibitor (Compound F) combination in mice. It was found that blocking TEAD was more beneficial than blocking EGFR in addition to Dabrafenib/Trametinib in this particular model.

Ref: Corcoran R B, Andre T, Yoshino T, et al. Efficacy and circulating tumor DNA (ctDNA) analysis of the BRAF inhibitor dabrafenib (D), MEK inhibitor trametinib (T), and anti-EGFR antibody panitumumab (P) in patients (pts) with BRAF V600E-mutated (BRAFm) metastatic colorectal cancer (mCRC) Ann Oncol. 2016; 27 (suppl_6):4550.

In the HT-29 rat model, the Dabrafenib/Trametinib therapy leads to 34% tumor regression after 16 days. The addition of the TEAD inhibitor Compound A, used at a weekly dose of 210 mg/kg with a daily administration of 30 mg/kg, improved the response slightly (−41% tumor regression).

To be able to increase Compound A dose/effect in rats without triggering tolerability issues, we used an intermittent schedule to administrate 300 mg/kg week: 100 mg/kg were administered with a 3 days on/4 days off schedule. At this higher dose, the regression observed was deeper, −73% at day 16.

These results demonstrate a potential therapeutic benefit of adding YAP/TAZ-TEADs inhibitors to Dabrafenib/Trametinib treatments to counter malignant tumors.

### Example 9

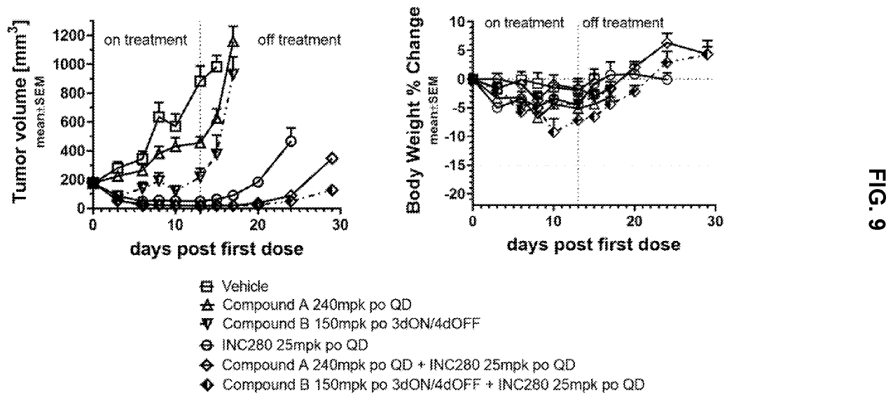

Anti-tumor efficacy of concomitant inhibition of TEADs (using Compound A or Compound B) and cMET inhibition (using INC280/capmatinib) was investigated using a syngeneic mouse model (FIG. 9), 24284-MA.

The 24284-MA allograft tumor model was developed at Novartis; it is a spontaneous tumor originated from a genetically engineered mouse strain with an Arf null mutation, thus also leading to copy number alterations. It was identified as a spindle cell sarcoma with a Met gene amplification. The mice were subjected to treatments for 13 days, after which recovery was monitored. The TEAD inhibitor Compound A had a limited effect as single agent, although with Compound B, tumor stasis was observed under treatment. After treatment was stopped, tumor growth rate increased. The cMET inhibitor capmatinib produced a deep regression (−70.3%, day 13) as a single agent. However, combining capmatinib with either Compound A or Compound B led to enhanced responses (−90.6% and −88.6% respectively, day 13). After treatments stopped, the tumors took longer to grow back in the combination regimens as opposed to in the monotherapy.

### Example 10

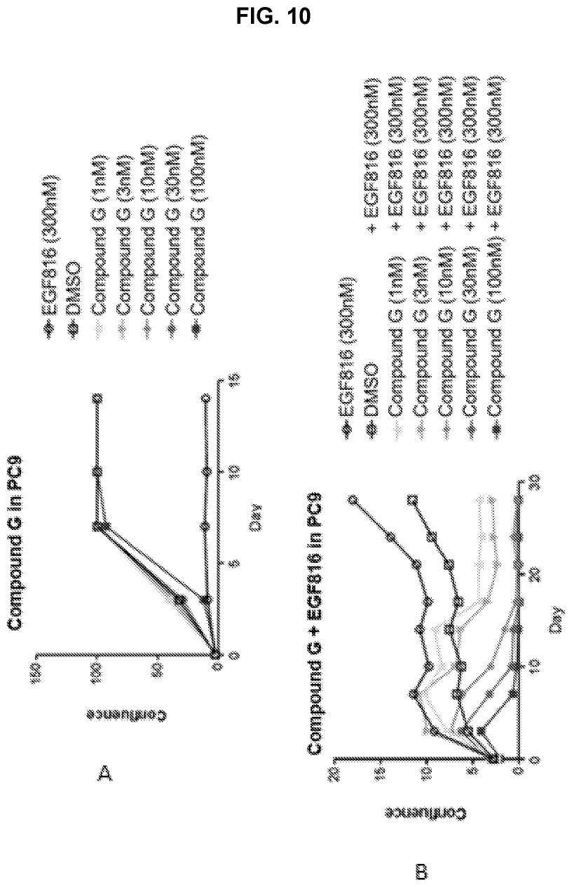

The TEAD inhibitor Compound G (2-((2S,4S)-5-chloro-6-fluoro-2-((((1r,4S)-4-hydroxycyclohexyl)amino)methyl)-2-phenyl-2,3-dihydrobenzofuran-4-yl)-4-(difluoromethoxy)-3-fluorobenzamide) was combined with the EGFR inhibitor EGF816 in the lung cancer cell line PC9 which carries an activating EGFR mutation. PC9 cells were found to be insensitive to Compound G single agent treatment up to 100 nM (FIG. 10A) but strongly inhibited by EGF816 single agent treatment for up to 15 days (FIG. 10A) after which proliferation resumes despite continuous EGF816 exposure (FIG. 10B, EGF816 300 nM). The combination of Compound G with EGF816 showed a dose-dependent benefit and completely prevented the emergence of cell regrowth observed with EGF816 single agent (FIG. 10B, 816+QHS 10-30-100 nM).

### Example 11

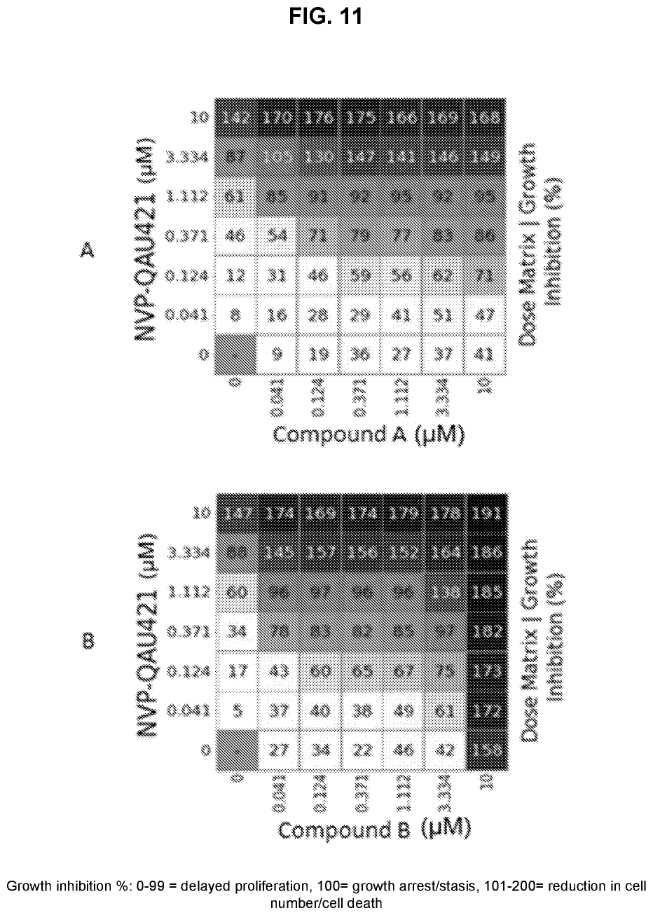

In vitro viability of the MCF7 breast cancer cell line (PIK3CA mutant) was assessed using the CellTiterGlo assay following 6-day treatment with TEAD inhibitor (Compound A or Compound B) in combination with the PI3K inhibitor QAU421 (an analog of BYL719/alpelisib/Piqray). Minimal growth inhibition was observed with Compound A or Compound B single agent treatment (up to 3 uM, FIG. 11A-B). In contrast, the combination of either Compound A or Compound B with QAU421 resulted in a dose-dependent induction of cell death.

### Example 12

In vitro viability of the lung KRAS G12C-mutant cell line LU-99 was assessed using the CellTiterGlo assay following 6-day treatment with the TEAD inhibitor Compound D, combined with various combinations of MAPK pathway inhibitors (MEK inhibitor MEKINIST/Trametinib/CFF272, BRAF/CRAF inhibitor LXH254 and ERK inhibitor LTT462).

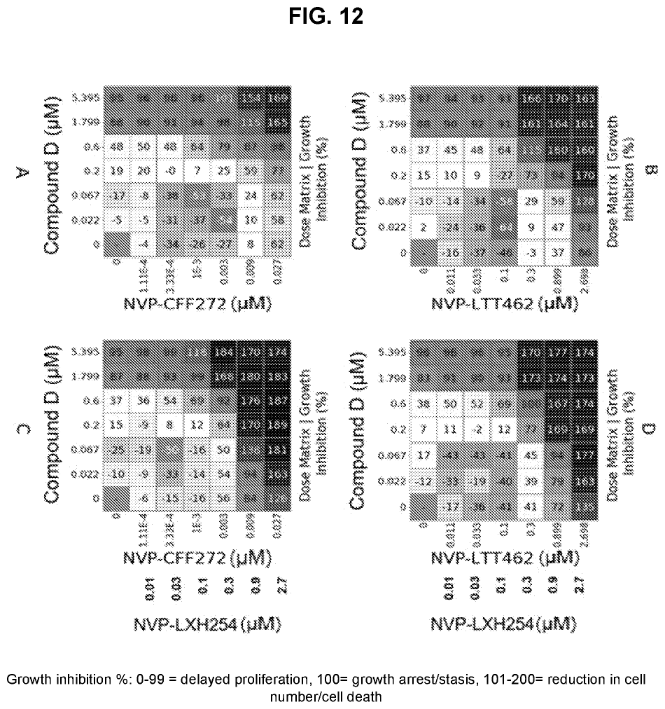

Combination of Compound D+CFF272 or Compound D+LTT462 were each sufficient to induce cell death at concentrations where single agent cause cell growth delay (FIG. 12 A-B). The combination benefit was enhanced upon deeper MAPK inhibition in the context of triple combinations with the BRAF/CRAF inhibitor LXH254 (FIG. 12 C-D).

### Example 13

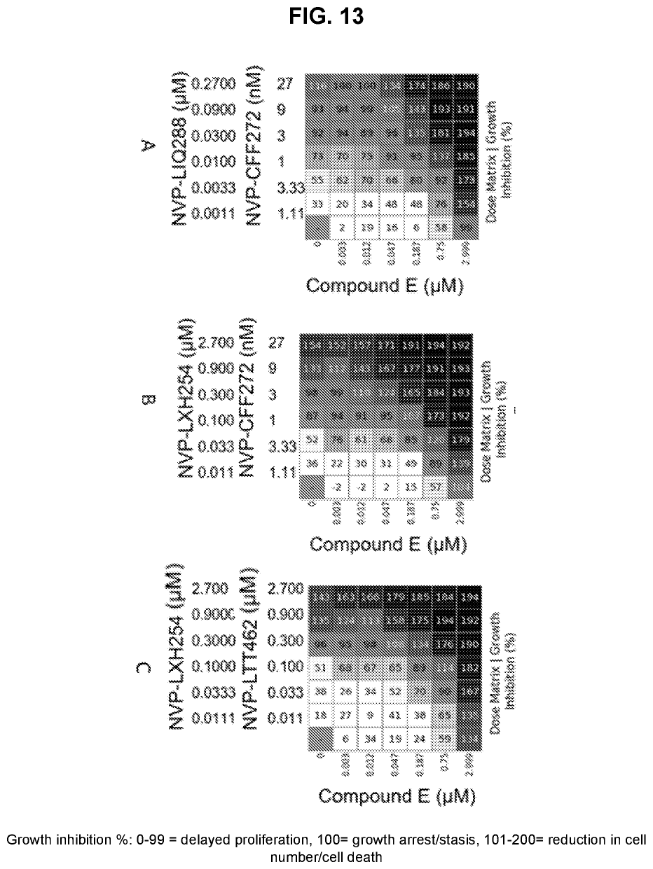

In vitro viability of the lung BRAF mutant colorectal cell line SW-1417 was assessed using the CellTiterGlo assay following 6-day treatment with the TEAD inhibitor Compound E, combined with various combinations of MAPK pathway inhibitors (BRAF inhibitor TAFINLAR/Dabrafenib/LIQ288, MEK inhibitor MEKINIST/Trametinib/CFF272, BRAF/CRAF inhibitor LXH254 and ERK inhibitor LTT462). Triple combinations involving Compound E+LIQ288+CFF272 (FIG. 13A), Compound E+LXH254+CFF272 (FIG. 13B) and Compound E+LXH254+LTT462 (FIG. 13C) were each sufficient to induce cell death at concentrations whereas Compound E single agent or double MAPK combinations caused cell growth delay (FIG. 13A-B).

### Example 14

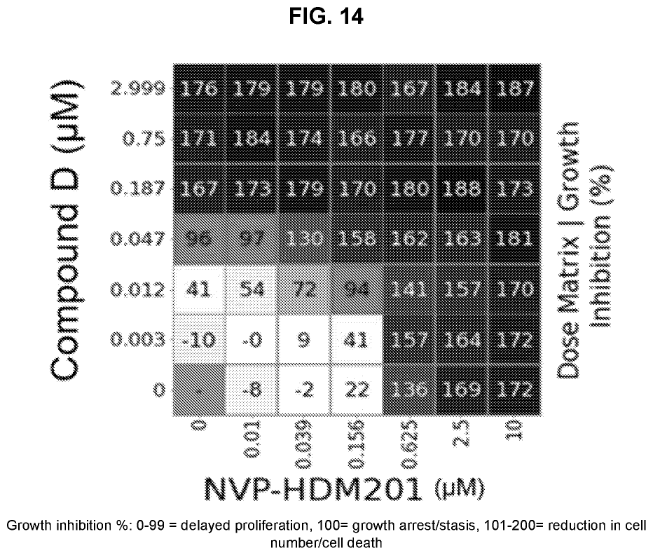

In vitro viability of the CDKN2A-deleted mesothelioma cell line MSTO-211H was assessed using the CellTiterGlo assay following 3-day treatment with the TEAD inhibitor Compound D, combined with the p53-HDM2 inhibitor HDM201. Combination benefit was observed at sub-efficacious concentrations for single agents (FIG. 14).

### Example 15

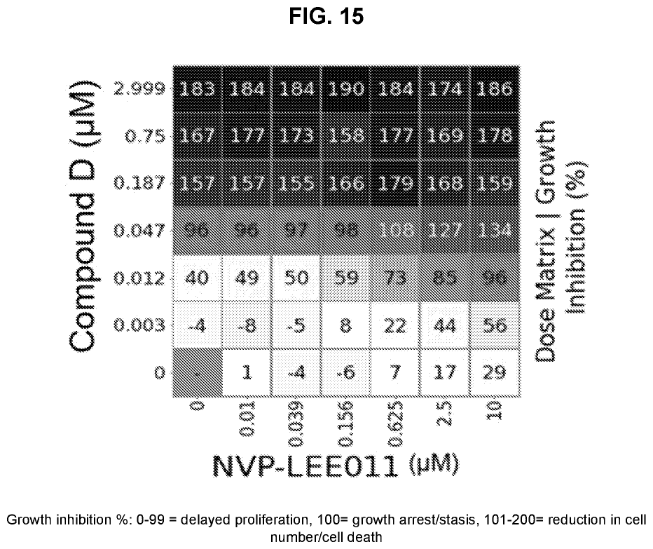

In vitro viability of the mesothelioma cell line MSTO-211H was assessed using the CellTiterGlo assay following 3-day treatment with the TEAD inhibitor Compound D, combined with the CDK4/6 inhibitor LEE011. MSTO-211H was insensitive to LEE011 single agent treatment but combination benefit was observed at sub-efficacious concentrations of Compound D (FIG. 15).

### Example 16

In vitro viability of the lung cancer cell line HCC44 was assessed using the CellTiterGlo following 7-day treatment with (i) the YAP/TEAD inhibitor Compound A combined with the KRAS G12C inhibitor JDQ443 and (ii) the YAP/TEAD inhibitor Compound A combined with the KRAS G12C inhibitor JDQ443 and the SHP2 inhibitor TNO155. Proliferation of HCC44 cells was inhibited by JDQ443 alone and JDQ443+TNO155, and Compound A also showed single agent activity. By comparison, combinations (i) and (ii) displayed synergistic growth inhibition compared to either treatment alone.

### Example 17

In vitro viability of the lung cancer cell line HCC44 was assessed using the CellTiterGlo following 7-day treatment with (i) the YAP/TEAD inhibitor Compound A combined with the KRAS G12C inhibitor Sotorasib and (ii) the YAP/TEAD inhibitor Compound A combined with the KRAS G12C inhibitor Sotorasib and the SHP2 inhibitor TNO155. Proliferation of HCC44 cells was inhibited by Sotorasib alone and Sotorasib+TNO155, and Compound A also showed single agent activity. By comparison, combinations (i) and (ii) displayed synergistic growth inhibition compared to either treatment alone.

### Example 18

In vitro viability of the lung cancer cell line HCC44 was assessed using the CellTiterGlo following 7-day treatment with (i) the YAP/TEAD inhibitor Compound A combined with the KRAS G12C inhibitor Adagrasib, (ii) the YAP/TEAD inhibitor Compound A combined with the KRAS G12C inhibitor Adagrasib and the SHP2 inhibitor TNO155, and (iii) the YAP/TEAD inhibitor Compound A combined with the KRAS G12C inhibitor Adagrasib and the SHP2 inhibitor RMC-4550. Proliferation of HCC44 cells was inhibited by Adagrasib alone or Adagrasib+RMC-4550, and Compound A also showed single agent activity. By comparison, combinations (i), (ii) and (ii) displayed synergistic growth inhibition.

### Example 19

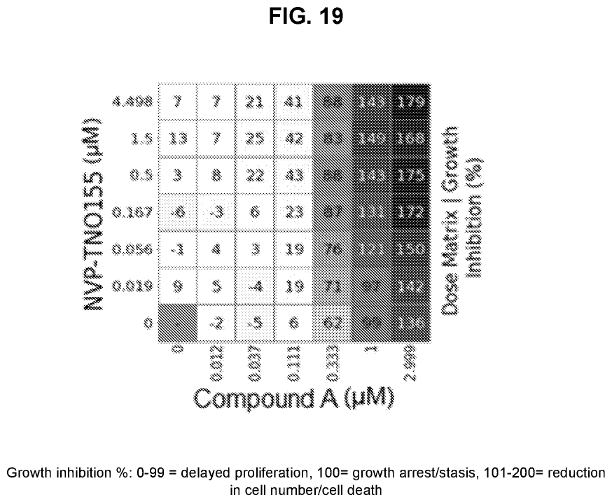

In vitro viability of the lung cancer cell line HCC44 was assessed using the CellTiterGlo following 7-day treatment with the YAP/TEAD inhibitor Compound A combined with the SHP2 inhibitor TNO-155. Proliferation of HCC44 cells was not significantly inhibited by TNO155 at the concentrations tested, while Compound A showed single agent activity. The combination displayed synergistic growth inhibition compared to either treatment alone. Also tested was the combination of the YAP/TEAD inhibitor Compound A and the SHP2 inhibitor RMC-4550 (not shown in FIG. 19). This combination was also synergistic.

### Example 20

In vitro viability of the lung cancer cell line HCC44 was assessed using the CellTiterGlo following 7-day treatment with (i) the YAP/TEAD inhibitor Compound B combined with the KRAS G12C inhibitor JDQ443 and (ii) the YAP/TEAD inhibitor Compound B combined with the KRAS G12C inhibitor JDQ443 and the SHP2 inhibitor TNO155. Proliferation of HCC44 cells was inhibited by JDQ443 alone and JDQ443+TNO155, and Compound B also showed single agent activity. By comparison, combinations (i) and (ii) displayed synergistic growth inhibition compared to either treatment alone.

### Example 21

In vitro viability of the lung cancer cell line HCC44 was assessed using the CellTiterGlo following 7-day treatment with (i) the YAP/TEAD inhibitor Compound B combined with the KRAS G12C inhibitor Sotorasib and (ii) the YAP/TEAD inhibitor Compound B combined with the KRAS G12C inhibitor Sotorasib and the SHP2 inhibitor TNO155. Proliferation of HCC44 cells was inhibited by Sotorasib alone and Sotorasib+TNO155, and Compound B also showed single agent activity. By comparison, combinations (i) and (ii) displayedsynergistic growth inhibition compared to either treatment alone.

### Example 22

In vitro viability of the lung cancer cell line HCC44 was assessed using the CellTiterGlo following 7-day treatment with (i) the YAP/TEAD inhibitor Compound B combined with the KRAS G12C inhibitor Adagrasib, (ii) the YAP/TEAD inhibitor Compound B combined with the KRAS G12C inhibitor Adagrasib and the SHP2 inhibitor TNO155, and (iii) the YAP/TEAD inhibitor Compound B combined with the KRAS G12C inhibitor Adagrasib and the SHP2 inhibitor RMC-4550. Proliferation of HCC44 cells was inhibited by Adagrasib alone and Adagrasib+RMC-4550, and Compound B also showed single agent activity. By comparison, combinations (i), (ii) and (ii) displayed synergistic growth inhibition.

### Example 23

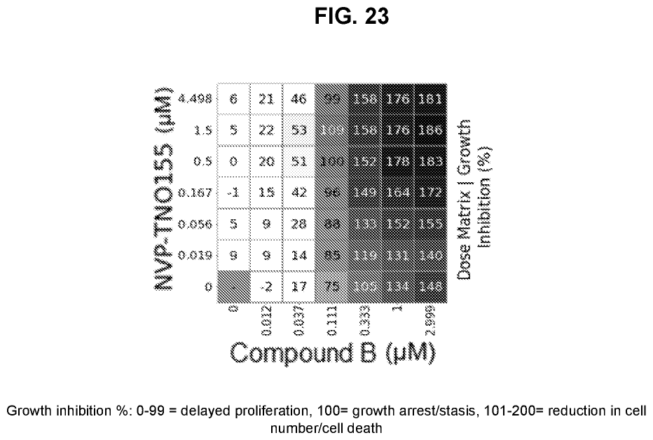

In vitro viability of the lung cancer cell line HCC44 was assessed using the CellTiterGlo following 7-day treatment with the YAP/TEAD inhibitor Compound B combined with the SHP2 inhibitor TNO-155. Proliferation of HCC44 cells was not significantly inhibited by TNO155, while Compound B showed single agent activity. By comparison, the combination displayed synergistic growth inhibition compared to either treatment alone. Also tested was the combination of the YAP/TEAD inhibitor Compound B and the SHP2 inhibitor RMC-4550 (not shown in FIG. 23). This combination was also synergistic.

### Example 24

In vitro viability of the lung cancer cell line HCC44 was assessed using the CellTiterGlo following 7-day treatment with (i) the YAP/TEAD inhibitor Compound H combined with the KRAS G12C inhibitor JDQ443 and (ii) the YAP/TEAD inhibitor Compound H combined with the KRAS G12C inhibitor JDQ443 and the SHP2 inhibitor TNO155. Proliferation of HCC44 cells was inhibited by JDQ443, JDQ443+TNO155 and Compound H, and synergistic combination benefitwas observed in (i) and (ii).

### Example 25

In vitro viability of the lung cancer cell line HCC44 was assessed using the CellTiterGlo following 7-day treatment with (i) the YAP/TEAD inhibitor Compound H combined with the KRAS G12C inhibitor Sotorasib and (ii) the YAP/TEAD inhibitor Compound H combined with the KRAS G12C inhibitor Sotorasib and the SHP2 inhibitor TNO155. Proliferation of HCC44 cells was inhibited by Sotorasib, Sotorasib+TNO155 and Compound H, and synergistic combination benefit was observed in (i) and (ii).

### Example 26

In vitro viability of the lung cancer cell line HCC44 was assessed using the CellTiterGlo following 7-day treatment with (i) the YAP/TEAD inhibitor Compound H combined with the KRAS G12C inhibitor Adagrasib, and (ii) the YAP/TEAD inhibitor Compound H combined with the KRAS G12C inhibitor Adagrasib and the SHP2 inhibitor RMC-4550. Proliferation of HCC44 cells was inhibited by Adagrasib, Adagrasib+TNO155 and Compound H, and synergistic combination benefit was observed in (i) and (ii).

### Example 27

In vitro viability of the colorectal cancer cell line SW1463 was assessed using the CellTiterGlo following 7-day treatment with (i) the YAP/TEAD inhibitor Compound A combined with the KRAS G12C inhibitor JDQ443 and (ii) the YAP/TEAD inhibitor Compound A combined with the KRAS G12C inhibitor JDQ443 and the SHP2 inhibitor TNO155. Proliferation of SW1463 cells was inhibited by JDQ443 alone and JDQ443+TNO155, and Compound A showed some single agent activity. By comparison, combinations (i) and (ii) displayed synergistic growth inhibition compared to either treatment alone.

### Example 28

In vitro viability of the colorectal cancer cell line SW1463 was assessed using the CellTiterGlo following 7-day treatment with (i) the YAP/TEAD inhibitor Compound A combined with the KRAS G12C inhibitor Sotorasib and (ii) the YAP/TEAD inhibitor Compound A combined with the KRAS G12C inhibitor Sotorasib and the SHP2 inhibitor TNO155. Proliferation of SW1463 cells was inhibited by Sotorasib alone and Sotorasib+TNO155, and Compound A also showed some single agent activity. By comparison, combinations (i) and (ii) displayed synergistic growth inhibition compared to either treatment alone.

### Example 29

In vitro viability of the colorectal cancer cell line SW1463 was assessed using the CellTiterGlo following 7-day treatment with (i) the YAP/TEAD inhibitor Compound A combined with the KRAS G12C inhibitor Adagrasib, (ii) the YAP/TEAD inhibitor Compound A combined with the KRAS G12C inhibitor Adagrasib and the SHP2 inhibitor TNO155, and (iii) the YAP/TEAD inhibitor Compound A combined with the KRAS G12C inhibitor Adagrasib and the SHP2 inhibitor RMC-4550. Proliferation of SW1463 cells was inhibited by Adagrasib, Adagrasib+TNO155 and Adagrasib+RMC-4550, and Compound A also showed some single agent activity. By comparison, combinations (i), (ii) and (iii) displayed synergistic growth inhibition compared to either treatment alone.

### Example 30

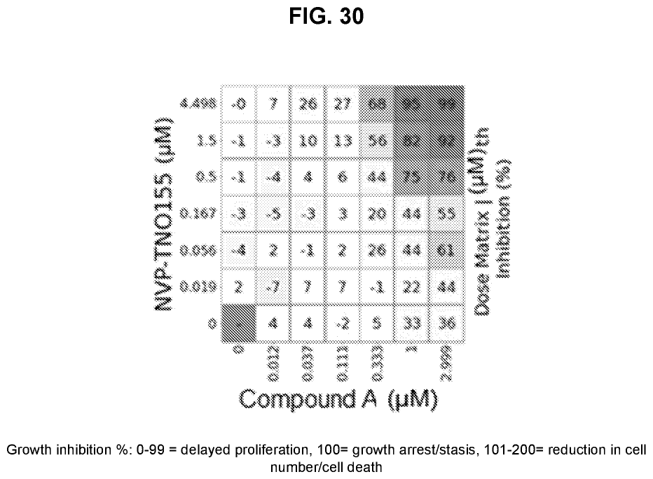

In vitro viability of the colorectal cancer cell line SW1463 was assessed using the CellTiterGlo following 7-day treatment with the YAP/TEAD inhibitor Compound A combined with the SHP2 inhibitor TNO-155. Proliferation of SW1463 cells was not inhibited by TNO-155 at the concentrations tested while Compound A showed some single agent activity. By comparison, the combination displayed synergistic growth inhibition compared to either treatment alone. Also tested was the combination of the YAP/TEAD inhibitor Compound A and the SHP2 inhibitor RMC-4550 (not shown in FIG. 30). This combination was also synergistic.

### Example 31

In vitro viability of the colorectal cancer cell line SW1463 was assessed using the CellTiterGlo following 7-day treatment with (i) the YAP/TEAD inhibitor Compound B combined with the KRAS G12C inhibitor JDQ443 and (ii) the YAP/TEAD inhibitor Compound B combined with the KRAS G12C inhibitor JDQ443 and the SHP2 inhibitor TNO155. Proliferation of SW1463 cells was inhibited by JDQ443 alone and JDQ443+TNO155, and Compound B also showed some single agent activity. By comparison, combinations (i) and (ii) displayed synergistic growth inhibition compared to either treatment alone.

### Example 32

In vitro viability of the colorectal cancer cell line SW1463 was assessed using the CellTiterGlo following 7-day treatment with (i) the YAP/TEAD inhibitor Compound B combined with the KRAS G12C inhibitor Sotorasib and (ii) the YAP/TEAD inhibitor Compound B combined with the KRAS G12C inhibitor Sotorasib and the SHP2 inhibitor TNO155. Proliferation of SW1463 cells was inhibited by Sotorasib alone and Sotorasib+TNO155, and Compound B also showed some single agent activity. By comparison, combinations (i) and (ii) displayed synergistic growth inhibition compared to either treatment alone.

### Example 33

In vitro viability of the colorectal cancer cell line SW1463 was assessed using the CellTiterGlo following 7-day treatment with (i) the YAP/TEAD inhibitor Compound B combined with the KRAS G12C inhibitor Adagrasib, (ii) the YAP/TEAD inhibitor Compound B combined with the KRAS G12C inhibitor Adagrasib and the SHP2 inhibitor TNO155, and (iii) the YAP/TEAD inhibitor Compound B combined with the KRAS G12C inhibitor Adagrasib and the SHP2 inhibitor RMC-4550. Proliferation of SW1463 cells was inhibited by Adagrasib, Adagrasib+TNO155 and Adagrasib+RMC-4550, and Compound B also showed some single agent activity. By comparison, combinations (i), (ii) and (iii) displayed synergistic growth inhibition compared to either treatment alone.

### Example 34

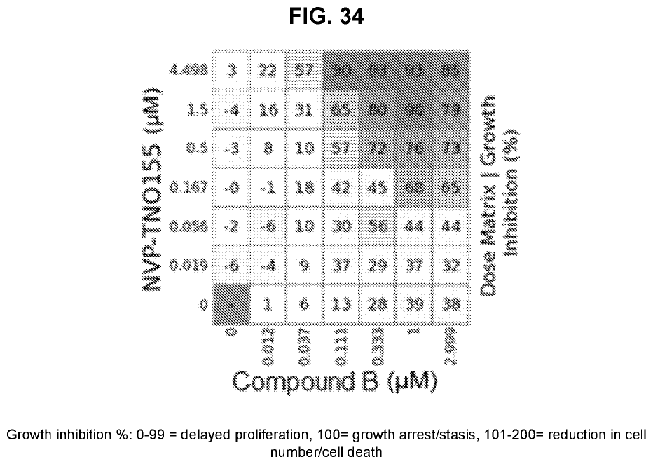

In vitro viability of the colorectal cancer cell line SW1463 was assessed using the CellTiterGlo following 7-day treatment with the YAP/TEAD inhibitor Compound B combined with the SHP2 inhibitor TNO-155. Proliferation of SW1463 cells was not inhibited by TNO-155 alone at the concentrations tested while Compound B showed some single agent activity. By comparison, the combination displayed synergistic growth inhibition compared to either treatment alone. Also tested was the combination of the YAP/TEAD inhibitor Compound B and the SHP2 inhibitor RMC-4550 (not shown in FIG. 34). This combination was also synergistic.

### Example 35

In vitro viability of the colorectal cancer cell line SW1463 was assessed using the CellTiterGlo following 7-day treatment with (i) the YAP/TEAD inhibitor Compound H combined with the KRAS G12C inhibitor JDQ443 and (ii) the YAP/TEAD inhibitor Compound H combined with the KRAS G12C inhibitor JDQ443 and the SHP2 inhibitor TNO155. Proliferation of SW1463 cells was inhibited by JDQ443 alone and JDQ443+TNO155, while Compound H showed some single agent activity. By comparison, combinations (i) and (ii) displayed synergistic growth inhibition compared to either treatment alone.

### Example 36

In vitro viability of the colorectal cancer cell line SW1463 was assessed using the CellTiterGlo following 7-day treatment with (i) the YAP/TEAD inhibitor Compound H combined with the KRAS G12C inhibitor Sotorasib and (ii) the YAP/TEAD inhibitor Compound H combined with the KRAS G12C inhibitor Sotorasib and the SHP2 inhibitor TNO155. Proliferation of SW1463 cells was inhibited by Sotorasib alone and Sotorasib+TNO155, and Compound H also showed some single agent activity. By comparison, combinations (i) and (ii) displayed synergistic growth inhibition compared to either treatment alone.

### Example 37

In vitro viability of the colorectal cancer cell line SW1463 was assessed using the CellTiterGlo following 7-day treatment with (i) the YAP/TEAD inhibitor Compound H combined with the KRAS G12C inhibitor Adagrasib, and (ii) the YAP/TEAD inhibitor Compound H combined with the KRAS G12C inhibitor Adagrasib and the SHP2 inhibitor RMC-4550. Proliferation of SW1463 cells was inhibited by Adagrasib alone and Adagrasib+RMC-4550, and Compound H also showed some single agent activity. By comparison, combinations (i) and (ii) displayed synergistic growth inhibition compared to either treatment alone.

### Example 38

In vitro viability of the lung cancer cell line NCI-H2122 was assessed using the CellTiterGlo following 7-day treatment with (i) the YAP/TEAD inhibitor Compound A combined with the KRAS G12C inhibitor Compound C and (ii) the YAP/TEAD inhibitor Compound A combined with the KRAS G12C inhibitor Compound C and the SHP2 inhibitor TNO155. Proliferation of NCI-H2122 cells was inhibited by Compound C alone and Compound C+TNO155, and Compound A also showed some single agent activity. By comparison, combinations (i) and (ii) displayed synergistic growth inhibtion compared to either treatment alone.

### Example 39

In vitro viability of the lung cancer cell line NCI-H2122 was assessed using the CellTiterGlo following 7-day treatment with (i) the YAP/TEAD inhibitor Compound B combined with the KRAS G12C inhibitor Compound C and (ii) the YAP/TEAD inhibitor Compound B combined with the KRAS G12C inhibitor Compound C and the SHP2 inhibitor TNO155. Proliferation of NCI-H2122 cells was inhibited by Compound C alone and Compound C+TNO155, and Compound B also showed some single agent activity. By comparison, combinations (i) and (ii) displayed synergistic growth inhibtion compared to either treatment alone.

### Example 40

In vitro viability of the lung cancer cell line NCI-H2122 was assessed using the CellTiterGlo following 7-day treatment with (i) the YAP/TEAD inhibitor Compound H combined with the KRAS G12C inhibitor Compound C and (ii) the YAP/TEAD inhibitor Compound H combined with the KRAS G12C inhibitor Compound C and the SHP2 inhibitor TNO155. Proliferation of NCI-H2122 cells was inhibited by Compound C alone and Compound C+TNO155, and Compound H also showed some single agent actiity. By comparison, combinations (i) and (ii) displayed snyergistic growth inhibition compared to either treatment alone.

### Example 41

In vitro viability of the lung cancer cell line NCI-H1373 was assessed using the CellTiterGlo following 7-day treatment with (i) the YAP/TEAD inhibitor Compound A combined with the KRAS G12C inhibitor JDQ443 and (ii) the YAP/TEAD inhibitor Compound A combined with the KRAS G12C inhibitor JDQ443 and the SHP2 inhibitor TNO155. Proliferation of NCI-H1373 cells was inhibited by JDQ443 alone and JDQ443+TNO155, and Compound A also showed single agent activity. By comparison, combinations (i) and (ii) displayed synergistic activity compared to either treatment alone.

### Example 42

In vitro viability of the lung cancer cell line NCI-H1373 was assessed using the CellTiterGlo following 7-day treatment with the YAP/TEAD inhibitor Compound A combined with the SHP2 inhibitor TNO155. Proliferation of NCI-H1373 cells was inhibited by Compound A and TNO155 also showed some single agent activity. By comparison the combination displayed synergistic activity compared to either treatment alone.

### Example 43

In vitro viability of the lung cancer cell line NCI-H1373 was assessed using the CellTiterGlo following 7-day treatment with (i) the YAP/TEAD inhibitor Compound B combined with the KRAS G12C inhibitor JDQ-443 and (ii) the YAP/TEAD inhibitor Compound B combined with the KRAS G12C inhibitor JDQ-443 and the SHP2 inhibitor TNO155. Proliferation of NCI-H1373 cells was inhibited by JDQ443 alone and JDQ443+TNO155, and Compound B also showed single agent activity. By comparison, combinations (i) and (ii) displayed synergistic activity compared to either treatment alone.

### Example 44

In vitro viability of the lung cancer cell line NCI-H1373 was assessed using the CellTiterGlo following 7-day treatment with the YAP/TEAD inhibitor Compound B combined with the SHP2 inhibitor TNO155. Proliferation of NCI-H1373 cells was inhibited by Compound B alone, and TNO155 also showed some single agent activity. By comparison the combination displayed synergistic activity compared to either treatment alone.

### Example 45

In vitro viability of the lung cancer cell line NCI-H1373 was assessed using the CellTiterGlo following 7-day treatment with (i) the YAP/TEAD inhibitor Compound H combined with the KRAS G12C inhibitor JDQ443 and (ii) the YAP/TEAD inhibitor Compound H combined with the KRAS G12C inhibitor JDQ443 and the SHP2 inhibitor TNO155. Proliferation of NCI-H1373 cells was inhibited by JDQ443 alone and JDQ443+TNO155, and Compound H also showed some single agent activity. By comparison, combinations (i) and (ii) displayed synergistic activity compared to either treatment alone.

### Example 46

In vitro viability of the lung cancer cell line NCI-H1373 was assessed using the CellTiterGlo following 7-day treatment with the YAP/TEAD inhibitor Compound H combined with the SHP2 inhibitor TNO155. Proliferation of NCI-H1373 cells was inhibited by Compound H alone, and TNO155 also showed some single agent activity. By comparison the combination displayed synergistic activity compared to either treatment alone.

### Example 47

In vitro viability of the lung cancer cell line HCC-1171 was assessed using the CellTiterGlo following 7-day treatment with (i) the YAP/TEAD inhibitor Compound A combined with the KRAS G12C inhibitor JDQ443 and (ii) the YAP/TEAD inhibitor Compound A combined with the KRAS G12C inhibitor JDQ443 and the SHP2 inhibitor TNO155. Proliferation of HCC-1171 cells was inhibited by JDQ443 alone and JDQ443+TNO155, and Compound A also showed single agent activity. By comparison, combinations (i) and (ii) displayed synergistic activity compared to either treatment alone.

### Example 48

In vitro viability of the lung cancer cell line HCC-1171 was assessed using the CellTiterGlo following 7-day treatment with the YAP/TEAD inhibitor Compound A combined with the SHP2 inhibitor TNO155. Proliferation of HCC-1171 cells was inhibtied by Compound A and TNO155 also showed single agent activity. By comparison the combination displayed synergistic activity compared to either treamtent alone.

### Example 49

In vitro viability of the lung cancer cell line HCC-1171 was assessed using the CellTiterGlo following 7-day treatment with (i) the YAP/TEAD inhibitor Compound B combined with the KRAS G12C inhibitor JDQ443 and (ii) the YAP/TEAD inhibitor Compound B combined with the KRAS G12C inhibitor JDQ443 and the SHP2 inhibitor TNO155. Proliferation of HCC-1771 cells was inhibited by JDQ443 alone and JDQ443+TNO155, and Compound B also showed single agent activity. By comparison, combinations (i) and (ii) displayed synergistic activity compared to either treatment alone.

### Example 50

In vitro viability of the lung cancer cell line HCC-1171 was assessed using the CellTiterGlo following 7-day treatment with the YAP/TEAD inhibitor Compound B combined with the SHP2 inhibitor TNO155. Proliferation of HCC-1171 cells was inhibited Compound B alone and TNO155 also showed single agent activity. By comparison the combinationdisplayed synergistic activity compared to either treatment alone.

### Example 51

In vitro viability of the lung cancer cell line HCC-1171 was assessed using the CellTiterGlo following 7-day treatment with (i) the YAP/TEAD inhibitor Compound H combined with the KRAS G12C inhibitor JDQ443 and (ii) the YAP/TEAD inhibitor Compound H combined with the KRAS G12C inhibitor JDQ443 and the SHP2 inhibitor TNO1 55. Proliferation of HCC-1171 was inhibited by JDQ443 alone and JDQ443+TNO155, and Compound H also showed single agent activity.

By comparison, combinations (i) and (ii) displayed synergistic activity compared to either treatment alone.

### Example 52

In vitro viability of the lung cancer cell line HCC-1171 was assessed using the CellTiterGlo following 7-day treatment with the YAP/TEAD inhibitor Compound H combined with the SHP2 inhibitor TNO155. Proliferation of HCC-1171 cells was inhibited by TNO155 alone, and Compound H also showed single agent activity. By comparison the combination displayed synergistic activity compared to either treatment alone.

### Example 53

In vitro viability of the lung cancer cell line NCI-H358 was assessed using the CellTiterGlo following 7-day treatment with (i) the YAP/TEAD inhibitor Compound A combined with the KRAS G12C inhibitor JDQ443 and (ii) the YAP/TEAD inhibitor Compound A combined with the KRAS G12C inhibitor JDQ443 and the SHP2 inhibitor TNO155. Proliferation of NCI-H358 cells was inhibited by JDQ443 alone and JDQ443+TNO155, and Compound A also showed single activity. By comparison, combinations (i) and (ii) displayed synergistic activity compared to either treatment alone.

### Example 54

In vitro viability of the lung cancer cell line NCI-H358 was assessed using the CellTiterGlo following 7-day treatment with the YAP/TEAD inhibitor Compound A combined with the SHP2 inhibitor TNO155. Proliferation of NCI-H358 cells was inhibited by TNO155 alone and Compound A also showed single agent activity. By comparison the combination displayed synergistic activity compared to either treatment alone.

### Example 55

In vitro viability of the lung cancer cell line NCI-H358 was assessed using the CellTiterGlo following 7-day treatment with (i) the YAP/TEAD inhibitor Compound B combined with the KRAS G12C inhibitor JDQ443 and (ii) the YAP/TEAD inhibitor Compound B combined with the KRAS G12C inhibitor JDQ443 and the SHP2 inhibitor TNO155. Proliferation of NCI-H358 cells was inhibited by JDQ443 alone and JDQ443+TNO155, and Compound B also showed single agent activity. By comparison, combinations (i) and (ii) displayed synergistic activity compared to either treatment alone.

### Example 56

In vitro viability of the lung cancer cell line NCI-H358 was assessed using the CellTiterGlo following 7-day treatment with the YAP/TEAD inhibitor Compound B combined with the SHP2 inhibitor TNO155. Proliferation of NCI-H358 cells was inhibited by Compound B alone and TNO155 also showed single agent activity. By comparison the combination displayed synergistic activity compared to either treatment alone.

### Example 57

In vitro viability of the lung cancer cell line NCI-H358 was assessed using the CellTiterGlo following 7-day treatment with (i) the YAP/TEAD inhibitor Compound H combined with the KRAS G12C inhibitor JDQ-443 and (ii) the YAP/TEAD inhibitor Compound H combined with the KRAS G12C inhibitor JDQ-443 and the SHP2 inhibitor TNO155. Proliferation of NCI-H358 cells was inhibited by JDQ443 alone and JDQ443+TNO155, and Compound H also showed single agent activity. By comparison, combinations (i) and (ii) displayed synergistic activity compared to either treatment alone.

### Example 58

In vitro viability of the lung cancer cell line NCI-H358 was assessed using the CellTiterGlo following 7-day treatment with the YAP/TEAD inhibitor Compound H combined with the SHP2 inhibitor TNO155. Proliferation of NCI-H358 cells was inhibited by TNO155 alone and Compound H also showed single agent activity. By comparison the combination displayed synergistic activity compared to either treatment alone.

### Example 59

In vitro viability of the lung cancer cell line LU65 was assessed using the CellTiterGlo following 7-day treatment with (i) the YAP/TEAD inhibitor Compound A combined with the KRAS G12C inhibitor sotorasib and (ii) the YAP/TEAD inhibitor Compound A combined with the KRAS G12C inhibitor sotorasib and the SHP2 inhibitor TNO155. Proliferation of LU65 cells was inhibited by sotorasib alone and sotorasib+TNO155, and Compound A also showed single agent activity. By comparison, cominations (i) and (ii) displayed synergistic activity compared to either treatment alone.

### Example 60

In vitro viability of the lung cancer cell line LU65 was assessed using the CellTiterGlo following 7-day treatment with (i) the YAP/TEAD inhibitor Compound A combined with the KRAS G12C inhibitor adagrasib and (ii) the YAP/TEAD inhibitor Compound A combined with the KRAS G12C inhibitor adagrasib and the SHP2 inhibitor TNO155. Proliferation of LU65 cells was inhibited by adagrasib alone and adagrasib+TNO155, and Compound A also showed single agent activity. By comparison, combinations (i) and (ii) displayed synergistic activity compared to either treatment alone.

### Example 61

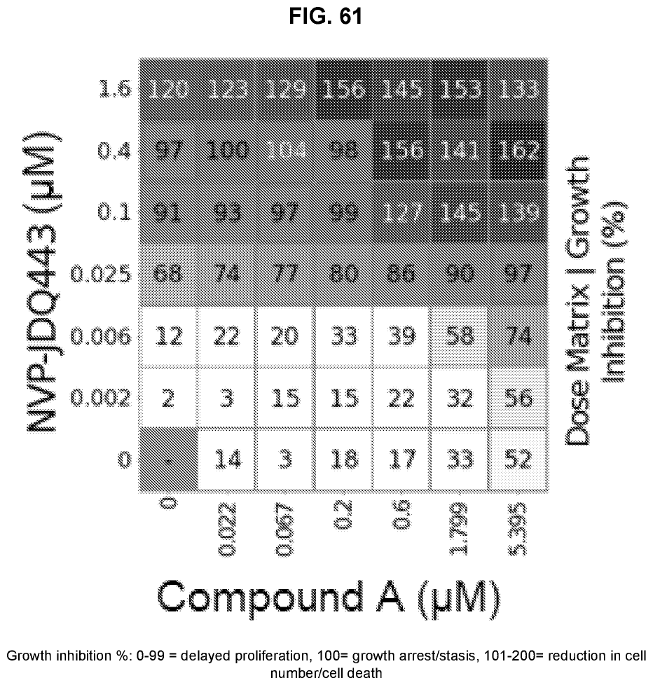

In vitro viability of the lung cancer cell line LU65 was assessed using the CellTiterGlo following 7-day treatment with the YAP/TEAD inhibitor Compound A combined with the KRAS G12C inhibitor JDQ443. Proliferation of LU65 cells was inhibited by JDQ443 and Compound A also showed single agent activity. By comparison the combination displayed synergistic activity compared to either treatment alone. Also tested was the combination of JDQ443+TNO155 and YAP/TEAD inhibitor Compound A (not shown in FIG. 61). This combination was also synergistic.

### Example 62

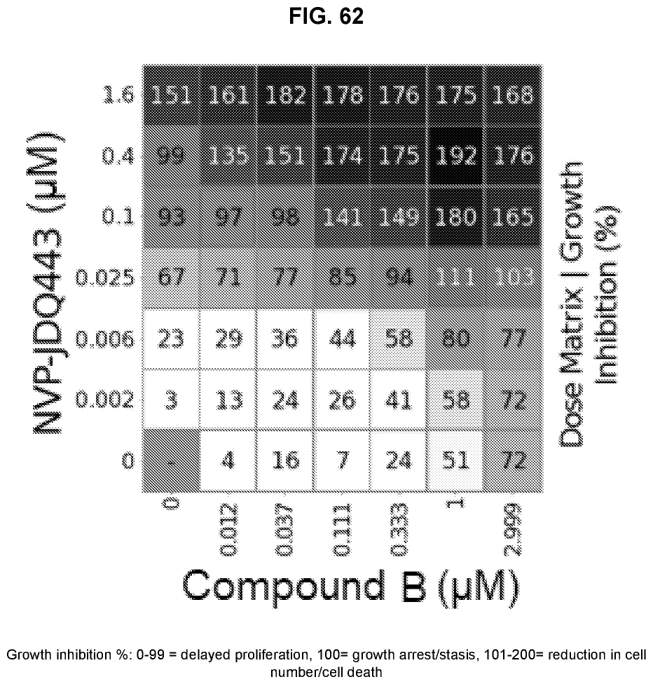

In vitro viability of the lung cancer cell line LU65 was assessed using the CellTiterGlo following 7-day treatment with the YAP/TEAD inhibitor Compound B combined with the KRAS G12C inhibitor JDQ443. Proliferation of LU65 cells was inhibited by JDQ443 and Compound B also showed single agent activity. By comparison the combination displayed synergistic activity compared to either treatment alone. Also tested was the combination of JDQ443+TNO155 and YAP/TEAD inhibitor Compound B (not shown in FIG. 62). This combination was also synergistic.

### Example 63

In vitro viability of the lung cancer cell line LU65 was assessed using the CellTiterGlo following 7-day treatment with (i) the YAP/TEAD inhibitor Compound H combined with the KRAS G12C inhibitor JDQ443 and (ii) the YAP/TEAD inhibitor Compound H combined with the KRAS G12C inhibitor JDQ443 and the SHP2 inhibitor TNO155. Proliferation of LU65 cells was inhibited by JDQ443 alone and JDQ443+TNO155, and Compound H also showed single agent activity. By comparison, combinations (i) and (ii) displayed synergistic activity compared to either treatment alone.

### Example 64

In vitro viability of the colorectal cancer cell line SW837 was assessed using the CellTiterGlo following 7-day treatment with (i) the YAP/TEAD inhibitor Compound A combined with the KRAS G12C inhibitor JDQ443 and (ii) the YAP/TEAD inhibitor Compound A combined with the KRAS G12C inhibitor JDQ443 and the SHP2 inhibitor TNO155. Proliferation of SW837 cell line was inhibited by JDQ443 alone and JDQ443+TNO155, and Compound A also showed single agent activity. By comparison, combinations (i) and (ii) displayed synergistic activity compared to either treatment alone.

### Example 65

In vitro viability of the colorectal cancer cell line SW837 was assessed using the CellTiterGlo following 7-day treatment with the YAP/TEAD inhibitor Compound A combined with the SHP2 inhibitor TNO155. Proliferation of SW837 cell line was inhibited by TNO155 and Compound A also showed single agent activity. By comparison the combination displayed synergistic activity compared to either treatment alone.

### Example 66

In vitro viability of the colorectal cancer cell line SW837 was assessed using the CellTiterGlo following 7-day treatment with (i) the YAP/TEAD inhibitor Compound B combined with the KRAS G12C inhibitor JDQ-443 and (ii) the YAP/TEAD inhibitor Compound B combined with the KRAS G12C inhibitor JDQ-443 and the SHP2 inhibitor TNO155. Proliferation of SW837 cells was inhibited by JDQ443 alone and JDQ443+TNO155, and Compound B also showed single agent activity. By comparison, combinations (i) and (ii) displayed synergistic activity compared to either treatment alone.

### Example 67

In vitro viability of the colorectal cancer cell line SW837 was assessed using the CellTiterGlo following 7-day treatment with the YAP/TEAD inhibitor Compound B combined with the SHP2 inhibitor TN0155. Proliferation of SW837 cells was inhibited TNO155 and Compound B also showed single agent activity. By comparison the combination displayed synergistic activity compared to either treatment alone.

### Example 68

In vitro viability of the colorectal cancer cell line SW837 was assessed using the CellTiterGlo following 7-day treatment with (i) the YAP/TEAD inhibitor Compound H combined with the KRAS G12C inhibitor JDQ-443 and (ii) the YAP/TEAD inhibitor Compound H combined with the KRAS G12C inhibitor JDQ-443 and the SHP2 inhibitor TNO155. Proliferation of SW837 cells was inhibited by JDQ443 alone and JDQ443+TNO155, and Compound H also showed single agent activity. By comparison, combinations (i) and (ii) displayed synergistic activity compared to either treatment alone.

### Example 69

In vitro viability of the colorectal cancer cell line SW837 was assessed using the CellTiterGlo following 7-day treatment with the YAP/TEAD inhibitor Compound H combined with the SHP2 inhibitor TNO155. Proliferation of SW837 cells was inhibited by TNO155 and Compound H also showed single agent activity. By comparison the combination displayed synergistic activity compared to either treatment alone.

### Example 70

In vitro viability of the colorectal cancer cell line LIM2099 was assessed using the CellTiterGlo following 7-day treatment with (i) the YAP/TEAD inhibitor Compound A combined with the KRAS G12C inhibitor JDQ-443 and (ii) the YAP/TEAD inhibitor Compound A combined with the KRAS G12C inhibitor JDQ-443 and the SHP2 inhibitor TNO155. Proliferation of LIM2099 cells was inhibited by JDQ443 alone and JDQ443+TNO155, and Compound A also showed single agent activity. By comparison, combinations (i) and (ii) displayed synergistic activity compared to either treatment alone.

### Example 71

In vitro viability of the colorectal cancer cell line LIM2099 was assessed using the CellTiterGlo following 7-day treatment with the YAP/TEAD inhibitor Compound A combined with the SHP2 inhibitor TNO155. Proliferation of LIM2099 cells was inhibited by TNO155 and Compound A also showed single agent activity. By comparison the combination displayed synergistic activity compared to either treatment alone.

### Example 72

In vitro viability of the colorectal cancer cell line LIM2099 was assessed using the CellTiterGlo following 7-day treatment with (i) the YAP/TEAD inhibitor Compound B combined with the KRAS G12C inhibitor JDQ-443 and (ii) the YAP/TEAD inhibitor Compound B combined with the KRAS G12C inhibitor JDQ-443 and the SHP2 inhibitor TNO155. Proliferation of LIM2099 cells was inhibited by JDQ443 alone and JDQ443+TNO155, and Compound B also showed single agent activity. By comparison, combinations (i) and (ii) displayed synergistic activity compared to either treatment alone.

### Example 73

In vitro viability of the colorectal cancer cell line LIM2099 was assessed using the CellTiterGlo following 7-day treatment with the YAP/TEAD inhibitor Compound B combined with the SHP2 inhibitor TNO155. Proliferation of SW837 cells was inhibited by TNO155 and Compound B also showed single agent activity. By comparison the combination displayed synergistic activity compared to either treatment alone.

### Example 74

In vitro viability of the colorectal cancer cell line LIM2099 was assessed using the CellTiterGlo following 7-day treatment with (i) the YAP/TEAD inhibitor Compound H combined with the KRAS G12C inhibitor JDQ-443 and (ii) the YAP/TEAD inhibitor Compound H combined with the KRAS G12C inhibitor JDQ-443 and the SHP2 inhibitor TNO155. Proliferation of LIM2099 cells was inhibited by JDQ443 alone and JDQ443+TNO155, and Compound H also showed single agent activity. By comparison, combinations (i) and (ii) displayed synergistic activity compared to either treatment alone.

### Example 75

In vitro viability of the colorectal cancer cell line LIM2099 was assessed using the CellTiterGlo following 7-day treatment with the YAP/TEAD inhibitor Compound H combined with the SHP2 inhibitor TNO155. Proliferation of LIM2099 cells was inhibited by TNO155 and Compound H also showed single agent activity. By comparison the combination displayed synergistic activity compared to treatment alone.

### Example 76

In vitro viability of the lung cancer cell line NCI-H1792 was assessed using Cyquant following 7-day treatment with (i) the YAP/TEAD inhibitor Compound A combined with the CDK4/6 inhibitor NVP-LEE011 (LEE011 or ribociclib), and (ii) the YAP/TEAD inhibitor Compound A combined with the CDK4/6 inhibitor NVP-LEE011 (LEE011 or ribociclib), and the KRAS G12C inhibitor JDQ443. Proliferation of NCI-H1792 cells was inhibited by LEE011 alone JDQ443+Compound A, and Compound A alone also showed some single agent activity. By comparison, combinations (i) and (ii) displayed synergistic activity compares to either treatment alone.

1. A method of treating cancer in a subject in need thereof, wherein the method comprises administering to the subject a therapeutically effective amount of a TEAD inhibitor in combination with a first additional therapeutically active agent, and optionally a second additional therapeutically active agent, wherein the first additional therapeutically active agent and the second additional therapeutically active agent (where present) are independently selected from the group consisting of a KRAS G12/G13 inhibitor, a SHP2 inhibitor, an EGFR inhibitor, a PI3K inhibitor, a MEK inhibitor, an ERK inhibitor, an MDM2 inhibitor, a Raf-inhibitor, a CDK4/6 inhibitor and a cMET inhibitor.

2. A TEAD inhibitor for use in the treatment of cancer, wherein the treatment further comprises administration of a first additional therapeutically active agent, and optionally a second therapeutically active agent, wherein the first additional therapeutically active agent and the second additional therapeutically active agent (where present) are independently selected from the group consisting of a KRAS G12/G13 inhibitor, a SHP2 inhibitor, an EGFR inhibitor, a PI3K inhibitor, a MEK inhibitor, an ERK inhibitor, an MDM2 inhibitor, a Raf-inhibitor, a CDK4/6 inhibitor and a cMET inhibitor.

3. A combination comprising i) a TEAD inhibitor, ii) a first additional therapeutically active agent, and optionally iii) a second additional therapeutically active agent, wherein the first additional therapeutically active agent and the second additional therapeutically active agent (where present) are independently selected from the group consisting of a KRAS G12/G13 inhibitor, a SHP2 inhibitor, an EGFR inhibitor, a PI3K inhibitor, a MEK inhibitor, an ERK inhibitor, an MDM2 inhibitor, a Raf-inhibitor, a CDK4/6 inhibitor and a cMET inhibitor.

4. The method according to claim 1, the TEAD inhibitor for use according to claim 2, or the combination according to claim 3, wherein the first additional therapeutically active agent is a KRAS G12/G13 inhibitor (e.g. a KRAS G12C inhibitor), e.g. where the second therapeutically active agent is absent.

5. The method according to claim 4, the TEAD inhibitor for use according to claim 4, or the combination according to claim 4, wherein a second additional therapeutically active agent is present, and is a SHP2 inhibitor.

6. The method according to claim 1, the TEAD inhibitor for use according to claim 2, or the combination according to claim 3, wherein the first additional therapeutically active agent is a SHP2 inhibitor, e.g. where the second therapeutically active agent is absent.

7. The method according to claim 1, the TEAD inhibitor for use according to claim 2, or the combination according to claim 3, wherein the first additional therapeutically active agent is an EGFR inhibitor, e.g. where the second therapeutically active agent is absent.

8. The method according to claim 1, the TEAD inhibitor for use according to claim 2, or the combination according to claim 3, wherein the first additional therapeutically active agent is a PI3K inhibitor, e.g. where the second therapeutically active agent is absent.

9. The method according to claim 1, the TEAD inhibitor for use according to claim 2, or the combination according to claim 3, wherein the first additional therapeutically active agent is an MDM2 inhibitor, e.g. where the second therapeutically active agent is absent.

10. The method according to claim 1, the TEAD inhibitor for use according to claim 2, or the combination according to claim 3, wherein the first additional therapeutically active agent is a CDK4/6 inhibitor, e.g. where the second therapeutically active agent is absent.

11. The method according to claim 1, the TEAD inhibitor for use according to claim 2, or the combination according to claim 3, wherein the first additional therapeutically active agent is a MEK inhibitor e.g. where the second therapeutically active agent is absent.

12. The method according to claim 1, the TEAD inhibitor for use according to claim 2, or the combination according to claim 3, wherein the first additional therapeutically active agent is an ERK inhibitor e.g. where the second therapeutically active agent is absent.

13. The method according to claim 11 or claim 12, the TEAD inhibitor for use according to claim 11 or claim 12, or the combination according to claim 11 or claim 12, wherein a second additional therapeutically active agent is present, and wherein the second additional therapeutically active agent is a Raf inhibitor.

14. The method according to claim 1, the TEAD inhibitor for use according to claim 2, or the combination according to claim 3, wherein the first additional therapeutically active agent is a cMET inhibitor, e.g. where the second therapeutically active agent is absent.

15. A cMET inhibitor for use in the treatment of cancer, wherein the treatment further comprises administration of a TEAD inhibitor.

16. A KRAS G12/G13 inhibitor (e.g. a KRAS G12C inhibitor) for use in the treatment of cancer, wherein the treatment further comprises administration of a TEAD inhibitor.

17. The KRAS G12/G13 inhibitor for use according to claim 16, wherein the treatment further comprises administration of a SHP2 inhibitor.

18. A SHP2 inhibitor for use in the treatment of cancer, wherein the treatment further comprises administration of a TEAD inhibitor.

19. The SHP2 inhibitor for use according to claim 18, wherein the treatment further comprises administration of a KRAS G12/G13 inhibitor (e.g. a KRAS G12C inhibitor).

20. A MEK inhibitor for use in the treatment of cancer, wherein the treatment further comprises administration of a TEAD inhibitor.

21. An ERK inhibitor for use in the treatment of cancer, wherein the treatment further comprises administration of a TEAD inhibitor.

22. The MEK inhibitor for use according to claim 20 or the ERK inhibitor for use according to claim 21, wherein the treatment further comprises administration of a Raf inhibitor.

23. A Raf inhibitor for use in the treatment of cancer, wherein the treatment further comprises administration of a TEAD inhibitor.

24. The Raf inhibitor for use according to claim 23, wherein the treatment further comprises administration of a MEK inhibitor.

25. The Raf inhibitor for use according to claim 23, wherein the treatment further comprises administration of an ERK inhibitor.

26. An EGFR inhibitor for use in the treatment of cancer, wherein the treatment further comprises administration of a TEAD inhibitor.

27. A PI3K inhibitor for use in the treatment of cancer, wherein the treatment further comprises administration of a TEAD inhibitor.

28. An MDM2 inhibitor for use in the treatment of cancer, wherein the treatment further comprises administration of a TEAD inhibitor.

29. A CDK4/6 inhibitor for use in the treatment of cancer, wherein the treatment further comprises administration of a TEAD inhibitor.

30. The method according to any one of claims 1 and 4 to 14, the TEAD inhibitor for use according to any one of claims 2 and 4 to 14, the combination according to any one of claims 3 to 14, the cMET inhibitor for use according to claim 15, the KRAS G12/G13 inhibitor for use according to claim 16 or claim 17, the SHP2 inhibitor for use according to claim 18 or claim 19, the MEK inhibitor for use according to claim 20 or claim 22, the ERK inhibitor for use according to claim 21 or claim 22, the Raf inhibitor for use according to any one of claims 23 to 25, the EGFR inhibitor for use according to claim 26, the PI3K inhibitor for use according to claim 27, the MDM2 inhibitor for use according to claim 28, or the CDK4/6 inhibitor for use according to claim 29, wherein the TEAD inhibitor is a YAP/TAZ-TEAD protein-protein interaction inhibitor.

31. The method according to claim 30, the TEAD inhibitor for use according to claim 30, the combination according to claim 30, the cMET inhibitor for use according to claim 30, the KRAS G12/G13 inhibitor for use according to claim 30, the SHP2 inhibitor for use according to claim 30, the MEK inhibitor for use according to claim 30, the ERK inhibitor for use according to claim 30, the Raf inhibitor for use according to claim 30, the EGFR inhibitor for use according to claim 30, the PI3K inhibitor for use according to claim 30, the MDM2 inhibitor for use according to claim 30, or the CDK4/6 inhibitor for use according to claim 30, wherein the TEAD inhibitor is a TEAD inhibitor of formula (I), or a pharmaceutically acceptable salt thereof, for example Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide) or a pharmaceutically acceptable salt thereof of or Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) or a pharmaceutically acceptable salt thereof.

32. The method according to any one of claims 1, 4, 5, 30 and 31, the TEAD inhibitor for use according to any one of claims 2, 4, 5, 30 and 31, the combination according to any one of claims 3 to 5, 30 and 31, the KRAS G12/G13 inhibitor for use according to any one of claims 16, 17, 30 and 31, or the SHP2 inhibitor for use according to any one of claims 19, 30 and 31, wherein the KRAS G12/G13 inhibitor is a KRAS G12C inhibitor selected from Compound C, sotorasib (Amgen), adagrasib (Mirati), D-1553 (InventisBio), B11701963 (Boehringer), GDC6036 (Roche), JNJ74699157 (J&J), X-Chem KRAS (X-Chem), LY3537982 (Lilly), B11823911 (Boehringer), AS KRAS G12C (Ascentage Pharma), SF KRAS G12C (Sanofi), RMC032 (Revolution Medicine), JAB-21822 (Jacobio Pharmaceuticals), AST-KRAS G12C (Allist Pharmaceuticals), AZ KRAS G12C (Astra Zeneca), NYU-12VC1 (New York University), and RMC6291 (Revolution Medicines), or a pharmaceutically acceptable salt thereof.

33. The method according to claim 32, the TEAD inhibitor for use according to claim 32, the combination according to claim 32, the KRAS G12/G13 inhibitor for use according to claim 32, or the SHP2 inhibitor for use according to claim 32, wherein the KRAS G12/G13 inhibitor is the KRAS G12C inhibitor Compound C (1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one) or AMG510, or a pharmaceutically acceptable salt thereof.

34. The method according to claim 33, the TEAD inhibitor for use according to claim 33, the combination according to claim 33, the KRAS G12/G13 inhibitor for use according to claim 33, or the SHP2 inhibitor for use according to claim 33, wherein the KRAS G12/G13 inhibitor is the KRAS G12C inhibitor Compound C, or a pharmaceutically acceptable salt thereof.

35. The method according to any one of claims 1, 5, 6, and 30 to 34, the TEAD inhibitor for use according to any one of claims 2, 5, 6, and 30 to 34, the combination according to any one of claims 3, 5, 6 and 30 to 34, the KRAS G12/G13 inhibitor for use according to any one of claims 17 and 30 to 34, or the SHP2 inhibitor for use according to any one of claims 18, 19 and 30 to 34, wherein the SHP2 inhibitor is selected from the group consisting of TNO155 (Novartis), JAB3068 (Jacobio), JAB3312 (Jacobio), RLY1971 (Roche), SAR442720 (Sanofi), RMC4550 (Revolution Medicines), RMC4630 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho & Otsuka Pharmas) and X-37-SHP2 (X-37), or a pharmaceutically acceptable salt thereof.

36. The method according to claim 35, the TEAD inhibitor for use according to claim 35, the combination for use according to claim 35, the KRAS G12/G13 inhibitor for use according to claim 35 or the SHP2 inhibitor for use according to claim 35 wherein the SHP2 inhibitor is TNO155, or a pharmaceutically acceptable salt thereof.

37. The method according to any one of claims 1, 14, 30 and 31, the TEAD inhibitor for use according to any one of claims 2, 4, 30 and 31, the combination according to any one of claims 3, 4, 30 and 31 or the cMET inhibitor for use according to claim 15, wherein the cMET inhibitor is selected from the group consisting of crizotinib, capmatinib, tepotinib, AMG337, cabozantinib, savolitinib (AZD6094, HMPL-504), tivantinib, foretinib, volitinib, SU11274, PHA 665752, SGX523, BAY-853474, KRC-408, T-1840383, MK-2461, BMS-777607, JNJ-38877605, tivantinib (ARQ 197), PF-04217903, MGCD265, BMS-754807, BMS-794833, AMG-458, NVP-BVU972, AMG-208, golvatinib, norcantharidin, S49076, SAR125844, merestinib (LY2801653), onartuzumab, emibetuzumab, SAIT301, ABT-700, DN30, LY3164530, rilotumumab, ficlatuzumab, TAK701, and YYB-101, or a pharmaceutically acceptable salt thereof.

38. The method according to claim 37, the TEAD inhibitor for use according to claim 37, the combination according to claim 37 or the cMET inhibitor for use according to claim 37, wherein the cMET inhibitor is i) tepotinib, or ii) capmatinib, or a pharmaceutically acceptable salt thereof.

39. The method according to any one of claims 1, 14, 30 and 31, the TEAD inhibitor for use according to any one of claims 2, 4, 30 and 31, the combination according to any one of claims 3, 4, 30 and 31 or the EGFR inhibitor for use according to claim 26, wherein the EGFR inhibitor is selected from the group consisting of cetuximab, panitumuab, erlotinib, gefitinib, osimertinib and nazartinib, or a pharmaceutically acceptable salt thereof.

40. The method according to claim 39, the TEAD inhibitor for use according to claim 39, the combination according to claim 39 or the EGFR inhibitor for use according to claim 39, wherein the EGFR inhibitor is nazartinib (also known as EGF816), or a pharmaceutically acceptable salt thereof.

41. The method according to any one of claims 1, 14, 30 and 31, the TEAD inhibitor for use according to any one of claims 2, 4, 30 and 31, the combination according to any one of claims 3, 4, 30 and 31 or the PI3K inhibitor for use according to claim 27, wherein the PI3K inhibitor is selected from the group consisting of AMG511, buparlisib, Idelalisib, Copanlisib, Duvelisib, Alpelisib, QAU421 and Umbralisib, or a pharmaceutically acceptable salt thereof.

42. The method according to any one of claims 1, 14, 30 and 31, the TEAD inhibitor for use according to any one of claims 2, 4, 30 and 31, the combination according to any one of claims 3, 4, 30 and 31 or the MDM2 inhibitor for use according to claim 28, wherein the MDM2 inhibitor is selected from the group consisting of nutlin-3a, idasanutlin (also known as RG7388), RG7112, AMG-232 (also known as KRT-232), APG-115, BI-907828, milademetan and HDM201 (also known as siremadlin), or a pharmaceutically acceptable salt thereof.

43. The method according to claim 42, the TEAD inhibitor for use according to claim 42, the combination according to claim 42, or the MDM2 inhibitor for use according to claim 42, wherein the MDM2 inhibitor is HDM201, or a pharmaceutically acceptable salt thereof.

44. The method according to any one of claims 1, 14, 30 and 31, the TEAD inhibitor for use according to any one of claims 2, 4, 30 and 31, the combination according to any one of claims 3, 4, 30 and 31 or the CDK4/6 inhibitor for use according to claim 29 wherein the CDK4/6 inhibitor is selected from the group consisting of ribociclib, palbociclib and abemaciclib, or a pharmaceutically acceptable salt thereof.

45. The method according to claim 44, the TEAD inhibitor for use according to claim 44, the combination according to claim 44, or the CDK4/6 inhibitor for use according to 44, wherein the CDK4/6 inhibitor is ribociclib, or a pharmaceutically acceptable salt thereof.

46. The method according to any one of claims 1, 14, 30 and 31, the TEAD inhibitor for use according to any one of claims 2, 4, 30 and 31, the combination according to any one of claims 3, 4, 30 and 31 or the MEK inhibitor for use according to claim 20 or claim 22 or the Raf inhibitor for use according to claim 24, wherein the MEK inhibitor is selected from the group consisting of pimasertib, PD-0325901, selumetinib, trametinib, binimetinib and cobimetinib, or a pharmaceutically acceptable salt thereof.

47. The method according to claim 46, the TEAD inhibitor for use according to claim 46, the combination according to claim 46, the MEK inhibitor for use according to 46, or the Raf inhibitor for use according to claim 46 wherein the MEK inhibitor is trametinib, or a pharmaceutically acceptable salt thereof.

48. The method according to any one of claims 1, 14, 30 and 31, the TEAD inhibitor for use according to any one of claims 2, 4, 30 and 31, the combination according to any one of claims 3, 4, 30 and 31 or the ERK inhibitor for use according to claim 21 or claim 22, or the Raf inhibitor for use according to claim 25 wherein the ERK inhibitor is selected from the group consisting of ulixertinib, GDC-0994, KO-947, Vtx-11e, SCH-772984, MK2853, LY3214996, MK08353, LTT462 and BVD-523, or a pharmaceutically acceptable salt thereof.

49. The method according to claim 48, the TEAD inhibitor for use according to claim 48, the combination according to claim 48, the ERK inhibitor for use according to 48, or the Raf inhibitor for use according to claim 48, wherein the ERK inhibitor is LTT462 (rineterkib) or ulixertinib, or a pharmaceutically acceptable salt thereof.

50. The method according to any one of claims 1, 14, 30, 31 and 46 to 49, the TEAD inhibitor for use according to any one of claims 2, 4, 30, 31 and 46 to 49, the combination according to any one of claims 3, 4, 30, 31 and 46 to 49, the MEK inhibitor for use according to claim 22, the ERK inhibitor for use according to claim 22 or the Raf inhibitor for use according to any one of claims 23 to 25, wherein the Raf inhibitor is selected from the group consisting of belvarafenib, naporafenib (also known as LXH254), Encorafenib, vemurafenib and dabrafenib, or a pharmaceutically acceptable salt thereof.

51. The method according to claim 50, the TEAD inhibitor for use according to claim 50, the combination according to claim 50, the MEK inhibitor for use according to claim 50, the ERK inhibitor for use according to claim 50 or the Raf inhibitor for use according to claim 50, wherein the Raf inhibitor is dabrafenib or LXH254 (naporafenib), or a pharmaceutically acceptable salt thereof.

52. The method according to any one of claims 1, 4 to 14 and 30 to 51, the TEAD inhibitor for use according to any one of claims 2, 4 to 14 and 30 to 51, the cMET inhibitor for use according to any one of claims 15, 30, 31, 37 and 38, the KRAS G12/G13 inhibitor for use according to any one of claims 16, 17 and 30 to 36, the SHP2 inhibitor for use according to any one of claims 18, 19 and 30 to 36, the MEK inhibitor for use according to any one of claims 20, 22, 30, 31, 46, 47, 50 and 51, the ERK inhibitor for use according to any one of claims 21, 22, 30, 31 and 48 to 51, the Raf inhibitor for use according to any one of claims 23 to 25, 30, 31, and 46 to 51, the EGFR inhibitor for use according to any one of claims 26, 30, 31, 39 and 40, the PI3K inhibitor for use according to any one of claims 27, 30, 31 and 41, the MDM2 inhibitor for use according to any one of claims 28, 30, 31, 42 and 43, or the CDK416 inhibitor for use according to any one of claims 29 to 31, 44 and 45, wherein the cancer is a TEAD dependent cancer.

53. The method according to any one of claims 1, 4 to 14 and 30 to 52, the TEAD inhibitor for use according to any one of claims 2, 4 to 14 and 30 to 52, the cMET inhibitor for use according to any one of claims 15, 30, 31, 37, 38 and 52, the KRAS G12/G13 inhibitor for use according to any one of claims 16, 17, 30 to 36 and 52 the SHP2 inhibitor for use according to any one of claims 18, 19, 30 to 36, and 52 the MEK inhibitor for use according to any one of claims 20, 22, 30, 31, 46, 47, and 50 to 52, the ERK inhibitor for use according to any one of claims 21, 22, 30, 31 and 48 to 52, the Raf inhibitor for use according to any one of claims 23 to 25, 30, 31, and 46 to 52, the EGFR inhibitor for use according to any one of claims 26, 30, 31, 39, 40 and 52, the PI3K inhibitor for use according to any one of claims 27, 30, 31, 41 and 52, the MDM2 inhibitor for use according to any one of claims 28, 30, 31, 42, 43 and 52, or the CDK4/6 inhibitor for use according to any one of claims 29 to 31, 44, 45 and 52, wherein the cancer is selected from the group consisting of breast cancer, lung cancer, ovarian cancer, kidney cancer, uterine cancer, colorectal cancer, malignant pleural mesothelioma, pancreatic cancer, prostate cancer, gastric cancer, gastrointestinal stromal tumor, esophageal cancer, liver cancer, medullobastoma, head and neck cancer, sarcoma, squamous cell carcinoma, epithelioid hemangioendothelioma, ependymal tumor and bone cancer.

54. The method according to any one of claims 1, 4 to 14 and 30 to 53, the TEAD inhibitor for use according to any one of claims 2, 4 to 14 and 30 to 53, the cMET inhibitor for use according to any one of claims 15, 30, 31, 37, 38, 52 and 53 the KRAS G12/G13 inhibitor for use according to any one of claims 16, 17, 30 to 36, 52 and 53, the SHP2 inhibitor for use according to any one of claims 18, 19, 30 to 36, 52 and 53, the MEK inhibitor for use according to any one of claims 20, 22, 30, 31, 46, 47 and 50 to 53, the ERK inhibitor for use according to any one of claims 21, 22, 30, 31 and 48 to 53, the Raf inhibitor for use according to any one of claims 23 to 25, 30, 31, and 46 to 53, the EGFR inhibitor for use according to any one of claims 26, 30, 31, 39, 40, 52 and 53, the PI3K inhibitor for use according to any one of claims 27, 30, 31, 41, 52 and 53, the MDM2 inhibitor for use according to any one of claims 28, 30, 31, 42, 43, 52 and 53, or the CDK4/6 inhibitor for use according to any one of claims 29 to 31, 44, 45, 52 and 53 wherein the TEAD inhibitor is administered on each of the first 3 days of a 7 day treatment cycle, and wherein the treatment is composed of at least two treatment cycles.

55. The method according to claim 54, the TEAD inhibitor according to claim 54, the cMET inhibitor according to claim 54, the KRAS G12/G13 inhibitor for use according to claim 54, the SHP2 inhibitor for use according to claim 54, the MEK inhibitor for use according to claim 54, the ERK inhibitor for use according to claim 54, the Raf inhibitor for use according to claim 54, the EGFR inhibitor for use according to claim 54, the PI3K inhibitor for use according to claim 54, the MDM2 inhibitor for use according to claim 54, or the CDK4/6 inhibitor for use according to claim 54, wherein the daily dose of the TEAD inhibitor on each administration day is from 15 mg to 100 mg.

56. The method according to claim 55, the TEAD inhibitor according to claim 55, the cMET inhibitor according to claim 55, the KRAS G12/G13 inhibitor for use according to claim 55, the SHP2 inhibitor for use according to claim 54, the MEK inhibitor for use according to claim 55, the ERK inhibitor for use according to claim 55, the Raf inhibitor for use according to claim 55, the EGFR inhibitor for use according to claim 55, the PI3K inhibitor for use according to claim 55, the MDM2 inhibitor for use according to claim 55, or the CDK4/6 inhibitor for use according to claim 55, wherein the daily dose of the TEAD inhibitor on each administration day is 15, 30, 45, 60, 75 mg, 90 mg or 100 mg.
